(19) United States
(12) Patent Application Publication
(10) Pub. No.: US 2009/0029992 A1
(43) Pub. Date: Jan. 29, 2009

(54) SUBSTITUTED PYRAZOLE COMPOUNDS

(76) Inventors: GREGORY E. AGOSTON, Rockville, MD (US); Anthony M. Treston, Rockville, MD (US); Gaetan Ladouceur, Rockville, MD (US); Lita Suwandi, Alexandria, VA (US)

Correspondence Address: KING & SPALDING LLP, 1180 PEACHTREE STREET, ATLANTA, GA, 30309-3521, US

(21) Appl. No.: 12/137,355

(22) Filed: Jun. 11, 2008

Related U.S. Application Data
(60) Provisional application No. 60/934,062, filed on Jun. 11, 2007.
(60) Provisional application No. 60/990,475, filed on Nov. 27, 2007.

(51) Int. Cl. A61K 31/5377; C07D 403/14; C07D 403/02; A61P 35/00; A61K 31/506; A61K 31/496; C07D 413/14

(52) U.S. Cl. 514/235.8; 544/295; 544/328; 544/122; 514/252.19; 514/252.14; 514/256

(57) ABSTRACT

Disclosed are protein kinase inhibitors, compositions comprising such inhibitors, and methods of use thereof. More particularly, disclosed are inhibitors of Aurora A (Aurora-2) protein kinase. Also disclosed are methods of treating diseases associated with protein kinases, especially diseases associated with Aurora-2, such as cancer.

### CROSS REFERENCE TO RELATED APPLICATIONS

This application claims priority to U.S. Provisional Patent Application No. 60/934,062, filed Jun. 11, 2007, and U.S. Provisional Patent Application No. 60/990,475, filed Nov. 27, 2007, both of which are hereby incorporated by reference in their entirety.

### FIELD OF THE INVENTION

This invention is directed to protein kinase inhibitors, compositions comprising such inhibitors, and methods of use thereof. More particularly, the invention relates to inhibitors of Aurora A (Aurora-2) protein kinase. The invention also relates to pharmaceutical compositions, as well as to methods of treating diseases associated with protein kinases, especially diseases associated with Aurora A, such as cancer.

### BACKGROUND OF THE INVENTION

The search for new therapeutic agents has been greatly aided in recent years by better understanding of the structure of enzymes and other biomolecules associated with target diseases. One important class of enzymes that has been the subject of extensive study is the protein kinases.

Protein kinases mediate intracellular signal transduction by effecting a phosphoryl transfer from a nucleoside triphosphate to a protein acceptor that is involved in a signaling pathway. There are a number of kinases and pathways through which extracellular and other stimuli cause a variety of cellular responses to occur inside the cell. Examples of such stimuli include environmental and chemical stress signals (e.g. osmotic shock, heat shock, ultraviolet radiation, bacterial endotoxin, H₂O₂), cytokines (e.g. interleukin-1 (IL-1) and tumor necrosis factor alpha (TNF-alpha)), and growth factors (e.g. granulocyte macrophage-colony-stimulating factor (GM-CSF), and fibroblast growth factor (FGF). An extracellular stimulus may effect one or more cellular responses related to cell growth, migration, differentiation, secretion of hormones, activation of transcription factors, muscle contraction, glucose metabolism, control of protein synthesis and regulation of cell cycle.

Many diseases are associated with abnormal cellular responses triggered by protein kinase-mediated events. These diseases include autoimmune diseases, inflammatory diseases, neurological and neurodegenerative diseases, cancer, cardiovascular diseases, allergies and asthma, Alzheimer's disease or hormone-related diseases. Accordingly, there has been a substantial effort in medicinal chemistry to find protein kinase inhibitors that are effective as therapeutic agents.

In humans, there are three highly related Aurora kinases that are all serine/threonine protein kinases (see Andrews, P. D., et al., Curr. Opin. Cell. Biol. 2003, 15, 672-683; Carmena, M., Earnshaw, W. C., Nat. Rev. Mol. Cell. Biol. 2003, 4, 842-854; Brown, J. R., et al., BMC Evol. Biol. 2004, 4, 39, Andrews, P. D., Oncogene 2005, 24, 5005-5015). Despite the sequence relatedness of Aurora A, B and C, the localization and function of these kinases is quite distinct. As a result, overexpression or activation of each of these kinases can be associated with different disease states, including proliferative diseases such as cancer.

Members of the family demonstrate distinct subcellular localization during mitosis and are degraded by the proteosome following exit from mitosis (Graham et al. (2002) J. Biol. Chem. 277:42419-22). The kinases are often found complexed with other proteins, including cytoskeletal structures.

The Aurora kinases share a conserved C-terminal catalytic domain, with greater variation being observed at the N-terminus. Aurora A (Aurora-2) is unique in the presence of two lysine residues in the nucleotide-binding domain of the kinase (Warner et al. (2003) Molecular Cancer Therapeutics 2:589-95).

Maximum levels of the Aurora A polypeptide, and maximum Aurora A activity, are observed at the G₂/M transition leading into mitotic prophase, with the polypeptide localizing to the mitotic spindle pole (Graham et al. (2002) J. Biol. Chem. 277:42419-22; Sakai et al. (2002) J. Biol. Chem. 277:48714-23). Aurora A appears to regulate chromosome duplication with aberrant expression being associated with aneuploidy and an aggressive clinical phenotype, particularly in solid tumors. Such observations, and additional experimental data, suggest that Aurora A disrupts the signaling cascade that regulates chromosome segregation (Sen et al. (2002) J. Nat. Cancer. Inst. 94:1320-29).

Aurora A also appears to function in meiosis, likely in separating homologous chromosomes and in spindle rotation. Injection of antibodies against Aurora A into Xenopus oocytes prevents first polar body extrusion and causes arrest at meiosis I (Castro et al. (2003) J. Biol. Chem. 2236-41). The Xenopus kinesin-like protein, Eg5, is known to be a substrate for Aurora-2 (Castro et al. (2003) J. Biol. Chem. 2236-41).

In addition, in vitro studies show that Aurora A is incorporated into chromatin and phosphorylates histone H3, and possibly histone H2B (Scrittori et al. (2001) J. Biol. Chem. 276:30002-10). H3 phosphorylation, e.g., at serine-10, during chromosome assembly, appears to be a conserved event in eukaryotic cell division. Inhibition of H3 phosphorylation leads to chromosome condensation, abnormal segregation, and the loss of chromosomes during mitosis and meiosis (Scrittori et al. (2001) J. Biol. Chem. 276:30002-10).

Accordingly, the emerging model for histone phosphorylation is analogous to that of histone acetylation, wherein partially redundant enzymatic activities are associated with histone modifications but different enzymes may function in different cellular contexts. For example, some enzymes may modify histones in bulk, while other enzymes modify histones in a targeted manner, i.e., in a sequence or domain-specific manner in the context of assembled chromatin (see, e.g., Scrittori et al. (2001) J. Biol. Chem. 276:30002-10). According to this model, Aurora A would appear to be a kinase responsible for targeted histone modification, in the context of assembled or assembling chromatin.

Other members of the Aurora kinase family are also associated with mitosis and meiosis. Aurora B, like Aurora A, is involved in distinct protein phosphorylation events that regulate the cell cycle. Unlike Aurora A, Aurora B is localized to inner-centromeric chromatin from prophase until the metaphase-anaphase transition, relocalizes to the microtubules in the spindle midzone during telophase, and subsequently is found in the midbody throughout cytokinesis (See Andrews, P. D., Oncogene 2005, 24, 5005-5015, loc. cit.). The function of Aurora B is to ensure accurate chromosome segregation and appropriate cytokinesis. Aurora B appears to associate with a survivin, a polypeptide that associates with the inner centromere and undergoes a significant degree of stretching during mitosis. Survivin appears to be involved with inhibition of apoptosis as well as cell cycle control. Interestingly, both Aurora B and survivin are delocalized during megakaryocyte endomitosis, a process by which late anaphase and cytokinesis are skipped, leading to megakaryocyte polyploidy (Zhang et al. (2004) Blood 103:3717-26). Inhibitors of this function in a proliferative disease such as cancer would lead to stasis and cell death, making such inhibitors useful in cancer chemotherapy.

Aurora C (Aurora-3) is the least studied, known member of the family. Aurora C localizes to centrosomes from anaphase until telophase (or even cytokinesis), and is highly expressed in the testis (Brown et al. (2004) BMC Evolutionary Biology 4:39).

As noted above, Aurora kinases are overexpressed in certain types of cancers, including colon, breast, and other solid-tumor cancers. The genes encoding the Aurora B and A kinases tend to be amplified in certain types of cancers, while the gene encoding the Aurora C kinase resides in a region of the chromosome that is subject to rearrangement and deletion. Aurora A has been associated with a variety of malignancies, including primary colon, colorectal, breast, stomach, ovarian, prostate, and cervical cancer, neuroblastoma, and other solid-tumor cancers (Warner et al. (2003) Molecular Cancer Therapeutics 2:589-95).

Inhibitors of Aurora A have been described. For example, Harrington et al. ((2004) Nat. Med. 10:262-67) have described VX-680, a small-molecule inhibitor that blocks cell-cycle progression and induces apoptosis in certain types of tumors in in vivo xenograft models. A pyrazole Aurora A kinase inhibitor is also described in U.S. Pat. No. 6,653,301 (Bebbington et al., issued Nov. 25, 2003).

Hauf et al. ((2003) J. Cell. Biol. 161:281-294) identified the indolinone (Hesperadin) as an inhibitor of Aurora B, which causes cells to enter anaphase with monooriented chromosomes, having both sister kinetochores attached to a single spindle pole (a condition known as syntelic attachment).

Ditchfield et al. ((2003) J. Cell. Biol. 161:267-280) described ZM447-439 ((4-(4-(N-benzoylamino)anilino)-6-methoxy-7-(3-(1-morpholino)propoxy)quina-zoline), an Aurora kinase inhibitor which interferes with chromosome alignment, segregation, and cytokinesis.

Accordingly, kinase inhibitors, particularly inhibitors of Aurora kinases, are of particular interest in treating certain disorders, including cancer. Compounds exhibiting such inhibition are of particular value.

### SUMMARY OF THE INVENTION

The present invention provides compounds or pharmaceutically acceptable derivatives or prodrugs thereof, compositions, and methods for treating diseases mediated by kinases. Such diseases include primary, secondary, and metastatic cancers such as melanoma, lymphoma, leukemia, colon, colorectal, breast, lung, kidney, pancreatic, renal, CNS, stomach, ovarian, prostate, cervical, and neuroblastoma.

In one embodiment, the invention provides a compound of the Formula I:

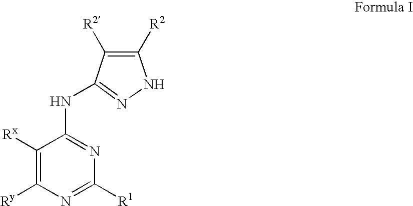

or a pharmaceutically acceptable derivative or prodrug thereof, wherein:

R^{x} is hydrogen, N(R⁴)₂, NO₂ or a C₁₋₁₂ aliphatic group;

R^{y} is hydrogen, N(R⁴)₂, OR, SR, S(O)R, S(O)₂R, N(R⁷)C(═O)R; an optionally substituted 3-10 membered monocyclic or bicyclic heterocyclyl or heteroaryl ring, wherein the 3-10 membered heterocyclyl or heteroaryl ring may have 1-4 ring heteroatoms selected from the group consisting of nitrogen, oxygen and sulfur; or alkyl or dialkyl amino wherein alkyl is optionally substituted with —OR, —SR, amino, alkylamino, dialkylamino or a C₃₋₈ heteroaryl or heterocyclyl ring having 1-4 heteroatoms selected from the group consisting of nitrogen, oxygen and sulfur;

R¹ is an optionally substituted 5-7 membered monocyclic or an 8-10 membered bicyclic aryl or heteroaryl ring, said heteroaryl ring having 1-4 ring heteroatoms selected from the group consisting of nitrogen, oxygen, and sulfur, wherein each substitutable ring carbon of R¹ is optionally independently substituted by oxo, R⁵, and each substitutable ring nitrogen of R¹ is optionally independently substituted by —R⁴;

R² and R^{2′} are independently selected from the group consisting of —R and N(R⁴)₂, OR, SR, S(O)R, S(O)₂R, or R² and R^{2′} taken together with their intervening atoms form a fused, 5-8 membered, unsaturated or partially unsaturated ring having 0-3 ring heteroatoms selected from the group consisting of nitrogen, oxygen, and sulfur, wherein each substitutable ring carbon of said fused ring formed by R² and R^{2′} is independently substituted by halo, oxo, —CN, —NO₂, or R⁷, and each substitutable ring nitrogen of said ring formed by R² and R^{2′} is independently substituted by —R⁴;

each R is independently hydrogen, R⁷ or an optionally substituted group selected from the group consisting of C₁₋₆ aliphatic, C₆₋₁₀ aryl, a heteroaryl ring having 5-10 ring atoms, and a heterocyclyl ring having 5-10 ring atoms;

each R⁴ is independently selected from the group consisting of —R⁷, —COR⁷, —CO₂ (optionally substituted C₁₋₆ aliphatic), —CON(R⁷)₂, and —SO₂R⁷;

each R⁵ is independently selected from the group consisting of —R, halo, —OR, —C(═O)R, —CO₂R, —COCOR, —NO₂, —CN, —S(O)R, —SO₂R, —SR, —N(R⁴)₂, —CON(R⁴)₂, —SO₂N(R⁴)₂, —OC(═O)R, —N(R⁴)COR, —N(R⁴)CO₂R, —N(R)SO₂N(R)₂, —N(R)CON(R)₂, OC(O)N(R)₂, —N(R⁴)N(R⁴)₂, —C═NN(R⁴)₂, —C═N—OR, —N(R⁴)CON(R⁴)₂, —N(R⁴)SO₂N(R⁴)₂, —N(R⁴)SO₂R, and —OC(═O)N(R⁴)₂; and

each R⁷ is independently selected from the group consisting of hydrogen, a C₁₋₆ aliphatic group which may optionally be substituted by OR, SR or N(R)₂; an optionally substituted 3-8 membered heterocyclyl or heteroaryl ring, wherein the 3-8 membered heterocyclyl or heteroaryl ring may have 1-4 ring heteroatoms selected from the group consisting of nitrogen, oxygen and sulfur; or two R⁷ on the same nitrogen are taken together with the nitrogen to form an optionally substituted 3-8 membered heterocyclyl or heteroaryl ring, wherein the 3-8 membered heterocyclyl or heteroaryl ring may have 1-4 ring heteroatoms selected from the group consisting of nitrogen, oxygen and sulfur;

with the proviso that when R^{x} is H and R^{y} is 4-methylpiperazinyl, and R² is methyl and R^{2′} is hydrogen, then R¹ is not unsubstituted indol-2-yl.

In another embodiment, a compound of Formula I is provided, wherein R^{x} is hydrogen.

In still another embodiment, a compound of Formula I is provided wherein R^{x} is N(R⁴)₂ or NO₂.

In another embodiment, a compound of Formula I is provided, wherein R^{x}=NR(CH₂)ₙN(R)₂, where n is more than 1.

In yet another embodiment, a compound of Formula I is provided, wherein:

R^{y} is hydrogen, N(R⁴)₂, OR, SR, an optionally substituted 4-8 membered heterocyclyl or heteroaryl ring, or alkyl or dialkyl amino wherein alkyl is optionally substituted with —OR, —SR, amino, alkylamino, dialkylamino or a C₃₋₈ heteroaryl or heterocyclyl ring having 1-4 heteroatoms selected from the group consisting of nitrogen, oxygen and sulfur.

In another embodiment of the invention, a compound of Formula I is provided, wherein R² and R^{2′} are independently hydrogen, alkyl or amino.

In another embodiment, the invention provides a compound of Formula I, wherein:

R² and R^{2′} are independently hydrogen or alkyl;

R^{x} is hydrogen or N(R⁴)₂; and

R^{y} is hydrogen, N(R⁴)₂, alkyl or dialkyl amino wherein alkyl is optionally substituted with —OR, —SR, amino, alkylamino, dialkylamino or a C₃₋₈ heteroaryl or heterocyclyl ring having 1-4 heteroatoms selected from the group consisting of nitrogen, oxygen and sulfur; or an optionally substituted 4-8 membered heterocyclyl or heteroaryl ring.

In another embodiment, a compound of Formula I is provided, wherein R¹ is an optionally substituted 8-10 membered bicyclic heteroaryl ring or R¹ is an optionally substituted monocyclic or bicyclic aryl ring.

In various embodiments of the invention, R¹ in the compound of Formula I is one of the following groups:

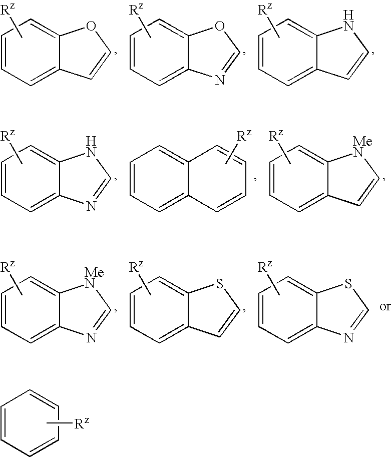

wherein R^{z} is selected from the group consisting of H, alkyl, alkoxy, halogen, CF₃, amino, alkylamino, dialkylamino, cyano and nitro.

In another embodiment of the invention, a compound of Formula I is provided, wherein R^{y} is 4-aminotetrahydropyran, 2-methoxyethyl amine, 2-dimethylaminoethyl amine, 2-morpholinoethylamine, 2-(4-methylpiperazin-1-yl)ethylamine, 4-aminotetrahydropyran, 1-pyrrolidinyl, 1-piperidinyl, 1-piperazinyl, 4-methyl-1-piperazinyl, 4-acyl-1-piperazinyl or 4-morpholinyl.

In yet another embodiment, a compound of Formula I is provided, wherein R^{y} is optionally substituted alkylamino or dialkylamino.

In another embodiment, a compound of Formula I is provided, wherein R¹ is optionally substituted a 5-7 membered monocyclic heteroaryl ring.

In still another embodiment, a compound of Formula I is provided, wherein:

R¹ is optionally substituted phenyl, N-methylindolyl, indolyl or benzofuranyl;

R^{x} is hydrogen;

R^{y} is 4-aminotetrahydropyran, N-methyl-N-(2-methoxy)ethyl-amine, N-methyl-N-(2-(dimethylamino)ethyl amine, 1-piperidinyl, 1-piperazinyl or 4-morpholinyl;

R² is alkyl; and

R^{2′} is hydrogen.

In particular embodiments, the invention provides compounds of Formula I that have the structures presented in Table 1 below, or biologically acceptable salts or prodrug thereof.

In another embodiment of the invention, a pharmaceutical composition comprising an Aurora kinase A inhibition effective amount of the compound of Formula I in combination with a pharmaceutically acceptable carrier, adjuvant or vehicle is provided. The composition may comprise particles that are less than about 2 microns average particle size. In some embodiments, the composition may be incorporated into a biodegradable or non-biodegradable polymer.

In one embodiment, the compositions comprise a compound of Formula I and an additive. The additive may be an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof. The composition may include a carrier that is suitable for oral, parenteral, inhalation, topical, or intradermal administration.

In another embodiment of the invention, a method of treating a patient with a disease comprising administering to the patient with the disease an effective amount of a compound of Formula I is provided, wherein the disease is an autoimmune disease, inflammatory disease, neurological or neurodegenerative disease, cancer, cardiovascular disease, allergy, asthma, or a hormone-related disease.

In one embodiment, a method of treating a patient with a cancer comprising administering to the patient having the cancer an effective cancer-treating amount of a compound selected from the group of compounds of Formula I is provided. The cancer may be a solid tumor, blood borne tumor, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity, pharynx, lip, tongue, mouth, pharynx, small intestine, colon-rectum, large intestine, rectum, brain and central nervous system, or leukemia.

In another embodiment, the invention provides a method of treating a patient with a disease associated with undesirable neovascularization comprising administering to the patient with the undersirable neovascularization an effective amount of a composition comprising a compound of Formula I.

The disease associated with undesirable neovasculariation may comprise ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, or corneal graph rejection, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, or post-laser complications.

In still another embodiment, the invention provides a method of treating a patient with an inflammatory disease associated with inflammation comprising administering to the patient with the inflammatory disease an effective amount of a compound of Formula I.

The inflammatory disease may be excessive or abnormal stimulation of endothelial cells, atherosclerosis, vascular malfunctions, abnormal wound healing, inflammatory and immune disorders, Bechet's disease, gout or gouty arthritis, abnormal angiogenesis accompanying rheumatoid arthritis, skin diseases, psoriasis, diabetic retinopathy, retinopathy of prematurity, retrolental fibroplasia, macular degeneration, corneal graft rejection, neovascular glaucoma or Osler Weber syndrome.

In still another embodiment, the invention provides a method of treating patient with a GSK-3 mediated disease comprising administering to the patient with the GSK-3 mediated disease an effective amount of a compound of Formula I.

In various embodiments, the GSK-3 mediated disease is diabetes, Alzheimer's disease, Huntington's Disease, Parkinson's Disease, AIDS-associated dementia, amyotrophic lateral sclerosis (AML), multiple sclerosis (MS), schizophrenia, cardiomycete hypertrophy, reperfusion/ischemia, or baldness.

In some embodiments, the compound may be administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

It has been found that compounds of general formula I wherein R¹ is an aryl ring, a 5-7 membered monocyclic heteroaryl group or an 8-10 membered bicyclic heteroaryl group that is directly linked to the pyrimidine ring are particulary efficient inhibitors of Aurora kinase A.

### DETAILED DESCRIPTION

As used herein, the following definitions shall apply unless otherwise indicated. The phrase “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

The term “acetamido” refers to the group —NHC(═O)CH₃.

The term “aliphatic” as used herein means straight-chain, branched or cyclic C₁-C₁₂ hydrocarbons which are completely saturated or which contain one or more units of unsaturation but which are not aromatic. For example, suitable aliphatic groups include substituted or unsubstituted linear, branched or cyclic alkyl, alkenyl, alkynyl groups and hybrids thereof such as (cycloalkyl)alkyl, (cycloalkenyl)alkyl or (cycloalkyl)alkenyl. The terms “alkyl”, “alkoxy”, “hydroxyalkyl”, “alkoxyalkyl”, and “alkoxycarbonyl”, used alone or as part of a larger moiety includes both straight and branched chains containing one to twelve carbon atoms. The terms “alkenyl” and “alkynyl” used alone or as part of a larger moiety shall include both straight and branched chains containing two to twelve carbon atoms. The term “cycloalkyl” used alone or as part of a larger moiety shall include cyclic C₃-C₁₂ hydrocarbons which are completely saturated or which contain one or more units of unsaturation, but which are not aromatic.

The term “amino” refers to an NH₂ group.

The term “alkylamino” refers to an amino group wherein one of the hydrogen atoms is replaced by an alkyl group.

The term “dialkylamino” refers to an amino group wherein the hydrogen atoms are replaced by alkyl groups, wherein the alkyl group may be the same or different.

The terms “haloalkyl”, “haloalkenyl” and “haloalkoxy” means alkyl, alkenyl or alkoxy, as the case may be, substituted with one or more halogen atoms.

The term “halogen” means F, Cl, Br, or I.

The term “heteroatom” means nitrogen, oxygen, or sulfur and includes any oxidized form of nitrogen and sulfur, and the quaternized form of any basic nitrogen. Also the term “nitrogen” includes a substitutable nitrogen of a heterocyclic ring. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl) or NR⁺ (as in N-substituted pyrrolidinyl).

The terms “carbocycle”, “carbocyclyl”, “carbocyclo”, or “carbocyclic” as used herein means an aliphatic ring system having three to fourteen members. The terms “carbocycle”, “carbocyclyl”, “carbocyclo”, or “carbocyclic” whether saturated or partially unsaturated, also refers to rings that are optionally substituted. The terms “carbocycle”, “carbocyclyl”, “carbocyclo”, or “carbocyclic” also include aliphatic rings that are fused to one or more aromatic or nonaromatic rings, such as in a decahydronaphthyl or tetrahydronaphthyl, where the radical or point of attachment is on the aliphatic ring.

The term “aryl” used alone or as part of a larger moiety as in “aralkyl”, “aralkoxy”, or “aryloxyalkyl”, refers to aromatic ring groups having six to fourteen members, such as phenyl, benzyl, phenethyl, 1-naphthyl, 2-naphthyl, 1-anthracyl and 2-anthracyl. The term “aryl” also refers to rings that are optionally substituted. The term “aryl” may be used interchangeably with the term “aryl ring”. “Aryl” also includes fused polycyclic aromatic ring systems in which an aromatic ring is fused to one or more rings. Examples include 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in an indanyl, phenanthridinyl, or tetrahydronaphthyl, where the radical or point of attachment is on the aromatic ring.

The term “heterocycle”, “heterocyclyl”, or “heterocyclic” as used herein includes non-aromatic ring systems having three to fourteen members, preferably five to ten, in which one or more ring carbons, preferably one to four, are each replaced by a heteroatom. Examples of heterocyclic rings include 3-1H-benzimidazol-2-one, (1-substituted)-2-oxo-benzimidazol-3-yl, 2-tetrahydro-furanyl, 3-tetrahydrofuranyl, 2-tetrahydropyranyl, 3-tetrahydropyranyl, 4-tetra-hydropyranyl, [1,3]-dioxalanyl, [1,3]-dithiolanyl, [1,3]-dioxanyl, 2-tetra-hydro-thiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

The term “heteroaryl”, used alone or as part of a larger moiety as in “heteroaralkyl” or “heteroarylalkoxy”, refers to heteroaromatic ring groups having five to fourteen members. Examples of heteroaryl rings include 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, and benzoisoxazolyl. Also included within the scope of the term “heteroaryl”, as it is used herein, is a group in which a heteroatomic ring is fused to one or more aromatic or nonaromatic rings where the radical or point of attachment is on the heteroaromatic ring. Examples include tetrahydroquinolinyl, tetrahydroisoquino-linyl, and pyrido[3,4-d]pyrimidinyl. The term “heteroaryl” also refers to rings that are optionally substituted. The term “heteroaryl” may be used interchangeably with the term “heteroaryl ring” or the term “heteroaromatic”.

An aryl (including aralkyl, aralkoxy, aryloxyalkyl and the like) or heteroaryl (including heteroaralkyl and heteroarylalkoxy and the like) group may contain one or more substituents. Examples of suitable substituents on any unsaturated carbon atom of an aryl, heteroaryl, aralkyl, or heteroaralkyl group include a halogen, CF₃, —R⁰, —OR⁰, —SR⁰, 1,2-methylene-dioxy, 1,2-ethylenedioxy, protected OH (such as acyloxy), phenyl (Ph), substituted Ph, —O(Ph), substituted —O(Ph), —CH₂(Ph), substituted —CH₂(Ph), —CH₂CH₂(Ph), substituted —CH₂CH₂(Ph), —NO₂, —CN, —N(R⁰)₂, —NR⁰C(O)R⁰, —NR⁰C(O)N(R⁰)₂, —NR⁰CO₂R⁰, —NR⁰NR⁰C(O)R⁰, —NR⁰NR⁰C(O)N(R⁰)₂, —NR⁰NR⁰C₂R⁰, —C(O)C(O)R⁰, —C(O)CH₂C(O)R⁰, —CO₂R⁰, —C(O)R⁰, —C(O)N(R⁰)₂, —OC(O)N(R⁰)₂, —S(O)₂R⁰, —SO₂N(R⁰)₂, —S(O)R⁰, —NR⁰SO₂N(R⁰)₂, —NR⁰SO₂R⁰, —C(═S)N(R⁰)₂, —C(═NH)—N(R⁰)₂, —(CH₂)_{y}NHC(O)R⁰, and —(CH₂)_{y}NHC(O)CH(V—R⁰)(R⁰); wherein each R⁰ is independently selected from hydrogen, a substituted or unsubstituted aliphatic group, an unsubstituted heteroaryl or heterocyclic ring, phenyl (Ph), substituted Ph, —O(Ph), substituted —O(Ph), —CH₂ (Ph), or substituted —CH₂(Ph); y is 0-6; and V is a linker group. Examples of substituents on the aliphatic group or the phenyl ring of R⁰ include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

An aliphatic group or a non-aromatic heterocyclic ring or a fused aryl or heteroaryl ring may contain one or more substituents. Examples of suitable substituents on any saturated carbon of an aliphatic group or of a non-aromatic heterocyclic ring or a fused aryl or heteroaryl ring include those listed above for the unsaturated carbon of an aryl or heteroaryl group and the following: ═O, ═S, ═NNHR*, ═NN(R*)₂, ═N—, ═NNHC(O)R*, ═NNHCO₂ (alkyl), ═NNHSO₂ (alkyl), or ═NR*, where each R* is independently selected from hydrogen, an unsubstituted aliphatic group, or a substituted aliphatic group. Examples of substituents on the aliphatic group include amino, alkylamino, dialkylamino, aminocarbonyl, halogen, alkyl, CF₃, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

Suitable substituents on the nitrogen of a non-aromatic heterocyclic ring include —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, and —NR⁺SO₂R⁺; wherein each R⁺ is independently selected from hydrogen, an aliphatic group, a substituted aliphatic group, phenyl (Ph), substituted Ph, —O(Ph), substituted —O(Ph), —CH₂(Ph), substituted —CH₂(Ph), —CH(Ph)₃ or an unsubstituted heteroaryl or heterocyclic ring. Examples of substituents on the aliphatic group or the phenyl ring include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

The term “linker group” or “linker” means an organic moiety that connects two parts of a compound. Linkers are typically comprised of an atom such as oxygen or sulfur, a unit such as —NH—, —CH₂—, —C(O)—, —C(O)NH—, or a chain of atoms, such as an alkylidene chain. The molecular mass of a linker is typically in the range of about 14 to 200, preferably in the range of 14 to 96 with a length of up to about six atoms. Examples of linkers include a saturated or unsaturated C₁₋₆ alkylidene chain which is optionally substituted, and wherein one or two saturated carbons of the chain are optionally replaced by —C(O)—, —C(O)C(O)—, —CONH—, —CONHNH—, —CO₂—, —OC(O)—, —NHCO₂—, —O—, —NHCONH—, —OC(O)NH—, —NHNH—, —NHCO—, —S—, —SO—, —SO₂—, —NH—, —SO₂NH—, or —NHSO₂—.

The term “alkylidene chain” refers to an optionally substituted, straight or branched carbon chain, that may be fully saturated or have one or more units of unsaturation. The optional substituents are as described above for an aliphatic group.

A combination of substituents or variables is permissible only if such a combination results in a stable or chemically feasible compound. A stable compound or chemically feasible compound is one in which the chemical structure is not substantially altered when kept in the dark at a temperature of 40° C. or less, in the absence of moisture or other chemically reactive conditions, for at least a week.

It is understood that in all substituted groups defined herein, polymers arrived at by defining substituents with further substituents to themselves (e.g., substituted phenyl having a substituted phenyl as a substituent which is itself substituted with a substituted phenyl, etc.) are not intended for inclusion herein. In such cases, the maximum number of such substituents is three. For example phenyl substituted with a substituted phenyl is limited to—substituted phenyl-(substituted phenyl)-(substituted phenyl).

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C— or ¹⁴C-enriched carbon are within the scope of this invention.

Whenever a range is referred to herein, it includes independently and separately every member of the range. As a non-limiting example, the term “C₁-C₁₀ alkyl” is considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities. Similarly, as another non-limiting example, 1-10% includes independently, 1%, 2%, 3%, 4%, 5%, 6%, 7%, 8%, 9% and 10%, as well as ranges in between such as 1-2%, 2-3%, etc.

Compounds of formula I, or salts thereof, may be formulated into compositions. In one embodiment, the composition is a pharmaceutical composition. In one embodiment, the composition comprises an amount of the protein kinase inhibitor effective to inhibit a protein kinase in a biological sample or in a patient. Compounds of this invention and pharmaceutical compositions thereof, which comprise an amount of the protein kinase inhibitor effective to treat or prevent a kinase mediated condition and a pharmaceutically acceptable carrier, adjuvant, or vehicle, may be formulated for administration to a patient.

Another aspect of this invention relates to a method of treating or preventing a kinase mediated disease. In one embodiment, the disease is a Aurora A-mediated disease, which method comprises administering to a patient in need of such a treatment a therapeutically effective amount of a compound of formula I or a pharmaceutical composition thereof.

The term “Aurora A-mediated disease” or “Aurora A-mediated condition”, as used herein, means any disease or other deleterious condition in which Aurora is thought to play a role. The terms “Aurora A-mediated disease” or “Aurora A-mediated condition” also mean those diseases or conditions that are alleviated by treatment with an Aurora A inhibitor. Such conditions include cancer.

The term “cancer” includes, but is not limited to, solid tumors and blood borne tumors and include, but is not limited to, the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia. The term “cancer” includes primary cancer, cancers secondary to treatment, and metastatic cancers.

An aspect of the invention relates to compounds and compositions that are useful for treating cancer.

Another aspect of the invention relates to the treatment of the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia.

Another aspect of the invention is a method for treating cancer comprising administering one or more of the compounds described herein to a patient with cancer.

Angiogenesis is characterized by the proliferation of endothelial cells to form new blood vessels (often called neovascularization). Inhibition of mitosis of endothelial cells results in inhibition of angiogenesis. Another aspect of this invention therefore relates to inhibition of undesirable mitosis, including undesirable angiogenesis. A mammalian disease characterized by undesirable cell mitosis, as defined herein, includes, but is not limited to, excessive or abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying rheumatoid arthritis, skin diseases, such as psoriasis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease).

Other undesired angiogenesis involves normal processes including ovulation and implantation of a blastula. The compositions described above can be used as a birth control agent by reducing or preventing uterine vascularization required for embryo implantation. Accordingly, the compositions described above can be used to block ovulation and implantation of a blastula or to block menstruation (induce amenorrhea).

Diseases associated with undesirable mitosis including neovascularization can be treated according to the present invention. Such diseases include, but are not limited to, ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, and corneal graph rejection.

Other diseases associated with undesirable mitosis including neovascularization can be treated according to the present invention. Such diseases include, but are not limited to, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, and post-laser complications. Other diseases include, but are not limited to, diseases associated with rubeosis (neovascularization of the iris and the angle) and diseases caused by the abnormal proliferation of fibrovascular or fibrous tissue including all forms of proliferative vitreoretinopathy, whether or not associated with diabetes.

Another aspect of the invention relates to the treatment of inflammatory diseases including, but not limited to, excessive or abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying rheumatoid arthritis, skin diseases, such as psoriasis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease). Other undesired angiogenesis involves normal processes including ovulation and implantation of a blastula. Accordingly, the compositions described above can be used to block ovulation and implantation of a blastula or to block menstruation (induce amenorrhea).

Another aspect of the invention relates to inhibiting Aurora A activity in a biological sample, which method comprises contacting the biological sample with the Aurora A inhibitor of formula I, or a composition thereof.

Another aspect of this invention relates to a method of inhibiting Aurora A activity in a patient, which method comprises administering to the patient a compound of formula I or a composition comprising said compound.

In another aspect of this invention, compounds of formula I are more potent inhibitors of Aurora A compared to Aurora B.

Another aspect of this invention relates to a method of treating or preventing a GSK-3-mediated disease with a GSK-3 inhibitor, which method comprises administering to a patient in need of such a treatment a therapeutically effective amount of a compound of formula I or a pharmaceutical composition thereof.

The terms “GSK-3-mediated disease, or “GSK-3-mediated condition”, as used herein, mean any disease or other deleterious condition or state in which GSK-3 is known to play a role. Such diseases or conditions include, without limitation, diabetes, Alzheimer's disease, Huntington's Disease, Parkinson's Disease, AIDS-associated dementia, amyotrophic lateral sclerosis (AML), multiple sclerosis (MS), schizophrenia, cardiomycete hypertrophy, reperfusion/ischemia, and baldness.

One aspect of this invention relates to a method of enhancing glycogen synthesis and/or lowering blood levels of glucose in a patient in need thereof, which method comprises administering to the patient a therapeutically effective amount of a compound of formula I or a pharmaceutical composition thereof. This method is especially useful for diabetic patients. Another method relates to inhibiting the production of hyperphosphorylated Tau protein, which is useful in halting or slowing the progression of Alzheimer's disease. Another method relates to inhibiting the phosphorylation of .beta.-catenin, which is useful for treating schizophrenia.

Another aspect of the invention relates to inhibiting GSK-3 activity in a biological sample, which method comprises contacting the biological sample with a GSK-3 inhibitor of formula I.

Another aspect of this invention relates to a method of inhibiting GSK-3 activity in a patient, which method comprises administering to the patient a compound of formula I or a composition comprising said compound.

Another aspect of this invention relates to a method of treating or preventing a CDK-2-mediated disease with a CDK-2 inhibitor, which method comprises administering to a patient in need of such a treatment a therapeutically effective amount of a compound of formula I or a pharmaceutical composition thereof.

The terms “CDK-2-mediated disease” or CDK-2-mediated condition”, as used herein, mean any disease or other deleterious condition in which CDK-2 is known to play a role. The terms “CDK-2-mediated disease” or “CDK-2-mediated condition” also mean those diseases or conditions that are alleviated by treatment with a CDK-2 inhibitor. Such conditions include, without limitation, cancer, Alzheimer's disease, restenosis, angiogenesis, glomerulonephritis, cytomegalovirus, HIV, herpes, psoriasis, atherosclerosis, alopecia, and autoimmune diseases such as rheumatoid arthritis, such as are described for example in Fischer, P. M. and Lane, D. P., Current Medicinal Chemistry, 7, 1213-1245 (2000); Mani, S., Wang, C., Wu, K., Francis, R. and Pestell, R., Exp. Opin. Invest. Drugs, 9, 1849 (2000); Fry, D. W. and Garrett, M. D., Current Opinion in Oncologic, Endocrine & Metabolic Investigational Drugs, 2, 40-59 (2000).

Another aspect of the invention relates to inhibiting CDK-2 activity in a biological sample or a patient, which method comprises administering to the patient a compound of formula I, or a composition comprising said compound.

Another aspect of this invention relates to a method of treating or preventing an ERK-2-mediated diseases with an ERK-2 inhibitor, which method comprises administering to a patient in need of such a treatment a therapeutically effective amount of a compound of formula I or a pharmaceutical composition thereof.

The terms “ERK-mediated disease” or “ERK-mediated condition”, as used herein mean any disease or other deleterious condition in which ERK may play a role. The terms “ERK-2-mediated disease” or “ERK-2-mediated condition” also mean those diseases or conditions that are alleviated by treatment with a ERK-2 inhibitor. Such conditions include, without limitation, cancer, stroke, diabetes, hepatomegaly, cardiovascular disease including cardiomegaly, Alzheimer's disease, cystic fibrosis, viral disease, autoimmune diseases, atherosclerosis, restenosis, psoriasis, allergic disorders including asthma, inflammation, neurological disorders and hormone-related diseases. ERK-2 protein kinase and its implication in various diseases has been described for example in Bokemeyer et al. 1996, Kidney Int. 49, 1187; Anderson et al., 1990, Nature 343, 651; Crews et al., 1992, Science 258, 478; Bjorbaek et al., 1995, J. Biol. Chem. 270, 18848; Rouse et al., 1994, Cell 78, 1027; Raingeaud et al., 1996, Mol. Cell. Biol. 16, 1247; Raingeaud et al. 1996; Chen et al., 1993 Proc. Natl. Acad. Sci. USA 90, 10952; Oliver et al., 1995, Proc. Soc. Exp. Biol. Med. 210, 162; Moodie et al., 1993, Science 260, 1658; Frey and Mulder, 1997, Cancer Res. 57, 628; Sivaraman et al., 1997, J Clin. Invest. 99, 1478; Whelchel et al., 1997, Am. J. Respir. Cell Mol. Biol. 16, 589.

Another aspect of the invention relates to inhibiting ERK-2 activity in a biological sample or a patient, which method comprises administering to the patient a compound of formula I, or a composition comprising said compound.

Another aspect of this invention relates to a method of treating or preventing an AKT-mediated diseases with an AKT inhibitor, which method comprises administering to a patient in need of such a treatment a therapeutically effective amount of a compound of formula I or a pharmaceutical composition thereof.

The terms “AKT-mediated disease” or “AKT-mediated condition”, as used herein, mean any disease or other deleterious condition in which AKT is known to play a role. The terms “AKT-mediated disease” or “AKT-mediated condition” also mean those diseases or conditions that are alleviated by treatment with a AKT inhibitor. AKT-mediated diseases or conditions include, but are not limited to, proliferative disorders, cancer, and neurodegenerative disorders. The association of AKT, also known as protein kinase B, with various diseases has been described for example in Khwaja, A., Nature, pp. 33-34, 1990; Zang, Q. Y., et al, Oncogene, 19 2000; Kazuhiko, N., et al, The Journal of Neuroscience, 20 2000.

Another aspect of the invention relates to inhibiting AKT activity in a biological sample or a patient, which method comprises administering to the patient a compound of formula I, or a composition comprising said compound.

Another aspect of this invention relates to a method of treating or preventing a Src-mediated disease with a Src inhibitor, which method comprises administering to a patient in need of such a treatment a therapeutically effective amount of a compound of formula I or a pharmaceutical composition thereof.

The terms “Src-mediated disease” or “Src-mediated condition”, as used herein mean any disease or other deleterious condition in which Src is known to play a role. The terms “Src-mediated disease” or “Src-mediated condition” also mean those diseases or conditions that are alleviated by treatment with a Src inhibitor. Such conditions include, without limitation, hypercalcemia, osteoporosis, osteoarthritis, cancer, symptomatic treatment of bone metastasis, and Paget's disease. Src protein kinase and its implication in various diseases has been described for example in Soriano, Cell, 69, 551 (1992); Soriano et al., Cell, 64, 693 (1991); Takayanagi, J. Clin. Invest., 104, 137 (1999); Boschelli, Drugs of the Future 2000, 25(7), 717, (2000); Talamonti, J. Clin. Invest., 91, 53 (1993); Lutz, Biochem. Biophys. Res. 243, 503 (1998); Rosen, J. Biol. Chem., 261, 13754 (1986); Bolen, Proc. Natl. Acad. Sci. USA, 84, 2251 (1987); Masaki, Hepatology, 27, 1257 (1998); Biscardi, Adv. Cancer Res., 76, 61 (1999); Lynch, Leukemia, 7, 1416 (1993); Wiener, Clin. Cancer Res., 5, 2164 (1999); Staley, Cell Growth Diff., 8, 269 (1997).

Another aspect of the invention relates to inhibiting Src activity in a biological sample or a patient, which method comprises administering to the patient a compound of formula I, or a composition comprising said compound.

Another aspect of this invention relates to a method of treating or preventing an Lck-mediated disease with an Lck inhibitor, which method comprises administering to a patient in need of such a treatment a therapeutically effective amount of a compound of formula I, or a pharmaceutical composition thereof.

The terms “Lck-mediated disease” or “Lck-mediated condition”, as used herein, mean any disease state or other deleterious condition in which Lck is known to play a role. The terms “Lck-mediated disease” or “Lck-mediated condition” also mean those diseases or conditions that are alleviated by treatment with an Lck inhibitor. Lck-mediated diseases or conditions include, but are not limited to, autoimmune diseases such as transplant rejection, allergies, rheumatoid arthritis, and leukemia. The association of Lck with various diseases has been described for example in Molina et al., Nature, 357, 161 (1992).

Another aspect of the invention relates to inhibiting Lck activity in a biological sample or a patient, which method comprises administering to the patient a compound of formula I, or a composition comprising said compound.

Another aspect of this invention relates to a method of treating or preventing an Abl-mediated disease with an Abl inhibitor, which method comprises administering to a patient in need of such a treatment a therapeutically effective amount of a compound of formula I, or a pharmaceutical composition thereof.

The terms “Abl-mediated disease” or “Abl-mediated condition”, as used herein, mean any disease state or other deleterious condition in which Abl is known to play a role. The terms “Abl-mediated disease” or “Abl-mediated condition” also mean those diseases or conditions that are alleviated by treatment with an Abl inhibitor. Abl-mediated diseases or conditions include, but are not limited to, leukemias, particularly chronic myeloid leukemia. The association of Abl with various diseases has been described for example in Druker, et al., N. Engl. J. Med. 2001, 344, 1038-1042.

Another aspect of the invention relates to inhibiting Abl activity in a biological sample or a patient, which method comprises administering to the patient a compound of formula I, or a composition comprising said compound.

Another aspect of this invention relates to a method of treating or preventing a cKit-mediated disease with an cKit inhibitor, which method comprises administering to a patient in need of such a treatment a therapeutically effective amount of a compound of formula I, or a pharmaceutical composition thereof.

The terms “cKit-mediated disease” or “cKit-mediated condition”, as used herein, mean any disease state or other deleterious condition in which cKit is known to play a role. The terms “cKit-mediated disease” or “cKit-mediated condition” also mean those diseases or conditions that are alleviated by treatment with an cKit inhibitor. cKit-mediated diseases or conditions include, but are not limited to, mastocytosis/mast cell leukemia, gastrointestinal stromal tumor, sinonasal natural killer/T-cell lymphoma, seminoma/dysgerminoma, throid carcinoma, small-cell lung carcinoma, malignant melanoma, adenoid cystic carcinoma, ovarian carcinoma, acute myelogenious leukemia, anaplastic large-cell lymphoma, angiosarcoma, endometrial carcinom, pediatric T-cell ALL/lymphoma, breast carcinoma and prostate carcinoma. The association of cKit with various diseases has been described for example in Heinrich, et al., J. Clinical Oncology 2002, 20, 1692-1703.

Another aspect of the invention relates to inhibiting cKit activity in a biological sample or a patient, which method comprises administering to the patient a compound of formula I, or a composition comprising said compound.

Another aspect of this invention relates to a method of treating or preventing a Flt3-mediated disease with an Flt3 inhibitor, which method comprises administering to a patient in need of such a treatment a therapeutically effective amount of a compound of formula I, or a pharmaceutical composition thereof.

The terms “Flt3-mediated disease” or “Flt3-mediated condition”, as used herein, mean any disease state or other deleterious condition in which Flt3 is known to play a role. The terms “Flt3-mediated disease” or “Flt3-mediated condition” also mean those diseases or conditions that are alleviated by treatment with an Flt3 inhibitor. Flt3-mediated diseases or conditions include, but are not limited to, acute myelogenous leukemia, mixed lineage leukemia and acute lymphocytic leukemia. The association of Flt3 with various diseases has been described for example in Sternberg and Licht, Curr. Opin Hematol. 2004, 12, 7-13.

Another aspect of the invention relates to inhibiting Flt3 activity in a biological sample or a patient, which method comprises administering to the patient a compound of formula I, or a composition comprising said compound.

Another aspect of this invention relates to a method of treating or preventing a KDR-mediated disease with a KDR inhibitor, which method comprises administering to a patient in need of such a treatment a therapeutically effective amount of a compound of formula I, or a pharmaceutical composition thereof.

The terms “KDR-mediated disease” or “KDR-mediated condition”, as used herein, mean any disease state or other deleterious condition in which KDR is known to play a role. The terms “KDR-mediated disease” or “KDR-mediated condition” also mean those diseases or conditions that are alleviated by treatment with an KDR inhibitor. KDR-mediated diseases or conditions include, but are not limited to, carcinoma of the lung, breast, gastrointestinal tract, kidney, bladder, ovary and endometrium, intracranial tumors including glioblatoma multiforme, sporadic capillary hemangioblastoma, hematological malignancies, including T cell lymphoma, acute lymphoblastic leukemia, Burkitt's lymphoma and promyelocytic leukemia, age-related macular degeneration, herpetic ocular disease, rheumatoid arthritis, cerebral ischemia and endometriosis. The association of KDR with various diseases has been described for example in Ferrara, Endocrine Reviews 2004, 25, 581-611.

Another aspect of the invention relates to inhibiting KDR activity in a biological sample or a patient, which method comprises administering to the patient a compound of formula I, or a composition comprising said compound.

The term “patient” includes human and veterinary subjects.

The term “biological sample”, as used herein, includes, without limitation, cell cultures or extracts thereof; preparations of an enzyme suitable for in vitro assay; biopsied material obtained from a mammal or extracts thereof, and blood, saliva, urine, feces, semen, tears, or other body fluids or extracts thereof.

An amount effective to inhibit protein kinase, for example, Aurora A, is an amount that causes measurable inhibition of the kinase activity when compared to the activity of the enzyme in the absence of an inhibitor. Any method may be used to determine inhibition, such as, for example, the Biological Testing Examples described below.

The term “pharmaceutically acceptable carrier, adjuvant, or vehicle” refers to a non-toxic carrier, adjuvant, or vehicle that may be administered to a patient, together with a compound of this invention, and which does not destroy or reduce the pharmacological activity thereof.

Pharmaceutically acceptable carriers that may be used in these pharmaceutical compositions are generally known in the art. They include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, solvents, salts or electrolytes, such as protamine sulfate, disodium hydrogen phosphate, potassium hydrogen phosphate, sodium chloride, zinc salts, silicates, colloidal silica, magnesium trisilicate, polyvinyl pyrrolidone, cellulose-based substances, polyethylene glycol, sodium carboxymethylcellulose, polyacrylates, waxes, oils, carbohydrate polymers, polyethylene-polyoxypropylene-block polymers, polyethylene glycol and wool fat. Pharmaceutically accepted vehicles can contain mixtures of more than one excipient in which the components and the ratios can be selected to optimize desired characteristics of the formulation including but not limited to shelf-life, stability, drug load, site of delivery, dissolution rate, self-emulsification, control of release rate and site of release, and metabolism.

The compositions of the present invention may be administered orally, parenterally, by inhalation, topically, rectally, nasally, buccally, vaginally, transdermally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, sub-cutaneously, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other surface-active emulsifying agents or bioavailability enhancers which are commonly used in the manufacture of pharmaceutically acceptable solid, liquid, or other dosage forms may also be used for the purposes of formulation.

The pharmaceutical compositions of this invention may be prepared by techniques known in the art and may be orally administered in any orally acceptable dosage form including, but not limited to, capsules, tablets, aqueous suspensions or solutions. In the case of tablets for oral use, carriers commonly used include but are not limited to celluloses, lactose, or corn starch. Lubricating agents, such as magnesium stearate, are also typically added. For oral administration in a capsule form, useful diluents or carriers include lactose and dried cornstarch. When aqueous suspensions or solutions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared using techniques known in the art including for example by mixing the agent with a suitable non-irritating excipient, which is solid at room temperature but liquid at rectal temperature, and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, the airways, or the lower intestinal tract. Suitable topical formulations are readily prepared for each of these areas or organs using techniques known in the art. For example, topical application for the lower intestinal tract can be effected in a rectal suppository formulation (see above) or in a suitable enema formulation. Topically-transdermal patches may also be used.

For topical or transdermal applications, the pharmaceutical compositions may be formulated by techniques known in the art in a suitable ointment or base containing the active component suspended or dissolved in one or more carriers. Carriers for topical administration of the compounds of this invention are well known in the art and include, but are not limited to, mineral oil, liquid petrolatum, white petrolatum, propylene glycol, polyoxyethylene, polyoxypropylene compound, emulsifying wax, and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated by techniques known in the art as micronized or nanometer-sized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as suspensions or solutions in saline, optionally employing benzyl alcohol or other suitable preservatives, absorption promoters to enhance bioavailability, fluorocarbons, and/or other conventional solubilizing or dispersing agents.

The present invention can be used to treat inflammatory or immune mediated diseases in humans or animals, wherein the inflammatory or immune mediated diseases include, but are not limited to, rheumatoid arthritis, osteoarthritis, ulcerative colitis, Crohn's disease, Mooren's ulcer, arthritis, sarcoidosis, inflammatory or immune mediated bowel disease, systemic lupus, Wegener's syndrome, Stevens-Johnson disease, Behcet's disease, pemphigoid, Lyme's disease, asthma or acquired immune deficiency syndrome.

The present invention can be used to treat infectious diseases in humans or animals, wherein the infectious diseases include, but are not limited to syphilis, a bacterial infection, a Mycobacterial infection, a bacterial ulcer, a fungal ulcer, a Herpes simplex infection, a Herpes zoster infection, a protozoan infection, malaria, a Bartonellosis infection, or toxoplasmosis.

The present invention can be used to treat blood or blood vessel diseases in humans or animals, wherein the blood or blood vessel diseases include, but are not limited to, vein occlusion, artery occlusion, carotid obstructive disease, polyarteritis, atherosclerosis, Osler-Weber-Rendu disease, sickle cell anemia, leukemia, acute or chronic neoplastic disease of the bone marrow, hemangiomas, hereditary hemorrhagic telangiectasia, disease of the bone marrow, anemia, impaired blood clotting or enlargement of the lymph nodes, liver, or spleen. The present invention can also be used to treat chronic neoplastic disease of the bone marrow, wherein those diseases include, but are not limited to, multiple myeloma and myelo dysplastic syndrome.

The present invention can be used to treat skin conditions in a humans or an animals, wherein the skin conditions include, but are not limited to, abnormal wound healing, acne rosacea, chemical burns of the skin, dermatitis or psoriasis.

In addition, the invention can be used to treat a variety of post-menopausal symptoms, osteoporosis, cardiovascular disease, myocardial angiogenesis, plaque neovascularization, hemophiliac joints, angiofibroma, wound granulation, intestinal adhesions, scleroderma, hypertrophic scars; i.e., keloids. They are also useful in the treatment of diseases that have angiogenesis as a pathologic consequence, such as cat scratch disease, and Helicobacter pylori ulcers. The invention can also be used to treat Alzheimer's disease, to reduce the incidence of stroke, and as an alternative to prior estrogen replacement therapies. The compounds of the present invention can work by estrogenic and non-estrogenic biochemical pathways.

Additionally, the compounds of the present invention can be used to treat endometriosis. Endometriosis is the abnormal growth of endometrial cells; the same cells that line the uterus that are shed monthly in the menstrual process. Wayward endometrial cells can position themselves in the lower abdomen on areas such as the cul-de-sac, the recto-vaginal septum, the stomach, the fallopian tubes, the ovaries, and the bladder. During menstruation, the normal uterine lining is sloughed off and expelled through the vagina, but transplanted endometrial tissue has no means of exiting the body; instead the endometrial tissue and cells adhere and grow where positioned. The results are internal bleeding, inflammation, and scarring. One of the serious consequences of endometrial scarring is infertility. The endometrial growths are generally not malignant or cancerous. Among other complications, the growths can rupture and can spread the endometriosis to new areas of the lower abdomen. Endometriosis is a progressive disease. The growths or lesions are first seen as clear vesicles, then become red, and finally progress to black lesions over a period of seven to ten years.

In addition, the compounds of this invention, can be formulated to increase the bioavailability of the compound by methods well known to those of ordinary skill in the art. Methods of formulating the compounds of this invention and examples of formulations are described in “Water-Insoluble Drug Formulation” Rong Liu editor, CRC Press LLC, 2000, which is incorporated herein by reference in its entirety.

Formulations contemplated as part of this invention include, but are not limited to, nanoparticles formulations made by controlled precipitation methods and by methods disclosed in U.S. patent application Ser. No. 10/392,403 (Publication No. 2004/0033267), which is hereby incorporated by reference in its entirety. Common excipients for nanoparticles known in the art include water, surface active agents such as sugar polymers (modified celluloses) and detergents, and also optionally preservatives such as benzalkonium salts, benzoic acid or salts thereof, or parabens. By forming nanoparticles, the compositions disclosed herein have increased bioavailability. Preferably, the particles of the compounds of the present invention have an effective average particle size of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods well known to those of ordinary skill in the art. Nanoparticle preparations can be incorporated into many of the formulation approaches described here, including for example suspensions or creams or ointments for topical or transdermal administration, suspensions or powders or tablets or capsules or pellets for suppositories or for oral administration, suspensions for sterile injectable formulations, and polymer formulations.

The compounds that make up this invention can be incorporated into biodegradable or non-biodegradable polymers allowing for sustained release of the compound. The polymers can be implanted so that the drug is delivered parenterally throughout the body or the polymers with the compounds that make up this invention can be implanted in the vicinity of the tumor. A review of polymers in controlled drug delivery can be found for example in “Biodegradable Polymers as Drug Delivery Systems, Chasin M and Langer R (eds), New York, Marcel Dekker, 1990, which is incorporated herein by reference in its entirety. Another review can be found in “Handbook of Biodegradable Polymers”, D. Weseman, J. Kost and A. Domb, Taylor & Francis, 1998, which is incorporated herein by reference in its entirety.

A “pharmaceutically acceptable derivative or prodrug” means any pharmaceutically acceptable salt, ester, amide, salt of an ester or amide, or other derivative of a compound of this invention which, upon administration to a recipient, is capable of providing, either directly or indirectly, a compound of this invention or an inhibitorily active metabolite or residue thereof. Particularly favored derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Pharmaceutically acceptable prodrugs of the compounds of this invention include, without limitation, the following derivatives of the present compounds: esters, amino acid esters, amino acid amides, phosphate esters, metal salts, sulfonate esters, carbamates, and amides.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

Compounds of this invention can also be formulated as mixtures or complexes, including but not limited to host-guest complexes with molecules such as cyclodextrins, non-ionic complexes, stabilized amorphous solids, glasses, solid solutions, and co-precipitates. The compound in these formulations can be dispersed to individual molecules, amorphous particles, or crystalline particles. These formulations can be prepared by techniques known to those skilled in the art, including but not limited to solvent-mediated co-precipitation, spray-drying, grinding, hot-melt extrusion, and granulation.

The amount of the protein kinase inhibitor that may be combined with the carrier materials to produce a single dosage form will vary depending upon the patient treated and the particular mode of administration. Preferably, the compositions should be formulated so that a dosage of between 0.01-100 mg/kg body weight/day of the inhibitor can be administered to a patient receiving these compositions. The compound is convieniently administered in any suitable dosage form, including but not limited to one containing 7-3000 mg or 70-1400 mg of active ingredient per unit dosage form. An oral dosage of 50-1000 mg is usually convenient.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of the inhibitor will also depend upon the particular compound in the composition.

Depending upon the particular protein kinase-mediated condition to be treated or prevented, additional therapeutic agents, which are normally administered to treat or prevent that condition, may be administered together with the inhibitors of this invention. For example, in the treatment of cancer, other kinase inhibitors, chemotherapeutic agents, anti-angiogenesis agents, anti-nausea agents, colony-stimulating factors, or other anti-proliferative agents may be combined with the present compounds to treat cancer as is known in the art. These agents include, without limitation, bevacizumab, adriamycin, dexamethasone, vincristine, cyclophosphamide, fluorouracil, topotecan, taxanes, interferons, and platinum derivatives.

Other examples of agents the inhibitors of this invention may also be combined with include, without limitation, agents for treating diabetes such as insulin or insulin analogues, in injectable or inhalation form, glitazones, alpha glucosidase inhibitors, biguanides, insulin sensitizers, and sulfonyl ureas; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; therapeutic antibodies such as bevacizumab; and agents for treating immunodeficiency disorders such as gamma globulin.

Those additional agents may be administered separately from the protein kinase inhibitor-containing composition, or as part of a multiple dosage regimen. Alternatively, those agents may be part of a single dosage form, mixed together with the protein kinase inhibitor of this invention in a single composition.

Compounds of this invention may exist in alternative tautomeric forms, for example as in tautomers shown below. Unless otherwise indicated, the representation of any tautomer is meant to include any other tautomers.

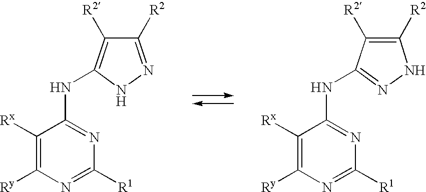

In one embodiment, the present invention provides a compound of formula I or a pharmaceutically acceptable derivative or prodrug thereof,

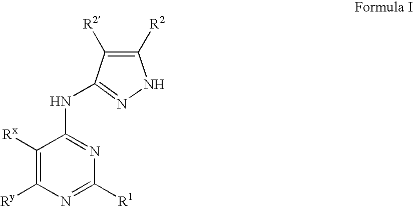

wherein:

R^{x} and R^{y} are independently R³; an optionally substituted 3-10 membered monocyclic or bicyclic heterocyclyl or heteroaryl ring, wherein the 3-10 membered heterocyclyl or heteroaryl ring may have 1-4 ring heteroatoms selected from the group consisting of nitrogen, oxygen and sulfur; or alkyl or dialkyl amino wherein alkyl is optionally substituted with —OR, —SR, amino, alkylamino, dialkylamino or a C₃₋₈ heteroaryl or heterocyclyl ring having 1-4 heteroatoms selected from the group consisting of nitrogen, oxygen and sulfur;

R¹ is an optionally substituted 5-7 membered monocyclic ring or 8-10 membered bicyclic ring selected from the group consisting of aryl, heteroaryl, heterocyclyl, and carbocyclyl, said heteroaryl or heterocyclyl ring having 0-4 ring heteroatoms selected from the group consisting of nitrogen, oxygen, and sulfur, wherein each substitutable ring carbon of R¹ is optionally independently substituted by oxo, R⁵, and each substitutable ring nitrogen of R¹ is optionally independently substituted by —R⁴;

R² and R^{2′} are independently selected from the group consisting of —R—OR, —SR, —SOR, —SO₂R, —N(R)SO₂R, —SO₂N(R)₂, —N(R)₂, —COR, —CO₂R, —N(R)COR, —N(R)C(O)OR, —N(R)CON(R)₂, —N(R)SO₂N(R)₂, —N(R⁶)N(R)₂, —C(O)N(R)₂, —OC(O)N(R)₂, —C(R)₂OR, —C(R)₂SR, —C(R)₂SOR, —C(R)₂SO₂—, —C(R)₂SO₂N(R)₂, —C(R)₂N(R)₂, —C(R)₂N(R)C(O)R, —C(R)₂N(R)C(O)OR, —C(R)═NN(R)₂, —C(R)═N—OR, —C(R)₂N(R)N(R)₂, —C(R)₂N(R)SO₂N(R)₂, and —C(R)₂N(R)CON(R)₂, or R² and R^{2′} taken together with their intervening atoms form a fused, 5-8 membered, unsaturated or partially unsaturated ring having 0-3 ring heteroatoms selected from the group consisting of nitrogen, oxygen, and sulfur, wherein each substitutable ring carbon of said fused ring formed by R² and R^{2′} is independently substituted by halo, oxo, —CN, —NO₂, R⁷, —OR, —SR, —SOR, —SO₂R, —N(R)SO₂R, —SO₂N(R)₂, —N(R)₂, —COR, —CO₂R, —N(R)COR, —N(R)C(O)OR, —N(R)CON(R)₂, —N(R)SO₂N(R)₂, —N(R⁶)N(R)₂, —C(O)N(R)₂, —OC(O)N(R)₂, —C(R)₂OR, —C(R)₂SR, —C(R)₂SOR, —C(R)₂SO₂—, —C(R)₂SO₂N(R)₂, —C(R)₂N(R)₂, —C(R)₂N(R)C(O)R, —C(R)₂N(R)C(O)OR, —C(R)═NN(R)₂, —C(R)═N—OR, —C(R)₂N(R)N(R)₂, —C(R)₂N(R)SO₂N(R)₂, and —C(R)₂N(R)CON(R)₂; and each substitutable ring nitrogen of said ring formed by R² and R^{2′} is independently substituted by —R⁴;

R³ is selected from the group consisting of —R, -halo, —OR, —C(═O)R, —CO₂R, —COCOR, —COCH₂COR, —NO₂, —CN, —S(O)R, —S(O)₂R, —SR, —N(R⁴)₂, —CON(R⁷)₂, —SO₂N(R⁷)₂, —OC(═O)R, —N(R⁷)COR, —N(R⁷)CO₂(C₁₋₆ aliphatic), —N(R⁴)N(R⁴)₂, —C═NN(R⁴)₂, —C═N—OR, —N(R⁷)CON(R⁷)₂, —N(R⁷) SO₂N(R⁷)₂, —N(R⁴)SO₂R, and —OC(═O)N(R)₂; or a C₁-C₃ alkylidene chain wherein a methylene unit of said C₁₋₃ alkylidene chain is optionally replaced by —O—, —S—, —N(R⁴)—, —CO—, —CONH—, —NHCO—, —SO₂—, —SO₂NH—, —NHSO₂—, —CO₂—, —OC(O)—, —OC(O)NH—, or —NHCO₂;

each R is independently hydrogen, R⁷ or an optionally substituted group selected from the group consisting of C₁₋₆ aliphatic, C₆₋₁₀ aryl, a heteroaryl ring having 5-10 ring atoms, and a heterocyclyl ring having 5-10 ring atoms;

each R⁴ is independently selected from the group consisting of —R⁷, —COR⁷, —CO₂ (optionally substituted C₁₋₆ aliphatic), —CON(R⁷)₂, and —SO₂R⁷;

each R⁵ is independently selected from the group consisting of —R, halo, OR, —C(═O)R, —CO₂R, —COCOR, —NO₂, —CN, —S(O)R, —SO₂R, —SR, —N(R⁴)₂, —CON(R⁴)₂, —SO₂N(R⁴)₂, —OC(═O)R, —N(R⁴)COR, —N(R⁴)CO₂R, —N(R)SO₂N(R)₂, —N(R)CON(R)₂, —N(R)SO₂N(R)₂, OC(O)N(R)₂, —N(R⁴)N(R⁴)₂, —C═NN(R⁴)₂, —C═N—OR, —N(R⁴)CON(R⁴)₂, —N(R⁴)SO₂N(R⁴)₂, —N(R⁴)SO₂R, and —OC(═O)N(R⁴)₂, —C(R)₂OR, —C(R)₂SR, —C(R)₂SOR, —C(R)₂SO₂—, —C(R)₂SO₂N(R)₂, —C(R)₂N(R)₂, —C(R)₂N(R)C(O)R, —C(R)₂N(R)C(O)OR, —C(R)═NN(R)₂, —C(R)═N—OR, —C(R)₂N(R)N(R)₂, —C(R)₂N(R)SO₂N(R)₂, and —C(R)₂N(R)CON(R)₂;

each R⁶ is independently selected from the group consisting of hydrogen and an optionally substituted C₁₋₄ aliphatic group, or two R⁶ groups on the same nitrogen atom may be taken together with the nitrogen atom to form a 5-6 membered heterocyclyl or heteroaryl ring; and

each R⁷ is independently selected from the group consisting of hydrogen, a C₁₋₆ aliphatic group, which may optionally be substituted with OR, SR or N(R)₂; an optionally substituted 3-8 membered heterocyclyl or heteroaryl ring, wherein the 3-8 membered heterocyclyl or heteroaryl ring may have 1-4 ring heteroatoms selected from the group consisting of nitrogen, oxygen and sulfur; or two R⁷ on the same nitrogen are taken together with the nitrogen to form an optionally substituted 3-8 membered heterocyclyl or heteroaryl ring, wherein the 3-8 membered heterocyclyl or heteroaryl ring may have 1-4 ring heteroatoms selected from the group consisting of nitrogen, oxygen and sulfur.

In another embodiment a compound of the Formula I or a pharmaceutically acceptable derivative or prodrug thereof is provided:

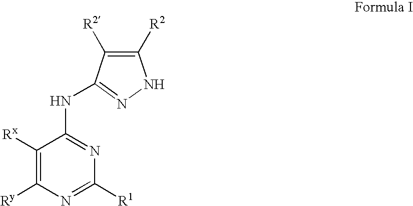

wherein:

R, R⁴, R⁵, R⁶, and R⁷ are as defined above;

R^{x} is hydrogen, N(R⁴)₂, NO₂ or a C₁₋₁₂ aliphatic group;

R^{y} is hydrogen, N(R⁴)₂, NO₂, OR, SR, S(O)R, S(O)₂R, N(R⁷)C(═O)R; an optionally substituted 3-10 membered monocyclic or bicyclic heterocyclyl or heteroaryl ring, wherein the 3-10 membered heterocyclyl or heteroaryl ring may have 1-4 ring heteroatoms selected from the group consisting of nitrogen, oxygen and sulfur; or alkyl or dialkyl amino wherein alkyl is optionally substituted with —OR, —SR, amino, alkylamino, dialkylamino or a C₃₋₈ heteroaryl or heterocyclyl ring having 1-4 heteroatoms selected from the group consisting of nitrogen, oxygen and sulfur;

R¹ is an optionally substituted 5-7 membered monocyclic or an 8-10 membered bicyclic heteroaryl ring, said heteroaryl ring having 0-4 ring heteroatoms selected from the group consisting of nitrogen, oxygen, and sulfur, wherein each substitutable ring carbon of R¹ is optionally independently substituted by oxo, or R⁵, and each substitutable ring nitrogen of R¹ is optionally independently substituted by —R⁴; and

R² and R^{2′} are independently selected from the group consisting of —R and N(R⁴)₂, OR, SR, S(O)R, S(O)₂R, or R² and R^{2′} taken together with their intervening atoms form a fused, 5-8 membered, unsaturated or partially unsaturated ring having 0-3 ring heteroatoms selected from the group consisting of nitrogen, oxygen, and sulfur, wherein each substitutable ring carbon of said fused ring formed by R² and R^{2′} is independently substituted by halo, oxo, —CN, —NO₂ or R⁷, and each substitutable ring nitrogen of said ring formed by R² and R^{2′} is independently substituted by —R⁴.

In a subembodiment R^{x} is hydrogen.

In another subembodiment, R^{x} is N(R⁴)₂ or NO₂.

In another embodiment, a compound of Formula I is provided, wherein:

R^{y} is hydrogen, N(R⁴)₂, OR, SR, or an optionally substituted 4-8 membered heterocyclyl or heteroaryl ring, wherein the 4-8 membered heterocyclyl or heteroaryl ring may have 1-4 ring heteroatoms selected from the group consisting of nitrogen, oxygen and sulfur.

In another subembodiment, a compound of Formula I is provided, wherein R² and R^{2′} are independently hydrogen, alkyl or amino.

In still another embodiment, a compound of Formula I is provided, wherein;

R¹ is an optionally substituted 5-7 membered monocyclic or an 8-10 membered bicyclic aryl or heteroaryl ring;

R² and R^{2′} are independently hydrogen or alkyl;

R^{x} is hydrogen or N(R⁴)₂; and

R^{y} is hydrogen, N(R⁴)₂, or an optionally substituted 4-8 membered heterocyclyl or heteroaryl ring, wherein the 4-8 membered heterocyclyl or heteroaryl ring may have 1-4 ring heteroatoms selected from the group consisting of nitrogen, oxygen and sulfur.

In one subembodiment, R¹ is an optionally substituted 8-10 membered bicyclic heteroaryl ring.

Another embodiment provides a compound of Formula I, wherein:

R¹ is selected from the group consisting of

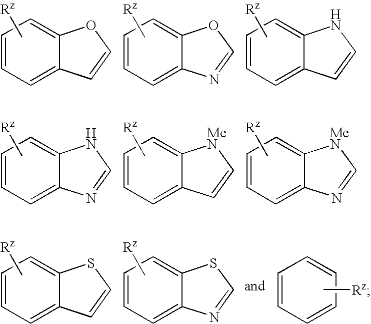

wherein R^{z} is selected from the group consisting of H, alkyl, alkoxy, halogen, CF₃, amino, alkylamino, dialkylamino, cyano and nitro; and wherein R¹ is linked to the pyrimidine ring at any substitutable ring carbon of R¹.

In one subembodiment, a compound of Formula I is provided, wherein:

R^{y} is 1-pyrrolidinyl, 1-piperidinyl, 1-piperazinyl or 4-morpholinyl.

In another subembodiment, a compound of Formula I is provided wherein R^{y} is optionally substituted alkylamino or dialkylamino.

In another subembodiment, R¹ is an optionally substituted 5-7 membered monocyclic heteroaryl ring.

In still another embodiment, a compound of Formula I is provided, wherein:

R¹ is aryl, N-methylindolyl, indolyl or benzofuranyl;

R^{x} is hydrogen;

R^{y} is N-methyl-N-2-methoxyethyl-amine, N-methyl-N-2-dimethylaminoethyl amine, 1-piperidinyl, 1-piperazinyl, 4-methyl-1-piperazinyl, 4-aminotetrahydropyran, or 4-morpholinyl;

R² is alkyl; and

R^{2′} is hydrogen.

In one embodiment, a compound of Formula I is provided wherein R², R^{2′}, R^{x}, R^{y}, are as defined above; and R¹ is a 9-membered bicyclic heteroaryl ring having 1-2 ring heteroatoms selected from the group consisting of nitrogen, oxygen and sulfur.

In another embodiment, the present invention provides a compound of formula I or a pharmaceutically acceptable derivative or prodrug thereof,

wherein R², R^{2′}, R⁴ and R⁵ are as defined above, R¹ is an optionally substituted 5-7 membered monocyclic heteroaryl ring having 1-4 ring heteroatoms selected from the group consisting of nitrogen, oxygen and sulfur, wherein each substitutable ring carbon of R¹ is optionally independently substituted by oxo or R⁵ and each substitutable ring nitrogen is optionally independently substituted by R⁴; R^{x} is hydrogen and R^{y} is a 5-7 membered ring heterocycle having 1-3 heteroatoms selected from the group consisting of nitrogen, oxygen and sulfur.

R^{x} groups in the compounds of formula I include hydrogen, alkyl, amino, nitro, alkyl- or dialkylamino, or a C₁₋₄ aliphatic group such as methyl, ethyl cyclopropyl, or isopropyl.

R^{y} groups in the compounds of formula I include hydrogen, N(R⁴)₂, NO₂, OR, SR, S(O)R, S(O)₂R and N(R⁷)C(═O)R. R^{y} groups also include 5-6 membered heteroaryl or non-aromatic heterocyclic rings, such as 2-pyridyl, 3-pyrididyl, 4-pyridyl, pyrrolidinyl, piperidinyl, morpholinyl, hydroxypiperidinyl, N-(4-hydroxypiperidin)-yl, O-(4-piperidinyl), piperazinyl, alkylpiperazinyl, or 4-methylpiperazinyl, N-acetylpiperizinyl, N-alkylcarboxamidpiperizinyl, N-(methylsulfone)piperizinyl, thiophene, furan, and tetrahydrofuran. In other embodiments, R^{y} groups include alkoxyalkylamino such as methoxyethylamino; amino, alkyl- or dialkylamino such as ethylamino or dimethylamino; alkyl- or dialkylaminoalkoxy such as dimethylaminopropyloxy. For amine nitrogens, the N can be in the free base form, a pharmaceutically acceptable salt or the quaternary salt.

In one embodiment of formula I, the optionally substituted or fused-ring amino-pyrazole can for example be selected from the following structures:

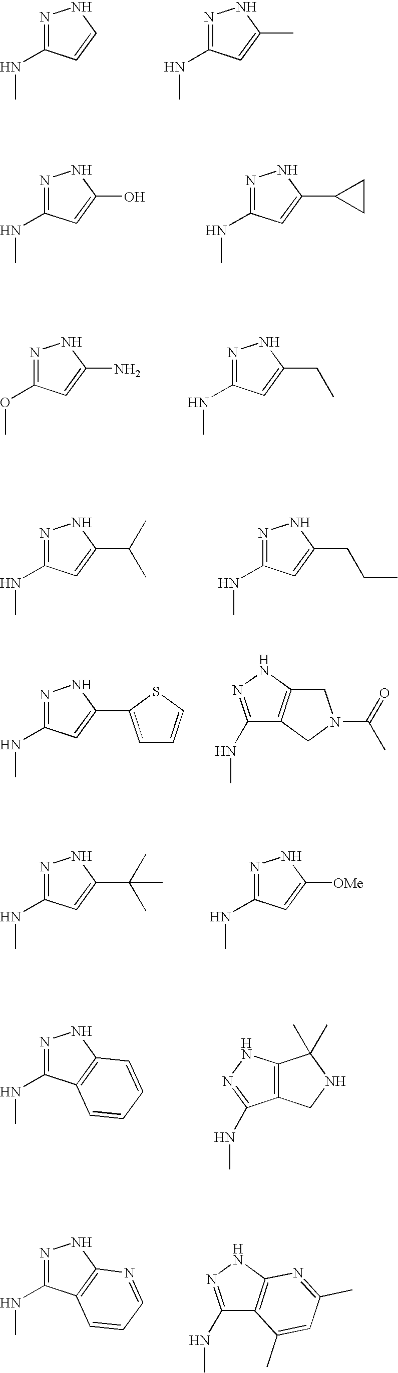

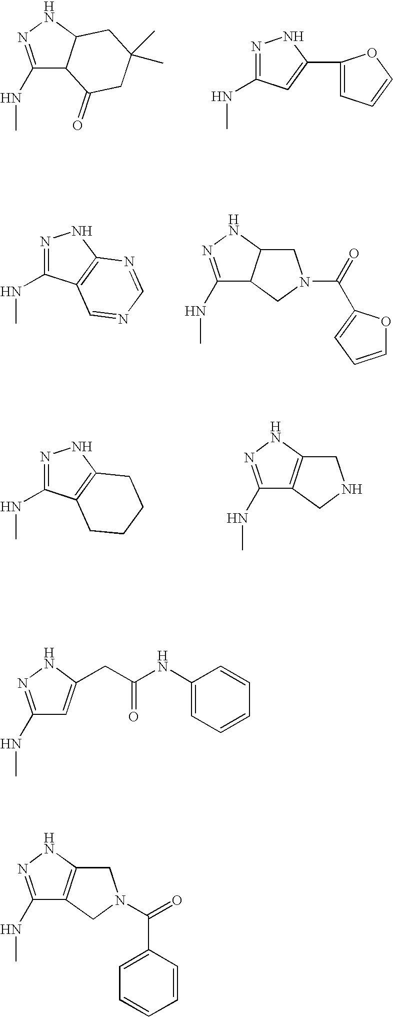

R² and R^{2′} may be taken together to form a fused ring, thus providing a bicyclic ring system containing a pyrazole ring. Fused rings include benzo, pyrido, pyrimido, a partially unsaturated 6-membered carbocyclo ring, wherein said fused ring is optionally substituted. Fused 5-membered rings are also envisioned and include but are not limited to pyrrolo, tetrahydrofuran, tetrahydrothiofuran imidazolidine and pyrazolidine. These are exemplified in the following formula I compounds having a pyrazole-containing bicyclic ring system:

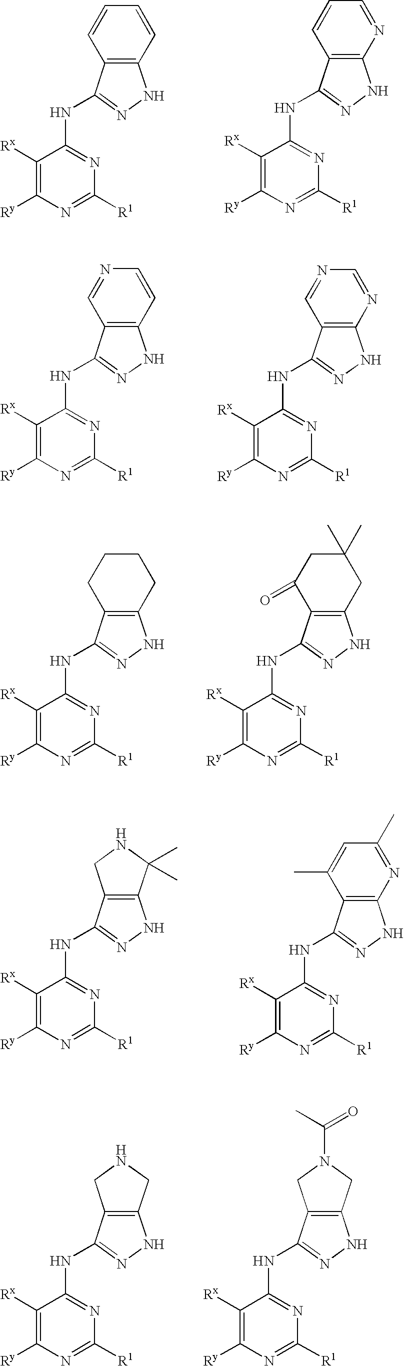

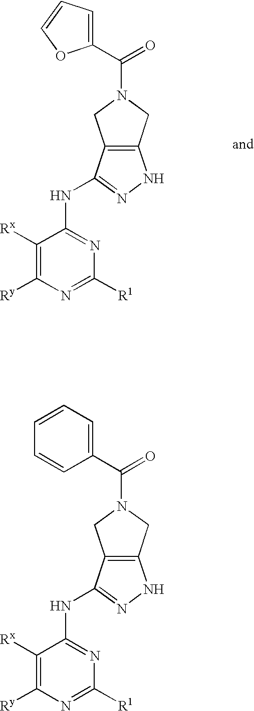

Substituents on the R²/R^{2′} fused rings include one or more of the following: -halo, —N(R⁴)₂, —C₁₋₃ alkyl, —C₁₋₃ haloalkyl, —NO₂, —O(C₁₋₃ alkyl), —CO₂(C₁₋₃ alkyl), —CN, —SO₂(C₁₋₃ alkyl), —SO₂NH₂, —OC(O)NH₂, —NH₂SO₂(C₁₋₃ alkyl), —NHC(O)(C₁₋₃ alkyl), —C(O)NH₂, and —CO(C₁₋₃ alkyl), in one embodiment, the C₁₋₃ alkyl is methyl.

When the pyrazole ring system is monocyclic, R² groups include hydrogen, C₁₋₄ aliphatic, alkoxy, alkoxycarbonyl, (un)substituted phenyl, hydroxyalkyl, alkoxyalkyl, aminocarbonyl, mono- or dialkylaminocarbonyl, aminoalkyl, alkylaminoalkyl, dialkylaminoalkyl, phenylaminocarbonyl, and (N-heterocyclyl)carbonyl. Examples of such R² substituents include methyl, cyclopropyl, ethyl, isopropyl, propyl, t-butyl, cyclopentyl, phenyl, CO₂H, CO₂CH₃, CH₂OH, CH₂OCH₃, CH₂CH₂CH₂OH, CH₂CH₂CH₂OCH₃, CH₂CH₂CH₂OCH₂Ph, CH₂CH₂CH₂NH₂, CH₂CH₂CH₂NHCOOC(CH₃)₃, CONHCH(CH₃)₂, CONHCH₂CH═CH₂, CONHCH₂CH₂OCH₃, CONHCH₂Ph, CONH(cyclohexyl), CON(Et)₂, CON(CH₃)CH₂Ph, CONH(n-C₃H₇), CON(Et)CH₂CH₂CH₃, CONHCH₂CH(CH₃)₂, CON(n-C₃H₇)₂, CO(3-methoxymethylpyrrolidin-1-yl), CONH(3-tolyl), CONH(4-tolyl), CONHCH₃, CO(morpholin-1-yl), CO(4-methylpiperazin-1-yl), CONHCH₂CH₂OH, CONH₂, and CO(piperidin-1-yl). In one embodiment R² is hydrogen.

When R¹ of formula I is monocyclic, R¹ groups include optionally substituted phenyl, pyridyl, pyridazinyl, pyrimidinyl, and pyrazinyl.

When R¹ of formula I is bicyclic, optionally substituted bicyclic R¹ groups include naphthyl, anthracenyl, tetrahydronaphthyl, indanyl, benzimidazolyl, quinolinyl, indolyl, isoindolyl, indolinyl, benzo[b]furyl, benzo[b]thiophenyl, indazolyl, benzothiazolyl, cinnolinyl, phthalazinyl, quinazolinyl, quinoxazolinyl, 1,8-naphthyridinyl and isoquinolinyl.

On R¹ of formula I, R⁵ substituents include -halo, —CN, —NO₂, —N(R⁴)₂, optionally substituted C₁₋₆ aliphatic group, —OR, —C(O)R, —CO₂R, —CONH(R⁴), —N(R⁴)COR, —N(R⁴)CO₂R, —SO₂N(R⁴)₂, —N(R⁴)SO₂R, —N(R⁶)COCH₂N(R⁴)₂, —N(R⁶)COCH₂CH₂N(R⁴)₂, —N(R)CON(R)₂, —N(R)SO₂N(R)₂, OC(O)N(R)₂, and —N(R⁶)COCH₂CH₂CH₂N(R⁴)₂, wherein R is selected from hydrogen, C₁₋₆ aliphatic, phenyl, a 5-6 membered heteroaryl ring, or a 5-6 membered heterocyclic ring. Other R⁵ substituents include —Cl, —Br, —F, —CN, —CF₃, —COOH, —CONHMe, —CONHEt, —NH₂, —NHAc, —NHSO₂Me, —NHSO₂Et, —NHSO₂(n-propyl), —NHSO₂(isopropyl), —NHCOEt, —NHCOCH₂NHCH₃, —NHCOCH₂N(CO₂ t-Bu)CH₃, —NHCOCH₂N(CH₃)₂, —NHCOCH₂CH₂N(CH₃)₂, —NHCOCH₂CH₂CH₂N(CH₃)₂, —NHCO(cyclopropyl), —NHCO(isobutyl), —NHCOCH₂(morpholin-4-yl), —NHCOCH₂CH₂(morpholin-4-yl), —NHCO—CH₂CH₂CH₂(morpholin-4-yl), —NHCO₂(t-butyl), —NH(C₁₋₄ aliphatic) such as —NHMe, —N(C₁₋₄ aliphatic)₂ such as —NMe₂, OH, —O(C₁₋₄ aliphatic) such as —OMe, C₁₋₄ aliphatic such as methyl, ethyl, cyclopropyl, isopropyl, or t-butyl, and —CO₂ (C₁₋₄ aliphatic).

In some embodiments of formula I, compounds have one, two, three, four, or all of the features selected from the group consisting of:

(a) R^{x} is hydrogen, nitro, amino, alkyl- or dialkylamino or a C₁₋₄ aliphatic group;

(b) R^{y} is —R, —N(R⁴)₂, —OR, or SR;

(c) R¹ is an optionally substituted 5-7 membered monocyclic aryl or heteroaryl ring; and

(d) R² is —R and R^{2′} is hydrogen, or R² and R^{2′} are taken together to form an optionally substituted benzo ring.

Additional formula I compounds have one, two, three, four or all of the features selected from the group consisting of:

(a) R^{y} is OR or N(R⁴)₂;

(b) R¹ is an optionally substituted 8-10 membered aryl or heteroaryl ring;

(c) R^{x} is N(R⁴)₂ or NO₂; and

(d) R² is —R and R^{2′} is hydrogen, wherein —R is independently hydrogen or an optionally substituted group selected from the group consisting of C₁₋₆ aliphatic, C₆₋₁₀ aryl, a heteroaryl ring having 5-10 ring atoms, and a heterocyclyl ring having 5-10 ring atoms.

Other compounds of formula I have one, two, three, four or all of the features selected from the group consisting of:

(a) R^{x} is hydrogen, methyl, ethyl, propyl, cyclopropyl, or isopropyl,;

(b) R^{y} is selected from 2-pyridyl, 4-pyridyl, pyrrolidinyl, piperidinyl, morpholinyl, hydroxypiperidinyl, N-(4-hydroxypiperidin)-yl, O-(4-piperidinyl), piperazinyl, alkylpiperazinyl, 4-alkylpiperazinyl, alkoxyalkylamino, alkyl- or dialkylamino, alkyl- or dialkylaminoalkoxy;

(c) R¹ is a 5-6 membered aryl or heteroaryl ring optionally substituted with one to two groups selected from -halo, CF₃, —CN, —NO₂, —N(R⁴)₂, optionally substituted C₁₋₆ aliphatic group, —OR, —CO₂R, —CONH(R⁴), —N(R⁴)COR, —N(R⁴)SO₂R, —N(R⁴)COCH₂N(R⁶)₂, —N(R⁴)COCH₂CH₂N(R⁶)₂, and —N(R⁴)COCH₂CH₂CH₂N(R⁶)₂; and

(d) R² is hydrogen or a substituted or unsubstituted C₁₋₆ aliphatic.

Still other compounds of formula I have one, two, three, four or all of the features selected from the group consisting of:

(a) R^{x} is hydrogen or amino;

(b) R^{y} is selected from 2-pyridyl, 4-pyridyl, pyrrolidinyl, piperidinyl, morpholinyl, hydroxypiperidinyl, N-(4-hydroxypiperidin)-yl, O-(4-piperidinyl), piperazinyl, alkylpiperazinyl, 4-alkylpiperazinyl, 4-acylpiperazinyl, alkyl- or dialkylamino; and alkoxyamino;

(c) R¹ is a 9 membered bicyclic heteroaryl ring, wherein R¹ is optionally substituted with one to two groups selected from -halogen, —CN, —CF₃, —NO₂, —N(R⁴)₂, optionally substituted C₁₋₆ aliphatic group, —OR, —CO₂R, —CONH(R⁴), —N(R⁴)COR, —N(R⁴)SO₂R, —N(R⁴)COCH₂N(R⁶)₂, —N(R⁴)COCH₂CH₂N(R⁶)₂, and —N(R⁴)COCH₂CH₂CH₂N(R⁶)₂; and

(d) R² is hydrogen or a substituted or unsubstituted C₁₋₆ aliphatic

In another embodiment, the present invention provides compounds of formula II or a pharmaceutically acceptable derivative or prodrug thereof:

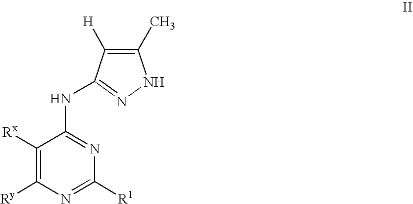

wherein

R^{x} is hydrogen, nitro, amino, alkyl- or dialkylamino, or a C₁₋₄ aliphatic group;

R^{y} is 2-pyridyl, 4-pyridyl, pyrrolidinyl, piperidinyl, N(-4-hydroxypiperidinyl)-yl, morpholinyl, piperazinyl, 4-alkyllpiperazinyl, 4-acylpiperazinyl, alkoxyalkylamino, alkyl- or dialkylamino, heterocyclylamino, heterocyclylalkylamino, alkyl- or dialkylaminoalkoxy; and

R¹ is an optionally substituted 5-7 membered monocyclic or an 8-10 membered bicyclic aryl or heteroaryl ring having 1-4 ring heteroatoms selected from nitrogen, oxygen, and sulfur, optionally substituted with one to two groups selected from the group consisting of -halogen, —CN, —CF₃, —NO₂, —N(R⁴)₂, optionally substituted C₁₋₆ aliphatic group, —OR, —CO₂R, —CONH(R⁴), —N(R⁴)COR, —N(R⁴)SO₂R, —N(R⁴)COCH₂N(R⁶)₂, —N(R⁴)COCH₂CH₂N(R⁶)₂, and —N(R⁴)COCH₂CH₂CH₂N(R⁶)₂ wherein R, R⁴, and R⁶ are defined as in formula I.

In other embodiments of the compounds of formula I or II, R^{x} is hydrogen.

In still other embodiments, R^{y} is selected from pyrrolidinyl, piperidinyl, morpholinyl, hydroxypiperidinyl, N-(4-hydroxypiperidin)-yl, O-(4-piperidinyl), piperazinyl, alkylpiperazinyl, 4-alkylpiperazinyl, 4-acylpiperazinyl, alkoxyalkylamino, including N-methyl-N′-2-methoxyethyl-amine; alkyl- or dialkylamino, including N-methyl-N′-2-(dimethylaminoethyl amine; or alkyl- or dialkylaminoalkoxy.

In some embodiments, R^{y} is 4-alkylpiperazinyl or 4-acylpiperazinyl. In other embodiments R^{y} is 4-methylpiperazinyl. In still another embodiment, R^{y} is 4-acetylpiperazinyl.

In some embodiments R^{y} is hydroxypiperidinyl. In other embodiments R^{y} is N-(4-hydroxypiperidin)-yl or O-(4-piperidinyl).

In some embodiments, the invention provides compounds of formula I or II wherein R¹ is selected from the following group:

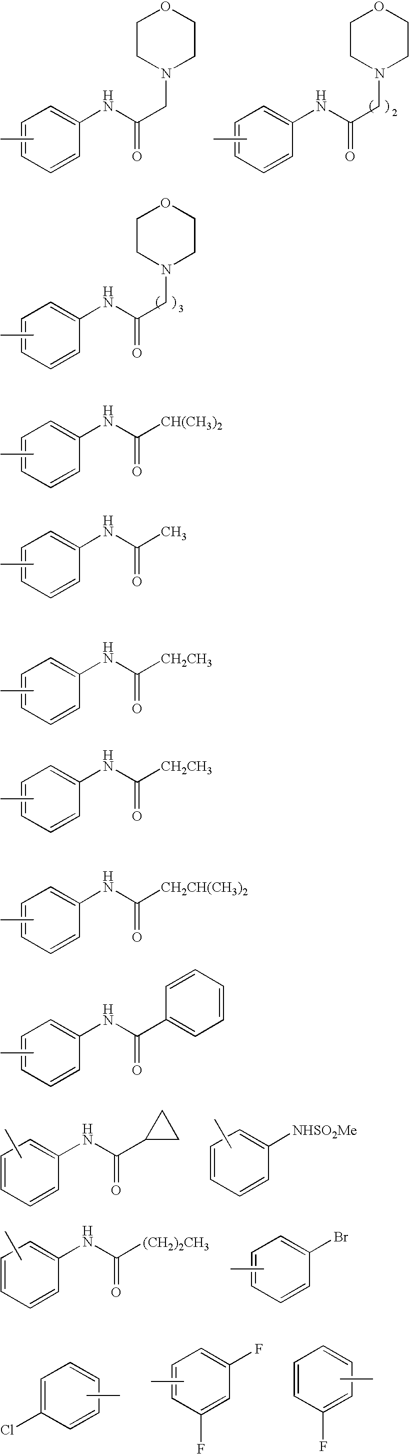

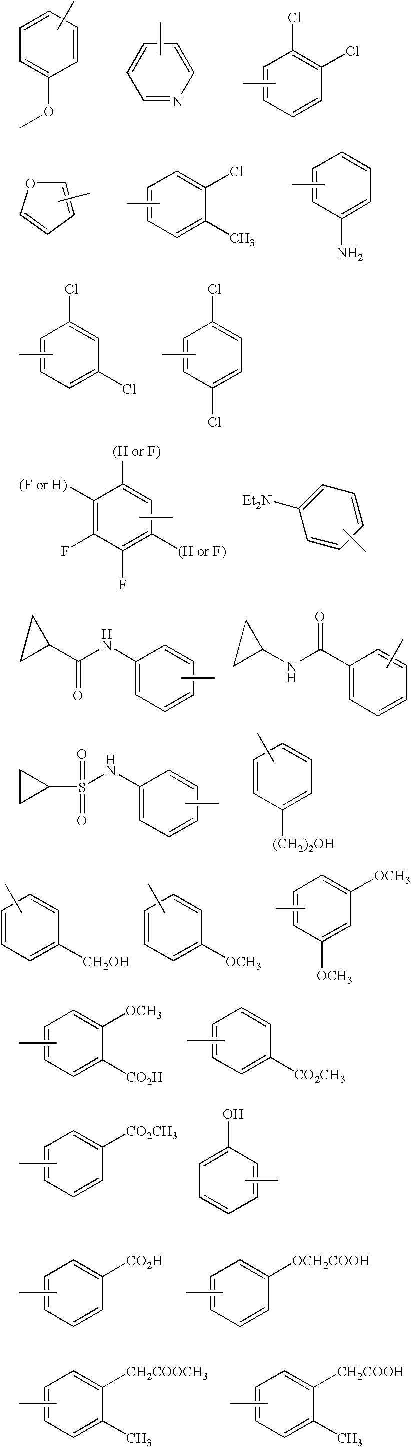

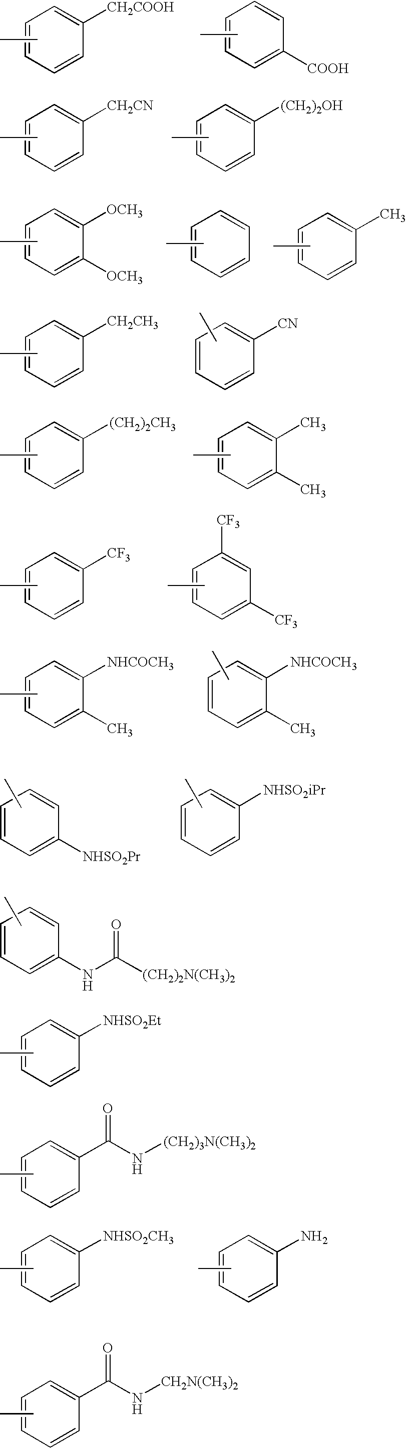

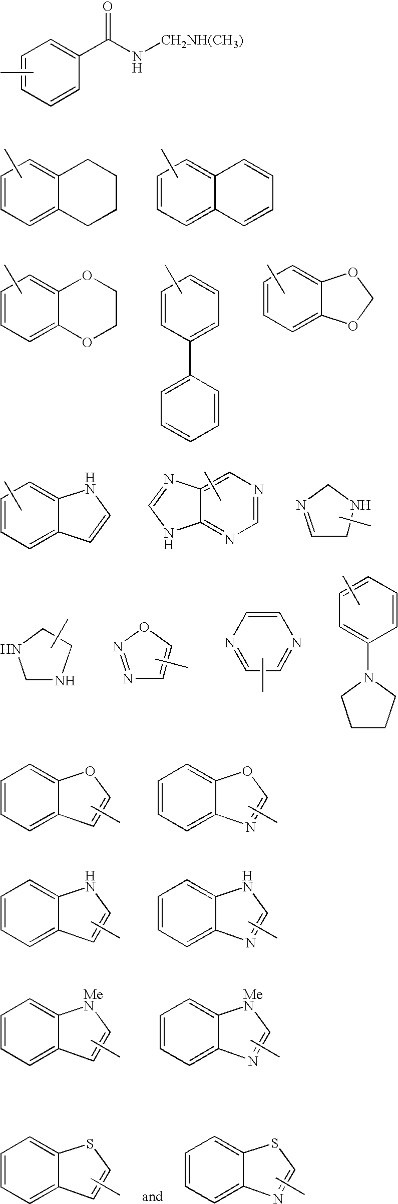

where the line drawn through the side of the substituent indicates that the substituent can be joined to the pyrimidine ring at any substitutable ring atom. Any of the embodiments described herein include the proviso that when R^{x} is H and R^{y} is 4-methylpiperazinyl, and R² is methyl, and R^{2′} is hydrogen, then R¹ is not unsubstituted indol-2-yl.

In some embodiments of the compounds of formula I or II, R^{x} is hydrogen.

In some embodiments of the compounds of formula I or II, R^{y} is 4-methylpiperazinyl.

In some embodiments of the compounds of formula I or II, R^{y} is N-(4-hydroxypiperidin)-yl or O-(4-piperidinyl).

In still other embodiments, Ry is N-methyl-N′-2-methoxyethyl-amine, N-methyl-N′-2-dimethylaminoethyl amine or 4-aminotetrahydropyran.

In other embodiments of the compounds of formula I or II, R¹ is N-methylindolyl.

In other embodiments of the compounds of formula I or II, R¹ is benzofuranyl.

In some embodiments, R^{y} is selected from the group consisting of:

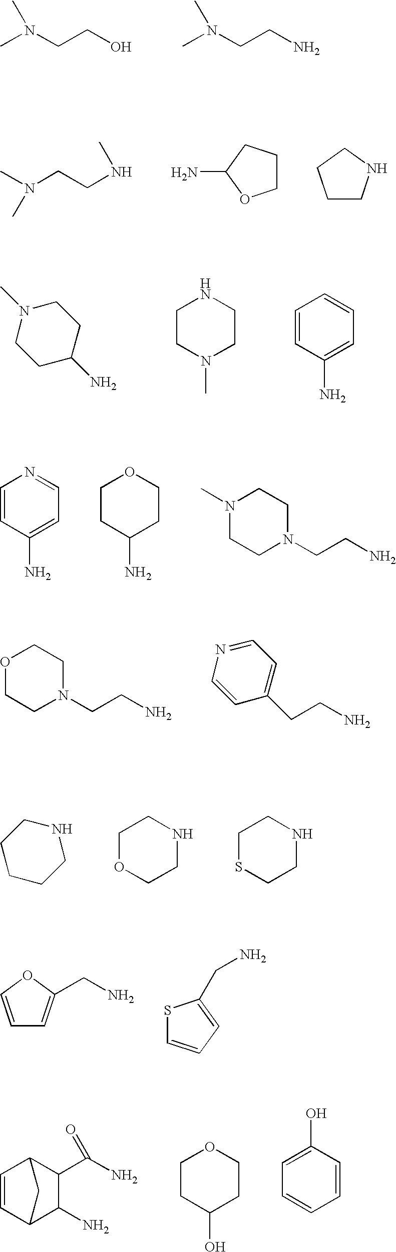

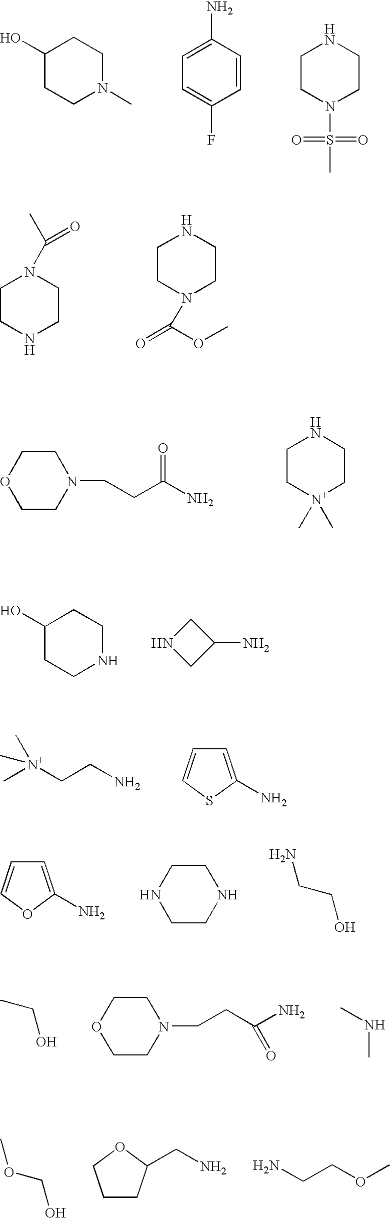

Wherein the group can be joined to the pyrimidine ring at any available heteroatom.

In any of the embodiments described herein, R¹ is not indol-2-yl when R^{x} is H and R^{y} is 4-methylpiperazinyl.

In still other embodiments, the invention provides the compounds shown in Table 1, or a pharmaceutically acceptable salt, derivative or prodrug thereof.

TABLE 1

| Com- | |
|---|---|
| pound | |
| No. | Structure |
| 1 | 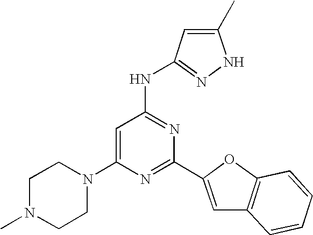 |
| 2 | 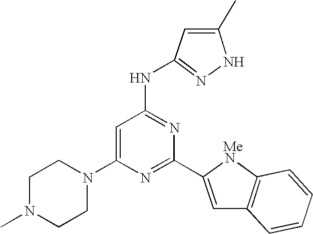 |
| 3 | 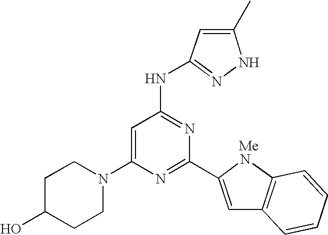 |
| 4 | 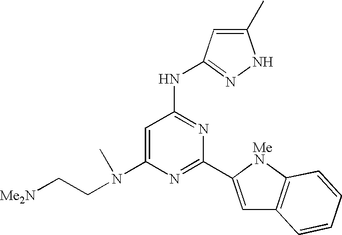 |
| 5 | 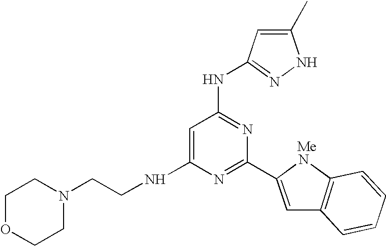 |
| 6 | 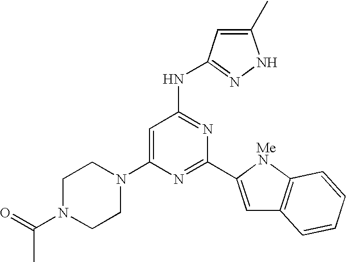 |
| 7 | 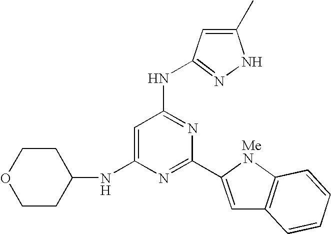 |
| 8 | 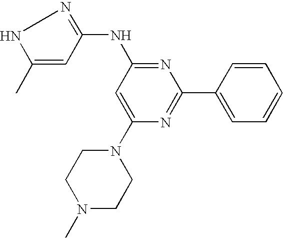 |
| 9 | 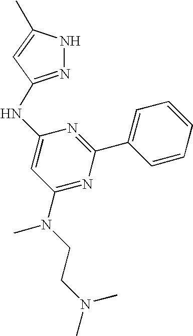 |
| 10 | 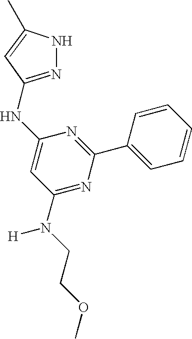 |
| 11 | 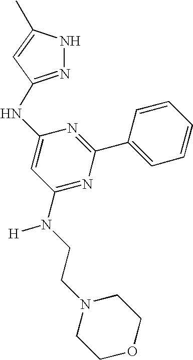 |
| 12 | 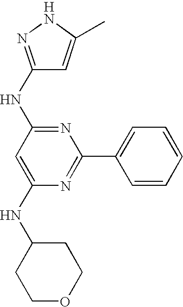 |
| 13 | 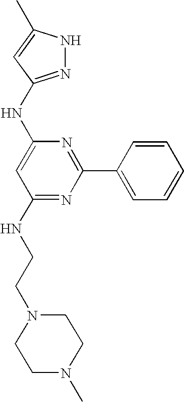 |
| 14 | 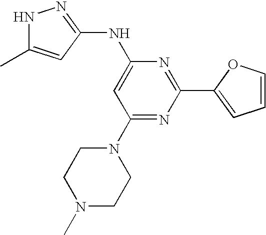 |
| 15 | 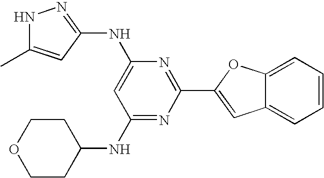 |
| 16 | 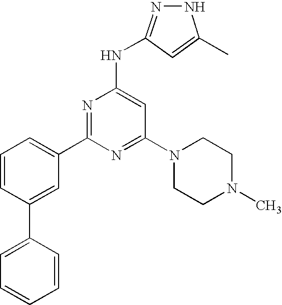 |
| 17 | 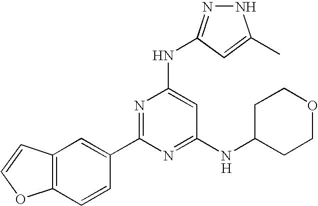 |
| 18 | 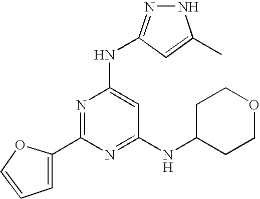 |
| 19 | 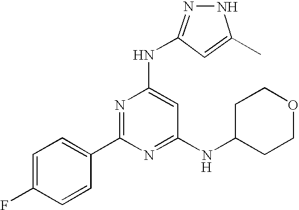 |
| 20 | 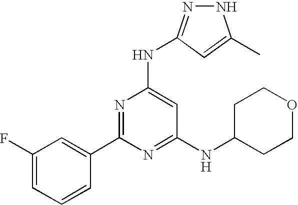 |
| 21 | 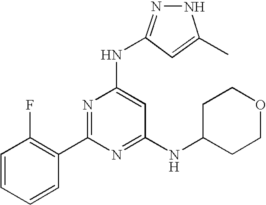 |
| 22 | 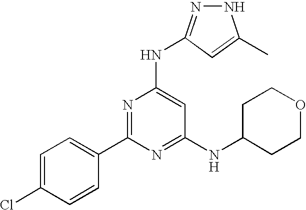 |
| 23 | 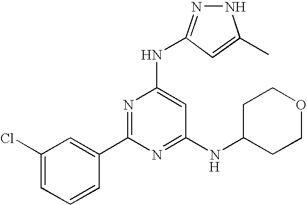 |
| 24 | 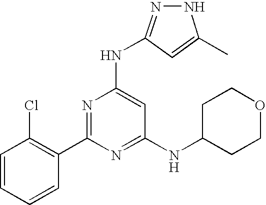 |
| 25 | 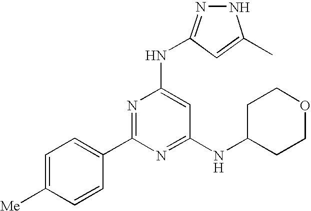 |
| 26 | 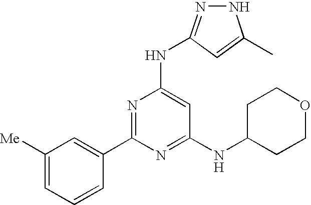 |
| 27 | 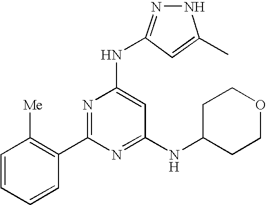 |
| 28 | 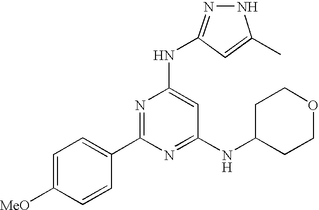 |
| 29 | 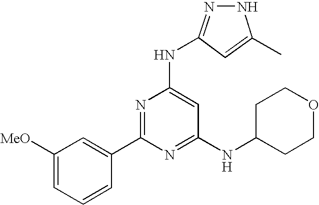 |
| 30 | 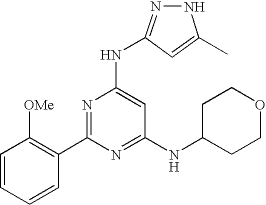 |
| 31 | 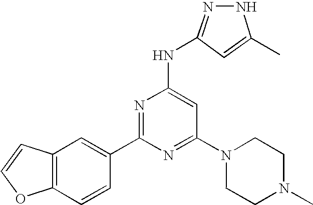 |
| 32 | 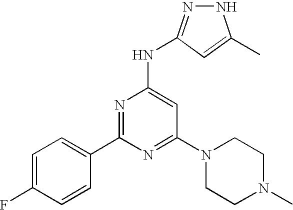 |
| 33 | 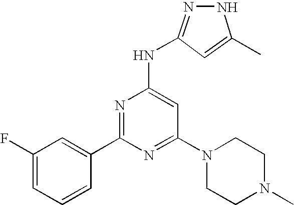 |
| 34 | 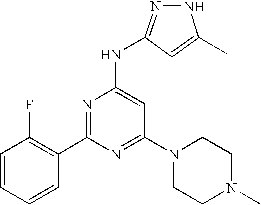 |
| 35 | 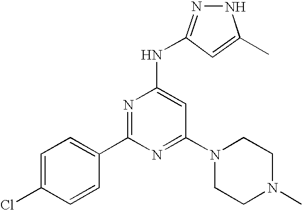 |
| 36 | 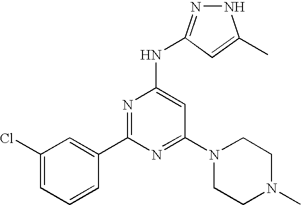 |
| 37 | 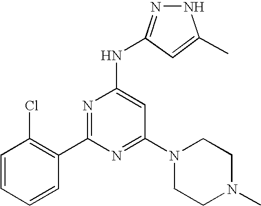 |
| 38 | 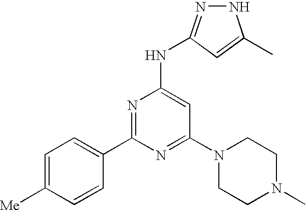 |
| 39 | 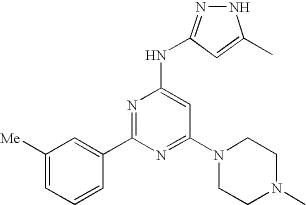 |
| 40 | 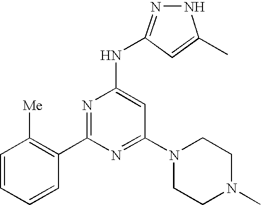 |
| 41 | 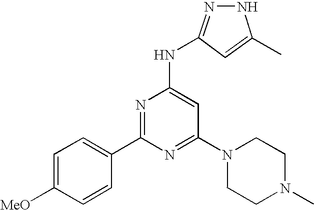 |
| 42 | 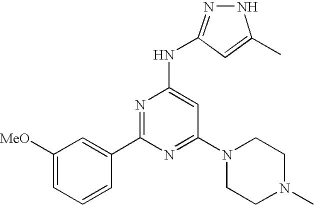 |
| 43 | 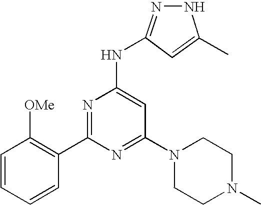 |
| 44 | 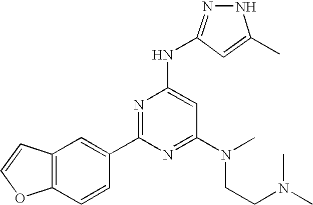 |
| 45 | 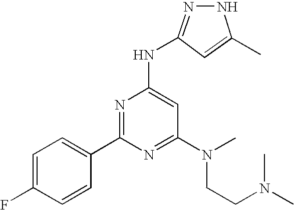 |
| 46 | 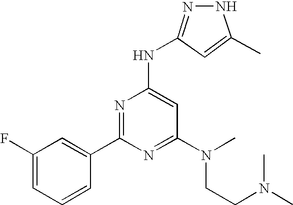 |
| 47 | 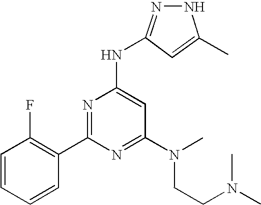 |
| 48 | 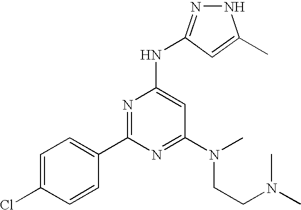 |
| 49 | 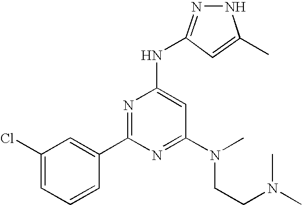 |
| 50 | 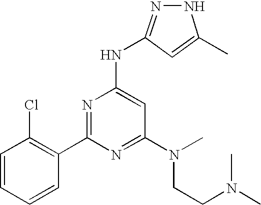 |
| 51 | 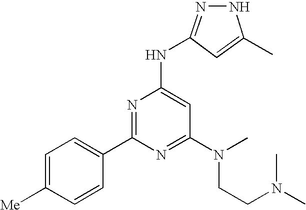 |
| 52 | 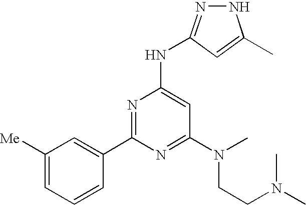 |
| 53 | 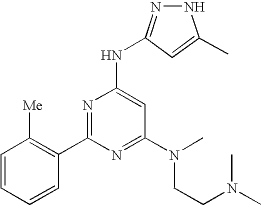 |
| 54 | 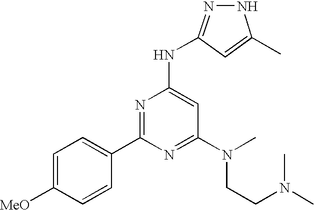 |
| 55 | 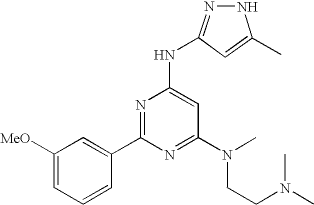 |
| 56 | 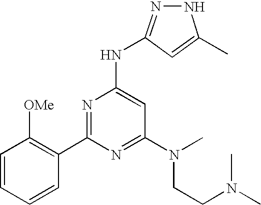 |
| 57 | 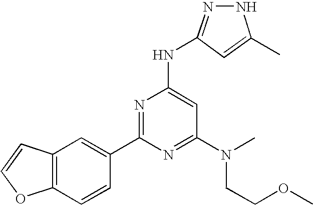 |
| 58 | 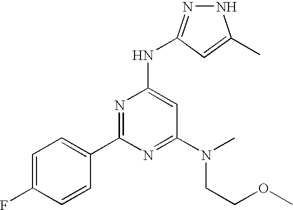 |
| 59 | 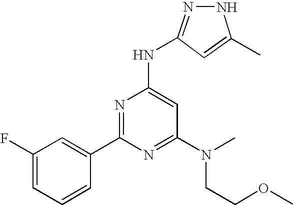 |
| 60 | 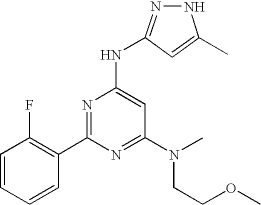 |
| 61 | 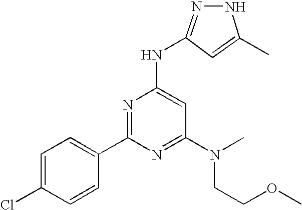 |
| 62 | 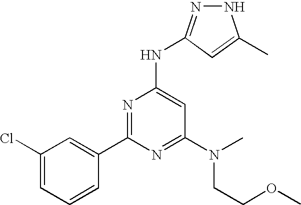 |
| 63 | 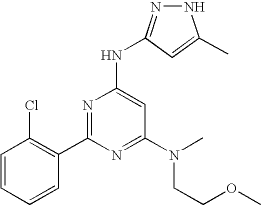 |
| 64 | 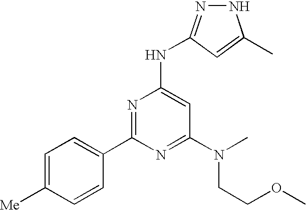 |
| 65 | 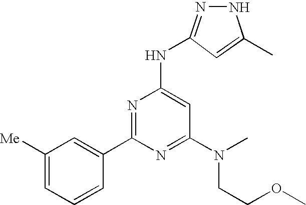 |
| 66 | 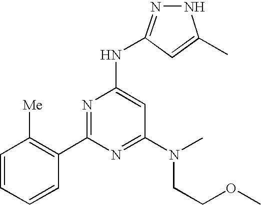 |
| 67 | 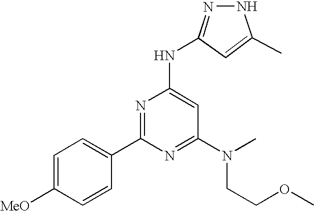 |
| 68 | 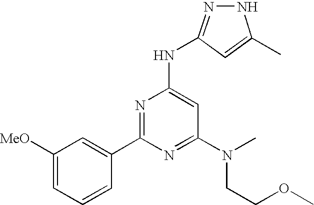 |
| 69 | 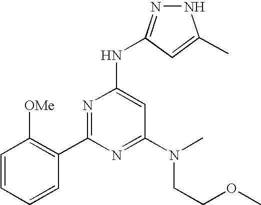 |
| 70 | 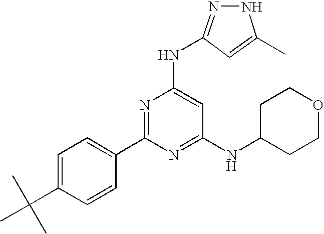 |
| 71 | 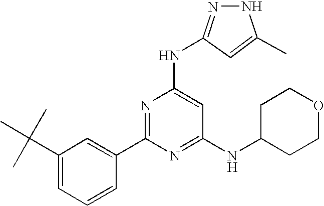 |
| 72 | 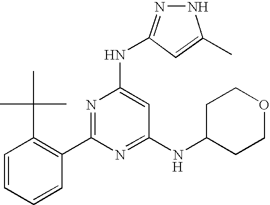 |
| 73 | 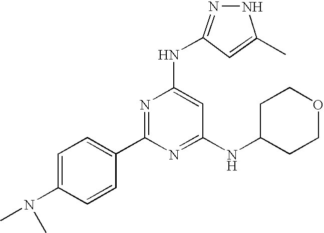 |
| 74 | 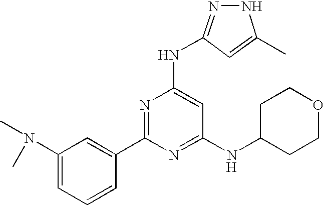 |
| 75 | 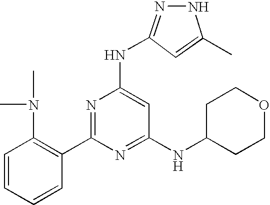 |
| 76 | 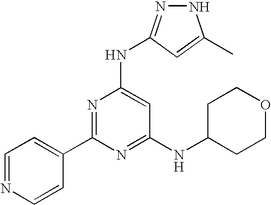 |
| 77 | 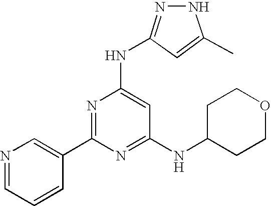 |
| 78 | 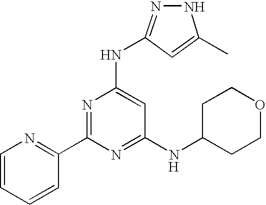 |
| 79 | 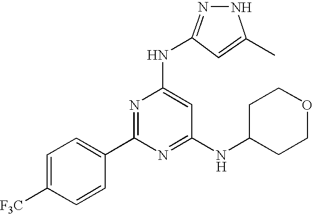 |
| 80 | 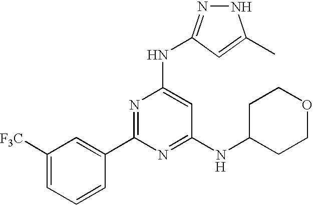 |
| 81 | 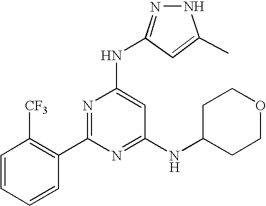 |
| 82 | 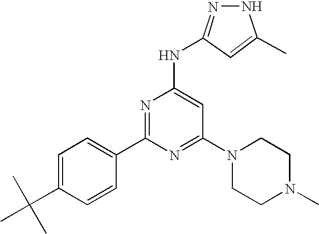 |
| 83 | 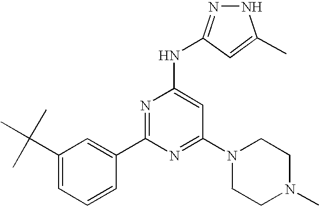 |
| 84 | 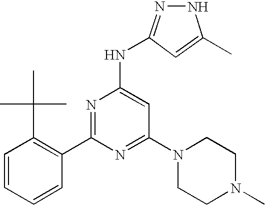 |
| 85 | 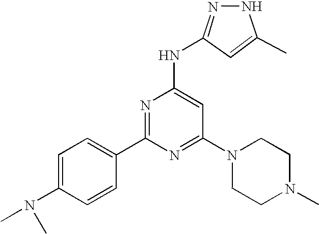 |
| 86 | 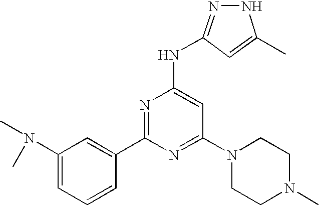 |
| 87 | 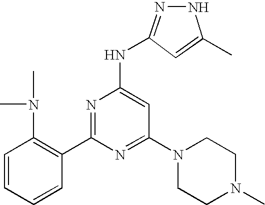 |
| 88 | 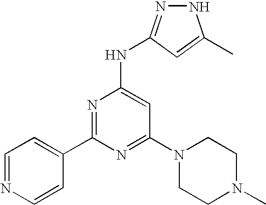 |
| 89 | 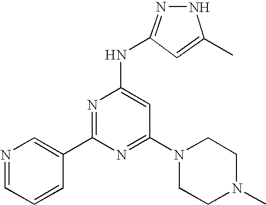 |
| 90 | 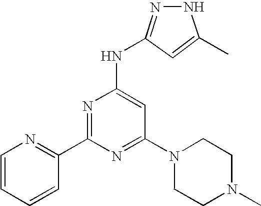 |
| 91 | 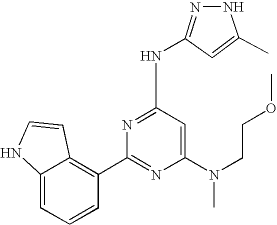 |
| 92 | 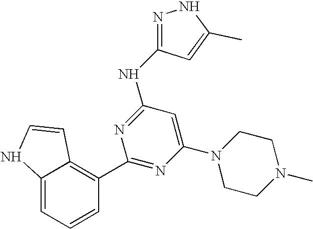 |
| 93 | 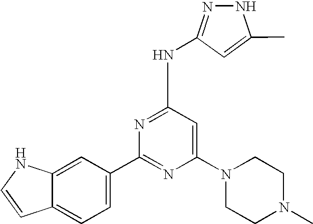 |
| 94 | 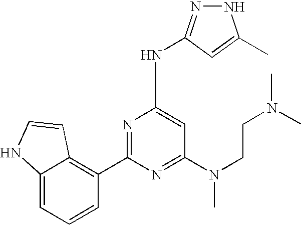 |
| 95 | 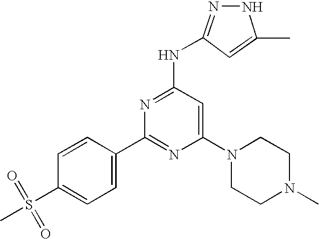 |
| 96 | 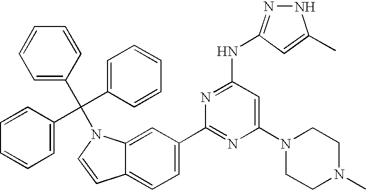 |
| 97 | 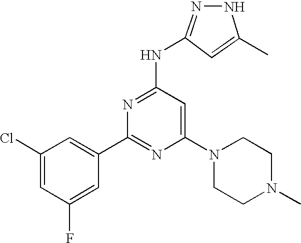 |
| 98 | 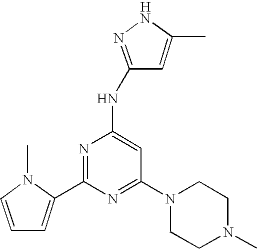 |
| 99 | 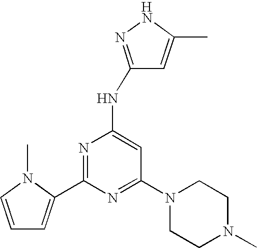 |
| 100 | 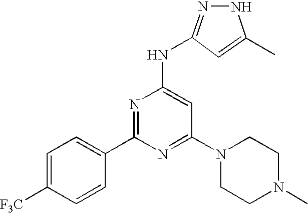 |
| 101 | 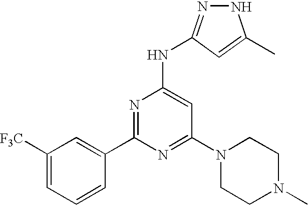 |
| 102 | 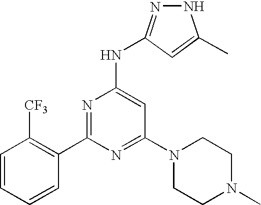 |
| 103 | 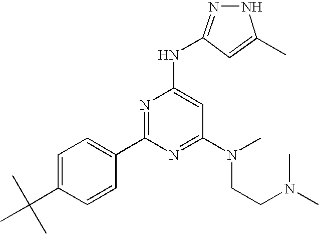 |
| 104 | 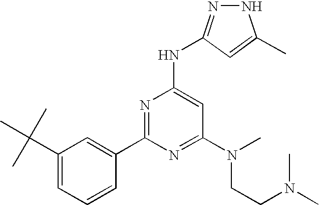 |
| 105 | 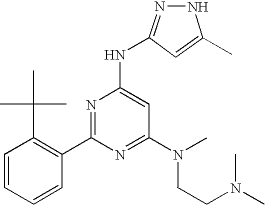 |
| 106 | 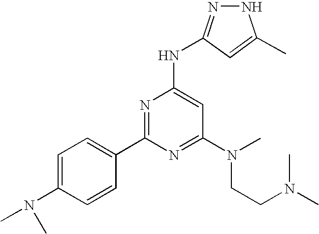 |
| 107 | 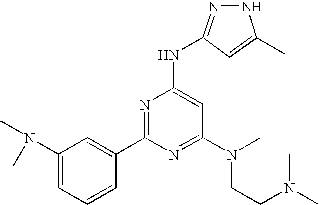 |
| 108 | 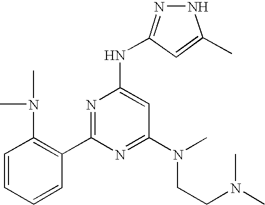 |
| 109 | 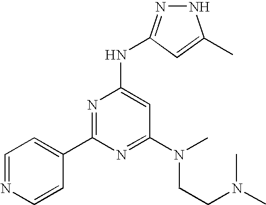 |
| 110 | 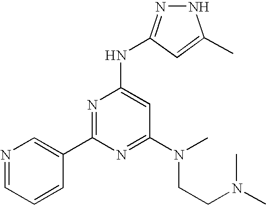 |
| 111 | 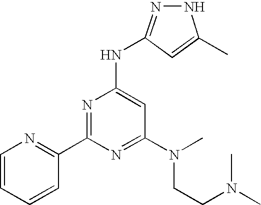 |
| 112 | 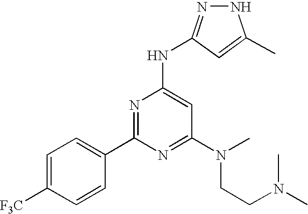 |
| 113 | 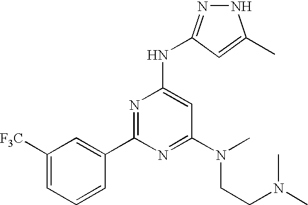 |
| 114 | 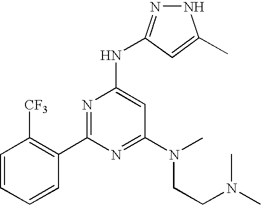 |
| 115 | 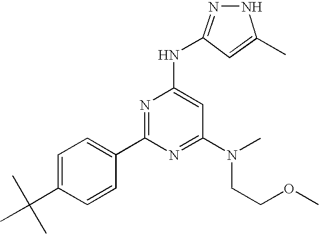 |
| 116 | 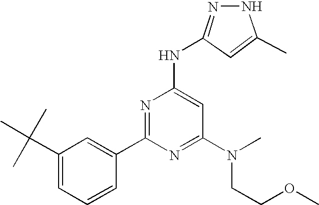 |
| 117 | 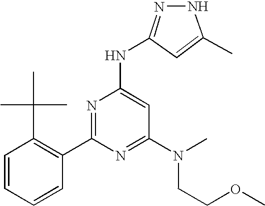 |
| 118 | 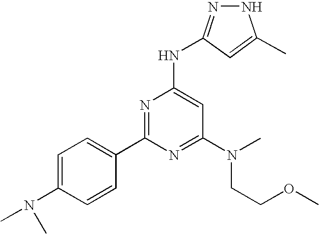 |
| 119 | 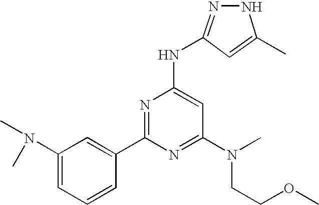 |
| 120 | 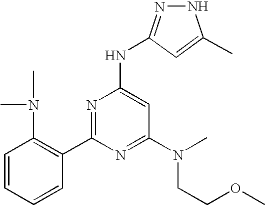 |
| 121 | 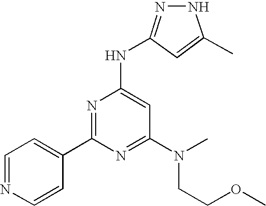 |
| 122 | 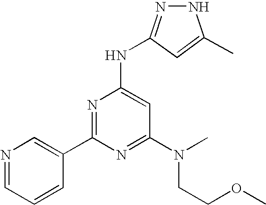 |
| 123 | 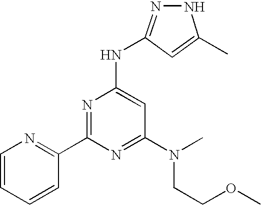 |
| 124 | 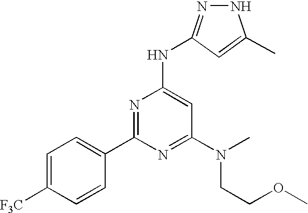 |
| 125 | 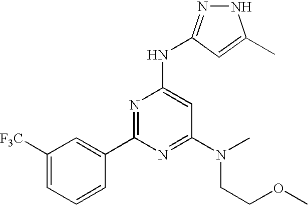 |
| 126 | 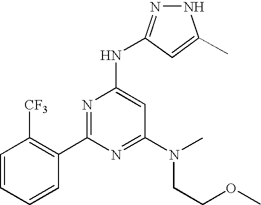 |
| 127 | 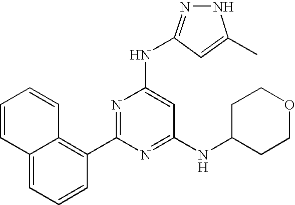 |
| 128 | 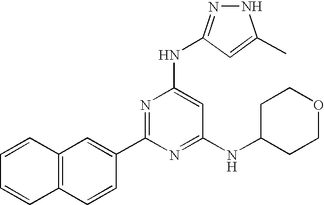 |
| 129 | 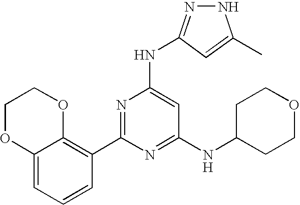 |
| 130 | 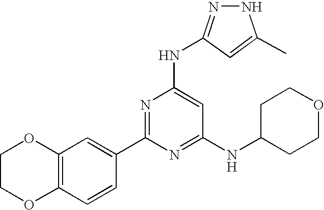 |
| 131 | 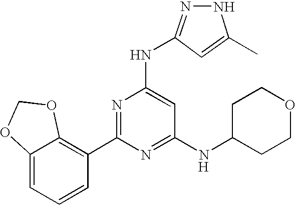 |
| 132 | 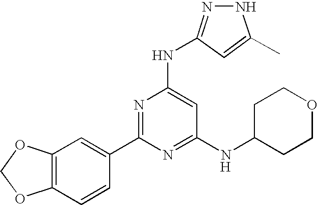 |
| 133 | 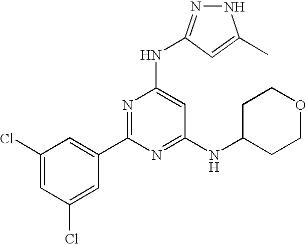 |
| 134 | 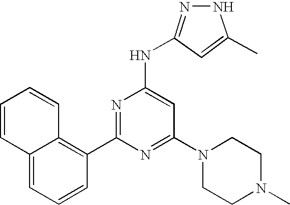 |
| 135 | 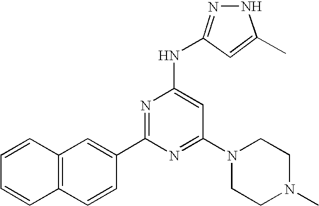 |
| 136 | 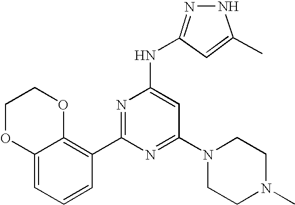 |
| 137 | 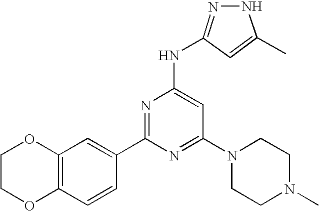 |
| 138 | 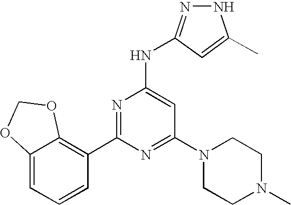 |
| 139 | 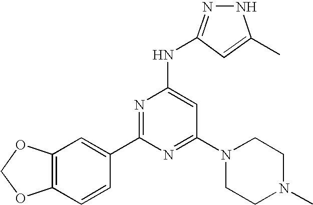 |
| 140 | 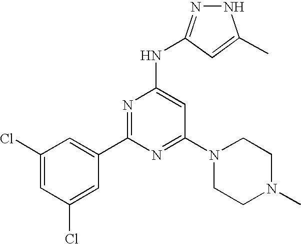 |
| 141 | 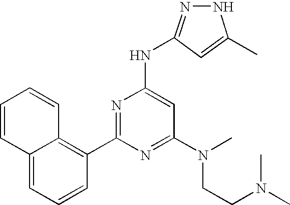 |
| 142 | 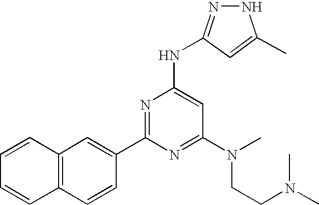 |
| 143 | 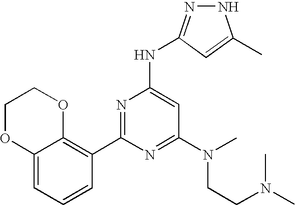 |
| 144 | 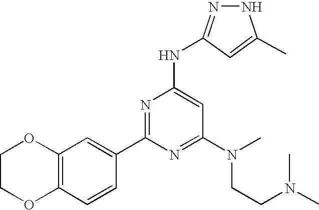 |
| 145 | 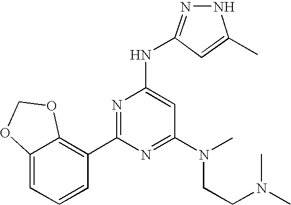 |
| 146 | 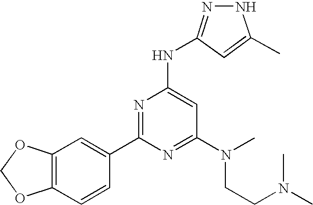 |
| 147 | 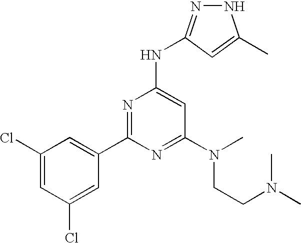 |
| 148 | 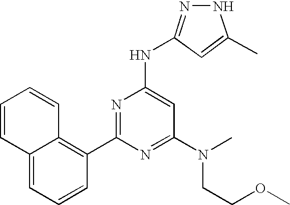 |
| 149 | 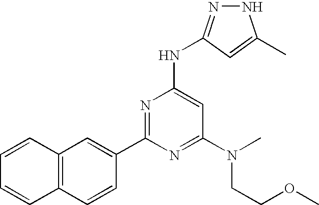 |
| 150 | 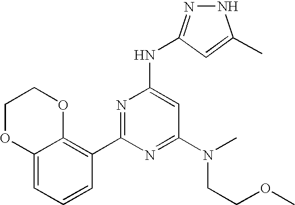 |
| 151 | 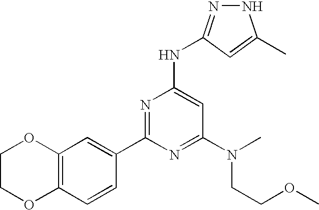 |
| 152 | 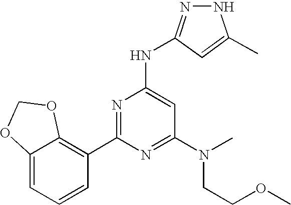 |
| 153 | 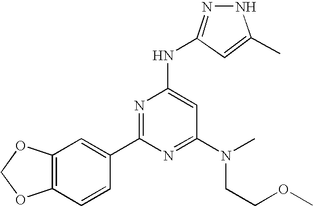 |
| 154 | 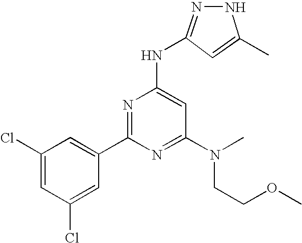 |
| 155 | 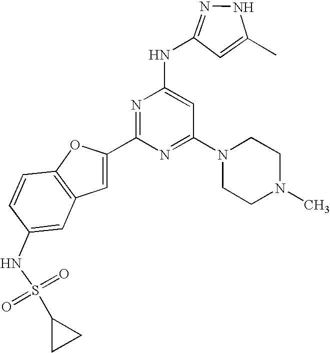 |
| 156 | 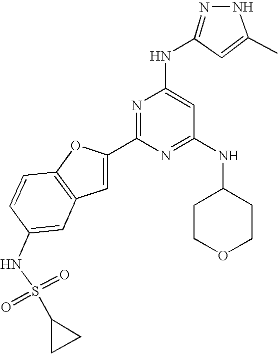 |
| 157 | 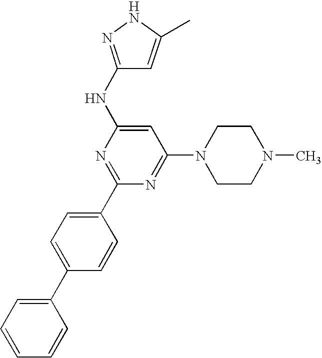 |
| 158 | 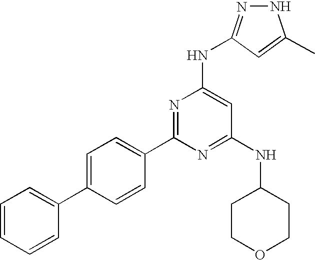 |
| 159 | 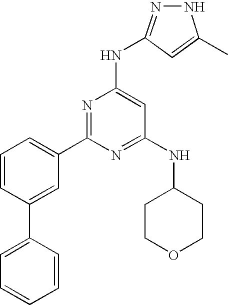 |
| 160 | 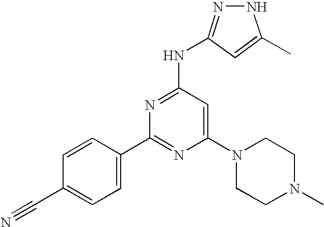 |
| 161 | 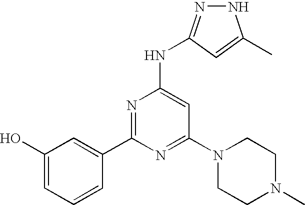 |
| 162 | 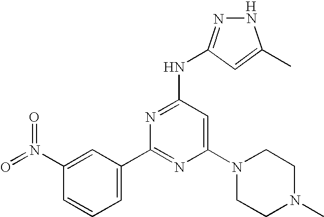 |
| 163 | 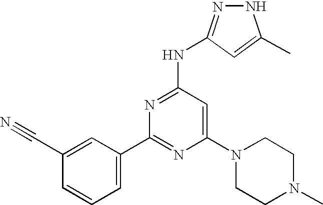 |
| 164 | 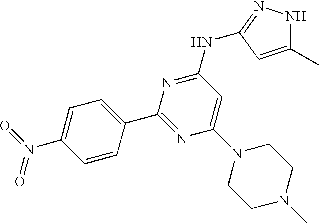 |
| 165 | 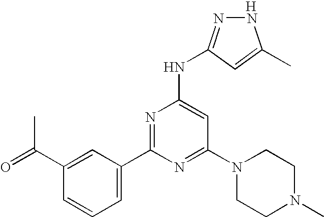 |
| 166 | 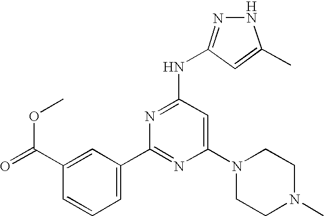 |
| 167 | 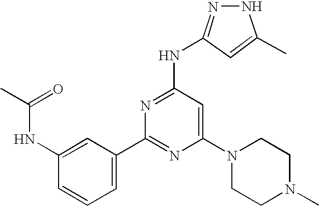 |
| 168 | 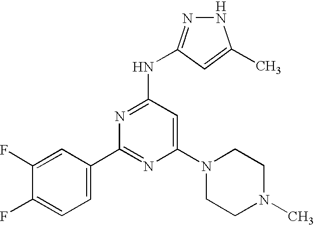 |
| 169 | 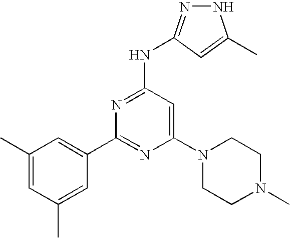 |
| 170 | 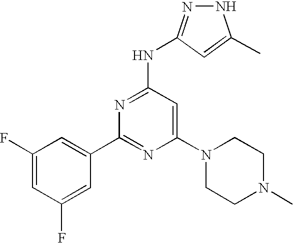 |
| 171 | 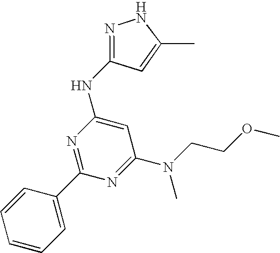 |
| 172 | 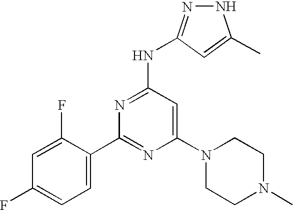 |
| 173 | 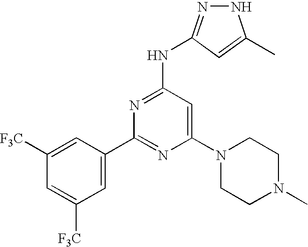 |
| 174 | 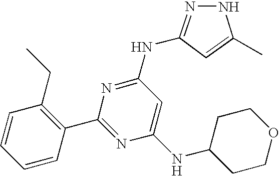 |
| 175 | 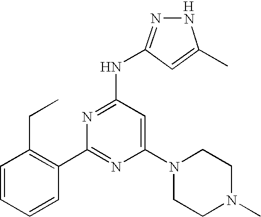 |
| 178 | 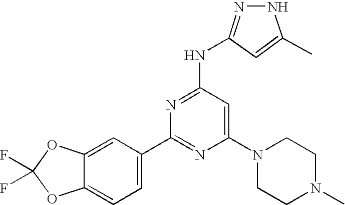 |
| 179 | 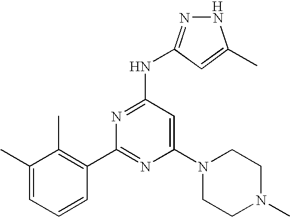 |
| 180 | 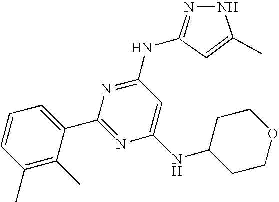 |
| 181 | 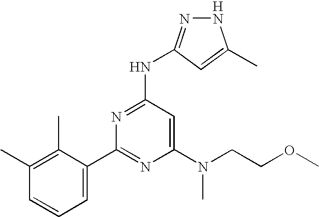 |
| 182 | 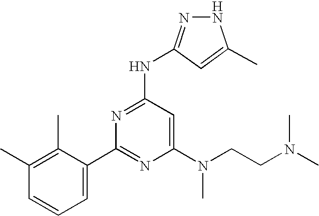 |
| 183 | 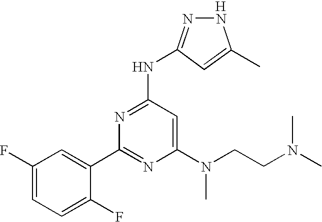 |
| 184 | 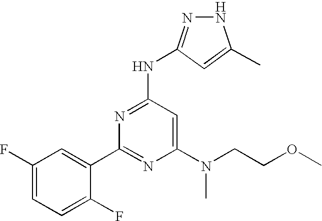 |
| 185 | 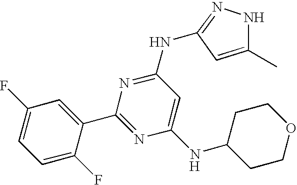 |
| 186 | 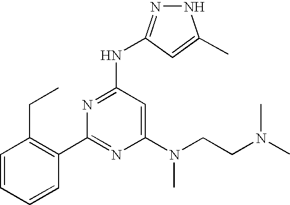 |
| 187 | 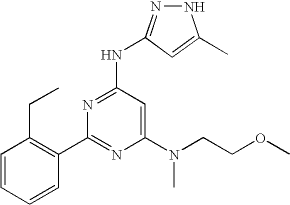 |
| 188 | 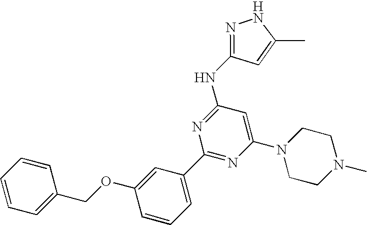 |
| 189 | 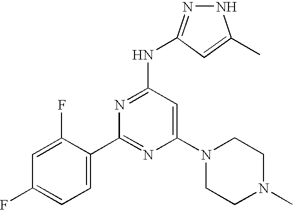 |
| 190 | 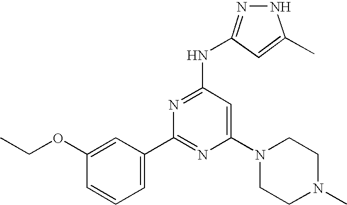 |
| 191 | 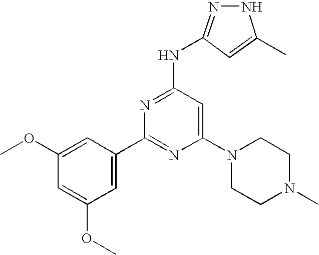 |
| 192 | 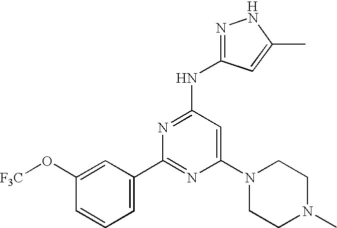 |
| 193 | 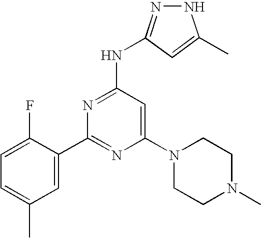 |
| 194 | 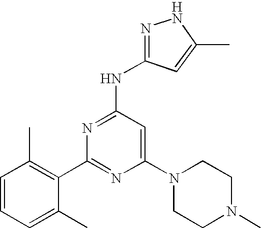 |
| 195 | 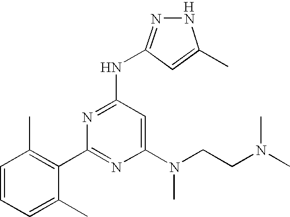 |
| 196 | 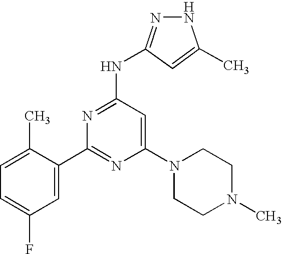 |
| 197 | 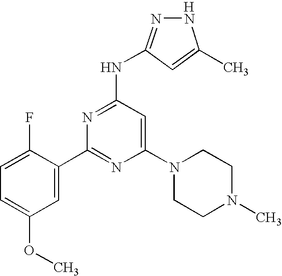 |
| 198 | 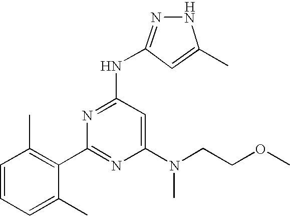 |
| 199 | 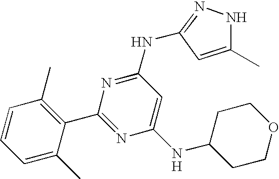 |
| 200 | 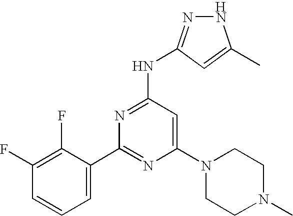 |
| 201 | 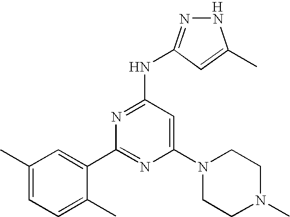 |
| 202 | 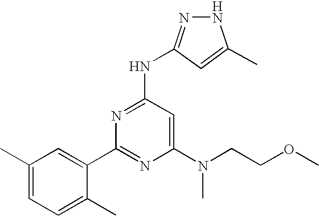 |
| 203 | 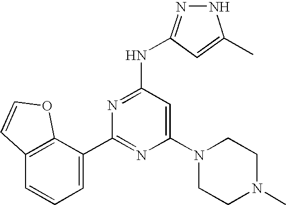 |
| 204 | 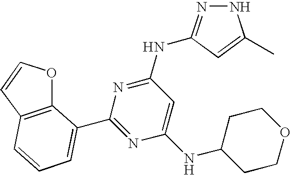 |
| 205 | 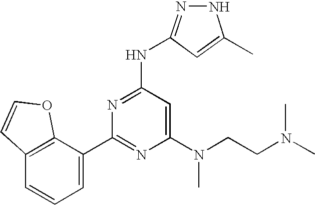 |
| 206 | 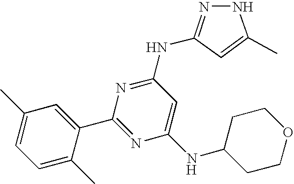 |
| 207 | 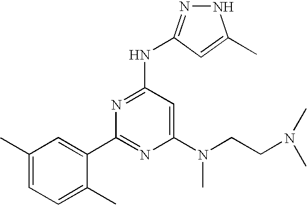 |
| 208 | 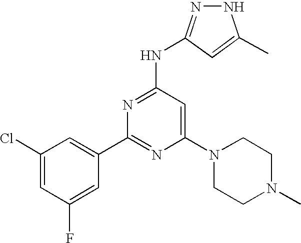 |

In one embodiment, this invention provides a composition comprising a compound of formula I or formula II, and a pharmaceutically acceptable carrier, adjuvant or vehicle. In some such embodiments, the composition is for treating or preventing a kinase mediated disorder.

In one embodiment, the carrier is suitable for oral, parenteral, inhalation, topical, or intradermal administration.

In another embodiment, the composition is incorporated into a biodegradable or non-biodegradable polymer.

In still another embodiment, the composition of comprises a compound of formula I and an additive. The additive may be selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

In another embodiment, this invention relates to a method of treating or preventing a kinase mediated disease, which method comprises administering to a patient in need of such a treatment a therapeutically effective amount of a compound of formula I, or a pharmaceutical composition thereof.

In some aspects of the aforementioned methods and compositions, the disorder is mediated by Aurora A, Aurora B, CDK-2, ERK-2, AKT, Src, Lck, Abl, cKit, Flt3, or KDR. In other aspects, the disorder is mediated by Aurora A, Src, Lck, Abl, cKit, Flt3, or KDR.

In one embodiment, a method of treating a patient with a cancer is provided comprising administering to the patient having the cancer an effective cancer-treating amount of a compound of formula I.

In another embodiment, the a method of treating a patient with a cancer is provided, wherein the cancer is a solid tumor, blood borne tumor, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity, pharynx, lip, tongue, mouth, pharynx, small intestine, colon-rectum, large intestine, rectum, brain and central nervous system, or leukemia.

In still another embodiment, a method of treating a patient with a disease associated with undesirable neovascularization is provided comprising administering to the patient with the undersirable neovascularization an effective amount of a composition comprising a compound of formula I.

In another embodiment, the disease associated with undesirable neovasculariation comprises ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, or corneal graph rejection, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, or post-laser complications.

Another aspect of this invention relates to a method of inhibiting Aurora A activity in a patient, which method comprises administering to the patient a compound of formula I or a composition comprising said compound.

Another aspect of this invention relates to a method of treating or preventing a GSK-3-mediated disease with a GSK-3 inhibitor, which method comprises administering to a patient in need of such a treatment a therapeutically effective amount of a compound of formula I or a pharmaceutical composition thereof.

Another embodiment comprises a method of treating a patient with an inflammatory disease associated with inflammation comprising administering to the patient with the inflammatory disease an effective amount of a compound of formula I. The inflammatory disease may be excessive or abnormal stimulation of endothelial cells, atherosclerosis, vascular malfunctions, abnormal wound healing, inflammatory and immune disorders, Bechet's disease, gout or gouty arthritis, abnormal angiogenesis accompanying rheumatoid arthritis, skin diseases, psoriasis, diabetic retinopathy, retinopathy of prematurity, retrolental fibroplasic), macular degeneration, corneal graft rejection, neovascular glaucoma or Osler Weber syndrome.

In still another embodiment, a method of treating patient with a GSK-3 mediated disease is provided comprising administering to the patient with the GSK-3 mediated disease an effective amount of a compound of formula I. In some embodiments, the GSK-3 mediated disease is diabetes, Alzheimer's disease, Huntington's Disease, Parkinson's Disease, AIDS-associated dementia, amyotrophic lateral sclerosis (AML), multiple sclerosis (MS), schizophrenia, cardiomycete hypertrophy, reperfusion/ischemia, or baldness.

In some embodiments, the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

One aspect of this invention relates to a method of enhancing glycogen synthesis and/or lowering blood levels of glucose in a patient in need thereof, which method comprises administering to the patient a therapeutically effective amount of a compound of formula I, or a pharmaceutical composition thereof. This method is especially useful for diabetic patients. Another method relates to inhibiting the production of hyperphosphorylated Tau protein, which is useful in halting or slowing the progression of Alzheimer's disease. Another method relates to inhibiting the phosphorylation of beta-catenin, which is useful for treating schizophrenia.

Another aspect of this invention relates to a method of inhibiting GSK-3 activity in a patient, which method comprises administering to the patient a compound of formula I, or a composition comprising said compound.

Another aspect of this invention relates to a method of treating or preventing a Src-mediated disease with a Src inhibitor, which method comprises administering to a patient in need of such a treatment a therapeutically effective amount of a compound of formula I or a pharmaceutical composition thereof.

Another aspect of the invention relates to inhibiting Src activity in a patient, which method comprises administering to the patient a compound of formula I, or a composition comprising said compound.

Another method relates to inhibiting Aurora A, GSK-3, or Src activity in a biological sample, which method comprises contacting the biological sample with the Aurora A, GSK-3, or Src inhibitor of formula I, or a pharmaceutical composition thereof, in an amount effective to inhibit Aurora-2, GSK-3, or Src.

Each of the aforementioned methods directed to the inhibition of Aurora A, GSK-3, or Src, or the treatment of a disease alleviated thereby, is carried out with a compound of formula I, as described above.

The present invention also relates to the processes for preparing the compounds of the invention and to the synthetic intermediates useful in such process, as described below and in the Examples.

### Synthetic Processes

Schemes 1 through 4 below and the experimental description in the examples outline the synthetic methods used to prepare the compounds of the present invention. It is understood that the synthetic transformations outlined below can be carried out with a variety of alternate reagents that function to achieve the desired reaction.

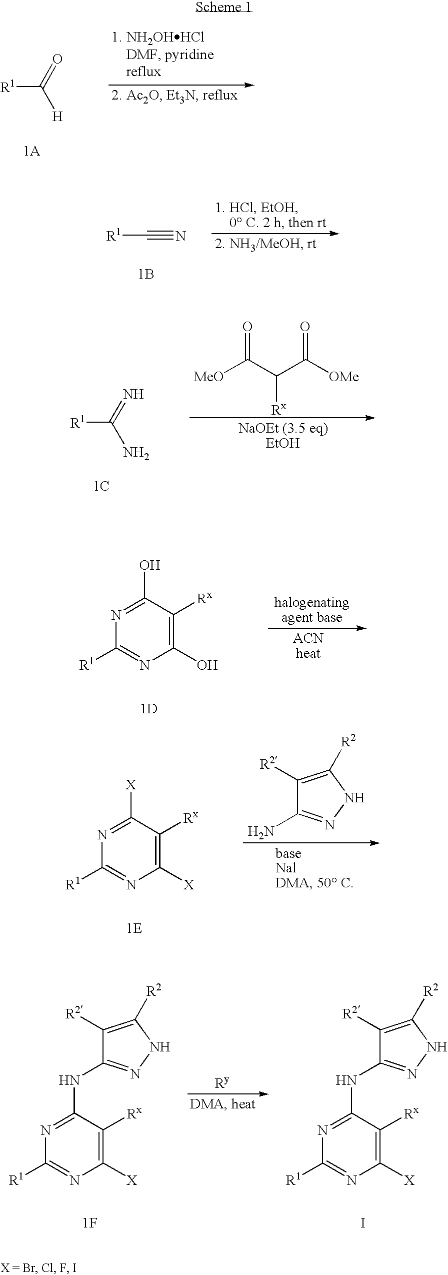

In another aspect of this invention, preferred embodiments of Formula I can be synthesized as shown in Scheme 1 wherein the variable substituents are as described above and examples of which are indicated by Table 1. Aldehyde 1-A can be converted to nitrile 1-B by a two step process (as described in Hilton et al Org. Lett. 2000, 2, 2639). The aldehyde is first converted to the corresponding oxime with hydroxylamine hydrochloride in ethanol. The resulting oxime is converted to the corresponding nitrile via an elimination reaction, using for example, acetic anhydride and triethylamine to give 1-B. Examples of 1-A include, but are not limited to, benzofuran-2-carboxaldehyde and 1-methylindole-2-carboxaldehyde. Nitrile 1-B is converted to an amidine via a Pinner reaction using, for example, anhydrous ethanol and dry HCl gas to give the corresponding ethyl amidate as an intermediate, which is then converted to amidine 1-C under basic conditions with, for example, methanolic ammonia or sodium methoxide. Pyrimidinone 1-D is prepared by condensation of 1-C, under basic conditions, with a reagent such as dimethylmalonate. Position 5 of the pyrimidinone can optionally be substituted by using a reagent such as a dialkylmalonate substituted by R^{x} as shown in Scheme 1. Pyrimidinone 1-D can be converted to 4,6-dihalogenpyrimidine 1-E using a halogenating reagent and a base. In one embodiment, the halogenating reagent is POCl₃ and the base is diisopropyl ethyl amine. The reaction can be carried out with or without the presence of an appropriate solvent, such as acetonitrile. Dihalogenpyrimidine 1-E can be substituted with a primary amine, including 3-amino-5-methyl-pyrazole to give pyrimidine 1-F. This substitution reaction can be done in a polar aprotic solvent, including for example dimethylacetamide, with a base, including diisopropylethylamine and optionally a catalyst, including NaI. Pyrimidine I is prepared by heating pyrimidine 1-F with an amine (R^{Y}), including for example N-methylpiperazine, either neat or in a high boiling aprotic solvent, including dimethyl acetamide.

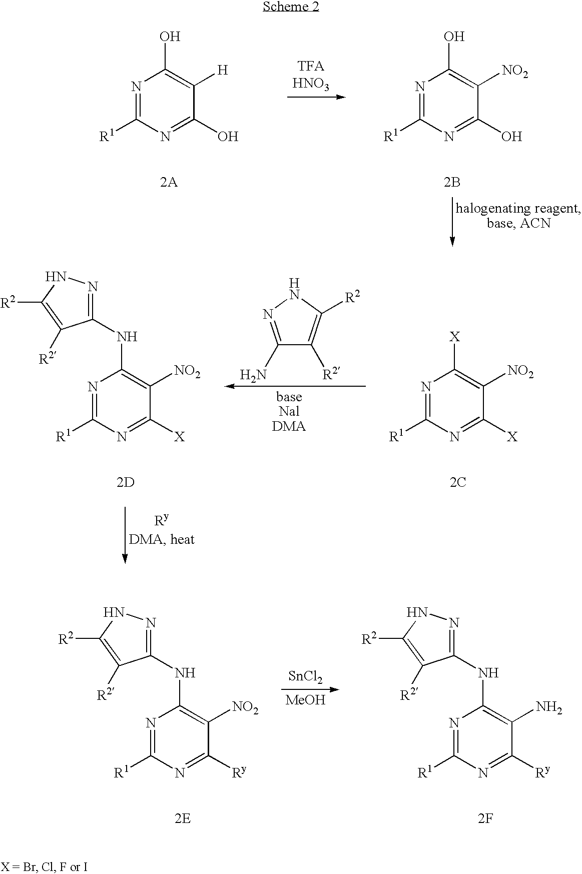

In still another aspect of this invention, embodiments of Formula I where R^{x} is NH₂ or NO₂ can be synthesized as shown in Scheme 2. Pyrimidinone 2A can be prepared as described in Scheme 1. Thus, pyrimidininone 2A can be converted to 5-nitro-pyrimidinone 2B using nitric acid and a acid such as, but not limited to, trifluoroacetic acid. Resulting intermediate 2B can be converted to the corresponding dichloropyrimidine 2C using a halogenating reagent and a base. In one embodiment, the halogenating reagent is POCl₃ and the base is diisopropyl ethyl amine. The reaction can be carried out with or without the presence of an appropriate solvent, such as acetonitrile. Dihalogenpyrimidine 2C can be substituted with a primary amine, including 3-amino-5-methylpyrazole to give substituted halogenpyrimidine 2D. Pyrimidine 2D can react with a nucleophile, such as N-methylpiperazine, either neat or in a high boiling solvent including dimethylacetamide to give 2E. Diaminopyrmidine 2E can be converted to 5-aminopyrimidine 2F using a suitable chemical reducing agent such as, but not limited to, tin (II) chloride or titanium (II) chloride, in dilute hydrochloric acid and a solvent such as, but not limited to, methanol. One skilled in the art can envision that the amine can serve as a synthetic handle to yield substituted amines or amides through known chemical reactions.

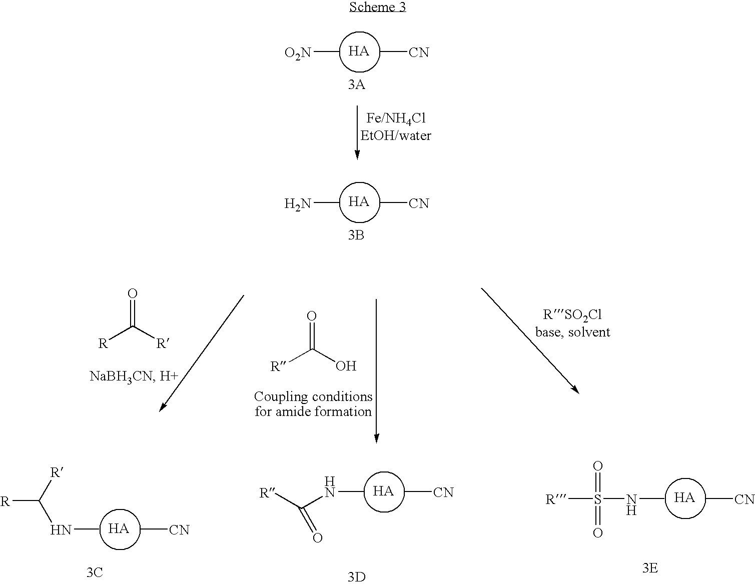

Scheme 3 can be used to prepare substituted heteroaryl systems. Heteroaryl (HA) can include examples such as, but not limited to, benzofurans, indoles and benzothiophenes. The nitro group in 3A can be reduced by Fe and ammonium chloride in an alcohol/water solvent system to give amine 3B. The resulting amine 3B can be alkylated by a standard reductive amination method to give 3C, or converted to amide 3D by a number of different coupling methods known to one skilled in the art; or alternatively, converted to sulfonamide 3D by coupling with the appropriate sulfonyl chloride under basic conditions. Nitro group reduction, reductive amination, amide coupling methods and preparation of sulfonamides are standard chemical reactions known to one skilled in the art. These and other standard organic synthesis reactions described herein are described in March's “Advanced Organic Chemistry” 5^{th} Edition, Wiley-Interscience NY, N.Y., 2001, pp. 1552, 1188, 1652-1653 and 1687 respectively.

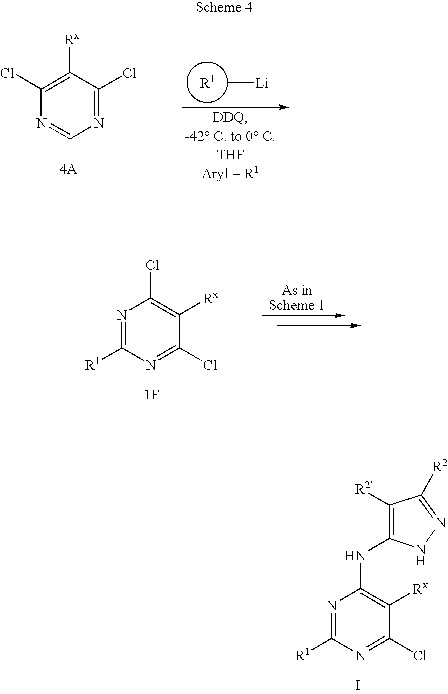

An alternate route to prepare substituted pyrimidines is shown in Scheme 4. The first step in this sequence is the substitution of position 2 of pyrimidine 4A with an aryl lithium (R¹Li) reagent (for a related reference see: Harden et al J. Org. Chem. 1988, 53, 4137) to give 2-aryl-4,6-dichloropyrimidine 1F. The aryl lithium reagent in this sequence can be purchased from a commercial source or generated via lithium-halogen exchange or heteroatom directed metallation chemistry from the appropriate aryl bromide or heteroaryl precursor using n-butyl lithium or another appropriate alkyl lithium base (a typical lithium halogen-exchange procedure can be found in: Harder et al Organomet. 1990, 9, 511; or Jiabi et al. J. Organomet. Chem. 1985, 286, 55. For a heteroatom directed metallation procedure: Harden et al J. Org. Chem. 1988, 53, 4137). Compound I can be prepared from 1F using the chemistry previously described in Scheme 1.

Scheme 5 can also be used to prepare pyrimidine analogs. In the first step, the most reactive halogen of 2,4,6 trichloropyrimidine (5A) can be replaced by an aminopyrazole to give pyrimidine 5B. The reaction can be done at room temperature in a solvent such as DMA and an added base such as N,N-diisopropyl ethylamine. In the second step a halogen is replaced with an amine (R^{y}) to give pyrimidine 5C. Regioisomers of 5C are possible and can be separated by standard purification techniques such as chromatography or crystallization. The last step of Scheme 5 uses Suzuki coupling conditions to couple 5C with the desired boronic acid [R¹—B(OH)₂] or boronic ester [R¹⁻B(Oalkyl)₂] to yield 5D. This reaction typically uses a palladium catalyst, a base and solvent, and can be done at elevated temperatures or in a microwave reactor (for a general reference on the Suzuki Reaction and other named reactions see: Laszlo Kurti, Barbara Czako “Strategic Applications of Named Reactions in Organic Synthesis” Elsevier Academic Press, NY, N.Y. 2005).

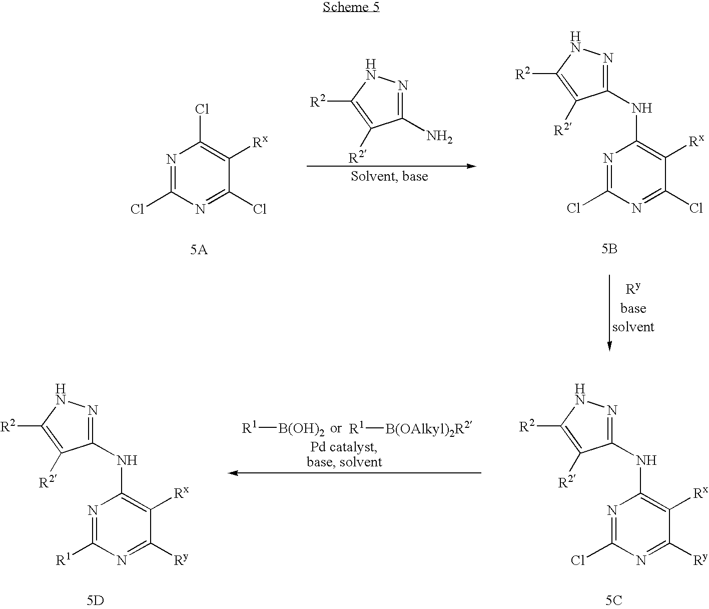

Where necessary in any of the synthetic procedures described herein, appropriate protecting groups may be used. Examples of protection groups can be found in the literature including “Protective Groups in Organic Synthesis—Third Edition” (T. W. Greene, P. G. M. Wuts, Wiley-Interscience, New York, N.Y., 1999). The present invention will be understood more readily by reference to the following examples, which are provided by way of illustration and are not intended to be limiting of the present invention.

### EXAMPLES

The following abbreviations are used in the examples:
ATP: adenosine triphosphate
ACN: Acetonitrile
Brij-35: polyoxyethyleneglycol dodecyl ether
° C.: degrees Celcius
DMEM: Dulbecco's Modified Eagle's Medium
DMF: N,N-dimethylformamide
DMA: N,N-dimethylacetamide
DMSO: dimethylsulfoxide
DTT: dithiothreitol
EtOAc: ethyl acetate
g: gram
h: hour
H¹ NMR: proton nuclear magnetic resonance
HEPES: 4-(2-hydroxyethyl)piperazine-1-ethanesulfonic acid
Hz; Hertz
hplc: high performance liquid chromatography
IC₅₀ value: concentration of an inhibitor that causes a 50% reduction in a measured activity.
mg: milligram
MHz: megaHertz
mL: milliliter
mmol: millimole
MS: mass spectrum
M/e: mass to charge ratio
Pz: optionally modified or substituted or fused pyrazole ring system
Pet ether: petroleum ether
ppt: precipitation
Rf: ratio to front (ratio of distance traveled by substance/distance traveled by solvent)
SRB: sulphorhodamine-B
TCA: trichloracetic acid
THF: tetrahydrofuran
tlc: thin layer chromatography
br: broad
s: singlet
d: doublet
t: triplet
q: quartet
dd: doublet of doublets
m: multiplet
J: coupling constant
RT: room temperature
δ: part per million
   Additional abbreviations used herein are described in The ACS Style Guide. 3rd Edition Edited by Anne M. Coghill and Lorrin Garson. Oxford University Press, New York. 2006. xiv +430 pp. 18×20.5 cm. ISBN 13: 978-0-8412-3999-9.

### Experimental

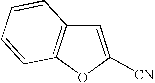

### benzofuran-2-carbonitrile

Pyridine (5.41 g, 0.0684 mol) was added to a stirred solution of 2-benzofurancarboxaldehyde (5 g, 34.2 mmol) in ethanol (100 ml) and hydroxylamine HCl (2.83 g, 41 mmol) at RT. The mixture was heated at 90° C. for 2 h until completion, cooled to rt, then concentrated. The resulting residue was taken up with water (200 ml), and extracted with EtOAc (3×200 ml), washed with brine (100 ml), dried (Na₂SO₄) and concentrated to afford benzofuran-2-oxime product (5.5 g, 99.8%) as a yellow liquid. R_{f}; 0.7 (Pet ether: EtOAc; 9.5:0.5). This product was converted to the nitrile without further purification. Acetic anhydride (3.8 g, 37.3 mmol) was added to a stirred solution of benzofuran-2-oxime (5.5 g, 186 mmol) in triethylamine (25 ml) over a period of 15 min at 0° C., and heated at 90° C. for 2 h. When the reaction was complete, it was cooled to RT, quenched with water (100 ml), extracted with ethyl acetate (2×100 ml), washed with brine (50 ml), dried (Na₂SO₄), and concentrated to afford crude benzofuran-2-carbonitrile (4.2 g, 87.5%) as a pale yellow solid. R_{f}: 0.8 (Pet ether:EtOAc; 9:1). ¹H NMR (300 MHz, CDCl₃): δ 7.6 (d, 1H, J=6 Hz); δ 7.59-7.35 (m, 4H).

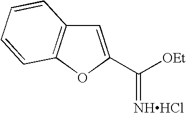

### benzofuran-2-ethyl amidate HCl salt

Absolute ethanol (14.88 ml, 26.4 mmol) was added to an ice cooled stirred solution of benzofuran-2-carbonitrile (4.2 g, 29.3 mmol) in toluene (40 ml). Dry HCl gas was purged to the reaction mixture for 2 h at 0° C., and continued stirring at RT for 36 h until complete. The reaction mixture was concentrated under vacuum at 40° C., and washed with pet ether (100 ml) and dried under vacuum for 10 h to afford benzofuran-2-ethylamidate HCl salt (5 g, 91%) as a off white solid. R_{f}; 0.4 (Pet ether:EtOAc; 8:2). ¹H NMR (300 MHz, DMSO-d₆): δ 8.35 (s, 1H), 8.1 (d, 1H, J=7.8 Hz), 7.79-7.31 (m, 4H), 4.66 (q, 2H, J=7 Hz), 1.47 (t, 3H, J=7 Hz). M/e (M+1): 190.

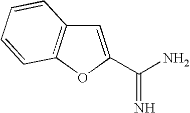

### benzofuran-2-carboxamidine

Methanolic ammonia (50 ml, 79.3 mmol) was added to a stirred solution of benzofuran-2-ethylamidate HCl salt (5 g, 26.4 mol) in methanol (25 ml) at RT and stirred for 36 h. The reaction mixture was concentrated when complete. The concentrate was dissolved in methanol (20 ml), and acidified with saturated methanolic HCl to adjust pH 1-2. Then, the reaction mixture was concentrated and dried under vacuum for 8 h to afford benzofuran-2-carboxamidine (4 g, quant.) as a pale yellow solid. R_{f}; 0.14 (Pet ether:EtOAc; 7:3). ¹H NMR (300 MHz, DMSO-d₆): δ 9.79 (br s, 2H), 9.42 (br s, 1H), 8.32 (br s 1H), 7.88 (m, 1H), 7.70 (m, 1H), 7.58 (m, 1H), 7.40 (m, 1H). M/e (M+1): 161.

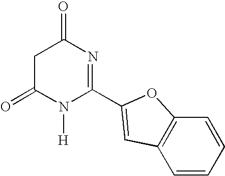

### 2-(benzofuran-2-yl)pyrimidine-4,6(1H,5H)-dione

Freshly prepared sodium methoxide (2.646 g, 49.0 mmol) in methanol was added dropwise to a stirred solution of benzofuran-2-carboxamidine (3 g, 12.3 mmol) in methanol (20 ml) over a period of 30 min at RT. Dimethylmalonate (1.4 ml, 12.3 mmol) was added to the reaction mixture and continued stirring at 40° C. for 12 h. When the reaction was complete, it was diluted with water (100 ml) and acidified with 1.5 N HCl to adjust pH 2-3. The solid obtained was filtered, washed with water (50 ml), diethyl ether (50 ml), chloroform (25 ml) and dried under vacuum to afford 2-(benzofuran-2-yl)pyrimidine-4,6(1H,5H)-dione (1.5 g, 51%) as a yellow solid. R_{f}; 0.1 (pet ether:EtOAc; 7:3). ¹H NMR (300 MHz, DMSO-d₆) δ 7.89 (s, 1H), 7.85 (d, 1H, J=7.7 Hz), 7.6 (d, 1H, J=8.4 Hz), 7.50-7.45 (m, 1H), 7.37-7.32 (m, 1H), 5.4 (br s, 1H), M/e (M+1): 229.

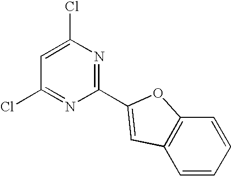

### 2-(benzofuran-2-yl)-4,6-dichloropyrimidine

Diethylisopropylamine (2.52 ml, 26.2 mmol) was added to a stirred solution of 2-(benzofuran-2-yl)pyrimidine-4,6(1H,5H)-dione (2 g, 8.8 mmol) in POCl₃ (7.33 ml, 79 mmol) at RT. When the addition was complete, the reaction mixture was heated at 90° C. for 16 h until completion. The reaction mixture was then cooled to RT, quenched with ice cold water (50 ml) and extracted with ethyl acetate (2×75 ml), washed with water (25 ml), 10% NaHCO₃ solution (25 ml), brine (25 ml), dried (Na₂SO₄) and concentrated to afford crude product. The crude product was stirred with methanol and the solid was filtered to afford 2-(benzofuran-2-yl)-4,6-dichloropyrimidine (1.5 g, 65%) as a yellow solid R_{f}; 0.3 (Pet ether: EtOAc; 9:1) ¹H NMR (300 MHz, DMSO-d₆): δ 7.99 (s, 1H), 7.93 (s, 1H), 7.82-7.76 (m, 2H), 7.53-7.48 (m, 1H), 7.39-7.34 (m, 1H). M/e (M+1): 265.

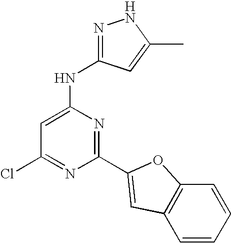

### 2-(benzofuran-2-yl)-6-chloro-N-(5-methyl-1H-pyrazol-3-yl)pyrimidin-4-amine

5-Methyl 3-amino pyrazole (0.65 g, 6.7 mmol) was added to a stirred solution of 2-(benzofuran-2-yl)-4,6-dichloropyrimidine (1.5 g, 5.7 mmol) in dimethylacetamide (10 ml). Diisopropyl ethylamine (0.812 ml, 8.5 mmol) was added to the reaction mixture followed by the sodium iodide (1.27 g, 8.5 mmol) at rt. The reaction mixture was then heated at 90° C. for 24 h. The reaction mixture was then cooled to rt and quenched with ice cold water (50 ml) extracted with ethyl acetate (2×100 ml), washed with 10% sodium bicarbonate solution (25 ml), brine (25 ml), dried over Na₂SO₄ and concentrated. The solid obtained was stirred with methanol filtered and washed with methanol (25 ml), dried under vacuum to afford 2-(benzofuran-2-yl)-6-chloro-N-(5-methyl-1H-pyrazol-3-yl)pyrimidin-4-amine (0.95 g, 44%) as a yellow solid. R_{f}; 0.4 (CHCl₃:MeOH; 9:1). ¹H NMR (300 MHz, DMSO-d₆): δ 12.15 (s, 1H), 10.47 (s, 1H), 7.85 (d, 1H, J=7.4 Hz), 7.73-7.65 (m, 3H), 7.48-7.43 (m, 1H), 7.36-7.31 (m, 1H), 7.09 (s, 1H), 7.04 (s, 1H), 2.26 (s, 3H). M/e (M+1): 326.

### Example 1

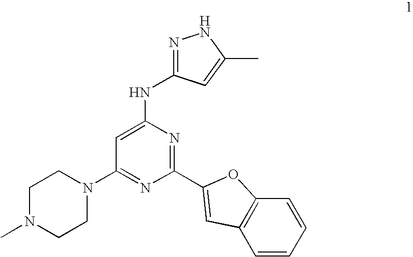

### 2-(benzofuran-2-yl)-N-(5-methyl-1H-pyrazol-3-yl)-6-(4-methylpiperazin-1-yl)pyrimidin-4-amine

N-methyl piperazine (1.5 ml) was added to 2-(benzofuran-2-yl)-6-chloro-N-(5-methyl-1H-pyrazol-3-yl)pyrimidin-4-amine (0.3 g, 0.9 mmol) at RT, and heated at 90° C. for 3 h. The reaction mixture was then cooled to RT, and quenched with water (25 ml). The solid obtained was filtered and washed with water (25 ml), and purified through column chromatography using 2% chloroform in methanol, to afford 2-(benzofuran-2-yl)-N-(5-methyl-1H-pyrazol-3-yl)-6-(4-methylpiperazin-1-yl)pyrimidin-4-amine (30 mg, 9.85%) as an off white solid. R_{f}; 0.5 (CHCl₃:MeOH; 9:1). ¹H NMR (400 MHz, CD₃OD): δ 7.73 (d, 1H, J=7.4 Hz), 7.62-7.60 (m, 2H), 7.44-7.41 (m, 1H), 7.3 4-7.32 (m, 1H), 6.4 (br s, 1H), 6.2 (br s, 1H), 3.92 (br s, 4H), 3.3 (br s, 4H), 2.75 (s, 3H), 2.34 (s, 3H). M/e (M+1): 390.3; HPLC purity: >92%.

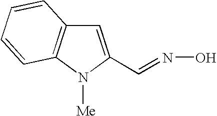

### 1-methyl-1H-indole-2-oxime

Pyridine (9.93 g, 125.6 mmol) followed by hydroxyl amine HCl (5.2 g, 75.3 mmol) was added to a stirred solution of 1-methyl indole-2-carboxaldehyde (10 g, 62.81 mmol) in ethanol (300 ml) at rt and heated at 90° C. for 2 h until complete. The reaction mixture was then concentrated. The concentrate was taken up with water (200 ml), and extracted with EtOAc (3×250 ml). The combined organics were washed with brine (200 ml), dried (Na₂SO₄) and concentrated to afford 1-methyl-1H-indole-2-oxime (11 g, 100%) as a yellow solid. R_{f}; 0.14 (Pet ether:EtOAc; 9:1). ¹H NMR (400 MHz, DMSO-d₆): δ 8.32 (s, 1H), 7.63-7.56 (m, 1H), 7.52-7.46 (m, 1H), 7.23-7.20 (m, 1H), 7.08-7.04 (m, 1H), 6.76 (s, 1H), 3.94 (s, 2H), 3.86 (s, 1H). M/e (M+1): 175.2.

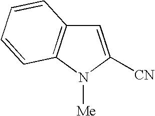

### 1-methyl-1H-indole-2-carbonitrile

Acetic anhydride (12.88 g, 126.2 mmol) was added to a stirred solution of 1-methyl indole-2-oxime (11 g, 63.1 mmol) in triethylamine (200 ml) and heated at 90° C. for 2 h until completion. The reaction mixture was cooled to RT, taken up with water (300 ml), extracted with ethyl acetate (2×250 ml). The combined organics were washed with brine (200 ml), dried (Na₂SO₄), and concentrated. The crude product was purified by column at 3% EtOAc in PE to afford 1-methyl-1H-indole-2-carbonitrile (7 g, 71%) as a white solid. R_{f}: 0.6 (Pet ether:EtOAc; 8:2). ¹H NMR (400 MHz, DMSO-d₆): δ 7.69-7.67 (m, 1H), 7.61-7.59 (m, 1H), 7.41-7.38 (m, 2H), 7.21-7.16 (m, 1H), 3.88 (s, 3H). M/e (M+1): 157.

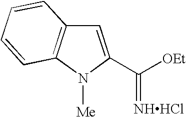

### 1-methyl-1H-indole-2-ethyl amidate HCl salt

Absolute ethanol (23.8 ml, 403.6 mol) was added to an ice cooled stirred solution of 1-methyl-1H-indole-2-carbonitrile (7 g, 44.8 mol) in toluene (200 ml). Dry HCl gas was bubbled through the reaction mixture for 2 h at 0° C., and stirring was continued RT for 36 h. The reaction mixture was concentrated under vacuum at 40° C., and washed with pet ether (200 ml) and dried under vacuum to afford 1-methyl-1H-indole-2-ethyl amidate HCl salt (9 g, 99%) as a dark brown solid. R_{f}; 0.5 (PE:EtOAc; 6:4). ¹H NMR (400 MHz, DMSO-d₆): δ 11.79 (br s, 1H), 7.85 (s, 1H), 7.75 (d, 1H, J=8 Hz), 7.66 (d, 1H, J=8 Hz), 7.46-7.42 (m, 1H), 7.21-7.17 (m, 1H), 4.72 (q, 2H, J=8 Hz), 3.95 (s, 3H), 1.41 (t, 3H, J=8 Hz). M/e (M+1): 203.1.

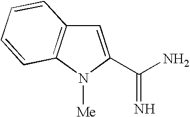

### 1-methyl-1H-indole-2-carboxamidine

Methanolic ammonia (27 ml, 222.4 mmol) was added to a stirred solution of 1-methyl-1H-indole-2-ethyl amidine HCl salt (9 g, 44.4 mmol) in methanol (50 ml) at RT and stirred for 36 h. The reaction mixture was concentrated then dissolved in methanol (50 ml), and acidified with saturated methanolic HCl to pH˜1-2. Then, the reaction mixture was concentrated and dried under vacuum to afford 1-methyl-1H-indole-2-carboxamidine (7.7 g, quant.) as dark brown solid. R_{f}; 0.4 (CHCl₃:MeOH; 8:2). ¹H NMR (400 MHz, DMSO-d₆): δ 9.48 (br s, 1H), 9.46 (br s 2H), 7.72 (d, 1H, J=8 Hz), 7.64 (d, 1H, J=8 Hz), 7.41-7.37 (m, 1H), 7.20-7.16 (m, 2H), 3.88 (s, 3H). M/e (M+1): 174.

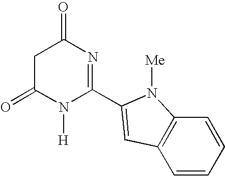

### 2-(1-methyl-1H-indol-2-yl)pyrimidine-4,6(1H,5H)-dione

Freshly prepared sodium methoxide (11.9 g, 222.2 mmol) in methanol (100 ml) was added dropwise to a stirred solution of 1-methyl-1H-indole-2-carboxamidine (7.7 g, 44.4 mmol) and dimethylmalonate (7.04 g, 53.3 mmol) in methanol (100 ml) over a period of 30 min at RT. Then the reaction mixture was heated to 45° C., and stirred for 12 h. The reaction mixture was then concentrated under reduced pressure and diluted with water (150 ml) and acidified with 1.5 N HCl to adjust pH 2-3. The precipitate was isolated in a Buchner funnel, washed with water (200 mL) followed by diethyl ether (100 ml) and dried under vacuum to afford 2-(1-methyl-1H-indol-2-yl)pyrimidine-4,6(1H,5H)-dione (7 g, 65%) as a brown solid. R_{f}; 0.5 (CHCl₃:MeOH; 9:1). ¹H NMR (400 MHz, DMSO-d₆): δ 12.4 (br s 1H), 11.43 (br s, 1H), 7.64 (d, 1H, J=8 Hz), 7.56 (d, 1H, J=8.4 Hz), 7.39 (s, 1H), 7.33-7.29 (m, 1H), 7.13-7.09 (m, 1H), 5.33 (s, 1H), 4.09 (s, 3H). M/e (M+1): 242.

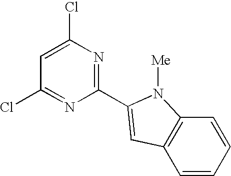

### 2-(4,6-dichloropyrimidin-2-yl)-1-methyl-1H-indole

Diethylisopropylamine (10 ml) was added to a stirred solution of 2-(1-methyl-1H-indol-2-yl)pyrimidine-4,6(1H,5H)-dione (7 g, 29 mmol) in POCl₃ (150 ml) at 0° C., and heated to 90° C. for 16 h. The reaction mixture was concentrated, taken up with ice cold water (200 ml) and extracted with ethyl acetate (3×200 ml). The combined organics were washed with 1.5N HCl (2×200 ml) and brine (200 ml), dried (Na₂SO₄) and concentrated to afford crude product. The crude product was purified by column chromatography using 20% EtOAc in pet. ether to afford 2-(4,6-dichloropyrimidin-2-yl)-1-methyl-1H-indole (4 g, 50%) as light yellow solid R_{f}; 0.7 (Pet ether:EtOAc; 9:1) ¹H NMR (400 MHz, DMSO-d₆): δ 7.87 (s, 1H), 7.68 (d, 1H, J=7.6 Hz), 7.58 (d, 1H, J=8 Hz), 7.48 (s, 1H), 7.35-7.33 (m, 1H), 7.14-7.10 (m, 1H), 4.15 (s, 3H). M/e (M+1): 279.

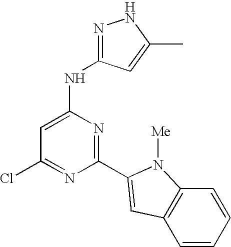

### 6-chloro-2-(1-methyl-1H-indol-2-yl)-N-(5-methyl-1H-pyrazol-3-yl)pyrimidin-4-amine

5-Methyl-3-amino pyrazole (0.2 g, 2.1 mmol) was added to a stirred solution of 2-(4,6-dichloropyrimidin-2-yl)-1-methyl-1H-indole (0.5 g, 1.7 mmol) in dimethylacetamide (15 ml). Diisopropyl ethylamine (0.4.6 ml, 2.6 mmol) was then added to the reaction mixture followed by KI (0.44 g, 2.6 mmol) at rt. The mixture was heated at 90° C. for 24 h and cooled to rt. The mixture was quenched with ice cold water (50 ml) extracted with ethyl acetate (3×100 ml) and the combined organics were washed with 10% sodium bicarbonate solution (100 ml), brine (50 ml), dried over Na₂SO₄ and concentrated. The solid obtained was purified by column using 30% EtOAc in pet. ether to afford 6-chloro-2-(1-methyl-1H-indol-2-yl)-N-(5-methyl-1H-pyrazol-3-yl)pyrimidin-4-amine (0.42 g, 69%) as light yellow solid. R_{f}: 0.5 (PE:EtOAc; 1:1). ¹H NMR (400 MHz, CD₃OD): δ 7.62 (d, 1H, J=8 Hz), 7.44 (d, 1H, J=8.4 Hz), 7.35 (s, 1H), 7.30-7.25 (m, 1H), 7.11-7.07 (m, 2H), 6.20 (br s, 1H), 4.19 (s, 3H), 2.33 (s, 3H). M/e (M+1): 339.

### Example 2

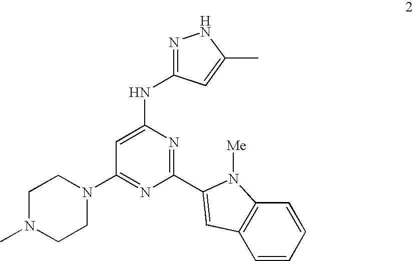

### 2-(1-methyl-1H-indol-2-yl)-N-(5-methyl-1H-pyrazol-3-yl)-6-(4-methylpiperazin-1-yl)pyrimidin-4-amine

N-Methyl piperazine (0.6 ml) was added to 6-chloro-2-(1-methyl-1H-indol-2-yl)-N-(5-methyl-1H-pyrazol-3-yl)pyrimidin-4-amine (0.4 g, 1.2 mmol) at RT, and heated at 90° C. for 2 h then cooled to RT. The reaction was quenched with water (25 ml) and stirred for 2 h. The resulting ppt was isolated with a Buchner funnel and washed with water (25 ml). The ppt was purified further by triturated with MeOH and diethyl ether to afford 2-(1-methyl-1H-indol-2-yl)-N-(5-methyl-1H-pyrazol-3-yl)-6-(4-methylpiperazin-1-yl)pyrimidin-4-amine (30 mg, 9.85%) as an off white solid. R_{f}: 0.2 (CHCl₃:MeOH; 9:1). ¹H NMR (400 MHz, CD₃OD): δ 7.60 (d, 1H, J=8 Hz), 7.44 (d, 1H, J=8 Hz), 7.26-7.23 (m, 2H), 7.09-7.05 (m, 1H), 6.39 (br s, 1H), 6.19 (br s, 1H), 4.19 (s, 3H), 3.7 (br s, 4H), 2.57 (br s, 4H), 2.36 (s, 3H), 2.30 (s, 3H). M/z (M+1): 403; HPLC purity >99%.

### Example 3

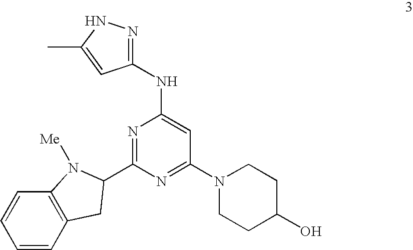

### 1-(6-(5-methyl-1H-pyrazol-3-ylamino)-2-(1-methyl-1H-indol-2-yl)pyrimidin-4-yl)piperidin-4-ol

Piperazin-1-ol (0.5 ml) was added to 6-chloro-2-(1-methyl-1H-indol-2-yl)-N-(5-methyl-1H-pyrazol-3-yl)pyrimidin-4-amine (0.2 g, 0.6 mmol) at RT, and the reaction mixture was heated to 90° C. for 2 h. The reaction mixture was quenched with water (10 ml) to give a ppt which was isolated with a Buchner funnel giving 1-(6-(5-methyl-1H-pyrazol-3-ylamino)-2-(1-methyl-1H-indol-2-yl)pyrimidin-4-yl)piperidin-4-ol (175 mg, 70.07%) as a R_{f} 0.4 (CHCl₃:MeOH; 9:1). ¹H NMR (400 MHz, DMSO-d₆): δ 11.89 (br s, 1H), 9.21 (s, 1H), 7.61 (d, 1H, J=8 Hz), 7.51 (d, 1H, J=8 Hz), 7.24-7.20 (m, 2H), 7.17 (s, 1H), 7.08-7.04 (m, 1H), 6.70 (br s, 1H), 6.02 (br s, 1H), 4.76 (s, 1H), 4.19 (s, 3H), 4.04-4.01 (m, 2H), 3.76-3.72 (m, 1H), 3.22 (t, 2, J=12 Hz), 2.21 (s, 3H), 1.98 (br s, 2H), 1.42-1.37 (m, 2H). M/e (M+1): 404.1; HPLC purity: >94%.

### Example 4

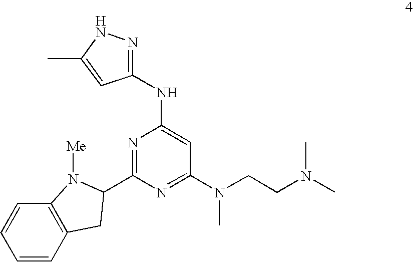

### N⁴-(2-(dimethylamino)ethyl)-N⁴-methyl-2-(1-methyl-1H-indol-2-yl)-N⁶-(5-methyl-1H-pyrazol-3-yl)pyrimidine-4,6-diamine

N,N,N′-Trimethylethane-1,2-diamine (0.5 ml) was added to 6-chloro-2-(1-methyl-1H-indol-2-yl)-N-(5-methyl-1H-pyrazol-3-yl)pyrimidin-4-amine (0.2 g, 0.6 mmol) at RT, and heated to 90° C. for 2 h. The reaction mixture was quenched with water (10 ml). The solid obtained was isolated with a Buchner funnel and washed with CH₂Cl₂ to afford N⁴-(2-(dimethylamino)ethyl)-N⁴-methyl-2-(1-methyl-1H-indol-2-yl)-N⁶-(5-methyl-1H-pyrazol-3-yl)pyrimidine-4,6-diamine (13 mg, 5.65%) R_{f} 0.4 (CHCl₃:MeOH; 9:1). ¹H NMR (400 MHz, DMSO-d₆): δ 11.87 (s, 1H), 9.16 (s, 1H), 7.61 (d, 1H, J=8 Hz), 7.51 (d, 1H, J=8 Hz), 7.23-7.18 (m, 2H), 7.07-7.05 (m, 1H), 6.50 (br s, 1H), 6.03 (br s, 1H), 4.20 (s, 3H), 3.66 (br s, 2H), 3.03 (s, 3H), 2.46-2.43 (m, 2H), 2.20 (s, 9H). M/e (M+1): 405.1; HPLC purity: >98%.

### Example 5

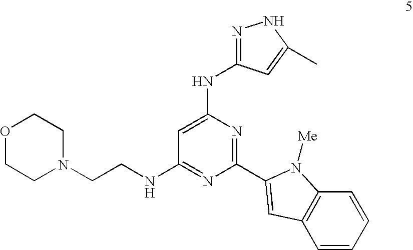

### 2-(1-methyl-1H-indol-2-yl)-N⁴-(5-methyl-1H-pyrazol-3-yl)-N⁶-(2-morpholinoethyl)pyrimidine-4,6-diamine

4-(2-Aminoethyl) morpholine (0.5 ml) was added to 6-chloro-2-(1-methyl-1H-indol-2-yl)-N-(5-methyl-1H-pyrazol-3-yl)pyrimidin-4-amine (0.20 g, 0.6 mmol) at RT, and heated at 90° C. for 2 h. The reaction mixture was cooled to RT, and quenched with water (25 ml). The solid obtained was stirred, isolated with a Buchner funnel and washed with water (25 ml). The crude product was purified through column chromatography using 2.5% methanol in chloroform to afford 2-(1-methyl-1H-indol-2-yl)-N⁴-(5-methyl-1H-pyrazol-3-yl)-N⁶-(2-morpholinoethyl)pyrimidine-4,6-diamine (145 mg, 69%) as an off white solid. R_{f}: 0.5 (CHCl₃:MeOH; 9:1). ¹H NMR (400 MHz, CD₃OD): δ 7.60 (d, 1H, J=7.6 Hz), 7.43 (d, 1H, J=7.6 Hz), 7.26-2.41 (m, 1H), 7.17 (s, 1H), 7.09-7.05 (m, 1H), 6.13 (br s, 1H), 6.05 (br s, 1H), 4.17 (s, 3H), 3.71 (t, 4H, J=4 Hz), 3.52 (br s, 2H), 2.64 (t, 2H, J=4 Hz), 2.55 (br s, 4H), 2.28 (s, 3H). M/e (M−1): 431.6. HPLC purity: >97%.

### Example 6

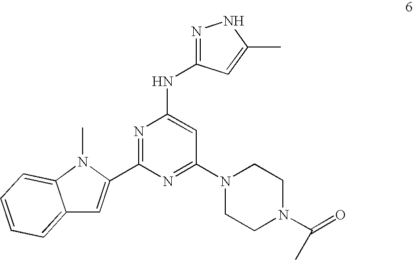

### 1-(4-(6-(5-methyl-1H-pyrazol-3-ylamino)-2-(1-methyl-1H-indol-2-yl)pyrimidin-4-yl)-piperazin-1-yl)ethanone

N-Acetyl piperazine (0.5 ml) was added to 6-chloro-2-(1-methyl-1H-indol-2-yl)-N-(5-methyl-1H-pyrazol-3-yl)pyrimidin-4-amine (0.2 g, 0.6 mmol) at RT, and then the reaction mixture was heated to 90° C. for 2 then quenched with water. The solid obtained was isolated with a Buchner funnel and dried under vacuum to afford 1-(4-(6-(5-methyl-1H-pyrazol-3-ylamino)-2-(1-methyl-1H-indol-2-yl)pyrimidin-4-yl)piperazin-1-yl)ethanone (200 mg, 74.07%) R_{f} 0.4 (CHCl₃:MeOH; 9:1). ¹H NMR (400 MHz, DMSO-d₆): δ 11.92 (s, 1H), 9.30 (s, 1H), 7.62 (d, 1H, J=8 Hz), 7.52 (d, 1H, J=8 Hz), 7.24-7.20 (m, 2H), 7.08 (t, 1H, J=7.6 Hz), 6.63 (br s, 1H), 6.03 (br s, 1H), 4.18 (s, 3H), 3.63-3.57 (m, 8H), 2.21 (s, 3H), 2.05 (s, 3H). M/e (M+1): >97%.

### Example 7

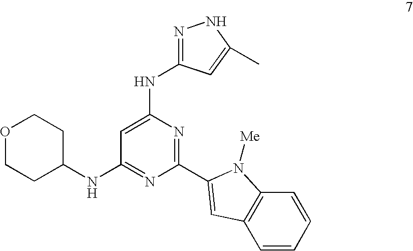

### N⁴-(tetrahydro-2H-pyran-4-yl)-2-(1-methyl-1H-indol-2-yl)-N⁶-(5-methyl-1H-pyrazol-3-yl)pyrimidine-4,6-diamine

4-Aminotetrahydropyran (0.4 ml) was added to 6-chloro-2-(1-methyl-1H-indol-2-yl)-N-(5-methyl-1H-pyrazol-3-yl)pyrimidin-4-amine (0.20 g, 0.6 mmol) at RT, and heated at 90° C. for 14 h then cooled to RT, and quenched with water (25 ml). The resulting ppt was stirred and isolated using a Buchner funnel and washed with water (25 ml). The crude product was purified using column chromatography using 50% EtOAc in pet ether to afford N⁴-(tetrahydro-2H-pyran-4-yl)-2-(1-methyl-1H-indol-2-yl)-N⁶-(5-methyl-1H-pyrazol-3-yl)pyrimidine-4,6-diamine (75 mg, 86%) as an off white solid. R_{f}: 0.2 (PE:EtOAc; 9:1). ¹H NMR (400 MHz, CD₃OD): δ 7.60 (d, 1H, J=8 Hz), 7.43 (d, 1H, J=8 Hz), 7.26-7.7.22 (m, 1H), 7.17 (s, 1H), 7.07 (m, 1H), 6.10 (br s, 1H), 5.99 (br s, 1H), 4.19 (s, 3H), 4.00-3.98 (m, 3H), 3.56 (t, 2H, J=8 Hz), 2.29 (s, 3H), 2.03-2.00 (m, 2H), 1.62-1.57 (m, 2H). M/e (M+1): 404. HPLC purity: >96%.

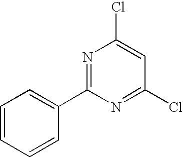

### 4,6-dichloro-2-phenylpyrimidine

N-Butyl lithium (3.22 g, 50.3 mmol, and 1.6N in hexane) was added dropwise to a stirred solution of bromobenzene (7.9 g, 50.3 mmol) in THF (70 mL) over a period of 30 min at −78° C., and reaction was continued stirring for 2 h. The generated phenyl lithium was added dropwise to a stirred solution of 4,6-dichloropyrimidine (5 g, 33.5 mmol) in THF (50 mL) over a period of 45 min at −78° C., and reaction was continued stirring for 30 min. Then, the reaction mixture was slowly heated to 0° C. and quenched with water (100 ml), DDQ (7 g, 30.8 mmol) dissolved in THF (70 mL) was added portionwise and stirred for 10 min. Then, the reaction mixture was washed with 10% NaOH (50 mL), extracted with CH₂Cl₂ (3×100 mL), washed with brine (100 mL), dried (Na₂SO₄) and concentrated. The concentrated product was purified through silica column chromatography using pet. ether to afford 4,6-dichloro-2-phenylpyrmimidine (example 21, 2.6 g, 35%) as a white solid. R_{f}: 0.3 (100% PE). ¹H NMR (400 MHz, CD₃OD): δ 8.41-8.39 (m, 2H), 7.60 (s, 1H), 7.58-7.50 (m, 3H). m/e (M+1): 224.8.

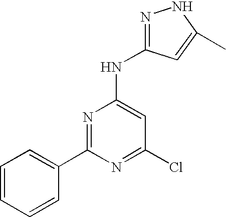

### 6-chloro-N-(5-methyl-1H-pyrazol-3-yl)-2-phenylpyrimidin-4-amine

3-Amino 5-methyl-pyrazole (1.3 g, 13.3 mmol) was added to a stirred solution of mixture of Example 21 (2.5 g, 11.1 mmol) in dimethylacetamide (25 mL), and diisopropyl ethylamine (2.15 g, 16.7 mmol) at RT. Then, sodium iodide (2.5 g, 16.7 mmol) was added to the reaction mixture at the same temperature. After addition, reaction mixture was heated at 80° C. for 16 h. After completion of reaction, reaction mixture was cooled to RT, and quenched with ice cold water, and extracted with EtOAc (3×100 mL), washed with water (100 mL), brine (100 mL), dried (Na₂SO₄) and concentrated. Purification of concentrated product was done through silica column chromatography using 25% ethyl acetate in pet ether to afford 6-chloro-N-(5-methyl-1H-pyrazol-3-yl)-2-phenylpyrimidin-4-amine (2 g, 63%) as an off white solid. R_{f}; 0.3 (PE:EtOAc; 6:4). ¹H NMR (400 MHz, CD₃OD); δ 8.37-8.34 (m, 2H), 7.49-7.47 (m, 3H), 7.10 (br s, 1H), 6.23 (br s, 1H), 2.33 (s, 3H). m/e (M+1): 285.9.

### Example 8

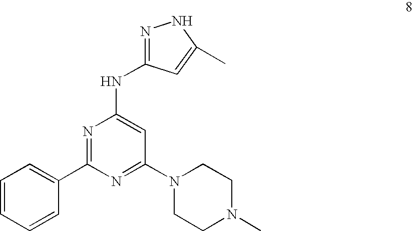

### N-(5-methyl-1H-pyrazol-3-yl)-6-(4-methylpiperazin-1-yl)-2-phenyl pyrimidin-4-amine

N-Methyl pipeprazine (0.3 mL) was added to 6-chloro-N-(5-methyl-1H-pyrazol-3-yl)-2-phenylpyrimidin-4-amine (100 mg, 0.4 mmol) and heated at 90° C. for 5 h. After completion of reaction, the reaction mixture was cooled to RT and quenched with water (15 mL), filtered and washed with water (10 mL), PE (50 mL). The solid obtained was triturated with DCM: PE to afford N-(5-methyl-1H-pyrazol-3-yl)-6-(4-methylpiperazin-1-yl)-2-phenylpyrimidin-4-amine (80 mg, 66%) as an off white solid. R_{f}; 0.3 (CHCl₃:MeOH; 9:1). ¹H NMR (400 MHz, CD₃OD); δ: 8.35-8.33 (m, 2H), 7.45-7.43 (m, 3H), 6.37 (br s, 1H), 6.15 (br s, 1H), 3.74 (br s, 4H), 2.62-2.60 (m, 4H), 2.39 (s, 3H), 2.30 (s, 3H). m/e (M+1): 350.2; HPLC purity: >98%.

### Example 9

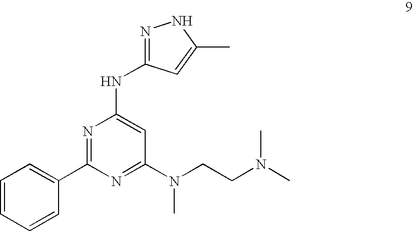

### N⁴-(2-(dimethylamino)ethyl)-N⁴-methyl-N⁶-(5-methyl-1H-pyrazol-3-yl)-2-phenylpyrimidine-4,6-diamine

N,N,N′-Trimethylethylenediamine (0.3 mL) was added to 6-chloro-N-(5-methyl-1H-pyrazol-3-yl)-2-phenylpyrimidin-4-amine (0.100 g, 0.4 mmol) at RT, and heated at 100° C. for 6 h. After completion of reaction, the reaction mixture was cooled to RT and quenched with water (15 mL), stirred for 10 min, and filtered. The resultant filtered product was triturated with dichloromethane: pet. ether to afford N⁴-(2-(dimethylamino)ethyl)-N⁴-methyl-N⁶-(5-methyl-1H-pyrazol-3-yl)-2-phenylpyrimidine-4,6-diamine (50 mg, 35%) as an off white solid. R_{f}: 0.25 (CHCl₃:MeOH; 9:1). ¹H NMR (400 MHz, CD₃OD): δ 8.34 (br s, 2H), 7.43 (br s, 3H), 6.26 (br s, 1H), 6.15 (br s, 1H), 3.86 (t, 2H, J=7.4 Hz), 3.10 (s, 3H), 2.64 (t, 2H, J=7.4 Hz), 2.38 (s, 6H), 2.30 (s, 3H). m/e (M+1): 352.1. HPLC purity: 98%.

### Example 10

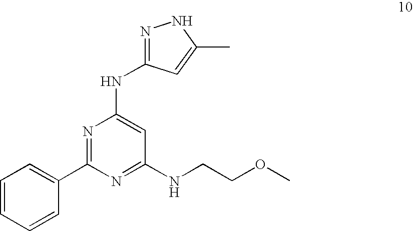

### N⁴-(2-methoxyethyl)-N⁶-(5-methyl-1H-pyrazol-3-yl)-2-phenylpyrimidine-4,6-diamine

2-Methoxyethylamine (0.2 mL) was added to 6-chloro-N-(5-methyl-1H-pyrazol-3-yl)-2-phenylpyrimidin-4-amine (0.100 g, 0.4 mmol) at RT, and heated at 95° C. for 16 h. After completion of reaction, the reaction mixture was cooled to RT, quenched with water (25 mL) and extracted with EtOAc (2×25 mL), washed with brine (25 mL), dried (Na₂SO₄) and concentrated. The concentrated product was purified through neutral alumina column chromatography using 90% EtOAc in PE to afford N⁴-(2-methoxyethyl)-N⁶-(5-methyl-1H-pyrazol-3-yl)-2-phenylpyrimidine-4,6-diamine (41 mg, 37%) as a pale brown solid. R_{f}: 0.4 (100% EtOAc). ¹H NMR (400 MHz, CD₃OD): δ 8.30-8.27 (m, 2H), 7.47-7.45 (m, 3H), 6.14 (br s, 1H), 6.04 (br s, 1H), 3.63-3.59 (m, 4H), 3.41 (s, 3H), 2.31 (s, 3H). m/e (M+1): 325.1; HPLC purity: >96%.

### Example 11

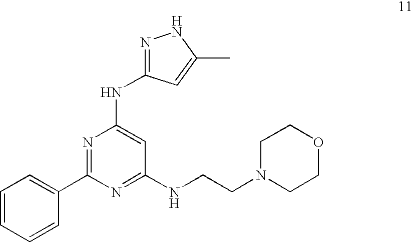

### N⁴-(5-methyl-1H-pyrazol-3-yl)-N⁶-(2-morpholinoethyl)-2-phenylpyrimidine-4,6-diamine

4-(2-Aminoethyl) morpholine (0.3 mL) was added to 6-chloro-N-(5-methyl-1H-pyrazol-3-yl)-2-phenylpyrimidin-4-amine (0.100 g, 0.4 mmol) at RT, and heated at 100° C. for 8 h. After completion of reaction, the reaction mixture was cooled to RT and quenched with water (15 mL), stirred and filtered. The filtered product was purified through silica gel column chromatography using 4% MeOH in chloroform to afford N⁴-(5-methyl-1H-pyrazol-3-yl)-N⁶-(2-morpholinoethyl)-2-phenylpyrimidine-4,6-diamine (45 mg, 30%) as an off white solid. R_{f}: 0.25 (CHCl₃:MeOH; 9:1). ¹H NMR (400 MHz, CD₃OD): δ 8.31-8.30 (m, 2H), 7.48-7.47 (m, 3H), 6.39 (br s, 1H), 6.17 (br s, 1H), 3.76-3.74 (m, 4H), 3.58 (br s, 2H), 2.70 (t, 2H, J=8 Hz), 2.62 (br s, 4H), 2.33 (s, 3H). m/e (M+1): 380.3. HPLC purity: 99%.

### Example 12

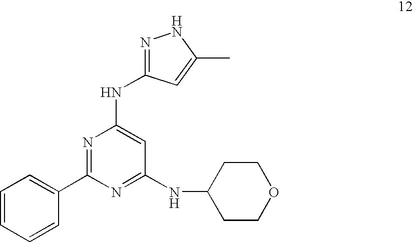

### N⁴-(tetrahydro-2H-pyran-4-yl)-N⁶-(5-methyl-1H-pyrazol-3-yl)-2-phenylpyrimidine-4,6-diamine

4-Aminotetrahydropyran (0.3 mL) was added to 6-chloro-N-(5-methyl-1H-pyrazol-3-yl)-2-phenylpyrimidin-4-amine (0.100 g, 0.4 mmol) at RT, and heated at 95° C. for 40 h. After completion of reaction, the reaction mixture was cooled to RT, and quenched with water (15 mL), stirred and filtered. The resultant filtered material was purified through silica gel column chromatography using 40% EtOAc in PE to afford N⁴-(tetrahydro-2H-pyran-4-yl)-N⁶-(5-methyl-1H-pyrazol-3-yl)-2-phenylpyrimidine-4,6-diamine (24 mg, 20%) as a pale brown solid. R_{f}: 0.4 (PE:EtOAc; 3:7). ¹H NMR (400 MHz, CD₃OD): δ 8.29-8.26 (m, 2H), 7.47-7.45 (m, 3H), 6.09 (br s, 1H), 6.01 (br s, 1H), 4.09 (br s, 1H), 4.02-3.98 (m, 2H), 3.62-3.56 (m, 2H), 2.30 (s, 3H), 2.06-2.03 (m, 2H), 1.65-1.55 (m, 2H). m/e (M+1): 351.2. HPLC purity: >97%

### Example 13

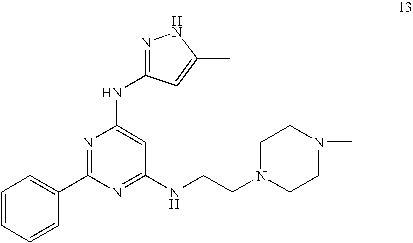

### N⁴-(5-methyl-1H-pyrazol-3-yl)-N⁶-(2-(4-methylpiperazin-1-yl)ethyl)-2-phenylpyrimidine-4,6-diamine

1-(2-Aminoethyl)-4-methylpiperazine (0.3 mL) was added to 6-chloro-N-(5-methyl-1H-pyrazol-3-yl)-2-phenylpyrimidin-4-amine (0.100 g, 0.4 mmol) at RT, and heated at 100° C. for 8 h. After completion of reaction, the reaction mixture was cooled to RT, and quenched with water (15 mL), stirred and filtered. The filtered product was purified through silica gel column chromatography using 5% MeOH in chloroform to afford N⁴-(5-methyl-1H-pyrazol-3-yl)-N⁶-(2-(4-methylpiperazin-1-yl)ethyl)-2-phenylpyrimidine-4,6-diamine (14 mg, 9%) as an off white solid. R_{f}: 0.25 (CHCl₃:MeOH; 9:1). ¹H NMR (400 MHz, CD₃OD): δ 8.29-8.27 (m, 2H), 7.48-7.44 (m, 3H), 6.12 (br s, 1H), 6.04 (br s, 1H), 3.54 (br s, 2H), 2.77-2.69 (m, 10H), 2.40 (s, 3H), 2.30 (s, 3H). m/e (M+1): 393.1 HPLC purity: >75%.

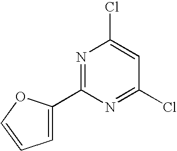

### 4,6-dichloro-2-(furan-2-yl)pyrimidine

Furan (214 mL, 30. mmol) was dissolved in diethyl ether (30 mL, anhydrous) and cooled to 0° C. n-Butyl lithium (Aldrich, 2.06M in hexanes, 14.8 mL, 1 eq) was added dropwise over 15 min. Following the addition, the ice bath was removed and the mixture was stirred at rt for 1 h. The 2-lithiofuran solution was cooled in a dry ice acetonitrile bath and 2,4-dichloropyrimidine (4.34 g, 29.4 mmol) in diethyl ether (anhydrous, 88 mL) was added dropwise over 15 min. The mixture was stirred for an 30 min in the dry ice bath, then at 0° C. for another 30 min. The reaction was quenched by addition of an acetic acid solution (1.5 mL acetic acid, 0.5 mL water, 5 mL THF) and DDQ (5.9 g in THF (25 mL)). The mixture was stirred for 5 min at rt, cooled in an ice bath and NaOH (3M, 10 mL) was added and stirred for another 5 min. The mixture was diluted with water and ether (˜100 mL each) and transferred to a separatory funnel. The ppt from the reaction was discarded. The layers were separated, and the aqueous layer was washed with ether (2×100 mL) and the combined organics were washed with water (100 mL) and brine (100 mL). The organics were decolorized with decolorizing charcoal and dried with Na₂SO₄, filtered, rotoevaped and dried under vacuum overnight. The product was purified by flash chromatography (hexane:ethyl acetate 98:2 to 90:10 gradient) to give 4,6-dichloro-2-(furan-2-yl)pyrimidine (1.79 g, 28% yield). ¹H-NMR (300 MHz, CDCl₃) δ 7.71-7.68 (m, 1H), 7.47 (dd, J=0.80, 3.4 Hz, 1H), 7.22 (s, 1H), 6.62 (dd, J=1.6, 3.4 Hz, 1H).

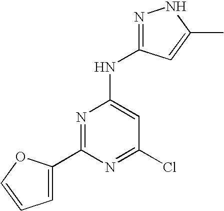

### 6-chloro-2-(furan-2-yl)-N-(5-methyl-1H-pyrazol-3-yl)pyrimidin-4-amine

4,6-dichloro-2-(furan-2-yl)pyrimidine (3.3 g, 15.3 mmol) was dissolved in N,N-dimethylacetamide

(anhydrous, 25 mL) and 5-methyl-3-aminopyrazole (1.78 g, 1.2 eq, 18.4 mmol), sodium iodide (3.45 g, 1.5 eq, 23 mmol) and diisopropylethyl amine (4 mL, 1.5 eq, 23 mmol) was added to the solution. The mixture was heated to 55° C. and stirred for 18 h. The mixture was cooled to rt, and diluted with water and ethyl acetate (˜50 mL each). The layers were separated and the organic layer was washed with NaHCO₃ (satd, 3×50 mL) and brine (50 mL). The organics were dried with Na₂SO₄ filtered and concentrated under reduced pressure. The crude product was crystallized with ethyl acetate and hexanes to give 6-chloro-2-(furan-2-yl)-N-(5-methyl-1H-pyrazol-3-yl)pyrimidin-4-amine (1.67 g, 6.1 mmol, 40%). ¹H-NMR (300 MHz, CDCl₃) δ 9.48 (br s, 1H), 7.65-7.60 (m, 1H), 7.55 (br s, 1H), 7.26 (s, 1H), 7.19 (br s, 1H), 6.60-6.55 (m, 1H), 6.0 (br s, 1H), 2.36 (s, 3H).

### Example 14

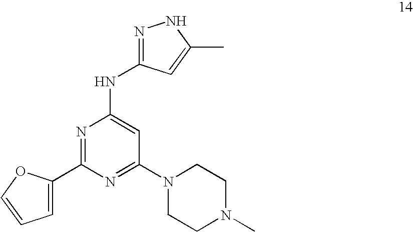

### 2-(furan-2-yl)-N-(5-methyl-1H-pyrazol-3-yl)-6-(4-methylpiperazin-1-yl)-pyrimidin-4-amine

6-chloro-2-(furan-2-yl)-N-(5-methyl-1H-pyrazol-3-yl)pyrimidin-4-amine (100 mg, 0.363 mmol) was dissolved in N,N-dimethylacetamide (anhydrous, 2 mL) and N-methylpiperazine (0.122 mL, 1.09 mmol, 3 eq) was added to the solution. The mixture was heated at 105° C. for 18 h. The reaction mixture was diluted with ethyl acetate and water (˜50 mL each) and the layers were separated. The aqueous layer was washed with ethyl acetate (2×50 mL) and the combined organics were washed with NaHCO₃ (satd, 2×50 mL) and brine (50 mL). The organic layer was dried with Na₂SO₄, filtered and rotoevaped. The crude product was purified by crystallization with methanol and ether followed by column chromatography with ethyl acetate/hexanes on silica gel to give 2-(furan-2-yl)-N-(5-methyl-1H-pyrazol-3-yl)-6-(4-methylpiperazin-1-yl)pyrimidin-4-amine (2.1 mg, 1.7% yield). ¹H-NMR (300 MHz, CDCl₃) δ 7.57 (m, 1H), 7.19 (dd, J=0.95, 3.39 Hz, 1H), 7.14 (br s, 1H), 6.52 (dd, J=1.7, 3.39 Hz, 1H), 5.82 (br s, 1H), 3.77-3.65 (m, 4H), 2.56-2.46 (m, 4H), 2.31 (s, 3H), 2.10 (s, 3H).

### Example 15

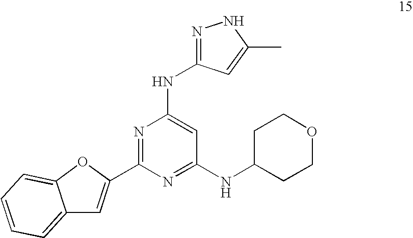

### 2-(benzofuran-2-yl)-N⁴-(tetrahydro-2H-pyran-4-yl)-N⁶-(5-methyl-1H-pyrazol-3-yl)-pyrimidine-4,6-diamine

4-Aminotetrahydropyran (1.5 ml) was added to 2-(benzofuran-2-yl)-6-chloro-N-(5-methyl-1H-pyrazol-3-yl)pyrimidin-4-amine (3 g, 0.0436 mol) at RT and heated at 90° C. for 48 h. Then, the reaction mixture was cooled to RT, quenched with water (100 ml), extracted with chloroform (3×50 ml), and concentrated. The resultant concentrated product was purified through flash column chromatography using 1% MeOH in chloroform to afford 2-(benzofuran-2-yl)-N⁴-(tetrahydro-2H-pyran-4-yl)-N⁶-(5-methyl-1H-pyrazol-3-yl)pyrimidine-4,6-diamine (1.5 g, 41.7%) as an off white solid. R_{f}; 0.3 (CHCl₃:MeOH; 9:1). ¹H NMR (400 MHz, CD₃OD); δ 7.70 (d, 1H, J=7.6 Hz), 7.59 (d, 1H, J=7.6 Hz), 7.54 (s, 1H), 7.41-7.37 (m, 1H), 7.31-7.27 (m, 1H), 6.20 (br s, 1H), 6.05 (br s, 1H), 4.02-3.97 (m, 3H), 3.63-3.56 (m, 2H), 2.30 (s, 3H), 2.05-2.01 (m, 2H), 1.70-1.55 (m, 2H). m/e (M+1): 391.1; HPLC purity: >98%.

### Example 16

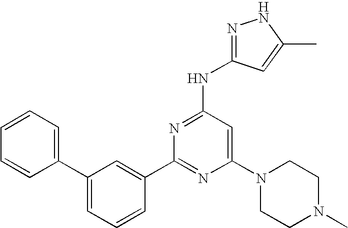

### 2-(biphenyl-3-yl)-N-(5-methyl-1H-pyrazol-3-yl)-6-(4-methylpiperazin-1-yl)pyrimidin-4-amine

Example 19 was prepared following the same general procedure as in Example 8. R_{f}; 0.4 (CHCl₃:MeOH; 9:1). ¹H NMR (400 MHz, CD₃OD); δ 8.63 (s, 1H), 8.33 (d, 1H, J=7.8 Hz), 7.72-7.70 (m, 3H), 7.55-7.46 (m, 3H), 7.39-7.35 (m, 1H), 6.35 (brs, 1H), 6.18 (brs, 1H), 3.76 (brs, 4H), 2.61 (brs, 4H), 2.38 (s, 3H), 2.31 (s, 3H). m/e (M+1): 426.2; HPLC purity: >98%

### Example 19

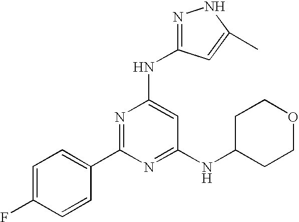

### 2-(4-fluorophenyl)-N⁴-(5-methyl-1H-pyrazol-3-yl)-N⁶-(tetrahydro-2H-pyran-4-yl)pyrimidine-4,6-diamine

Example 19 was prepared following the same general procedure as in Example 196. ¹H NMR (300 MHz, CDCl₃): δ 8.37-8.25 (m, 2H), 7.11 (t, 2H, J=8.7 Hz), 6.28 (brs, 1H), 5.91 (s, 1H), 4.78-4.58 (m, 5H), 4.00 (dt, 2H, J=11.6, 3.4 Hz), 4.07-3.83 (brs, 1H), 3.55 (td, 2H, J=11.5, 2.0 Hz), 2.31 (s, 3H), 2.12-1.98 (m, 2H), 1.65-1.47 (m, 2H). HPLC purity>97%.

### Example 20

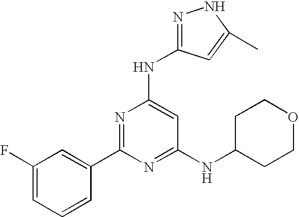

### 2-(3-fluorophenyl)-N⁴-(5-methyl-1H-pyrazol-3-yl)-N⁶-(tetrahydro-2H-pyran-4-yl)pyrimidine-4,6-diamine

Example 20 was prepared following the same general procedure as in Example 196. ¹H NMR (300 MHz, CDCl₃): δ 8.10 (app. d, 1H, J=7.8 Hz), 8.00 (app. d, 1H, J=10.6 Hz), 7.47-7.32 (m, 1H), 7.18-7.05 (m, 1H), 6.31 (s, 1H), 5.92 (s, 1H), 4.08-3.85 (m, 3H), 3.55 (t, 2H, J=11.5 Hz), 2.31 (s, 3H), 2.12-1.97 (m, 2H), 1.65-1.45 (m, 2H). HPLC purity>98%.

### Example 22

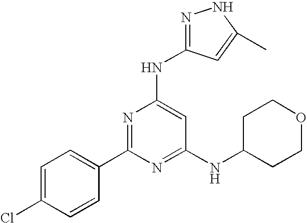

### 2-(4-chlorophenyl)-N⁴-(5-methyl-1H-pyrazol-3-yl)-N⁶-(tetrahydro-2H-pyran-4-yl)pyrimidine-4,6-diamine

Example 22 was prepared following the same general procedure as in Example 8. ¹H NMR (300 MHz, CDCl₃): 8.29 (d, J=9 Hz, 2H), 7.43 (d, J=9 Hz, 2H), 6.93 (s, exchangeable H, 1H), 6.30 (s, 1H), 5.92 (s, 1H), 4.78 (s, exchangeable H, 1H), 4.03 (m, 3H), 3.58 (m, 2H), 2.30 (s, 3H), 2.13-2.02 (m, 2H), 1.68-1.49 (m, 2H).

### Example 23

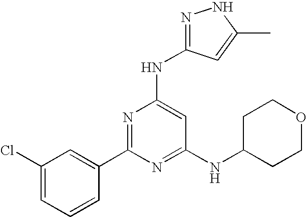

### 2-(3-chlorophenyl)-N⁴-(5-methyl-1H-pyrazol-3-yl)-N⁶-(tetrahydro-2H-pyran-4-yl)pyrimidine-4,6-diamine

Example 23 was prepared following the same general procedure as in Example 8. R_{f}. 0.4 (100% EtOAc). ¹H NMR (400 MHz, CD₃OD): δ 8.30 (s, 1H), 8.24-8.22 (m, 1H), 7.43-7.42 (m, 2H), 6.15 (br s, 1H), 6.06 (br s, 1H), 4.02-3.98 (m, 3H), 3.62-3.56 (m, 2H), 2.29 (s, 3H), 2.05-2.02 (m, 2H), 1.61-1.57 (m, 2H). m/e (M+1): 386.3; HPLC purity: >96%

### Example 24

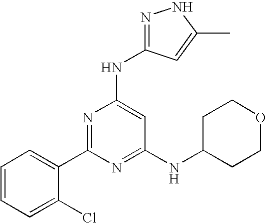

### 2-(2-chlorophenyl)-N⁴-(5-methyl-1H-pyrazol-3-yl)-N⁶-(tetrahydro-2H-pyran-4-yl)pyrimidine-4,6-diamine

Example 24 was prepared following the same general procedure as in Example 8. R_{f}: 0.4 (CHCl₃:MeOH; 9:1). ¹H NMR (400 MHz, CD₃OD): δ 7.61 (br s, 1H), 7.50-7.48 (m, 1H), 7.42-7.38 (m, 2H), 6.25 (br s, 1H), 5.93 (br s, 1H), 3.97-3.94 (m, 3H), 3.55-3.49 (m, 2H), 2.26 (s, 3H), 2.00-1.98 (m, 2H), 1.59-1.55 (m, 2H). m/e (M+1): 385.1; HPLC purity: >90%

### Example 26

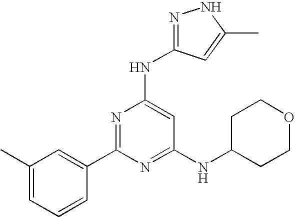

### N⁴-(5-methyl-1H-pyrazol-3-yl)-N⁶-(tetrahydro-2H-pyran-4-yl)-2-m-tolylpyrimidine-4,6-diamine

Example 26 was prepared following the same general procedure as in Example 8. R_{f}: 0.4 (100% EtOAc). ¹H NMR (400 MHz, CD₃OD): δ 8.09 (s, 1H), 8.05 (d, 1H, J=7.6 Hz), 7.34-7.26 (m, 2H), 6.13 (br s, 1H), 6.02 (br s, 1H), 4.01-3.98 (m, 3H), 3.64-3.56 (t, 2H, J=9 Hz), 2.42 (s, 3H), 2.29 (s, 3H), 2.05-2.02 (m, 2H), 1.64-1.54 (m, 2H). m/e (M+1): 365.1; HPLC purity: >98%.

### Example 28

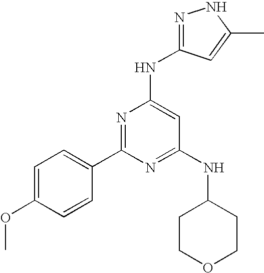

### 2-(4-methoxyphenyl)-N⁴-(5-methyl-1H-pyrazol-3-yl)-N⁶-(tetrahydro-2H-pyran-4-yl)pyrimidine-4,6-diamine

Example 28 was prepared following the same general procedure as in Example 8. R_{f}; 0.5 (100% ethyl acetate). ¹H NMR (400 MHz, CD₃OD): δ 8.23 (d, 2H, J=8.9 Hz), 6.99 (d, 2H, J=8.9 Hz), 6.05 (br s, 2H), 4.01-3.98 (m, 3H), 3.86 (s, 3H), 3.86-3.55 (m, 2H), 2.33 (s, 3H), 2.05-2.02 (m, 2H), 1.64-1.55 (m, 2H). m/e (M+1): 381; HPLC purity: >93%

### Example 29

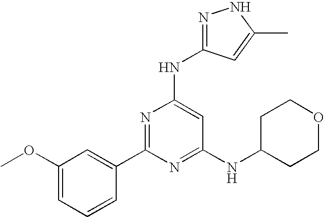

### 2-(3-methoxyphenyl)-N⁴-(5-methyl-1H-pyrazol-3-yl)-N⁶-(tetrahydro-2H-pyran-4-yl)pyrimidine-4,6-diamine

Example 29 was prepared following the same general procedure as in Example 8. R_{f}: 0.25 (100% EtOAc). ¹H NMR (400 MHz, CD₃OD): δ 7.88-7.86 (m, 2H), 7.36-7.32 (m, 1H), 7.00 (d, 1H, J=7.6 Hz), 6.17 (br s, 1H), 6.11 (br s, 1H), 4.01-3.97 (m, 3H), 3.87 (s, 3H), 3.61-3.55 (m, 2H), 2.29 (s, 3H), 2.05-2.02 (m, 2H), 1.64-1.57 (m, 2H). m/e (M+1): 381.2; HPLC purity: >93%

### Example 30

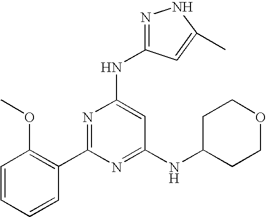

### 2-(2-methoxyphenyl)-N⁴-(5-methyl-1H-pyrazol-3-yl)-N⁶-(tetrahydro-2H-pyran-4-yl)pyrimidine-4,6-diamine

Example 30 was prepared following the same general procedure as in Example 8. R_{f}: 0.4 (100% EtOAc). ¹H NMR (400 MHz, CD₃OD): δ 7.82 (br s, 1H), 7.44-7.40 (s, 1H), 7.14 (d, J=7.7, 1H), 7.06-7.02 (m, 1H), 6.10 (br s, 1H), 5.80 (br s, 1H), 3.98-3.93 (m, 6H), 3.58-3.52 (m, 2H), 2.25 (s, 3H), 2.03-2.00 (m, 2H), 1.63-1.53 (m, 2H). m/e (M+1): 381.2; HPLC purity: >98%

### Example 31

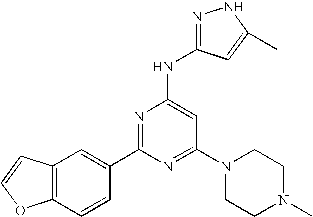

### 2-(benzofuran-5-yl)-N-(5-methyl-1H-pyrazol-3-yl)-6-(4-methylpiperazin-1-yl)pyrimidin-4-amine

Example 31 was prepared following the same general procedure as in Example 196. ¹H NMR (300 MHz, CDCl3₃): δ 8.64 (s, 1H), 8.39 (dd, J=2, 9 Hz, 1H), 7.66 (d, J=2 Hz, 1H), 7.58-7.50 (m, 1H), 7.26 (br s, 1H), 6.87-6.85 (M, 1H), 6.45 (s, 1H), 5.93 (s, 1H), 3.82-3.70 (m, 4H), 2.59 (m, 4H), 2.37 (s, 3H), 2.32 (s, 3H).

### Example 32

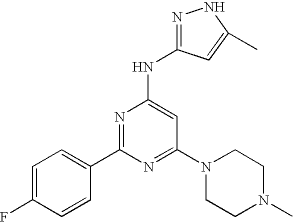

### 2-(4-fluorophenyl)-N-(5-methyl-1H-pyrazol-3-yl)-6-(4-methylpiperazin-1-yl)pyrimidin-4-amine

Example 32 was prepared following the same general procedure as in Example 196. ¹H NMR (300 MHz, CDCl₃): δ 8.43-8.28 (m, 2H), 7.10 (app. t, 2H, J=8.8 Hz), 6.84 (s, 1H), 6.42 (brs, 1H), 5.91 (s, 1H), 3.80-3.64 (m, 4H), 2.58-2.44 (m, 4H), 2.35 (s, 3H), 2.32 (s, 3H). HPLC purity>98%.

### Example 33

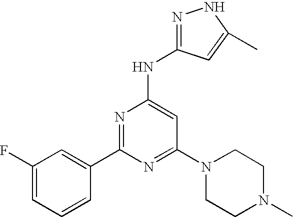

### 2-(3-fluorophenyl)-N-(5-methyl-1H-pyrazol-3-yl)-6-(4-methylpiperazin-1-yl)pyrimidin-4-amine

Example 33 was prepared following the same general procedure as in Example 196. ¹H NMR (300 MHz, CDCl₃): δ 9.35 (brs, 1H), 8.19-8.11 (m, 1H), 8.10-8.01 (m, 1H), 7.45-7.33 (m, 1H), 7.17-7.06 (m, 1H), 6.92 (s, 1H), 6.48 (brs, 1H), 5.92 (s, 1H), 3.80-3.66 (m, 4H), 2.56-2.44 (m, 4H), 2.35 (s, 3H), 2.32 (s, 3H). HPLC purity>98%.

### Example 34

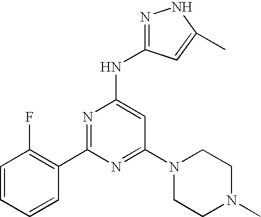

### 2-(2-fluorophenyl)-N-(5-methyl-1H-pyrazol-3-yl)-6-(4-methylpiperazin-1-yl)pyrimidin-4-amine

Example 34 was prepared following the same general procedure as in Example 196. ¹H NMR (300 MHz, CDCl₃): δ 8.05 (t, 1H, J=7.1 Hz), 7.43-7.28 (m, 2H), 7.25-7.08 (m, 2H), 6.33 (brs, 1H), 5.82 (s, 1H), 3.78-3.60 (m, 4H), 2.59-2.40 (m, 4H), 2.33 (s, 3H), 2.25 (s, 3H).

### Example 35

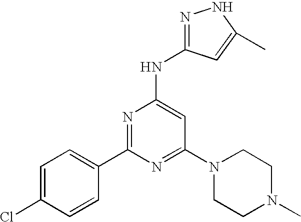

### 2-(4-chlorophenyl)-N-(5-methyl-1H-pyrazol-3-yl)-6-(4-methylpiperazin-1-yl)pyrimidin-4-amine

Example 35 was prepared following the same general procedure as in Example 8. ¹H NMR (300 MHz, CD₃OD): δ 8.31 (d, J=8 Hz, 2H), 7.42 (d, J=8 Hz, 2H), 6.35 (m, 1H), 6.11 (m, 1H), 3.74-3.68 (m, 2H), 2.59-2.48 (m, 2H), 2.35 (s, 3H), 2.28 (s, 3H).

### Example 36

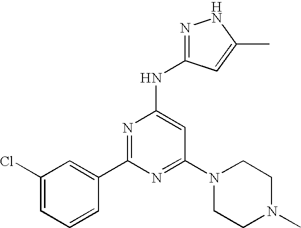

### 2-(3-chlorophenyl)-N-(5-methyl-1H-pyrazol-3-yl)-6-(4-methylpiperazin-1-yl)pyrimidin-4-amine

Example 36 was prepared following the same general procedure as in Example 8. ¹H NMR (400 MHz, CD₃OD): δ 8.33 (s, 1H), 8.29 (br s, 1H), 7.43 (br s, 2H), 6.41 (s, 1H), 6.13 (s, 1H), 3.73 (br s, 4H), 2.58-2.56 (m, 4H), 2.37 (s, 3H), 2.31 (s, 3H). m/e (M+1): 384.1; HPLC purity: >97%

### Example 37

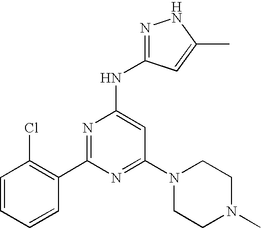

### 2-(2-chlorophenyl)-N-(5-methyl-1H-pyrazol-3-yl)-6-(4-methylpiperazin-1-yl)pyrimidin-4-amine

Example 37 was prepared following the same general procedure as in Example 8. R_{f}: 0.3 (CHCl₃:MeOH; 9:1). ¹H NMR (400 MHz, DMSO-d₆): δ 9.31 (br s, 1H), 7.63 (br s, 1H), 7.50-7.48 (m, 1H), 7.42-7.37 (m, 2H), 6.70 (br s, 1H), 5.94 (br s, 1H), 3.50 (br s, 4H), 2.36 (br s, 4H), 2.19 (m, 3H), 2.12 (s, 3H). m/e (M+1): 384.1; HPLC purity: >95%

### Example 38

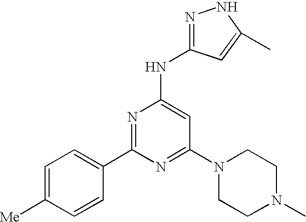

### N-(5-methyl-1H-pyrazol-3-yl)-6-(4-methylpiperazin-1-yl)-2-p-tolylpyrimidin-4-amine

Example 38 was prepared following the same general procedure as in Example 196. ¹H NMR (300 MHz, CDCl₃): δ 9.53 (brs, 1H), 8.24 (d, 2H, J=8.1 Hz), 7.24 (d, 2H, J=8.1 Hz), 7.00 (s, 1H), 6.39 (brs, 1H), 5.89 (s, 1H), 3.79-3.66 (m, 4H), 2.56-2.43 (m, 4H), 2.40 (s, 3H), 2.35 (s, 3H), 2.30 9s, 3H). HPLC purity>95%.

### Example 39

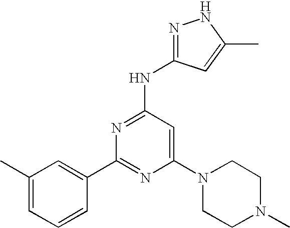

### N-(5-methyl-1H-pyrazol-3-yl)-6-(4-methylpiperazin-1-yl)-2-m-tolylpyrimidin-4-amine

Example 39 was prepared following the same general procedure as Example 8. R_{f}: 0.4 (9:1 CHCl₃:MeOH). ¹H NMR (400 MHz, DMSO-d₆): δ 11.88 (s, 1H), 9.24 (s, 1H), 8.13 (s, 1 h), 8.09 (d, 1H, J=7.9 Hz), 7.35-7.32 (m, 1H), 7.25 (d, 1H, J-7.9 Hz), 6.75 (brs, 1H), 6.01 (brs, 1H), 3.58 (brs, 4H), 2.43 (brs, 4H), 2.38 (s, 3H), 2.20 (s, 3H), 2.16 (s, 3H). m/e (M+1): 364.2; HPLC purity: >93%

### Example 40

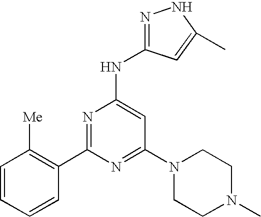

### N-(5-methyl-1H-pyrazol-3-yl)-6-(4-methylpiperazin-1-yl)-2-o-tolylpyrimidin-4-amine

Example 40 was prepared following the same general procedure as in Example 196. ¹H NMR (300 MHz, CD₃OD): δ 7.67-7.57 (m, 1H), 7.35-7.17 (m, 3H), 6.42 (brs, 1H), 5.99 (brs, 1H), 3.74-3.59 (m, 4H), 2.61-2.44 (m, 4H), 2.48 (d, 3H, J=3.3 Hz), 2.33 (d, 3H, J=3.5 Hz), 2.26 (s, 3H).

### Example 41

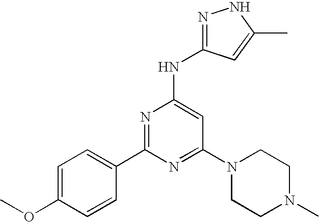

### 2-(4-methoxyphenyl)-N-(5-methyl-1H-pyrazol-3-yl)-6-(4-methylpiperazin-1-yl)pyrimidin-4-amine

Example 41 was prepared following the same general procedure as in Example 8. R_{f}; 0.4 (CHCl₃:MeOH; 9:1). ¹H NMR (400 MHz, CD₃OD): δ 8.29 (d, 1H, J=8.5 Hz), 6.98 (d, J=8.5 Hz, 1H), 6.35 (br s, 1H), 6.15 (br s, 1H), 3.86 (s, 3H), 3.72 (br s, 4H), 2.78 (br s, 4H), 2.57 (br s, 3H), 2.37 (br s, 3H). m/e (M+1): 380.2; HPLC purity: >98%

### Example 42

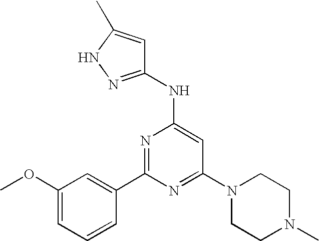

### 2-(3-methoxyphenyl)-N-(5-methyl-1H-pyrazol-3-yl)-6-(4-methylpiperazin-1-yl)pyrimidin-4-amine

Example 42 was prepared following the same general procedure as in Example 8. R_{f}: 0.4 (CHCl₃:MeOH; 9:1). ¹H NMR (400 MHz, CD₃OD): δ 7.93-7.91 (m, 2H), 7.37-7.33 (m, 1H), 7.01 (d, 1H, J=7.4 Hz), 6.37 (br s, 1H), 6.18 (br s, 1H), 3.87 (s, 3H), 3.72 (t, J=4.8 Hz, 4H), 2.57 (t, J=4.8 Hz, 4H), 2.36 (s, 3H), 2.30 (s, 3H). m/e (M+1): 380.2; HPLC purity: >98%

### Example 43

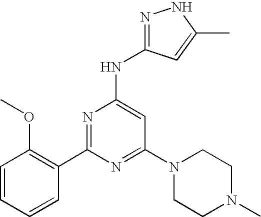

### 2-(2-methoxyphenyl)-N-(5-methyl-1H-pyrazol-3-yl)-6-(4-methylpiperazin-1-yl)pyrimidin-4-amine

Example 43 was prepared following the same general procedure as in Example 8. R_{f}; 0.4 (CHCl₃:MeOH; 9:1). ¹H NMR (400 MHz, DMSO-d₆): δ 11.82 (s, 1H), 9.21 (s, 1H), 7.43 (d, 1H, J=8.3 Hz, 1H), 7.35-7.34 (m, 1H), 7.06 (d, J=8.3 Hz, 1H), 7.05 (m, 1H), 6.97-6.90 (m, 1H), 6.70 (br s, 1H), 5.60 (br s, 1H), 3.74 (s, 3H), 3.49 (br s, 4H), 2.37 (br s, 4H), 2.21 (s, 3H), 2.16 (s, 1H). m/e (M+1): 380.2; HPLC purity: >97%

### Example 49

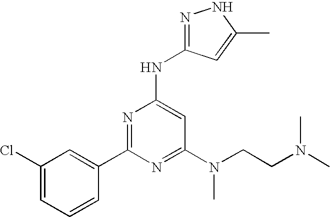

### 2-(3-chlorophenyl)-N⁴-(2-(dimethylamino)ethyl)-N⁴-methyl-N⁶-(5-methyl-1H-pyrazol-3-yl)pyrimidine-4,6-diamine

Example 49 was prepared following the same general procedure as in Example 8. R_{f}: 0.4 (DCM:MeOH; 9:1). ¹H NMR (400 MHz, CD₃OD): δ 8.36 (s, 1H), 8.30 (s, 1H), 7.43-7.41 (m, 2H), 6.28 (br s, 1H), 6.12 (br s, 1H), 3.87-3.83 (m, 2H), 3.10 (s, 3H), 2.65-2.64 (m, 2H), 2.38 (s, 6H), 2.31 (s, 3H). m/e (M+1): 385.1; HPLC purity: >98%

### Example 50

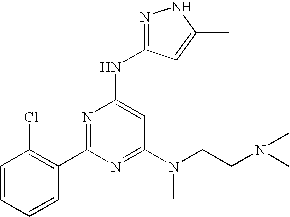

### 2-(2-chlorophenyl)-N4-(2-(dimethylamino)ethyl)-N4-methyl-N6-(5-methyl-1H-pyrazol-3-yl)pyrimidine-4,6-diamine

Example 50 was prepared following the same general procedure as in Example 8. R_{f}: 0.3 (CHCl₃:MeOH; 9:1). ¹H NMR (400 MHz, CD₃OD): δ 7.67 (br s, 1H), 7.50-7.47 (m, 1H), 7.40-7.37 (m, 2H), 6.26 (br s, 1H), 5.95 (br s, 1H), 3.78 (t, J=7.2 Hz, 2H), 3.07 (s, 3H), 2.66 (t, J=7.2 Hz, 2H), 2.34 (s, 6H), 2.26 (s, 3H). m/e (M+1): 386.2; HPLC purity: >95%

### Example 52

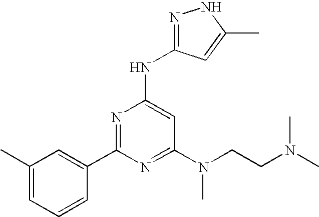

### N⁴-(2-(dimethylamino)ethyl)-N⁴-methyl-N⁶-(5-methyl-1H-pyrazol-3-yl)-2-m-tolylpyrimidine-4,6-diamine

Example 52 was prepared following the same general procedure as in Example 8. R_{f}: 0.3 (9:1 CHCl₃:MeOH). ¹H NMR (400 MHz, CD₃OD): δ 8.17 (s, 1H), 8.12 (d, 1H, J=7.6 Hz), 7.32-7.30 (m, 1H), 7.26 (d, 1H, J=7.6 Hz), 6.26 (br s, 1H), 6.13 (br s, 1H), 3.88 (t, J=7.2 Hz, 2H), 3.10 (s, 3H), 2.66 (t, J=7.2 Hz, 2H), 2.41 (s, 3H), 2.39 (s, 6H), 2.30 (s, 3H). m/e (M+1): 366.2; HPLC purity: >96%.

### Example 54

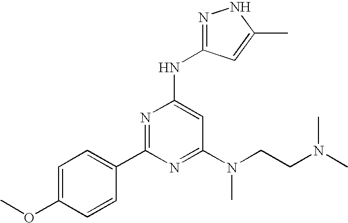

### N⁴-(2-(dimethylamino)ethyl)-2-(4-methoxyphenyl)-N⁴-methyl-N⁶-(5-methyl-1H-pyrazol-3-yl)pyrimidine-4,6-diamine

Example 54 was prepared following the same general procedure as in Example 8. R_{f}: 0.3 (CHCl₃:MeOH; 9:1). ¹H NMR (400 MHz, CD₃OD): δ 8.30 (d, J=8.4 Hz, 2H), 6.98 (d, J=8.4 Hz, 2H), 6.18 (br s, 1H), 6.14 (br s, 1H), 3.86-3.82 (m, 5H), 3.08 (s, 3H,), 2.62 (t, J=7.6 Hz, 2H), 2.36 (s, 6H), 2.29 (s, 3H). m/e (M+1): 382.2; HPLC purity: >98%

### Example 55

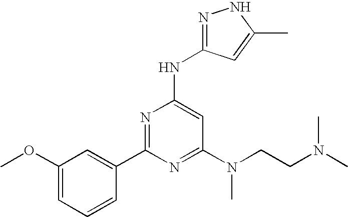

### N⁴-(2-(dimethylamino)ethyl)-2-(3-methoxyphenyl)-N⁴-methyl-N⁶-(5-methyl-1H-pyrazol-3-yl)pyrimidine-4,6-diamine

Example 55 was prepared following the same general procedure as in Example 8. R_{f}: 0.3 (CHCl₃:MeOH; 9:1). ¹H NMR (400 MHz, CD₃OD): δ 7.93 (d, J=6.8 Hz, 2H), 7.56-7.32 (m, 1H), 7.00 (d, J=8 Hz, 1H), 6.24 (br s, 1H), 6.15 (br s, 1H), 3.87-3.84 (m, 5H), 3.09 (s, 3H), 2.66 (t, J=7.2 Hz, 2H), 2.39 (s, 6H), 2.29 (s, 3H). m/e (M+1): 382.2; HPLC purity: >96%

### Example 56

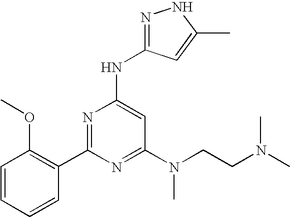

### N⁴-(2-(dimethylamino)ethyl)-2-(2-methoxyphenyl)-N⁴-methyl-N⁶-(5-methyl-1H-pyrazol-3-yl)pyrimidine-4,6-diamine

Example 56 was prepared following the same general procedure as in Example 8. R_{f}: 0.4 (9:1 DCM:MeOH). ¹H NMR (400 MHz, CD₃OD): δ 8.22 (br s, 1H), 7.44 (br s, 1H), 7.21 (br s, 1H), 7.04 (s, 1H), 5.68 (br s, 1H), 5.56 (br s, 1H), 4.07 (br s, 2H), 3.84 (br s, 3H), 3.07 (s, 3H), 2.63-2.59 (m, 2H), 2.33 (s, 6H), 2.24 (s, 3H). m/e (M+1): 382.1; HPLC purity: >98%

### Example 62

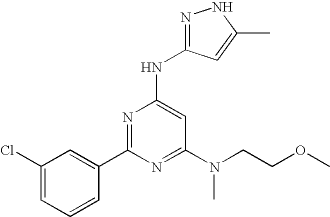

### 2-(3-chlorophenyl)-N⁴-(2-methoxyethyl)-N⁴-methyl-N⁶-(5-methyl-1H-pyrazol-3-yl)pyrimidine-4,6-diamine

Example 62 was prepared following the same general procedure as in Example 8: R_{f}. 0.4 (100% EtOAc). ¹H NMR (400 MHz, CD₃OD): δ 8.34-8.27 (m, 2H), 7.43 (br s, 2H), 6.28 (s, 1H), 6.13 (s, 1H), 3.92-3.80 (m, 2H), 3.70-3.60 (m, 2H), 3.38 (s, 3H), 3.14 (s, 3H), 2.31 (s, 3H). m/e (M+1): 373.3; HPLC purity: >95%

### Example 65

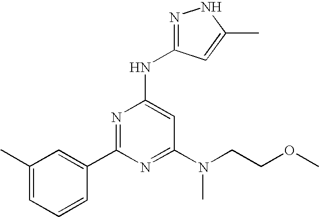

### N⁴-(2-methoxyethyl)-N⁴-methyl-N⁶-(5-methyl-1H-pyrazol-3-yl)-2-m-tolylpyrimidine-4,6-diamine

Example 65 was prepared following the same general procedure as in Example 8. R_{f}: 0.4 (100% EtOAc). ¹H NMR (400 MHz, CD₃OD): δ 8.14 (s, 1H), 8.10 (d, 1H, J=7.6 Hz), 7.34-7.30 (m, 1H), 7.26 (d, J=7.6 Hz, 1H), 6.19 (br s, 1H), 6.11 (br s, 1H), 3.87 (d, 2H, J=5.6 Hz), 3.66 (d, J=5.6 Hz, 2H), 3.38 (s, 3H), 3.31 (s, 3H), 2.42 (s, 3H), 2.30 (s, 3H). m/e (M+1): 353.2; HPLC purity: >99%.

### Example 67

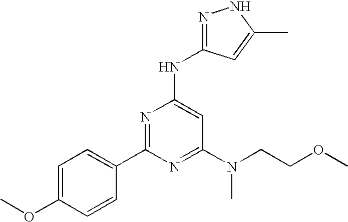

### N⁴-(2-methoxyethyl)-2-(4-methoxyphenyl)-N⁴-methyl-N⁶-(5-methyl-1H-pyrazol-3-yl)pyrimidine-4,6-diamine

Example 67 was prepared following the same general procedure as in Example 8. R_{f}: 0.4 (100% EtOAc). ¹H NMR (400 MHz, CD₃OD): δ 8.27 (d, 2H, J=8.8 Hz), 6.98 (d, J=8.8 Hz, 2H), 6.13 (br s, 2H), 3.85-3.83 (m, 5H), 3.65 (t, J=5.7 Hz, 2H), 3.67 (s, 3H), 3.11 (s, 3H), 2.29 (s, 3H). m/e (M+1): 369.2; HPLC purity: >95%

### Example 68

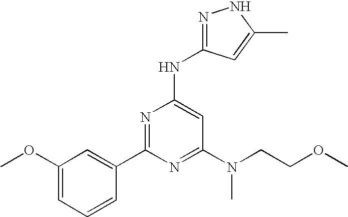

### N⁴-(2-methoxyethyl)-2-(3-methoxyphenyl)-N⁴-methyl-N⁶-(5-methyl-1H-pyrazol-3-yl)pyrimidine-4,6-diamine

Example 68 was prepared following the same general procedure as in Example 8. R_{f}: 0.3 (100% EtOAc). ¹H NMR (400 MHz, CD₃OD): δ 7.89 (d, 2H, J=7.9 Hz), 7.36-7.33 (m, 1H), 7.03-7.00 (m, 1H), 6.11 (s, 1H), 6.10 (s, 1H), 3.88-3.84 (m, 5H), 3.66 (t, J=5.6 Hz, 2H), 3.37 (s, 3H), 3.17 (s, 3H), 2.29 (s, 3H). m/e (M+1): 369.2; HPLC purity: >96%

### Example 69

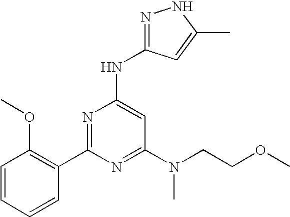

### N⁴-(2-methoxyethyl)-2-(2-methoxyphenyl)-N⁴-methyl-N⁶-(5-methyl-1H-pyrazol-3-yl)pyrimidine-4,6-diamine

Example 69 was prepared following the same general procedure as in Example 8. R_{f}: 0.4 (100% EtOAc). ¹H NMR (400 MHz, CD₃OD): δ 8.21 (br s, 1H), 7.46 (br s, 1H), 7.21-7.06 (m, 2H), 5.70 (br s, 1H), 5.55 (br s, 1H), 4.07 (br s, 2H), 3.84 (s, 3H), 3.64 (br s, 2H), 3.37 (s, 3H), 3.14 (s, 3H), 2.31 (s, 3H). m/e (M+1): 369.1; HPLC purity: >96%

### Example 70

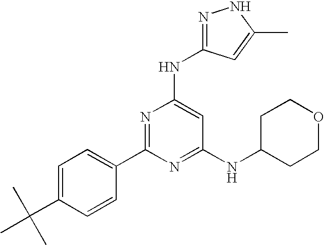

### 2-(4-tert-butylphenyl)-N⁴-(5-methyl-1H-pyrazol-3-yl)-N⁶-(tetrahydro-2H-pyran-4-yl)pyrimidine-4,6-diamine

Example 70 was prepared following the same general procedure as in Example 8. R_{f}: 0.5 (100% EtOAc). ¹H NMR (400 MHz, CD₃OD): δ 8.20 (d, 2H, J=8.5 Hz), 7.49 (d, J=8.5 Hz, 2H), 6.15 (br s, 1H), 6.10 (br s, 1H), 4.02-3.99 (m, 3H), 3.62-3.59 (m, 2H), 2.30 (s, 3H), 2.06-2.03 (m, 2H), 1.60-1.58 (m, 2H), 1.38 (s, 9H). m/e (M+1): 407.2; HPLC purity: >98%

### Example 74

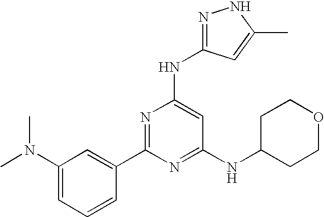

### 2-(3-(dimethylamino)phenyl)-N⁴-(5-methyl-1H-pyrazol-3-yl)-N⁶-(tetrahydro-2H-pyran-4-yl)pyrimidine-4,6-diamine

Example 74 was prepared following the same general procedure as in Example 8. R_{f}: 0.5 (100% EtOAc). ¹H NMR (400 MHz, CD₃OD): δ 7.78 (s, 1H), 7.63 (d, 1H, J=7.3 Hz), 7.30-7.26 (m, 1H), 6.91-6.90 (m, 1H), 6.15 (s, 2H), 4.01-3.99 (m, 3H), 3.61-3.60 (m, 2H), 3.01 (s, 6H), 2.29 (s, 3H), 2.07-2.04 (m, 2H), 1.64 (m, 2H). m/e (M+1): 394.1; HPLC purity: >95%

### Example 79

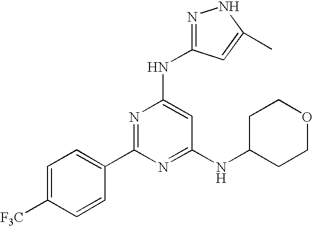

### N⁴-(5-methyl-1H-pyrazol-3-yl)-N⁶-(tetrahydro-2H-pyran-4-yl)-2-(4-(trifluoromethyl)phenyl)pyrimidine-4,6-diamine

Example 79 was prepared following the same general procedure as in Example 8. ¹H NMR (300 MHz, CDCl₃): δ 8.46 (d, J=8 Hz, 2H), 7.71 (d, J=8 Hz, 2H), 6.86 (br s, 1H), 6.37 (br s, 1H), 5.93 (s, 1H), 4.09-3.98 (m, 3H), 3.65-3.54 (m, 2H), 2.35 (s, 3H), 2.15-2.03 (m, 2H), 1.67-1.51 (m, 2H).

### Example 80

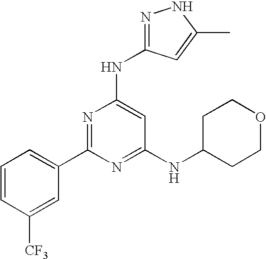

### N⁴-(5-methyl-1H-pyrazol-3-yl)-N⁶-(tetrahydro-2H-pyran-4-yl)-2-(3-(trifluoromethyl)phenyl)pyrimidine-4,6-diamine

Example 80 was prepared following the same general procedure as in Example 8. R_{f}: 0.3 (100% EtOAc). ¹H NMR (400 MHz, CD₃OD): δ 8.65 (s, 1H), 8.57 (d, 1H, J=7.6 Hz), 7.73 (d, J=7.6 Hz, 1H), 7.65-7.62 (m, 1H), 6.18 (br s, 1H), 6.05 (br s, 1H), 4.07-4.00 (m, 3H), 3.58 (t, J=11.2 Hz, 2H), 2.30 (s, 3H), 2.05-2.02 (m, 2H), 1.65-1.55 (m, 2H). m/e (M+1): 419; HPLC purity: >94%

### Example 81

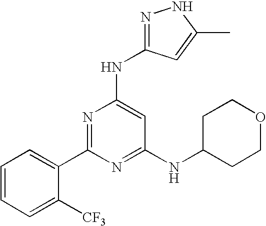

### N⁴-(5-methyl-1H-pyrazol-3-yl)-N⁶-(tetrahydro-2H-pyran-4-yl)-2-(2-(trifluoromethyl)phenyl)pyrimidine-4,6-diamine

Example 81 was prepared following the same general procedure as in Example 8. R_{f}: 0.5 (100% EtOAc). ¹H NMR (400 MHz, CD₃OD): δ 7.77 (d, J=7.8 Hz, 1H), 7.71-7.58 (m, 3H), 6.24 (br s, 1H), 5.93 (br s, 1H), 4.00 (br s, 1H), 3.96-3.94 (m, 2H), 3.53-3.3.46 (m, 2H), 2.26 (s, 3H), 1.97-1.93 (m, 2H), 1.59-1.49 (m, 2H). m/e (M+1): 419.1; HPLC purity: >97%

### Example 82

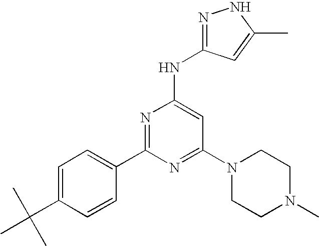

### 2-(4-tert-butylphenyl)-N-(5-methyl-1H-pyrazol-3-yl)-6-(4-methylpiperazin-1-yl)pyrimidin-4-amine

Example 82 was prepared following the same general procedure as in Example 8. R_{f}; 0.4 (CHCl₃:MeOH; 9:1). ¹H NMR (400 MHz, CD₃OD): δ 8.26 (d, 2H, J=7.7 Hz), 7.49 (d, J=7.7 Hz, 2H), 6.35 (br s, 1H), 6.17 (br s, 1H), 3.73 (br s, 4H), 2.58-2.56 (m, 4H), 2.30 (s, 3H), 2.19 (s, 3H), 1.32 (s, 9H). m/e (M+1): 406.2; HPLC purity: >98%

### Example 85

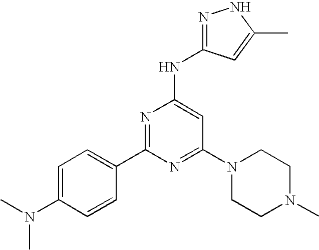

### 2-(4-(dimethylamino)prenyl)-N-(b-methyl-1H-pyrazol-3-yl)-6-(4-methylpiperazin-1-yl)pyrimidin-4-amine

Example 85 was prepared following the same general procedure as in Example 8. R_{f}: 0.3 (CHCl₃:MeOH; 9:1). ¹H NMR (400 MHz, DMSO-d₆): δ 11.83 (br s, 1H), 9.08 (br s, 1H), (8.15 (d, J=8.9 Hz, 2H), 6.75 (d, J=8.9 Hz, 1H,), 6.50 (br s, 1H), 6.07 (br s, 1H), 3.55 (br s, 4H), 3.00 (s, 3H), 2.40-2.38 (m, 4H), 2.20 (s, 3H), 2.15 (s, 3H). m/e (M+1): 393.2; HPLC purity: >95%

### Example 86

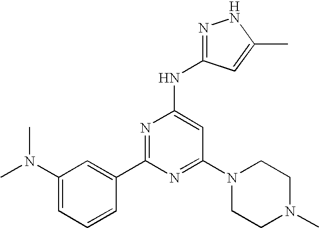

### 2-(3-(dimethylamino)phenyl)-N-(5-methyl-1H-pyrazol-3-yl)-b-(4-methylpiperazin-1-yl)pyrimidin-4-amine

Example 86 was prepared following the same general procedure as in Example 8. R_{f}: 0.4 (CHCl₃:MeOH; 9:1). ¹H NMR (400 MHz, CD₃OD): δ 7.84 (br s, 1H), 7.70 (br s, 1H), 7.28 (br s, 1H), 6.91-6.89 (m, 1H), 6.34 (s, 1H), 6.24 (s, 1H), 3.73 (br s, 4H), 3.01 (s, 6H), 2.58 (br s, 4H), 2.36 (s, 3H), 2.31 (s, 3H). m/e (M+1): 393.2; HPLC purity: >98%

### Example 89

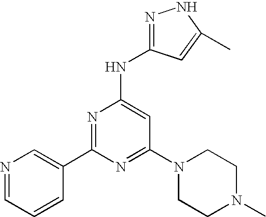

### N-(5-methyl-1H-pyrazol-3-yl)-6-(4-methylpiperazin-1-yl)-2-(pyridin-3-yl)pyrimidin-4-amine

Example 89 was prepared following the same general procedure as in Example 196. ¹H NMR (300 MHz, CDCl₃): δ 9.57 (s, 1H), 8.68-8.55 (m, 1H), 8.1 (br s, 1H), 7.39-7.30 (m, 1H), 6.4 (br s, 1H), 6.0 (br s, 1H), 3.75-3.59 (m, 4H), 2.54-2.38 (m, 4H), 2.29 (s, 3H), 2.26 (s, 3H).

### Example 95

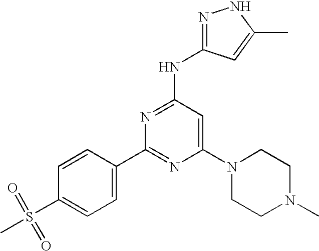

### N-(5-methyl-1H-pyrazol-3-yl)-6-(4-methylpiperazin-1-yl)-2-(4-(methylsulfonyl)phenyl)pyrimidin-4-amine

Example 95 was prepared following the same general procedure as in Example 196. ¹H NMR (300 MHz, CDCl₃): δ 8.57 (d, J=8 Hz, 2H0, 8.02 (d, J=8 Hz, 2H), 6.91 (s, 1H), 6.58 (s, 1H), 5.96 (s, 1H), 3.82-3.71 (m, 4H), 3.10 (s, 3H), 2.59-2.50 (m, 4H), 2.39 (s, 3H).

### Example 98

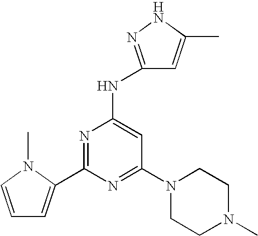

### N-(5-methyl-1H-pyrazol-3-yl)-2-(1-methyl-1H-pyrrol-2-yl)-6-(4-methylpiperazin-1-yl)pyrimidin-4-amine

Example 98 was prepared following the same general procedure as in Example 196. ¹H NMR (300 MHz, CD₃OD): δ 7.91 (s, 1H), 6.96-6.89 (m, 1H), 6.82-6.73 (m, 1H), 6.15 (br s, 1H), 6.12-6.05 (m, 1H), 6.02 (br s, 1H0, 3.69-3.59 (m, 4H), 2.60-2.49 (m, 4H0, 2.36 (s, 3H), 2.29 (s, 3H).

### Example 100

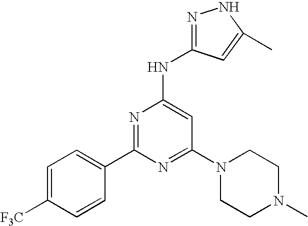

### N-(5-methyl-1H-pyrazol-3-yl)-(4-methylpiperazin-1-yl)-2-(4-(trifluoromethyl)phenyl)pyrimidin-4-amine

Example 100 was prepared following the same general procedure as in Example 196. ¹H NMR (300 MHz, CDCl₃): δ 8.51 (d, J=8 Hz, 2H), 7.72 (d, J=8 Hz, 2H), 6.35 (br s, 1H), 6.09 (br s, 1H), 3.78-3.68 (m, 4H), 2.63-2.56 (m, 4H), 2.38 (s, 3H), 2.30 (s, 3H).

### Example 101

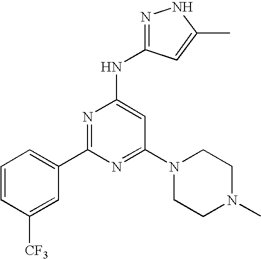

### N-(5-methyl-1H-pyrazol-3-yl)-6-(4-methylpiperazin-1-yl)-2-(3-(trifluoromethyl)phenyl)pyrimidin-4-amine

Example 101 was prepared following the same general procedure as in Example 8. R_{f}: 0.3 (CHCl₃:MeOH; 9:1). ¹H NMR (400 MHz, CD₃OD); δ 8.66 (s, 1H), 8.62 (d, J=7.5 Hz, 2H), 7.74 (d, J=7.5 Hz, 1H), 7.66-7.62 (m, 1H), 6.45 (br s, 1H), 6.13 (br s, 1H), 3.77 (br s, 4H), 2.68 (br s, 4H), 2.44 (s, 3H), 2.27 (s, 3H). m/e (M+1): 418.2; HPLC purity: >91%

### Example 102

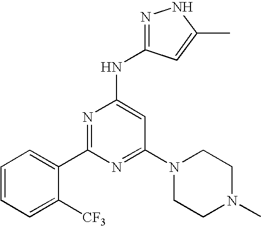

### N-(5-methyl-1H-pyrazol-3-yl)-6-(4-methylpiperazin-1-yl)-2-(2-(trifluoromethyl)phenyl)pyrimidin-4-amine

Example 102 was prepared following the same general procedure as in Example 8. R_{f}: 0.3 (CHCl₃:MeOH; 9:1). ¹H NMR (400 MHz, CD₃OD): δ 7.78 (d, J=7.8 Hz, 1H), 7.69-7.66 (m, 2H), 7.61-7.59 (m, 1H), 6.47 (br s, 1H), 6.02 (br s, 1H), 3.66 (br s, 4H), 2.53 (t, J=5.2 Hz, 4H), 2.33 (s, 3H), 2.27 (s, 3H). m/e (M+1): 418.2; HPLC purity: >99%

### Example 103

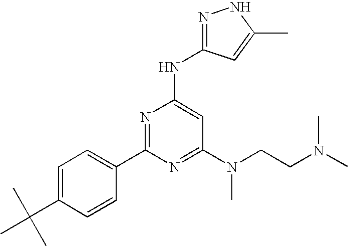

### 2-(4-tert-butylphenyl)-N⁴-(2-(dimethylamino)ethyl)-N⁴-methyl-N⁶-(5-methyl-1H-pyrazol-3-yl)pyrimidine-4,6-diamine

Example 103 was prepared following the same general procedure as in Example 8. R_{f}: 0.3 (CHCl₃:MeOH; 9:1). ¹H NMR (400 MHz, CD₃OD): δ 8.27 (d, J=8.3 Hz, 2H), 7.49 (d, J=8.3 Hz, 2H), 6.30 (br s, 1H), 6.15 (br s, 1H), 3.86 (t, J=7.4 Hz, 2H), 3.10 (s, 3H), 2.63 (t, J=7.4 Hz, 2H), 2.37 (s, 6H), 2.30 (s, 3H), 1.38 (s, 9H). m/e (M+1): 408.2; HPLC purity: >98%

### Example 107

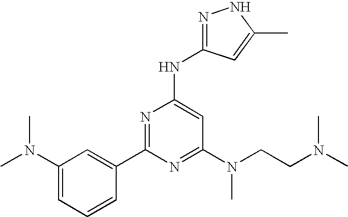

### N⁴-(2-(dimethylamino))ethyl)-2-(3-(dimethylamino)phenyl)-N⁴-methyl-N⁶-(5-methyl-1H-pyrazol-3-yl)pyrimidine-4,6-diamine

Example 107 was prepared following the same general procedure as in Example 8. R_{f}: 0.3 (CHCl₃:MeOH; 9:1). ¹H NMR (400 MHz, CD₃OD): δ 7.84 (s, 1H), 7.70 (br s, 1H), 7.30-7.26 (m, 1H), 6.91-6.89 (m, 1H), 6.21 (br s, 2H), 3.87 (t, 2H, J=6.8 Hz), 3.14 (s, 3H), 3.01 (s, 6H), 3.13 (s, 3H), 3.01 (s, 6H), 2.68 (t, J=6.8 Hz, 2H), 2.39 (s, 6H), 2.30 (s, 3H). m/e (M+1): 395.2; HPLC purity: >95%

### Example 113

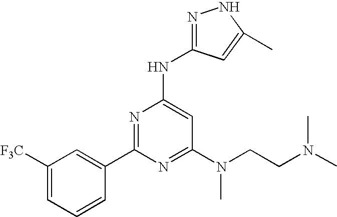

### N⁴-(2-(dimethylamino)ethyl)-N⁴-methyl-N⁶-(5-methyl-1H-pyrazol-3-yl)-2-(3-(trifluoromethyl)phenyl)pyrimidine-4,6-diamine

Example 113 was prepared following the same general procedure as in Example 8. R_{f}: 0.3 (CHCl₃:MeOH; 9:1). ¹H NMR (400 MHz, CD₃OD): δ 8.69 (s, 1H), 8.62 (d, J=8 Hz, 1H), 7.73 (d, J=8 Hz, 1H), 7.65-7.62 (m, 1H), 6.30 (s, 1H), 6.13 (s, 1H) 3.87-3.84 (t, J=7.2 Hz, 2H), 3.10 (s, 3H), 2.65 (t, J=7.2 Hz, 2H), 2.38 (s, 6H), 2.30 (s, 3H). m/e (M+1): 420.2; HPLC purity: >99%

### Example 114

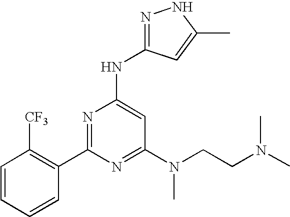

### N⁴-(2-(dimethylamino)ethyl)-N⁴-methyl-N⁶-(5-methyl-1H-pyrazol-3-yl)-2-(2-(trifluoromethyl)phenyl)pyrimidine-4,6-diamine

Example 114 was prepared following the same general procedure as in Example 8. R_{f}: 0.3 (CHCl₃:MeOH; 9:1). ¹H NMR (400 MHz, CD₃OD): δ 7.78 (d, J=7.7 Hz, 1H), 7.69-7.62 (m, 2H), 7.62 (d, J=6.5 Hz, 1H), 6.40 (br s, 1H), 6.00 (br s, 1H), 3.80 (t, J=6.9 Hz, 2H), 3.07 (s, 3H), 2.74 (br s, 2H), 2.40 (s, 6H), 2.26 (s, 3H). m/e (M+1): 419.8; HPLC purity: >99%

### Example 115

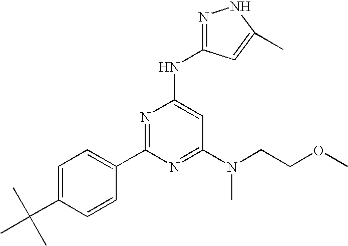

### 2-(4-tert-butylphenyl)-N⁴-(2-methoxyethyl)-N⁴-methyl-N⁶-(5-methyl-1H-pyrazol-3-yl)pyrimidine-4,6-diamine

Example 115 was prepared following the same general procedure as in Example 8. R_{f}: 0.3 (100% EtOAc). ¹H NMR (400 MHz, DMSO): δ 9.12 (br s, 1H,), 8.22 (d, 2H, J=8.4 Hz), 7.47 (d, 2H, J=8.4 Hz), 6.46 (br s, 1H), 6.08 (br s, 1H), 3.77 (br s, 2H), 3.56 (t, 2H, J=5.7 Hz), 3.27 (s, 3H), 3.03 (s, 3H), 2.20 (s, 3H), 1.31 (s, 9H). m/e (M+1): 395.2; HPLC purity: >99%

### Example 118

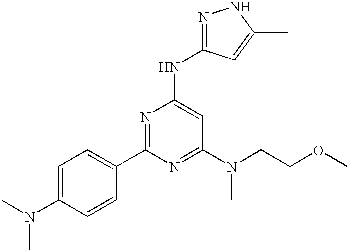

### 2-(4-(dimethylamino)phenyl)-N⁴-(2-methoxyethyl)-N⁴-methyl-N⁶-(5-methyl-1H-pyrazol-3-yl)pyrimidine-4,6-diamine

Example 118 was prepared following the same general procedure as in Example 8. R_{f}: 0.25 (100% EtOAc). ¹H NMR (400 MHz, CD₃OD): δ 8.18 (d, 2H, J=8.8 Hz), 6.81 (d, J=8.8 Hz, 2H), 6.09 (br s, 2H), 3.85 (br s, 2H), 3.67 (t, J=5.6 Hz, 2H), 3.38 (s, 3H), 3.13 (s, 3H), 3.03 (s, 6H), 2.30 (s, 3H). m/e (M+1): 382; HPLC purity: >93%

### Example 119

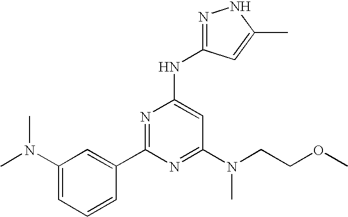

### 2-(3-(dimethylamino)phenyl)-N⁴-(2-methoxyethyl)-N⁴-methyl-N⁶-(5-methyl-1H-pyrazol-3-yl)pyrimidine-4,6-diamine

Example 119 was prepared following the same general procedure as in Example 8. R_{f}: 0.6 (PE:EtOAc; 7:3). ¹H NMR (400 MHz, CD₃OD): δ 7.84 (s, 1H), 7.70 (d, J=6.7 Hz, 1H,), 7.30-7.26 (m, 1H), 6.91-6.89 (m, 1H), 6.21 (br s, 2H), 3.86 (t, J=5.7 Hz, 2H), 3.67 (t, J=5.7 Hz, 2H), 3.38 (s, 3H), 3.13 (s, 3H), 3.01 (s, 6H), 2.29 (s, 3H). m/e (M+1): 382.1; HPLC purity: >98%

### Example 125

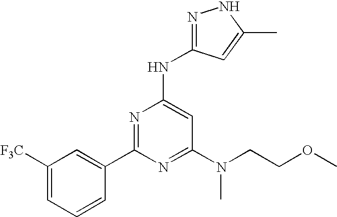

### N⁴-(2-methoxyethyl)-N⁴-methyl-N⁶-(5-methyl-1H-pyrazol-3-yl)-2-(3-(trifluoromethyl)phenyl)pyrimidine-4,6-diamine

Example 125 was prepared following the same general procedure as in Example 8. R_{f}: 0.3 (100% EtOAc). ¹H NMR (400 MHz, CD₃OD): δ 8.67 (s, 1H), 8.62 (d, J=7.8 Hz, 2H), 7.73 (d, J=7.8 Hz, 1H), 7.66-7.62 (m, 1H), 6.29 (br s, 1H), 6.13 (br s, 1H), 3.87 (t, J=5.6 Hz, 2H), 3.66 (t, J=5.6 Hz, 2H), 3.38 (s, 3H), 3.10 (s, 3H), 2.30 (s, 3H). m/e (M+1): 407.2; HPLC purity: >93%

### Example 126

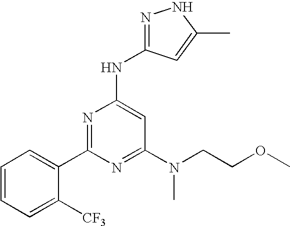

### N⁴-(2-methoxyethyl)-N⁴-methyl-N⁶-(5-methyl-1H-pyrazol-3-yl)-2-(2-(trifluoromethyl)phenyl)pyrimidine-4,6-diamine

Example 126 was prepared following the same general procedure as in Example 8. R_{f}: 0.5 (100% EtOAc). ¹H NMR (400 MHz, CD₃OD): δ 7.77 (d, J=7.6 Hz, 1H), 7.69-7.67 (m, 2H), 7.61-7.57 (m, 1H), 6.34 (br s, 1H), 6.00 (br s, 1H), 3.78 (t, J=5.6 Hz, 2H), 3.57 (t, J=5.6 Hz, 2H), 3.33 (s, 3H), 3.09 (s, 3H), 2.26 (s, 3H). m/e (M+1): 407; HPLC purity: >91%

### Example 127

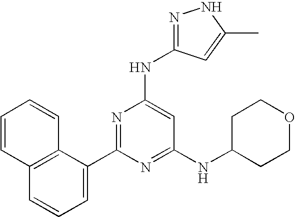

### N⁴-(5-methyl-1H-pyrazol-3-yl)-2-(naphthalen-1-yl)-N⁶-(tetrahydro-2H-pyran-4-yl)pyrimidine-4,6-diamine

Example 127 was prepared following the same general procedure as in Example 8. R_{f}: 0.4 (100% EtOAc). ¹H NMR (400 MHz, CD₃OD): δ 8.40 (br s, 1H), 7.97-7.92 (m, 2H), 7.81-7.80 (m, 1H), 7.80-7.50 (m, 3H), 6.37 (br s, 1H), 6.01 (br s, 1H), 3.98-3.95 (m, 3H), 3.54-3.49 (m, 2H), 2.27 (s, 3H), 2.03-1.99 (m, 2H), 1.63-1.57 (m, 2H). m/e (M+1): 401.2; HPLC purity: >99%

### Example 128

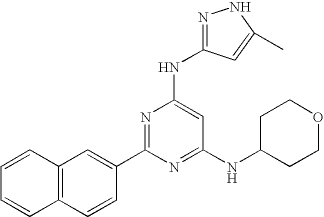

### N⁴-(5-methyl-1H-pyrazol-3-yl)-2-(naphthalen-2-yl)-N⁶-(tetrahydro-2H-pyran-4-yl)pyrimidine-4,6-diamine

Example 128 was prepared following the same general procedure as in Example 8. R_{f}: 0.4 (100% EtOAc). ¹H NMR (400 MHz, CD₃OD): δ 8.80 (s, 1H), 8.41 (d, 1H, J=8.6 Hz), 7.98-7.89 (m, 3H), 7.54-7.52 (m, 2H), 6.15 (br s, 1H), 6.06 (br s, 1H), 4.12 (br s, 1H), 4.03-4.00 (m, 2H), 3.65-3.60 (m, 2H), 2.31 (s, 3H), 2.09-2.06 (m, 2H), 1.64-1.62 (m, 2H). m/e (M+1): 401.2; HPLC purity: >98%

### Example 130

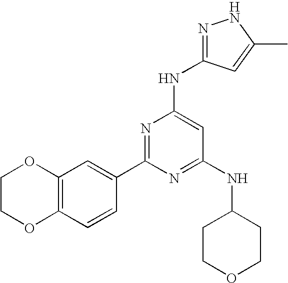

### 2-(2,3-dihydrobenzo[b][1,4]dioxin-6-yl)-N⁴-(5-methyl-1H-pyrazol-3-yl)-N⁶-(tetrahydro-2H-pyran-4-yl)pyrimidine-4,6-diamine

Example 130 was prepared following the same general procedure as in Example 8. R_{f}: 0.3 (CHCl₃:MeOH; 9:1). ¹H NMR (400 MHz, CD₃OD): δ 7.78 (br s, 2H), 6.88 (d, J=8.9 Hz, 1H,), 6.11 (br s, 1H), 6.08 (br s, 1H), 4.28 (br s, 4H), 4.00-3.97 (m, 3H), 3.61-3.55 (m, 2H), 2.29 (s, 3H), 2.04-2.00 (m, 2H), 1.63-1.53 (m, 2H). m/e (M+1): 409.1; HPLC purity: >91%

### Example 132

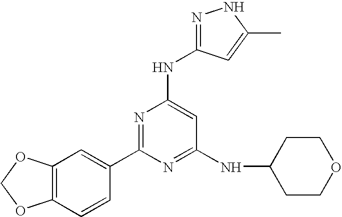

### 2-(benzo[d][1,3]dioxol-5-yl)-N⁴-(5-methyl-1H-pyrazol-3-yl)-N⁶-(tetrahydro-2H-pyran-4-yl)pyrimidine-4,6-diamine

Example 132 was prepared following the same general procedure as in Example 8. R_{f}: 0.5 (CHCl₃:MeOH; 9:1). ¹H NMR (400 MHz, DMSO-d₆): δ 11.79 (br s, 1H), 8.94 (br s, 1H), 7.89 (d, J=8.2 Hz, 1H), 7.76 (s, 1H), 6.98 (d, J=8.2 Hz, 1H), 6.90 (br s, 1H), 6.33 (br s, 1H), 6.06 (s, 2H), 5.95 (br s, 1H), 4.01 (br s, 1H), 3.88-3.85 (m, 2H), 3.45-3.40 (m, 2H), 2.20 (s, 3H), 1.89-1.86 (m, 2H), 1.50-1.41 (m, 2H). m/e (M+1): 395.1; HPLC purity: >98%

### Example 134

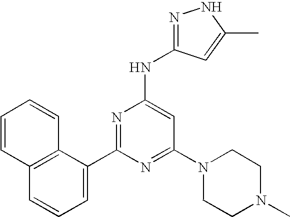

### N-(5-methyl-1H-pyrazol-3-yl)-6-(4-methylpiperazin-1-yl)-2-(naphthalen-1-yl)pyrimidin-4-amine

Example 134 was prepared following the same general procedure as in Example 8. R_{f}: 0.3 (CHCl₃:MeOH). ¹H NMR (400 MHz, CD₃OD): δ 8.57-8.55 (m, 1H), 7.97-7.89 (m, 3H), 7.58-7.49 (m, 3H), 6.54 (s, 1H), 6.06 (s, 1H), 3.71 (br s, 4H), 2.55 (t, 4H, J=5 Hz), 2.35 (s, 3H), 2.28 (s, 3H) m/e (M+1): 400.2; HPLC purity: >98%

### Example 135

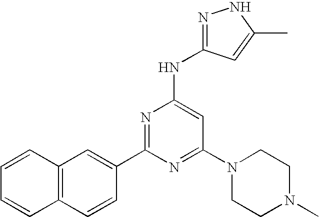

### N-(5-methyl-1H-pyrazol-3-yl)-6-(4-methylpiperazin-1-yl)-2-(naphthalen-2-yl)pyrimidin-4-amine

Example 135 was prepared following the same general procedure as in Example 8. R_{f}: 0.4 (100% EtOAc). ¹H NMR (400 MHz, CD₃OD): δ 8.86 (s, 1H), 8.46 (d, J=8.6 Hz, 1H), 7.99-7.97 (m, 1H), 7.93-7.89 (m, 2H), 7.54-7.51 (m, 2H), 6.45 (br s, 1H), 6.15 (br s, 1H), 3.78 (m, 4H), 2.62-2.60 (m, 4H), 2.33 (s, 3H), 2.30 (m, 3H). m/e (M+1): 400.2; HPLC purity: >98%

### Example 136

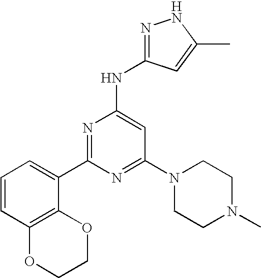

### 2-(2,3-dihydrobenzo[b][1,4]dioxin-5-yl)-N-(5-methyl-1H-pyrazol-3-yl)-6-(4-methylpiperazin-1-yl)pyrimidin-4-amine

Example 136 was prepared following the same general procedure as in Example 8. R_{f}: 0.4 (CHCl₃:MeOH; 9:1). ¹H NMR (400 MHz, CD₃OD): δ 7.77 (br s, 1H), 6.93-6.88 (m, 2H), 5.82 (br s, 1H), 5.64 (br s, 1H), 4.38 (br s, 4H), 3.68 (br s, 4H), 2.53 (br s, 4H), 2.34 (3H), 2.26 (s, 3H). m/e (M+1): 408.2; HPLC purity: >98%

### Example 137

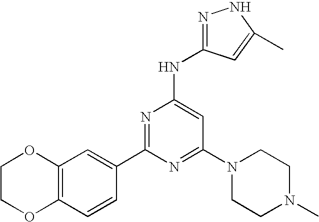

### 2-(2,3-dihydrobenzo[b][1,4]dioxin-6-yl)-N-(5-methyl-1H-pyrazol-3-yl)-6-(4-methylpiperazin-1-yl)pyrimidin-4-amine

Example 137 was prepared following the same general procedure as in Example 8. R_{f}: 0.3 (CHCl₃:MeOH; 9:1). ¹H NMR (400 MHz, CD₃OD); δ 7.83 (s, 2H), 6.87 (d, J=8.9 Hz, 1H), 6.30 (br s, 1H), 6.14 (br s, 1H), 4.29 (s, 4H), 3.71 (br s, 4H), 2.58-2.56 (m, 4H), 2.37 (s, 3H), 2.30 (s, 3H), m/e (M+1): 408.1; HPLC purity: >99%

### Example 139

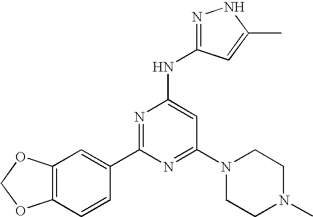

### 2-(benzo[d][1,3]dioxol-5-yl)-N-(5-methyl-1H-pyrazol-3-yl)-6-(4-methylpiperazin-1-yl)pyrimidin-4-amine

Example 139 was prepared following the same general procedure as in Example 8. R_{f}: 0.3 (CHCl₃:MeOH; 9:1). ¹H NMR (400 MHz, CD₃OD): δ 7.95 (d, J=8.1 Hz, 1H), 7.80 (br s, 1H), 6.88 (d, 1H, J=8.1 Hz), 6.32 (br s, 1H), 6.13 (br s, 1H), 6.01 (s, 2H), 3.71 (br s, 4H), 2.58 (br s, 4H), 2.37 (s, 3H), 2.30 (s, 3H). m/e (M+1): 394.2; HPLC purity: >94%

### Example 141

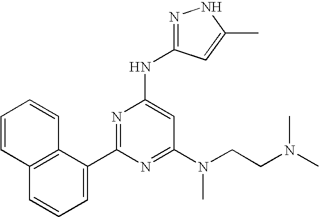

### N⁴-(2-(dimethylamino)ethyl)-N⁴-methyl-N⁶-(5-methyl-1H-pyrazol-3-yl)-2-(naphthalen-1-yl)pyrimidine-4,6-diamine

Example 141 was prepared following the same general procedure as in Example 8. R_{f}: 0.3 (CHCl₃:MeOH). ¹H NMR (400 MHz, CD₃OD): δ 8.55 (br s, 1H), 7.96-7.88 (m, 3H), 7.58-7.49 (m, 3H), 6.44 (br s, 1H), 6.05 (br s, 1H), 3.81-3.77 (m, 2H), 3.12 (s, 3H), 2.65-2.61 (m, 2H), 2.29 (s, 9H) m/e (M+1): 400.2; HPLC purity: >96%

### Example 142

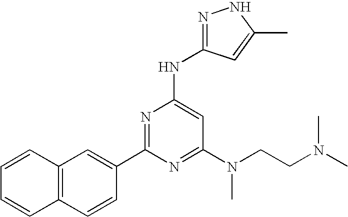

### N⁴-(2-(dimethylamino)ethyl)-N⁴-methyl-N⁶-(5-methyl-1H-pyrazol-3-yl)-2-(naphthalen-2-yl)pyrimidine-4,6-diamine

Example 142 was prepared following the same general procedure as in Example 8. R_{f}: 0.2 (CHCl₃:MeOH; 9:1). ¹H NMR (400 MHz, CD₃OD): δ 8.85 (s, 1H), 8.44 (d, J=8.6 Hz, 1H), 8.00-7.90 (m, 3H), 7.54-7.52 (m, 2H), 6.28 (s, 1H), 6.11 (s, 1H), 4.04 (t, J=6.8 Hz, 2H), 3.12 (s, 3H), 3.00 (t, J=6.8 Hz, 2H), 2.63 (s, 6H), 2.32 (s, 3H). m/e (M+1): 402.2; HPLC purity: >97%

### Example 143

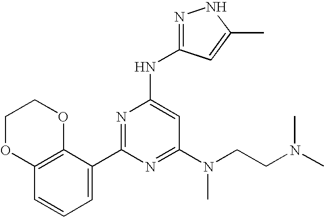

### 2-(2,3-dihydrobenzo[b][1,4]dioxin-5-yl)-N⁴-(2-(dimethylamino)ethyl)-N⁴-methyl-N⁶-(5-methyl-1H-pyrazol-3-yl)pyrimidine-4,6-diamine

Example 143 was prepared following the same general procedure as in Example 8. R_{f}: 0.3 (CHCl₃:MeOH). ¹H NMR (400 MHz, CD₃OD): δ 7.79 (br s, 1H), 6.93 (d, J=7.8 Hz, 1H), 6.89-6.85 (m, 1H), 6.75 (br s, 1H), 5.50 (s, 1H), 4.37 (br s, 4H), 3.77 (br s, 2H), 3.07 (s, 3H), 2.53 (t, J=7.4 Hz, 2H), 2.26 (s, 6H), 2.24 (s, 3H). m/e (M+1): 408.2; HPLC purity: >98%

### Example 146

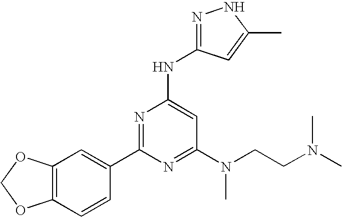

### 2-(benzo[d][1,3]dioxol-5-yl)-N⁴-(2-(dimethylamino)ethyl)-N⁴-methyl-N⁶-(5-methyl-1H-pyrazol-3-yl)pyrimidine-4,6-diamine

Example 146 was prepared following the same general procedure as in Example 8. R_{f}: 0.3 (CHCl₃:MeOH; 9:1). ¹H NMR (400 MHz, CD₃OD): δ 7.95 (d, J=8.2 Hz, 1H), 7.82 (s, 1H), 6.88 (d, J=8.2 Hz, 1H), 6.10 (br s, 2H), 6.00 (s, 2H), 3.84 (t, J=7.3 Hz, 2H), 3.07 (s, 3H), 2.66 (t, J=7.3 Hz, 2H), 2.39 (s, 6H), 2.30 (s, 3H). m/e (M+1): 396.2; HPLC purity: >90%

### Example 148

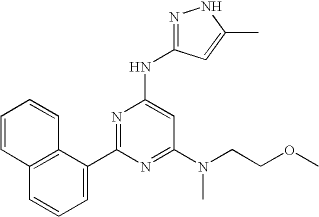

### N⁴-(2-methoxyethyl)-N⁴-methyl-N⁶-(5-methyl-1H-pyrazol-3-yl)-2-(naphthalen-1-yl)pyrimidine-4,6-diamine

Example 148 was prepared following the same general procedure as in Example 8. R_{f}: 0.3 (Hexane:EtOAc). ¹H NMR (400 MHz, CD₃OD): δ 8.56 (br s, 1H), 7.96-7.87 (m, 3H), 7.58-7.49 (m, 3H), 6.42 (s, 1H), 6.06 (s, 1H), 3.82-3.81 (m, 2H), 3.63 (m, J=5.6 Hz, 2H), 3.36 (s, 3H), 3.14 (s, 3H), 2.28 (s, 3H) m/e (M+1): 388.2; HPLC purity: >96%

### Example 149

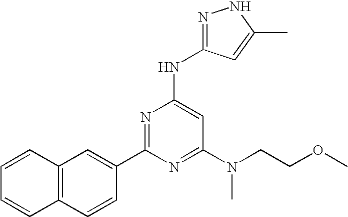

### N⁴-(2-methoxyethyl)-N⁴-methyl-N⁶-(5-methyl-1H-pyrazol-3-yl)-2-(naphthalen-2-yl)pyrimidine-4,6-diamine

Example 149 was prepared following the same general procedure as in Example 8. R_{f}: 0.3 (100% EtOAc). ¹H NMR (400 MHz, CD₃OD): δ 8.74 (s, 1H), 8.33 (d, J=8.6 Hz, 1H), 8.00-7.91 (m, 3H), 7.60-7.54 (m, 2H), 6.04 (s, 1H), 6.00 (s, 1H), 3.92 (br s, 2H), 3.70 (t, J=5.6 Hz, 2H), 3.39 (s, 3H), 3.21 (s, 3H), 2.33 (s, 3H). m/e (M+1): 389.2. HPLC purity: >98%

### Example 150

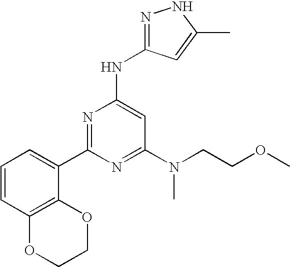

### 2-(2,3-dihydrobenzo[b][1,4]dioxin-5-yl)-N⁴-(2-methoxyethyl)-N⁴-methyl-N⁶-(5-methyl-1H-pyrazol-3-yl)pyrimidine-4,6-diamine

Example 150 was prepared following the same general procedure as in Example 8. R_{f}: 0.5 (100% EtOAc). ¹HNMR (400 MHz, CD₃OD): δ 7.79 (br s, 1H), 6.95-6.90 (m, 2H), 5.71 (br s, 1H), 5.55 (br s, 1H), 4.50 (br s, 2H), 4.40 (br s, 2H,), 3.82 (br s, 2H), 3.65 (t, J=5.6 Hz, 2H), 3.37 (s, 3H), 3.11 (s, 3H), 2.24 (s, 3H). m/e (M−1): 395; HPLC purity: >98%

### Example 153

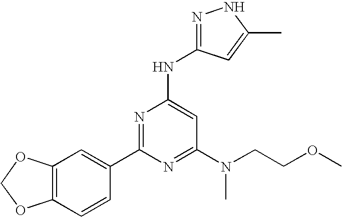

### 2-(benzo[d][1,3]dioxol-5-yl)-N⁴-(2-methoxyethyl)-N⁴-methyl-N⁶-(5-methyl-1H-pyrazol-3-yl)pyrimidine-4,6-diamine

Example 153 was prepared following the same general procedure as in Example 8. R_{f}: 0.6 (7:3 PE:EtOAc). ¹H NMR (400 MHz, CD₃OD): δ 7.94 (d, 1H, J=8.1 Hz), 7.80 (s, 1H), 6.88 (d, J=8.1 Hz, 1H), 6.10 (br s, 1H), 6.00 (s, 2H), 3.84 (br s, 2H), 3.66 (t, J=5.6 Hz, 1H), 3.37 (s, 3H), 3.12 (s, 3H), 2.30 (br s, 3H). m/e (M+1): 383; HPLC purity: >98%

### Example 155

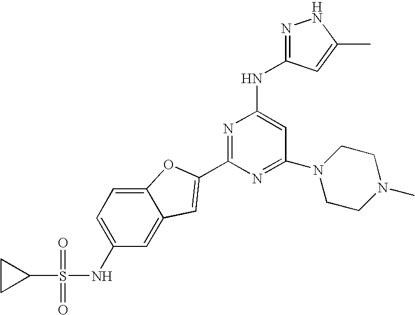

### N-(2-(4-(5-methyl-1H-pyrazol-3-ylamino)-6-(4-methylpiperazin-1-yl)pyrimidin-2-yl)benzofuran-5-yl)cyclopropanesulfonamide

Example 155 was prepared following the same general procedure as in Example 1. R_{f}: 0.5 (CHCl₃:MeOH; 9:1). ¹H NMR (400 MHz, CD₃OD): δ 7.64 (s, 1H), 7.56 (br s, 2H), 7.35-7.33 (m, 1H), 6.45 (br s, 1H), 6.14 (br s, 1H), 3.73 (br s, 4H), 2.59 (br s, 4H), 2.51 (m, 1H), 2.37 (s, 3H), 2.32 (br s, 3H), 1.02-1.00 (m, 2H), 0.97-0.89 (m, 2H). m/e (M+1): 509; HPLC purity: >98%

### Example 156

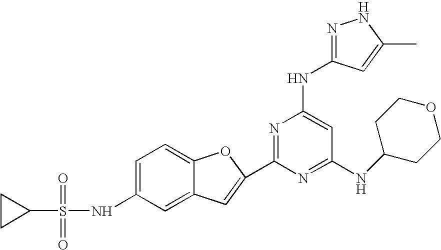

### N-(2-(4-(5-methyl-1H-pyrazol-3-ylamino)-6-(tetrahydro-2H-pyran-4-ylamino)pyrimidin-2-yl)benzofuran-5-yl)cyclopropanesulfonamide

Example 156 was prepared following the same general procedure as in Example 1. R_{f}; 0.5 (Pet ether:EtOAc; 9:1). ¹H NMR (400 MHz, CD₃OD): δ 911 (s, 1H), 7.64 (s, 1H), 7.57-7.53 (m, 2H), 7.53-7.32 (m, 1H), 6.20 (br s, 1H), 6.05 (br s, 1H), 4.01-3.98 (m, 3H), 3.62-3.57 (m, 2H), 2.55-2.50 (m, 1H), 2.30 (s, 3H), 2.04-2.02 (m, 2H), 1.70-1.55 (m, 2H), 1.01-0.96 (m, 4H). m/e (M+1): 510; HPLC purity: >92%

### Example 157

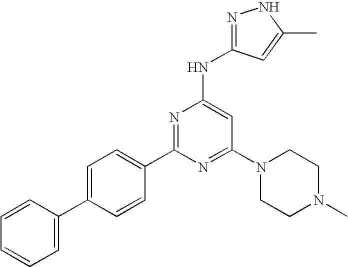

### 2-(biphenyl-4-yl)-N-(5-methyl-1H-pyrazol-3-yl)-6-(4-methylpiperazin-1-yl)pyrimidin-4-amine

Example 157 was prepared following the same general procedure as in Example 8. R_{f}; 0.4 (CHCl₃:MeOH; 9:1). ¹H NMR (400 MHz, CD₃OD): δ 8.42 (d, J=8.3 Hz, 2H), 7.73-7.69 (m, 4H), 7.47-7.45 (m, 2H), 7.40-7.35 (m, 1H), 6.35 (br s, 1H), 6.15 (br s, 1H), 3.76 (br s, 4H), 2.62 (br s, 4H), 2.40 (s, 3H), 2.32 (s, 3H). m/e (M+1): 426.2; HPLC purity: >98%

### Example 158

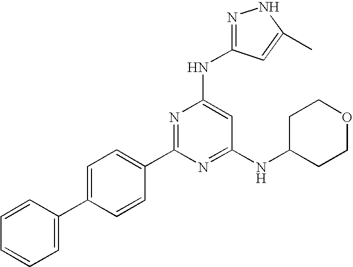

### 2-(biphenyl-4-yl)-N⁴-(5-methyl-1H-pyrazol-3-yl)-N⁶-(tetrahydro-2H-pyran-4-yl)pyrimidine-4,6-diamine

Example 158 was prepared following the same general procedure as in Example 8. R_{f}; 0.4 (100% EtOAc). ¹H NMR (400 MHz, CD₃OD): δ 8.38 (d, J=8.4 Hz, 2H), 7.73-7.70 (m, 4H), 7.49-7.45 (m, 2H), 7.38-7.370 (m, 1H), 6.19 (br s, 1H), 6.10 (br s, 1H), 4.20-4.10 (br s, 1H), 4.02-4.00 (m, 2H), 3.63-3.57 (m, 2H), 2.31 (s, 3H), 2.07-2.04 (m, 2H), 1.66-1.56 (m, 2H). m/e (M+1): 427.3; HPLC purity: >97%

### Example 159

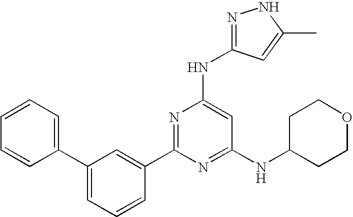

### 2-(biphenyl-3-yl)-N⁴-(5-methyl-1H-pyrazol-3-yl)-N⁶-(tetrahydro-2H-pyran-4-yl)pyrimidine-4,6-diamine

Example 159 was prepared following the same general procedure as in Example 8. R_{f}: 0.3 (PE:EtOAc; 2:8). ¹H NMR (400 MHz, CD₃OD): δ 8.60 (s, 1H), 8.27 (d, J=7.7 Hz, 1H), 7.72-7.70 (m, 3H), 7.54-7.45 (m, 3H), 7.38-7.35 (m, 1H), 6.15 (br s, 2H), 4.02-3.99 (m, 3H), 3.62-3.55 (m, 2H), 2.30 (s, 3H), 2.07-2.04 (m, 2H), 1.65-1.56 (m, 2H). m/e (M+1): 427.2; HPLC purity: >96%

### Example 160

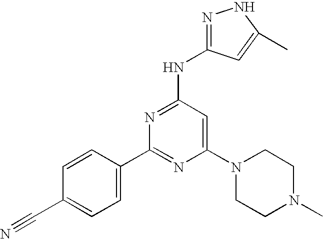

### 4-(4-(5-methyl-1H-pyrazol-3-ylamino)-6-(4-methylpiperazin-1-yl)pyrimidin-2-yl)benzonitrile

Example 160 was prepared following the same general procedure as in Example 196. ¹H NMR (300 MHz, CDCl₃): δ 9.53 (brs, 1H), 8.46 (d, 2H, J=8.3 Hz), 7.72 (d, 2H, J=8.6 Hz), 6.95 (brs, 1H), 6.55 (brs, 1H), 5.92 (s, 1H), 3.82-3.68 (m, 4H), 2.62-2.45 (m, 4H), 2.36 (s, 3H), 2.33 (s, 3H).

### Example 161

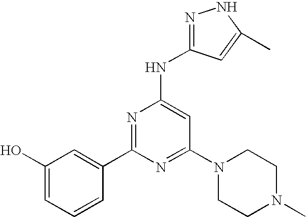

### 3-(4-(5-methyl-1H-pyrazol-3-ylamino)-6-(4-methylpiperazin-1-yl)pyrimidin-2-yl)phenol

Example 161 was prepared following the same general procedure as in Example 196. ¹H NMR (300 MHz, CD₃OD): δ 7.86-7.74 (m, 2H), 7.31-7.21 (m, 1H), 6.92-6.84 (m, 2H), 6.34 (br s, 1H), 6.19 (br s, 1H), 3.78-3.66 (m, 4H), 2.62-2.50 (m 4H), 2.73 (s, 3H), 2.31 (s, 3H).

### Example 162

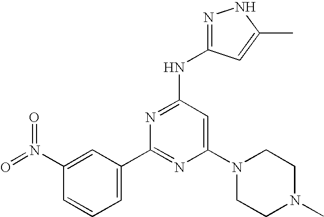

### N-(5-methyl-1H-pyrazol-3-yl)-6-(4-methylpiperazin-1-yl)-2-(3-nitrophenyl)pyrimidin-4-amine

Example 162 was prepared following the same general procedure as in Example 196. ¹H NMR (300 MHz, CDCl₃): δ 9.24 (s, 1H), 8.73 (d, J=8 Hz, 1H), 8.32-8.26 (m, 1H), 7.65-7.57 (m, 1H), 6.89 (br s, 1H), 6.60 (br s, 1H), 5.89 (br s, 1H), 3.83-3.71 (m, 4H), 2.61-2.49 (m, 4H), 2.39 (s, 3H), 2.37 (s, 3H).

### Example 163

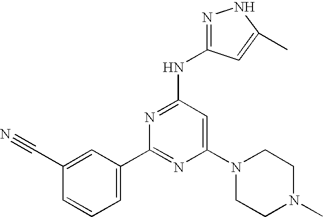

### 3-(4-(5-methyl-1H-pyrazol-3-ylamino)-6-(4-methylpiperazin-1-yl)pyrimidin-2-yl)benzonitrile

Example 163 was prepared following the same general procedure as in Example 196. ¹H NMR (300 MHz, CDCl₃): δ 9.45 (brs, 1H), 8.69 (t, 1H, J=1.4 Hz), 8.59 (dt, 1H, J=8.0, 1.4 Hz), 7.70 (dt, 1H, J=7.7, 1.4 Hz), 7.54 (t, 1H, J=7.7 Hz), 6.91 (brs, 1H), 6.56 (brs, 1H), 5.92 (s, 1H), 3.84-3.69 (m, 4H), 2.60-2.47 (m, 4H), 2.37 (s, 3H), 2.33 (s, 3H). HPLC purity >98%.

### Example 164

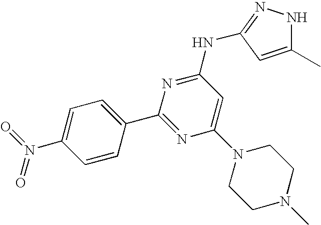

### N-(5-methyl-1H-pyrazol-3-yl)-6-(4-methylpiperazin-1-yl)-2-(4-nitrophenyl)pyrimidin-4-amine

Example 164 was prepared following the same general procedure as in Example 196. ¹H NMR (300 MHz, CDCl₃): δ 8.55 (d, J=8 Hz, 2H), 8.29 (d, J=8 Hz, 2H), 6.89 (br s, 1H), 6.59 (br s, 1H), 5.95 (br s, 1H), 3.81-3.71 (m, 4H), 2.58-2.48 (m, 4H), 2.38 (s, 3H), 2.36 (s, 3H).

### Example 165

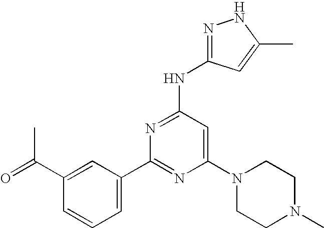

### 1-(3-(4-(5-methyl-1H-pyrazol-3-ylamino)-6-(4-methylpiperazin-1-yl)pyrimidin-2-yl)phenyl)ethanone

Example 165 was prepared following the same general procedure as in Example 196. ¹H NMR (300 MHz, CD₃OD): δ 8.92 (s, 1H), 8.55 (d, J=8 Hz, 1H), 8.03 (d, J=8 Hz, 1H), 7.53 (t, J=8 Hz, 1H), 6.29 (br s, 1H), 6.14 (br s, 1H), 3.36-3.30 (m, 4H), 2.66 (s, 3H), −2.50 (m 4H), 2.36 (s, 3H), 2.31 (s, 3H).

### Example 166

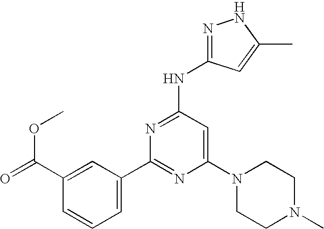

### methyl 3-(4-(5-methyl-1H-pyrazol-3-ylamino)-6-(4-methylpiperazin-1-yl)pyrimidin-2-yl)benzoate

Example 166 was prepared following the same general procedure as in Example 196. ¹H NMR (300 MHz, CD₃OD): δ 9.03-8.99 (m, 1H), 8.61-8.57 (m, 1H), 8.11-8.07 (m, 1H), 7.56 (t, J=8 Hz, 1H), 6.35 (br s, 1H), 6.14 (br s, 1H), 3.97 (s, 3H), 3.81-3.69 (m, 4H), 2.65-2.54 (m, 4H), 2.38 (s, 3H), 2.32 (s, 3H).

### Example 167

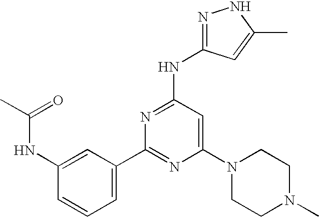

### N-(3-(4-(5-methyl-1H-pyrazol-3-ylamino)-6-(4-methylpiperazin-1-yl)pyrimidin-2-yl)phenyl)acetamide

Example 167 was prepared following the same general procedure as in Example 196. ¹H NMR (300 MHz, CD₃OD): δ 8.53 (s, 1H), 8.08 (d, J=8 Hz, 1H), 7.70 (d, J=8 Hz, 1H), 6.35 (br s, 1H), 6.18 (br s, 1H), 3.80-3.71 (m, 4H), 2.65-2.56 (m, 4H), 2.40 (s, 3H), 2.32 (s, 3H), 2.17 (s, 3H).

### Example 168

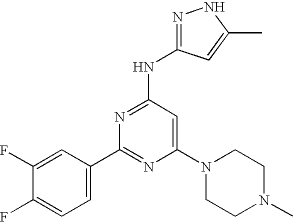

### 2-(3,4-difluorophenyl)-N-(5-methyl-1H-pyrazol-3-yl)-6-(4-methylpiperazin-1-yl)pyrimidin-4-amine

Example 168 was prepared following the same general procedure as in Example 196. ¹H NMR (300 MHz, CDCl₃): δ 8.26-8.06 (m, 2H), 7.23-7.14 (m, 1H), 6.81 (brs, 1H), 6.47 (brs, 1H), 5.91 (brs, 1H), 3.80-3.66 (m, 4H), 2.59-2.43 (m, 4H), 2.36 (s, 3H), 2.33 (s, 3H).

### Example 169

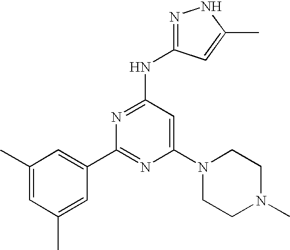

### 2-(3,5-dimethylphenyl)-N-(5-methyl-1H-pyrazol-3-yl)-6-(4-methylpiperazin-1-yl)pyrimidin-4-amine

Example 169 was prepared following the same general procedure as in Example 196. ¹H NMR (300 MHz, CDCl₃): δ 9.89 (brs, 1H), 7.95 (s, 2H), 7.07 (brs, 2H), 6.47 (brs, 1H), 5.87 (s, 1H), 3.82-3.66 (m, 4H), 2.58-2.47 (m, 4H), 2.39 (s, 6H), 2.35 (s, 3H), 2.29 (s, 3H).

### Example 170

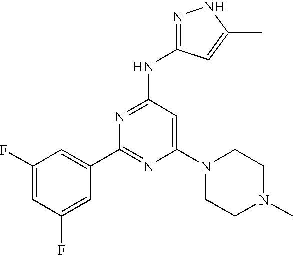

### 2-(3,5-difluorophenyl)-N-(5-methyl-1H-pyrazol-3-yl)-6-(4-methylpiperazin-1-yl)pyrimidin-4-amine

Example 170 was prepared following the same general procedure as in Example 196. ¹H NMR (300 MHz, CDCl₃): δ 7.95-7.83 (m, 2H), 6.93-6.78 (m, 2H), 6.53 (brs, 1H), 5.92 (s, 1H), 3.93-3.67 (m, 4H), 2.60-2.42 (m, 4H), 2.37 (brs, 3H), 2.33 (s, 3H).

### Example 171

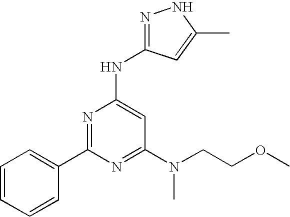

### N⁴-(2-methoxyethyl)-N⁴-methyl-N⁶-(5-methyl-1H-pyrazol-3-yl)-2-phenyl pyrimidine-4,6-diamine

Example 171 was prepared following the same general procedure as in Example 8. R_{f}: 0.3 (100% EtOAc). ¹H NMR (400 MHz, CD₃OD): δ 8.31-8.29 (m, 2H), 7.48-7.44 (m, 3H), 6.10 (s, 1H), 6.04 (s, 1H), 3.88 (t, 2H, J=5.5 Hz), 3.66 (t, 2H, J=5.5 Hz), 3.37 (s, 3H), 3.15 (s, 3H), 2.30 (s, 3H). m/e (M+1): 339.1; HPLC purity: >99%

### Example 172

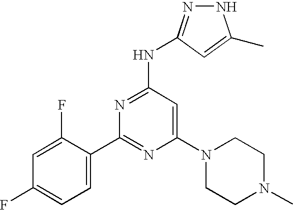

### 2-(2,4-difluorophenyl)-N-(5-methyl-1H-pyrazol-3-yl)-6-(4-methylpiperazin-1-yl)pyrimidin-4-amine

Example 172 was prepared following the same general procedure as in Example 196. ¹H NMR (300 MHz, CD₃OD): δ 8.51-8.38 (m, 1H), 7.37-7.20 (m, 2H), 6.24 (s, 1H), 5.92 (s, 1H), 4.95-4.69 (m, 4H), 3.82-3.44 (m, 4H), 3.00 (s, 3H), 2.33 (s, 3H). m/e (M+1): 386.

### Example 173

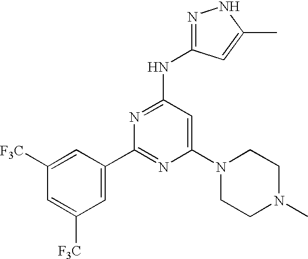

### 2-(3,5-bis(trifluoromethyl)phenyl)-N-(5-methyl-1H-pyrazol-3-yl)-6-(4-methylpiperazin-1-yl)pyrimidin-4-amine

Example 173 was prepared following the same general procedure as in Example 196. ¹H NMR (300 MHz, CDCl₃): δ 9.33 (brs, 1H), 8.84 (s, 2H), 7.91 (s, 1H), 6.91 (s, 1H), 6.66 (s, 1H), 5.93 (s, 1H), 3.83-3.69 (m, 4H), 2.60-2.47 (m, 4H), 2.37 (s, 3H), 2.34 (s, 3H). HPLC purity>99%.

### Example 174

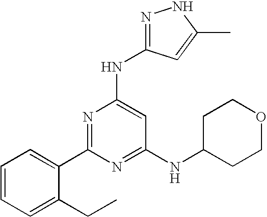

### 2-(2-ethylphenyl)-N⁴-(5-methyl-1H-pyrazol-3-yl)-N⁶-(tetrahydro-2H-pyran-4-yl)pyrimidine-4,6-diamine

Example 174 was prepared following the same general procedure as in Example 8. R_{f}; 0.6 (100% EtOAc). ¹H NMR (400 MHz, CD₃OD): δ 7.48 (d, 1H. J=6.9 Hz), 7.36-7.24 (m, 3H), 6.29 (br s, 1H), 5.96 (br s, 1H), 3.97-3.94 (m, 3H), 3.54-3.48 (m, 2H), 2.87 (q, 2H, J=7.5 Hz), 2.27 (s, 3H), 1.98-1.95 (m, 2H), 1.60-1.51 (m, 2H), 1.15 (t, 3H, J=7.5 Hz). m/e (M+1): 379.1; HPLC purity: >97%

### Example 175

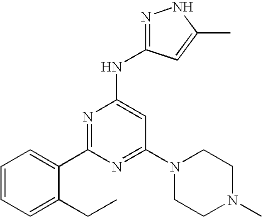

### 2-(2-ethyl phenyl)-N-(5-methyl-1H-pyrazol-3-yl)-6-(4-methylpiperazin-1-yl)pyrimidin-4-amine

Example 175 was prepared following the same general procedure as in Example 8. R_{f}; 0.3 (CHCl₃:MeOH; 9:1). ¹H NMR (400 MHz, CD₃OD): δ 7.57 (d, 1H, J=7.7 Hz), 7.36-7.23 (m, 3H), 6.48 (br s, 1H), 6.01 (br s, 1H), 3.68-3.66 (m, 4H), 2.91 (q, 2H, J=7.5 Hz), 2.55-2.53 (m, 4H), 2.35 (s, 3H), 2.28 (s, 3H), 1.14 (t, 3H, J=7.5 Hz). m/e (M+1): 378.2; HPLC purity: >95%

### Example 178

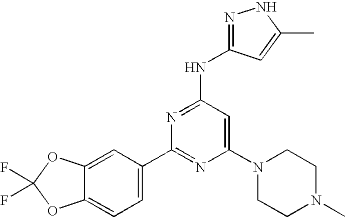

### 2-(2,2-difluorobezo[d][1,3]dioxol-5-yl)-N-(5-methyl-1H-pyrazol-3-yl)-6-(4-methylpiperazin-1-yl)pyrimidin-4-amine

Example 178 was prepared following the same general procedure as in Example 196. ¹H NMR (300 MHz, CD₃OD): δ 8.23 9dd, J=1, 9, Hz, 1H), 8.15-8.09 (m, 1H), 7.22 (d, J=9 Hz, 1H), 6.29 (br s, 1H), 6.06 (br s, 1H), 3.75-3.65 (m, 4H), 2.62-2.50 (m, 4H), 2.37 (s, 3H), 2.31 (s, 3H).

### Example 179

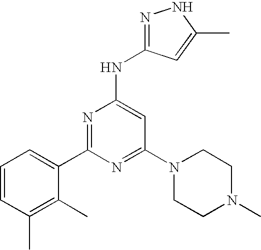

### 2-(2,3-dimethylphenyl)-N-(5-methyl-1H-pyrazol-3-yl)-6-(4-methylpiperazin-1-yl)pyrimidin-4-amine

Example 179 was prepared following the same general procedure as in Example 8. R_{f}: 0.3 (CHCl₃:MeOH; 9:1). ¹H NMR (400 MHz, CD₃OD): δ 7.36 (d, J=7.4 Hz, 1H), 7.21 (d, J=7.4 Hz, 1H), 7.16-7.12 (m, 1H), 6.40 (br s, 1H), 5.95 (s, 1H), 3.68-3.65 (m, 4H), 2.56-2.54 (m, 4H), 2.353 (s, 3H), 2.35 (s, 3H), 2.32 (s, 3H), 2.27 (s, 3H). m/e (M+1): 378.2; HPLC purity: >99%

### Example 180

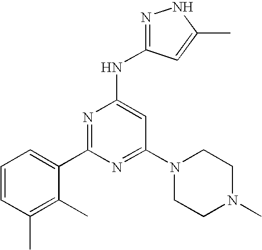

### 2-(2,3-dimethylphenyl)-N⁴-(5-methyl-1H-pyrazol-3-yl)-N⁶-(tetrahydro-2H-pyran-4-yl)pyrimidine-4,6-diamine

Example 180 was prepared following the same general procedure as in Example 8. R_{f}: 0.25 (100% EtOAc). ¹H NMR (400 MHz, CD₃OD): δ 7.28 (d, 1H, J=7.6 Hz), 7.21 (d, 1H, J=7.3 Hz), 7.17-7.13 (m, 1H), 6.21 (s, 1H), 5.91 (s, 1H), 3.97-3.94 (m, 3H), 3.55-3.49 (m, 2H), 2.34 (s, 3H), 2.27 (s, 3H), 2.23 (s, 3H), 1.98-1.95 (m, 2H), 1.61-1.52 (s, 2H). m/e (M+1): 379.1; HPLC purity: >97%

### Example 181

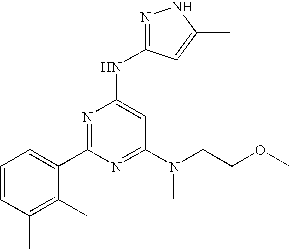

### 2-(2,3-dimethylphenyl)-N⁴-(2-methoxyethyl)-N⁴-methyl-N⁶-(5-methyl-1H-pyrazol-3-yl)pyrimidine-4,6-diamine

Example 181 was prepared following the same general procedure as in Example 8. R_{f}: 0.2 (100% EtOAc). ¹H NMR (400 MHz, CD₃OD): δ 7.35 (d, 1H, J=7.4 Hz), 7.22 (d, 1H, J=7.4 Hz), 7.16-7.14 (m, 1H), 6.25 (br s, 1H), 5.93 (s, 1H), 3.78 (t, 2H, J=5.6 Hz), 3.60 (t, 2H, J=5.6 Hz), 3.42 (s, 3H), 3.11 (s, 3H), 2.35 (s, 3H), 2.33 (s, 3H), 2.27 (s, 3H). m/e (M+1): 367.2; HPLC purity: >98%

### Example 182

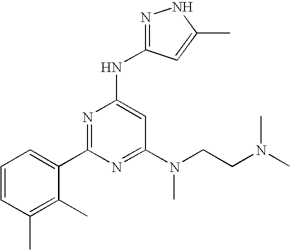

### N⁴-(2-(dimethylamino)ethyl)-2-(2,3-dimethyl phenyl)-N⁴-methyl-N⁶-(5-methyl-1H-pyrazol-3-yl)pyrimidine-4,6-diamine

Example 182 was prepared following the same general procedure as in Example 8. R_{f}: 0.3 (CHCl₃:MeOH; 9:1). ¹H NMR (400 MHz, CD₃OD): δ 7.33 (d, 1H, J=7 Hz), 7.20 (d, 1H, J=7.2 Hz), 7.16-7.11 (m, 1H), 6.39 (s, 1H), 5.98 (s, 1H), 3.76 (t, 2H, J=7.1 Hz), 3.08 (s, 3H), 2.63 (t, 2H, J=7.1 Hz), 2.35 (s, 6H), 2.33 (s, 6H), 2.27 (s, 3H). m/e (M+1): 380.2; HPLC purity: >97%

### Example 183

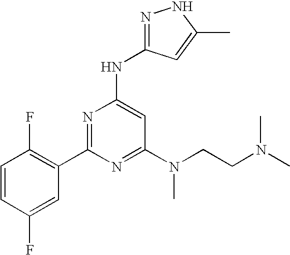

### 2-(2,5-difluorophenyl)-N⁴-(2-(dimethylamino)ethyl)-N⁴-methyl-N⁶-(5-methyl-1H-pyrazol-3-yl)pyrimidine-4,6-diamine

Example 183 was prepared following the same general procedure as in Example 8. R_{f}: 0.3 (CHCl₃:MeOH; 9:1). ¹H NMR (400 MHz, CD₃OD): δ 7.73 (br s, 1H), 7.19 (br s, 2H), 6.30 (br s, 1H), 6.13 (br s, 1H), 3.80 (br s, 2H), 3.13 (s, 3H), 2.61 (t, 2H, J=7.6 Hz), 2.35 (s, 6H), 2.27 (s, 3H). m/e (M+1): 388.2; HPLC purity: >94%

### Example 184

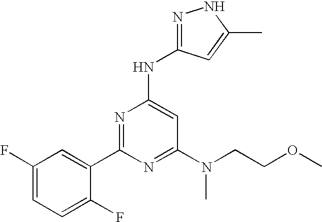

### 2-(2,5-difluorophenyl)-N⁴-(2-methoxyethyl)-N⁴-methyl-N⁶-(5-methyl-1H-pyrazol-3-yl)pyrimidine-4,6-diamine

Example 184 was prepared following the same general procedure as in Example 8. R_{f}: 0.25 (100% EtOAc). ¹H NMR (400 MHz, CD₃OD): δ 7.80 (br s, 1H), 7.23-7.20 (m, 2H), 6.09 (br s, 1H), 5.93 (br s, 1H), 3.82 (t, 2H, J=5.2 Hz), 3.64 (t, 2H, J=5.2 Hz), 3.36 (s, 3H), 3.12 (s, 3H), 2.26 (s, 3H). m/e (M+1): 375; HPLC purity: >97%

### Example 185

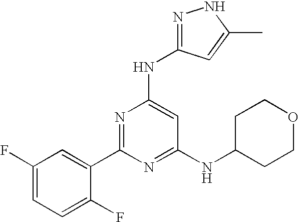

### 2-(2,5-difluorophenyl)-N⁴-(5-methyl-1H-pyrazol-3-yl)-N⁶-(tetrahydro-2H-pyran-4-yl)pyrimidine-4,6-diamine

Example 185 was prepared following the same general procedure as in Example 8. R_{f}: 0.25 (100% EtOAc). ¹H NMR (400 MHz, CD₃OD): δ 7.62 (br s, 1H), 7.20 (br s, 2H), 6.19 (br s, 1H), 6.01 (br s, 1H), 3.99-3.97 (m, 3H), 3.58-3.53 (m, 2H), 2.26 (s, 3H), 2.02-1.99 (m, 2H), 1.62-1.52 (m, 2H). m/e (M+1): 387; HPLC purity: >90%

### Example 186

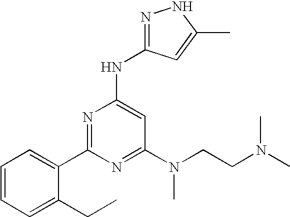

### N⁴-(2-(dimethylamino)ethyl)-2-(2-ethylphenyl)-N⁴-methyl-N⁶-(5-methyl-1H-pyrazol-3-yl)pyrimidine-4,6-diamine

Example 186 was prepared following the same general procedure as in Example 8. R_{f}: 0.3 (CHCl₃:MeOH; 9:1). ¹H NMR (400 MHz, CD₃OD): δ 7.53 (d, 1H, J=7.4 Hz), 7.35-7.24 (m, 3H), 6.38 (br s, 1H), 5.97 (br s, 1H), 3.78 (t, 2H, J=7 Hz), 3.08 (s, 3H), 2.92 (q, 2H, J=7.5 Hz), 2.74 (br s, 2H), 2.38 (s, 6H), 2.27 (s, 3H), 1.14 (t, 3H, J=7.5 Hz). m/e (M+1): 380.2; HPLC purity: >97%

### Example 187

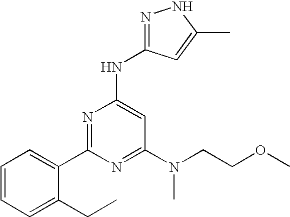

### 2-(2-ethyl phenyl)-N⁴-(2-methoxyethyl)-N⁴-methyl-N⁶-(5-methyl-1H-pyrazol-3-yl)pyrimidine-4,6-diamine

Example 187 was prepared following the same general procedure as in Example 8. R_{f}: 0.3 (CHCl₃:MeOH; 9:1). ¹H NMR (400 MHz, CD₃OD): δ 7.53 (d, 1H, J=7.6 Hz), 7.35-7.23 (m, 3H), 6.28 (br s, 1H), 5.95 (br s, 1H), 3.79 (t, 2H, J=5.6 Hz), 3.59 (t, 2H, J=5.6 Hz), 3.34 (s, 3H), 3.06 (s, 3H), 2.92 (q, 2H, J=7.5 Hz), 2.27 (s, 3H), 1.15 (t, 3H, J=7.5 Hz). m/e (M+1): 367.1; HPLC purity: >97%

### Example 188

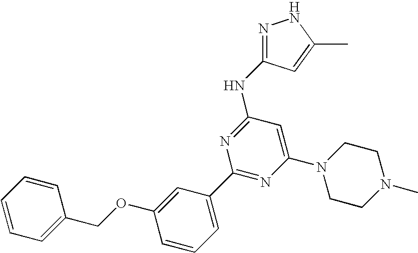

### 2-(3-(benzyloxy)phenyl)-N-(5-methyl-1H-pyrazol-3-yl)-6-(4-methylpiperazin-1-yl)pyrimidin-4-amine

Example 188 was prepared following the same general procedure as in Example 196. ¹H NMR (300 MHz, CDCl₃): δ 8.05-7.97 (m, 2H0, 7.54-7.31 (m, 6H), 7.08 (dd, J=2, 8 Hz, 1H), 6.8 (s, 1H), 6.4 (s, 1H), 5.9 (s, 1H), 5.19 (s, 2H), 3.78-3.70 (m, 4H), 2.57-2.49 (m, 4H), 2.38 (s, 3H), 2.34 (s, 3H).

### Example 189

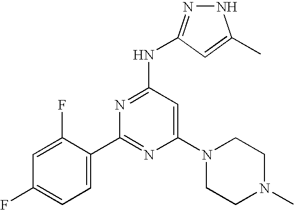

### 2-(2,4-difluorophenyl)-N-(5-methyl-1H-pyrazol-3-yl)-6-(4-methylpiperazin-1-yl)pyrimidin-4-amine

Example 189 was prepared following the same general procedure as in Example 196. ¹H NMR (300 MHz, CD₃OD): δ 8.29-8.15 (m, 1H), 7.18-7.02 (m, 2H), 6.21 (s, 1H), 5.88 (s, 1H), 4.93-4.66 (m, 4H), 3.56-3.10 (m, 4H), 2.89 (s, 3H), 2.22 (s, 3H). m/e (M+1): 386.

### Example 190

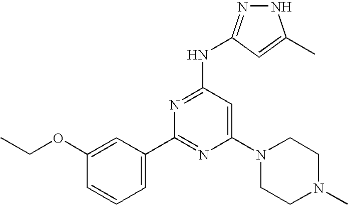

### 2-(3-ethoxyphenyl)-N-(5-methyl-1H-pyrazol-3-yl)-6-(4-methylpiperazin-1-yl)pyrimidin-4-amine

Example 190 was prepared following the same general procedure as in Example 196. ¹H NMR (300 MHz, CDCl₃): δ 7.99-7.87 (m, 2H), 7.34 (t, 1H, J=7.9 Hz), 7.09 (brs, 1H), 7.02-6.95 (m, 1H), 6.44 (s, 1H), 5.91 (s, 1H), 4.13 (q, 2H, J=7.0 Hz), 3.82-3.66 (m, 4H), 2.58-2.44 (m, 4H), 2.36 (s, 3H), 2.30 (s, 3H), 1.452 (t, 3H, J=7.0 Hz). m/e (M+1): 394; HPLC purity>96%.

### Example 191

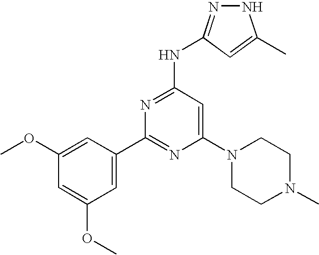

### 2-(3,5-dimethoxyphenyl)-N-(5-methyl-1H-pyrazol-3-yl)-6-(4-methylpiperazin-1-yl)pyrimidin-4-amine

Example 191 was prepared following the same general procedure as in Example 196. ¹H NMR (300 MHz, CDCl₃): δ 7.55 (d, 2H, J=2.4 Hz), 7.45 (brs, 1H), 6.56 (t, 1H, J=2.4 Hz), 6.50 (s, 1H), 5.91 (s, 1H), 3.86 (s, 6H), 3.78-3.66 (m, 4H), 2.60-2.43 (m, 4H), 2.35 (s, 3H), 2.27 (s, 3H). m/e (M+1): 410.

### Example 192

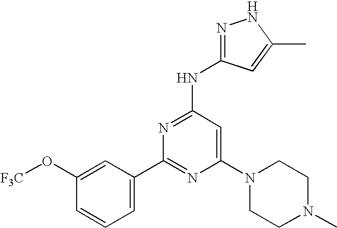

### N-(5-methyl-1H-pyrazol-3-yl)-6-(4-methylpiperazin-1-yl)-2-(3-(trifluoromethoxy)phenyl)pyrimidin-4-amine

Example 192 was prepared following the same general procedure as in Example 196. ¹H NMR (300 MHz, CDCl₃): δ 9.50 (brs, 1H), 8.34-8.26 (m, 1H), 8.25-8.20 (m, 1H), 7.45 (t, 1H, J=8.0 Hz), 7.30-7.24 (m, 1H), 6.96 (s, 1H), 6.49 (s, 1H), 5.95 (s, 1H), 3.82-3.67 (m, 4H), 2.60-2.44 (m, 4H), 2.36 (s, 3H), 2.32 (s, 3H). m/e (M+1): 434; HPLC purity>96%.

### Example 193

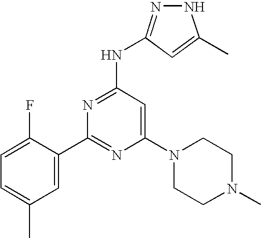

### 2-(2-fluoro-5-methylphenyl)-N-(5-methyl-1H-pyrazol-3-yl)-6-(4-methylpiperazin-1-yl)pyrimidin-4-amine

Example 193 was prepared following the same general procedure as in Example 196. ¹H NMR (300 MHz, CDCl₃): δ 7.88-7.76 (m, 1H), 7.22-7.11 (m, 1H), 7.09-6.97 (m, 2H), 6.32 (brs, 1H), 5.80 (s, 1H), 3.82-3.62 (m, 4H), 2.56-2.44 (m, 4H), 2.37 (s, 3H), 2.35 (s, 3H), 2.28 (s, 3H). m/e (M+1): 382.

### Example 194

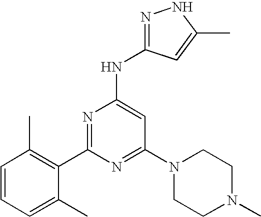

### 2-(2,6-dimethylphenyl)-N-(5-methyl-1H-pyrazol-3-yl)-6-(4-methylpiperazin-1-yl)pyrimidin-4-amine

Example 194 was prepared following the same general procedure as in Example 8. R_{f}: 0.3 (CHCl₃:MeOH; 9:1). ¹H NMR (400 MHz, CD₃OD): δ 7.19-7.17 (m, 1H), 7.08 (d, 2H, J=7.5 Hz), 6.54 (s, 1H), 5.95 (s, 1H), 3.63 (t, 4H, J=5 Hz), 2.52 (t, 4H, J=5 Hz), 2.34 (s, 3H), 2.26 (s, 3H), 2.16 (s, 6H). m/e (M+1): 378.2; HPLC purity: >94%

### Example 195

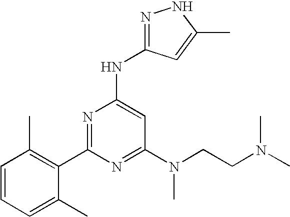

### N⁴-(2-(dimethylamino)ethyl)-2-(2,6-dimethylphenyl)-N⁴-methyl-N⁶-(5-methyl-1H-pyrazol-3-yl)pyrimidine-4,6-diamine

Example 195 was prepared following the same general procedure as in Example 8. R_{f}: 0.3 (CHCl₃:MeOH; 9:1). ¹H NMR (400 MHz, CD₃OD): δ 7.18-7.14 (m, 1H), 7.07 (d, 2H, J=7.6 Hz), 6.43 (s, 1H), 5.92 (s, 1H), 3.72 (t, 2H, J=7.2 Hz), 3.09 (s, 3H), 2.56 (t, 2H, J=7.2 Hz), 2.27 (s, 9H), 2.16 (s, 6H). m/e (M+1): 380.2; HPLC purity: >96%

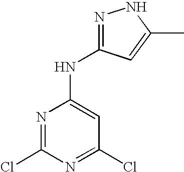

### 2,6-dichloro-N-(5-methyl-1H-pyrazol-3-yl)pyrimidin-4-amine

2,4,6-trichloropyrimidine (23.15 mL, 200 mmol) was dissolved in N,N-dimethylacetamide (anhydrous, 200 mL) and DIPEA (43.5 mL, 250 mmol) was added. The mixture was stirred at rt and the reaction was followed by TLC (1:1 Hex:EtOAc). After ˜18 h, the reaction was complete by TLC. Dilute reaction mixture with water (400 mL) and isolate resulting crystals with vacuum filtration. The product was dried under vacuum at 50° C. for 24 h to give 34.2 g final product (70% yield). ¹H-NMR (300 MHz, CDCl₃): δ 11.24 (br s, 1H), 9.61 (br s, 1H), 7.62 (s, 1H), 5.99 (s, 1H), 2.37 (s, 3H).

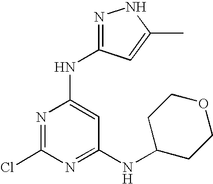

### 2-chloro-N⁴-(5-methyl-1H-pyrazol-3-yl)-N⁶-(tetrahydro-2H-pyran-4-yl)pyrimidine-4,6-diamine

2,6-dichloro-4-(amino-(5-methylpyrazole))pyrimidine (7.32 g, 29.9 mmol), 4-aminotetrahydropyran (3.3 g, 32.6 mmol) and DIPEA (10 mL, 57.5 mmol) was dissolved in DMA (anhydrous 60 mL). The reaction mixture was heated at 80° C. for 48 h. TLC (EtOAc) showed the formation of two new products and loss of starting material. The reaction was dilute with water and the product was extracted with ethyl acetate. The ethyl acetate was washed with water, brine and dried over Na2SO4 then rotoevaped to give a viscous solid. Ether was added to the solid and a solid formed. The ppt was isolated and confirmed to be 6-chloro-2-(4-aminopyran)-4-(amino(5-methylpyrazole))pyrimidine (2.6 g). The mother liquor was concentrated and purified by column chromatography (CHCL3 to 9:1 CHCl3 gradient) to give another 0.5 g of 6-chloro-2-(4-aminopyran)-4-(amino(5-methylpyrazole))pyrimidine and 0.483 g of 2-chloro-6-(4-aminopyran)-4-(amino(5-methylpyrazole))pyrimidine. ¹H-NMR (300 MHz, CDCl₃): δ 11.90 (s, 1H), 9.34 (s, 1H), 7.38 (d, J=8 Hz, 1H), 6.44 (s, 1H), 5.78 (s, 1H), 3.94-3.78 (m, 3H), 3.45-3.29 (m, 2H), 2.18 (s, 3H), 1.84-1.72 (m, 2H), 1.49-1.32 (m, 2H).

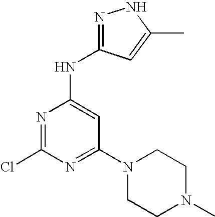

### 2-chloro-N-(5-methyl-1H-pyrazol-3-yl)-6-(4-methylpiperazin-1-yl)pyrimidin-4-amine

Reaction done as above except used 2,6-dichloro-4-(amino-(5-methylpyrazole))pyrimidine (2.44 g, 10 mmol), 1-methylpiperazine (1.2 mL, 11 mmol), DIPEA (3.6 mL, 15 mmol) in DMA (anhydrous 20 mL). The product was purified by column chromatography to (THF) to give 2 substituted product, the 6 substituted product was eluted with EtOAc:MeOH (1:1). The 6-substitued product crystallized from ether to give 164 mg. The 2-substituted product was a solid from the chromatography column (300 mg). ¹H-NMR (300 MHz, CDCl₃): δ 9.9 (br s, 1H), 5.80 (s, 1H), 3.68-3.58 (m, 4H), 2.51-2.42 (m, 4H), 2.35 (s, 3H), 2.32 (s, 3H).

### Example 196

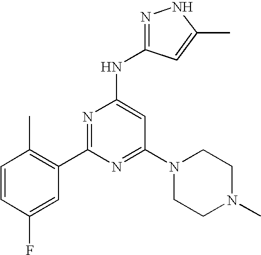

### 2-(5-fluoro-2-methylphenyl)-N-(5-methyl-1H-pyrazol-3-yl)-6-(4-methylpiperazin-1-yl)pyrimidin-4-amine

2-chloro-N-(5-methyl-1H-pyrazol-3-yl)-6-(4-methylpiperazin-1-yl)pyrimidin-4-amine (150 mg, 0.49 mmol) and 5-fluoro-2-methyl phenyl boronic acid (105 mg, 0.68 mmol), Bis(di-tert-butyl(4-dimethylaminophenyl)phosphine)dichloropalladium(II) (69 mg, 0.097 mmol) Na₂CO₃ (satd., 0.3 mL) and triethylamine (0.3 mL) were dissolved in DMF (anhydrous 2.5 mL) in a 10 ml microwave reactor. The reactor was flushed with argon, and heated at 130° C. for 40 min (reaction was followed by HPLC). The reaction was diluted with water (10 mL) and washed with CHCl₃; MeOH (9:1, 3×8 mL). The combined organics were washed with water (2×10 mL) and brine (10 mL). The organic layer was dried with Na₂SO₄, filtered and concentrated under reduced pressure. The crude product was purified using an ISCO Combiflash® SiO₂ column (CHCl₃, MeOH gradient) to give 71 mg final product (0.187 mmol, 38% yield). ¹H NMR (300 MHz, CDCl₃): δ 9.36 (brs, 1H), 7.48 (dd, 1H, J=9.9, 2.8 Hz), 7.16 (dd, 1H, J=8.5, 5.8 Hz), 6.98 (td, 1H, J=8.3, 2.9 Hz), 6.87 (brs, 1H), 6.51 (brs, 1H), 5.84 (s, 1H), 3.76-3.62 (m, 4H), 2.56-2.43 (m, 7H), 2.35 (s, 3H), 2.30 (s, 3H). m/e (M+1): 382; HPLC purity>968%.

### Example 197

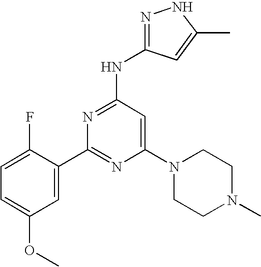

### 2-(2-fluoro-5-methoxyphenyl)-N-(5-methyl-1H-pyrazol-3-yl)-6-(4-methylpiperazin-1-yl)pyrimidin-4-amine

Example 197 was prepared following the same general procedure as in Example 196. ¹H NMR (300 MHz, CDCl₃): δ 7.64-7.55 (m, 1H), 7.13-7.01 (m, 1H), 6.97-6.86 (m, 2H), 6.30 (brs, 1H), 5.81 (s, 1H), 3.84 (s, 3H), 3.76-3.63 (m, 4H), 2.56-2.44 (m, 4H), 2.35 (s, 3H), 2.29 (s, 3H). m/e (M+1): 398; HPLC purity>98%.

### Example 198

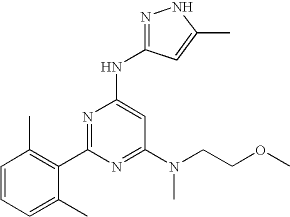

### 2-(2,6-dimethylphenyl)-N⁴-(2-methoxyethyl)-N⁴-methyl-N⁶-(5-methyl-1H-pyrazol-3-yl)pyrimidine-4,6-diamine

Example 198 was prepared following the same general procedure as in Example 8. R_{f}: 0.3 (100% EtOAc). ¹H NMR (400 MHz, DMSO-d₆): δ 11.87 (s, 1H), 9.18 (s, 1H), 7.15-7.11 (m, 1H), 7.05-7.03 (d, 2H, J=7.6 Hz), 6.57 (s, 1H), 5.83 (s, 1H), 3.66 (t, 2H, J=5.6 Hz), 3.47 (t, 2H, J=5.6 Hz), 3.23 (s, 3H), 2.99 (s, 3H), 2.51 (s, 3H), 2.09 (s, 6H). m/e (M+1): 367.2; HPLC purity: >99%

### Example 199

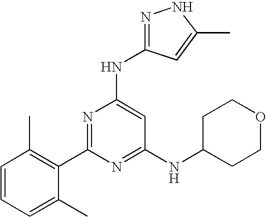

### 2-(2,6-dimethylphenyl)-N⁴-(5-methyl-1H-pyrazol-3-yl)-N⁶-(tetrahydro-2H-pyran-4-yl)pyrimidine-4,6-diamine

Example 199 was prepared following the same general procedure as in Example 8. R_{f}: 0.3 (100% EtOAc). ¹H NMR (400 MHz, CD₃OD): δ 7.20-7.16 (m, 1H), 7.08 (d, 2H, J=7.6 Hz), 6.35 (s, 1H), 5.92 (s, 1H), 3.96-3.93 (m, 3H), 3.50 (t, 2H, J=11.2 Hz), 2.28 (s, 3H), 2.17 (s, 6H), 1.96-1.93 (m, 2H), 1.58-1.51 (m, 2H). m/e (M+1): 379.2; HPLC purity: >97%

### Example 200

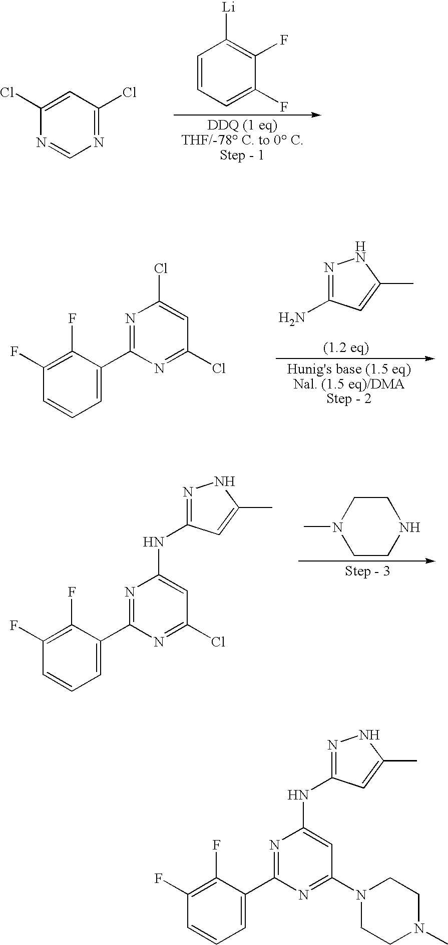

Step-1: n-Butyl lithium (0.895 g, 0.0140 mol, 1.6 M in hexane) was added dropwise to a stirred solution of 2,3-difluorobromobenzene (3.0 g, 0.0155 mol) in THF (40 ml) over a period of 20 min at −78° C., and reaction was continued stirring for 2 h at the same temperature. Then, 4,6-dichloropyrimidine (2.316 g, 0.0155 mol) in THF (20 ml) was added drop wise to the generated 2,3-difluorophenyl lithium mixture over a period of 15 min at −78° C., and reaction was continued stirring for 30 min. Then, the reaction mixture was slowly warmed to 0° C. and was quenched with water (30 ml), and then DDQ (3.53 g, 0.0155 mol) in THF (30 ml) was added portionwise with stirred for 10 min. The resultant reaction mixture was extracted with CH₂Cl₂ (3×50 ml), washed with brine (50 ml), dried (Na₂SO₄), and concentrated. The concentrated product was purified through silica column chromatography using pet. ether to afford step-1 product (1.1 g, 27.1%) as an off white solid. R_{f}: 0.3 (100% PE). ¹H NMR (400 MHz, CD₃OD): δ 8.82 (s, 1H), 7.91-7.78 (m, 1H), 7.48-7.45 (m, 1H), 7.32-7.26 (m, 1H). m/e (M+1): 260.8

Step-2: 3-Amino-5-methyl-pyrazole (0.493 g, 0.0051 mol) was added to a stirred solution of mixture of step-1 product (1.1 g, 0.0042 mol) in dimethylacetamide (20 ml), and diisopropyl ethylamine (0.816 g, 0.0063 mol) at RT. Then, potassium iodide (1.05 g, 0.0063 mol) was added to the reaction mixture at the same temperature, and heated at 55° C. for 72 h. After completion of reaction, reaction mixture was cooled to RT, and quenched with ice cold water, and extracted with EtOAc (3×60 ml), washed with water (100 ml), brine (100 ml), dried (Na₂SO₄), and concentrated. Purification of concentrated product was done through silica column chromatography using 20% ethyl acetate in pet ether to afford step-2 product (400 mg, 29.7%) as an off white solid. R_{f}: 0.5 (PE:EtOAc; 1:1). m/e (M+1): 321.9

Step-3: N-Methylpiperazine (0.1 ml) was added to step 2 product (100 mg, 0.0003 mol) at RT, and heated at 90° C. for 2 h. After completion of reaction, the reaction mixture was cooled to RT, and quenched with water (10 ml), filtered and washed with water (10 ml), P.E (20 ml). The solid obtained was purified through column chromatography using 8% methanol in dichloromethane to afford EXAMPLE 200 (10 mg, 8.5%) as a pale brown solid. H¹NMR is not clean. m/e (M+1): 386; HPLC purity: >96%.

### Example 201

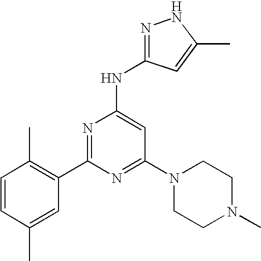

### 2-(2,5-dimethylphenyl)-N-(5-methyl-1H-pyrazol-3-yl)-6-(4-methylpiperazin-1-yl)pyrimidin-4-amine

Example 201 was prepared following the same general procedure as in Example 8. R_{f}: 0.4 (9:1 CHCl₃:MeOH). ¹H NMR (400 MHz, CD₃OD): δ 7.45 (s, 1H), 7.15-7.13 (m, 2H), 6.46 (br s, 1H), 6.01 (br s, 1H), 3.66 (t, 4H, J=4.8 Hz), 2.53 (t, 4H, J=4.8 Hz), 2.43 (s, 3H), 2.35 (s, 6H), 2.28 (s, 3H). m/e (M+1): 378.2; HPLC purity: >98%

### Example 202

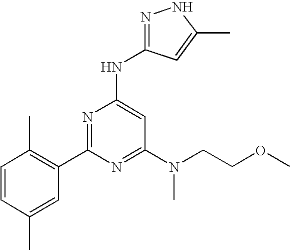

### 2-(2,5-dimethylphenyl)-N⁴-(2-methoxyethyl)-N⁴-methyl-N⁶-(5-methyl-1H-pyrazol-3-yl)pyrimidine-4,6-diamine

Example 202 was prepared following the same general procedure as in Example 8. R_{f}: 0.4 (100% EtOAc). ¹H NMR (400 MHz, DMSO-d₆): δ 11.85 (br s, 1H), 9.11 (br s, 1H), 7.53 (s, 1H), 7.12-7.07 (m, 2H), 6.57 (br s, 1H), 5.92 (br s, 1H), 3.70 (t, 2H, J=5.3 Hz), 3.50 (m, 2H, J=5.3 Hz), 3.25 (s, 3H), 3.17 (s, 3H), 2.50 (s, 3H), 2.49 (s, 3H), 2.17 (s, 3H). m/e (M+1): 367.2; HPLC purity: >98%

### Example 203

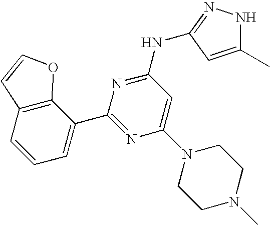

### 2-(benzofuran-7-yl)-N-(5-methyl-1H-pyrazol-3-yl)-6-(4-methylpiperazin-1-yl)pyrimidin-4-amine

Example 203 was prepared following the same general procedure as in Example 8. R_{f}: 0.3 (CHCl₃:MeOH). ¹H NMR (400 MHz, CD₃OD): δ 8.42 (br s, 1H), 8.26 (br s, 1H), 7.78 (d, 1H, J=7.6 Hz), 7.38-7.34 (m, 1H), 6.99 (br s, 1H), 5.86 (br s, 1H), 5.60 (br s, 1H), 3.75 (br s, 4H), 2.60 (br s, 4H), 2.38 (s, 3H), 2.27 (s, 3H). m/e (M+1): 390.2; HPLC purity: >98%

### Example 204

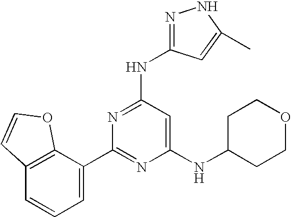

### 2-(benzofuran-7-yl)-N⁴-(5-methyl-1H-pyrazol-3-yl)-N⁶-(tetrahydro-2H-pyran-4-yl)pyrimidine-4,6-diamine

Example 204 was prepared following the same general procedure as in Example 8. R_{f}: 0.3 (100% EtOAc). ¹H NMR (400 MHz, CD₃OD): δ 8.40 (br s, 1H), 8.24 (s, 1H), 7.76 (d, 1H, J=7.6 Hz), 7.38-7.34 (m, 1H), 6.99 (s, 1H), 5.72 (s, 1H), 5.59 (s, 1H), 4.13-3.99 (m, 3H), 3.60 (t, 2H, J=9.6 Hz), 2.23 (s, 3H), 2.08-2.02 (m, 2H), 1.66-1.60 (m, 2H), (M+1)=391.1, HPLC purity>97%

### Example 205

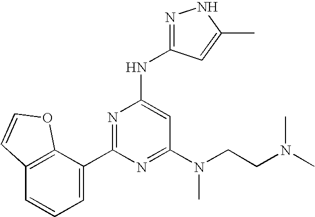

### 2-(benzofuran-7-yl)-N⁴-(2-(dimethylamino)ethyl)-N⁴-methyl-N⁶-(5-methyl-1H-pyrazol-3-yl)pyrimidine-4,6-diamine

Example 2431 was prepared following the same general procedure as in Example 8. R_{f}: 0.3 (CHCl₃:MeOH). ¹H NMR (400 MHz, CD₃OD): δ 8.47 (s, 1H), 8.28 (s, 1H), 7.78 (d, 1H, J=7.3 Hz), 7.38-7.34 (m, 1H), 7.01 (s, 1H), 5.75 (s, 1H), 5.60 (s, 1H), 3.93 (br s, 2H), 3.15 (s, 3H), 2.72 (br s, 2H), 2.44 (s, 6H), 2.27 (s, 3H). m/e (M+1): 392.2, HPLC purity: >97%\

### Example 206

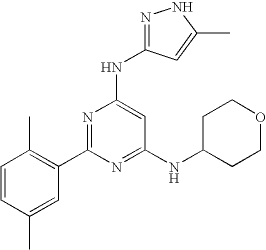

### 2-(2,5-dimethyl phenyl)-N⁴-(5-methyl-1H-pyrazol-3-yl)-N⁶-(tetrahydro-2H-pyran-4-yl)pyrimidine-4,6-diamine

Example 206 was prepared following the same general procedure as in Example 8. R_{f}: 0.4 (100% EtOAc). ¹H NMR (400 MHz, CD₃OD): δ 7.36 (s, 1H), 7.16-7.14 (m, 2H), 6.20 (br s, 1H), 5.91 (br s, 1H), 3.98-3.95 (m, 3H), 3.56-3.50 (m, 2H), 2.39 (s, 3H), 2.35 (s, 3H), 2.27 (s, 3H), 1.99-1.95 (m, 2H), 1.61-1.51 (m, 2H). m/e (M+1): 379.2; HPLC purity: >98%.

### Example 207

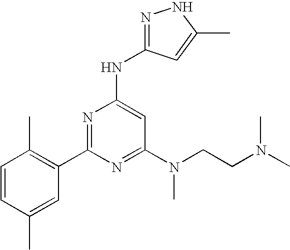

### N⁴-(2-(dimethylamino)ethyl)-2-(2,5-dimethylphenyl)-N⁴-methyl-N⁶-(5-methyl-1H-pyrazol-3-yl)pyrimidine-4,6-diamine

Example 207 was prepared following the same general procedure as in Example 8. R_{f}: 0.4 (9:1 CHCl₃:MeOH). ¹H NMR (400 MHz, CD₃OD): δ 7.43 (s, 1H), 7.16-7.13 (m, 2H), 6.30 (br s, 1H), 5.95 (br s, 1H), 3.79 (t, 2H, J=7 Hz), 3.09 (s, 3H), 2.71 (t, 2H, J=7 Hz), 2.44 (s, 3H), 2.38 (s, 6H), 2.35 (s, 3H), 2.28 (s, 3H). m/e (M+1): 380.2; HPLC purity: >98%.

### Example 208

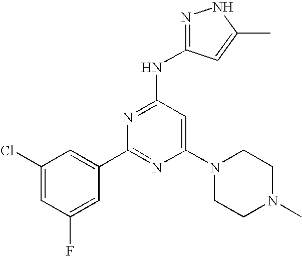

### 2-(3-chloro-5-fluorophenyl)-N-(5-methyl-1H-pyrazol-3-yl)-6-(4-methylpiperazin-1-yl)pyridine-4-amine

Example 208 was prepared following the same general procedure as in Example 196. ¹H NMR (300 MHz, CD₃OD): δ 8.11 (brs, 1H), 7.96 (br d, 1H, J=9.5 Hz), 7.42 (dt, 1H, J=8.3, 2.1 Hz), 6.33 (s, 1H), 6.12 (s, 1H), 4.86-4.45 (br s, 4H), 3.71-3.16 (br s, 4H), 2.98 (s, 3H), 2.37 (s, 3H). m/e (M+1): 402.

### Biological Testing

### Biological Testing Example 1

### Aurora A (Aurora 2) Inhibition Assay

Compounds were tested for their potency against recombinant Aurora A (Upstate, Lake Placid, N.Y.) using the PanVera Z′-Lyte kinase assay kit—Ser/Thr 1 peptide (Invitrogen, Carlsbad, Calif.). Assays were carried out in kinase assay buffer (50 mM HEPES, pH 7.5, 10 mM MgCl₂, 5 mM EGTA, 0.05% Brij-35, 2 mM DTT). Test compounds were initially dissolved in DMSO at 100× the highest tested concentration, then serially diluted to 4× test concentrations in kinase assay buffer. Next, Aurora A (final concentration 200-500 ng/mL), Z′-Lyte Ser/Thr 1 peptide (final concentration 2 μM) and ATP (final concentration 10 μM) were added according to the manufacturer's instructions. Assays were carried out in half-area 96-well white polystyrene assay plates (Corning, Corning, N.Y.) in a final volume of 20 μl. The reaction was allowed to proceed for 1 h at room temperature in the dark, at which point the development reagent and stop reagent were added according to the manufacturer's instructions. Coumarin (Ex. 400 nm, Em. 465 nm) and fluorescein (Ex. 400 nm, Em. 565 nm) fluorescence values were measured on a SpectraFluor Plus plate reader (Tecan, Durham, N.C.). The emission ratio (coumarin/fluorescein) was determined and used to calculate the percent phosphorylation for each well. Wells containing substrate but no kinase and wells containing a phosphopeptide control were used to set 0% and 100% phosphorylation values, respectively. Typically 20-40% of the substrate was phosphorylated in wells without inhibitor. Dose-response curves of relative Aurora A activity vs. inhibitor concentration were plotted with Grafit (Erithacus Software, Horley, Surrey, UK).

The compounds of the invention were shown to inhibit Aurora A using the method described above. For example, compounds 1, 2, 3, 6, 8, 10, 11, 12, 15, 16, 20, 23, 28, 29, 32, 33, 34, 35, 36, 38, 39, 41, 42, 74, 82, 85, 86, 89, 101, 119, 130, 132, 134, 135, 137, 139, 153, 155, 156 and 159 161, 162, 163, 165, 166, 167, 168, 169, 170, 171, 185, 188, 190, 191, 192, 193, 197, 203, were shown to have IC₅₀ values in this assay of less than or equal to 100 nM, and the compounds 4, 5, 7, 9, 13, 14, 19, 22, 24, 37, 40, 49, 52, 54, 62, 63, 65, 67, 68, 70, 79, 80, 81, 97, 100, 102, 103, 107, 113, 118, 125, 126, 127, 129, 136, 142, 146, 148, 149, 150, 157 and 158, 160, 164, 172, 174, 175, 178, 179, 180, 181, 184, 189, 196, 200, 201, 204, 205, 206, were shown to have IC₅₀ values in this assay of greater than 100 nM or less than or equal to 1 μM.

### Biological Testing Example 2

### Aurora B (Aurora 1) Inhibition Assay

Assays for Aurora B kinase inhibition were carried out similarly to those for Aurora A kinase (see above) with the following modifications. Aurora B kinase (BPS Biosciences, San Diego, Calif.) was used as the enzyme, at a concentration was 2.5 μg/mL. The ATP concentration was 50 μM, and the kinase reaction was allowed to proceed for 16 h. Sodium orthovanadate (20 μM) was added to the buffer to inhibit contaminating phosphatases. The compounds of the invention were shown to inhibit Aurora B using the method described above. The following compounds were shown to have IC₅₀ values equal to or less than 1 μM 8, 15, 16, 20, 33, 36, 42, 74, 130, 137, 139, 162, 163, 165, 166, 170, 190, and 191,

### Biological Testing Example 3

### Src Kinase Inhibition Assay

Compounds were assayed for Src kinase inhibitory activity using N-terminal His-tagged human Src (Upstate USA Inc, 706 Forest Street, Charlottesville, Va.). Serial dilutions of compound were assayed in a final reaction volume of 25 μL by incubating a solution of the above Src kinase (5-10 mU), 8 mM MOPS (3-(N-morpholino) propanesulfonic acid) pH 7.0, 0.2 mM EDTA (ethylenediamine tetracetic acid), 250 μM amino acid sequence KVEKIGEGTYGVVYK (Upstate USA Inc, 706 Forest Street, Charlottesville, Va.), and 10 mM magnesium acetate and [γ-³³P-ATP] (specific activity of about 500 cpm/μmol, concentration as required). The reaction was initiated by the addition of the magnesium acetate and [γ-³³P-ATP] mixture. After incubation for 40 minutes at room temperature, the reaction was stopped by the addition of 5 μL of a 3% phosphoric acid solution. A 10 μL aliquote of the reaction was then spotted onto a P30 filtermat (PerkinElmer, Wellesley, Mass.) and washed three times for five minutes in 75 mM phosphoric acid and once in methanol prior to drying and scintillation counting. Inhibition of Src activity was determined by comparison to assays that contained no inhibitor. The compounds of the invention were shown to inhibit Src kinase using the method described above. For example, compounds 1, 2, 3, 4, 5, 6, 8, 12, 15, 16, 20, 28, 29, 32, 33, 34, 35, 36, 37, 38, 39, 40, 41, 42, 49, 50, 54, 74, 82, 85, 86, 89, 103, 107, 113, 118, 119, 128, 130, 132, 134, 135, 137, 139, 142, 146, 155, 156, 157 160, 161, 162, 163, 164, 166, 167, 168, 170, 172, 178, 187, 190, 191, 192, 193, 196, 197, 200 and 203 were shown to have IC₅₀ values in this assay of less than or equal to 100 nM, and compounds 7, 9, 11, 13, 14, 19, 22, 23, 24, 26, 43, 50, 52, 62, 63, 65, 67, 68, 70, 79, 80, 81, 97, 100, 101, 102, 114, 115, 125, 127, 129, 136, 141, 148, 149, 153, 158, 159, 169, 171, 174, 175, 179, 180, 181, 182, 183, 184, 185, 186, 188, 189, 201, 204, 205 and 206, were shown to have an IC₅₀ value in this assay of greater than 100 nM to less than or equal to 1 μM.

### Biological Testing Example 4

### Flt3 Kinase Inhibition Assay

Compounds were assayed for Flt3 kinase inhibitory activity using N-terminal GST-tagged recombinant human Flt3, residues 564-end (Upstate USA Inc, 706 Forest Street, Charlottesville, Va.). Serial dilutions of compound were assayed in a final reaction volume of 25 μL by incubating a solution of the above Flt3 kinase (5-10 mU), 8 mM MOPS (3-(N-morpholino) propanesulfonic acid) pH 7.0, 0.2 mM EDTA (ethylenediamine tetracetic acid), 50 μM amino acid sequence EAIYAAPFAKKK (Upstate USA Inc, 706 Forest Street, Charlottesville, Va.), and 10 mM magnesium acetate and [γ-³³P-ATP] (specific activity of about 500 cpm/μmol, concentration as required). The reaction was initiated by the addition of the magnesium acetate and [γ-³³P-ATP] mixture. After incubation for 40 minutes at room temperature, the reaction was stopped by the addition of 5 μL of a 3% phosphoric acid solution. A 10 μL aliquote of the reaction was then spotted onto a P30 filtermat (PerkinElmer, Wellesley, Mass.) and washed three times for five minutes in 75 mM phosphoric acid and once in methanol prior to drying and scintillation counting. Inhibition of Flt3 activity was determined by comparison to assays that contained no inhibitor. The compounds of the invention were shown to inhibit Flt3 kinase using the method described above. For example, compounds 1, 2, 3, 4, 5, 6, 7, 8, 9, 10, 11, 12, 13, 14, 15, 16, 19, 20, 22, 29, 32, 33, 35, 36, 37, 38, 39, 40, 41, 42, 43, 49, 50, 50, 52, 54, 56, 62, 63, 65, 67, 68, 70, 74, 79, 80, 81, 82, 85, 86, 89, 97, 100, 101, 102, 103, 107, 113, 114, 115, 118, 119, 126, 127, 128, 129, 130, 132, 134, 135, 136, 137, 139, 141, 142, 143, 146, 148, 149, 150, 153, 155, 156, 157, 158, 159, 160, 161, 162, 163, 164, 165, 166, 167, 168, 169, 170, 171, 172, 173, 174, 175, 178, 179, 180, 181, 182, 183, 184, 185, 186, 187, 188, 189, 190, 191, 192, 193, 196, 197, 200, 201, 202, 203, 204, 205, 206 and 207, and 159 were shown to have IC₅₀ values in this assay of less than or equal to 100 nM and compounds 69, 125, 194, 195 and 199 were shown to have an IC₅₀ value in this assay of greater than 100 nM to less than or equal to 1 μM.,

### Biological Testing Example 5

### KDR Kinase Inhibition Assay

Compounds were assayed for KDR kinase inhibitory activity using N-terminal His₆-tagged recombinant human KDR, residues 790-end (Upstate USA Inc, 706 Forest Street, Charlottesville, Va.). Serial dilutions of compound were assayed in a final reaction volume of 25 μL by incubating a solution of the above KDR kinase (5-10 mU), 8 mM MOPS (3-(N-morpholino) propanesulfonic acid) pH 7.0, 0.2 mM EDTA (ethylenediamine tetracetic acid), 0.33 mg/mL myelin basic protein (Upstate USA Inc, 706 Forest Street, Charlottesville, Va.), and 10 mM magnesium acetate and [γ-³³P-ATP] (specific activity of about 500 cpm/μmol, concentration as required). The reaction was initiated by the addition of the magnesium acetate and [γ-³³P-ATP] mixture. After incubation for 40 minutes at room temperature, the reaction was stopped by the addition of 5 μL of a 3% phosphoric acid solution. A 10 μL aliquote of the reaction was then spotted onto a P30 filtermat (PerkinElmer, Wellesley, Mass.) and washed three times for five minutes in 75 mM phosphoric acid and once in methanol prior to drying and scintillation counting. Inhibition of KDR activity was determined by comparison to assays that contained no inhibitor. The compounds of the invention were shown to inhibit KDR kinase using the method described above. For example, compounds 1, 2, 3, 6, 8, 9, 10, 11, 12, 13, 15, 16, 20, 26, 29, 33, 34, 35, 36, 37, 38, 39, 40, 41, 42 49, 50, 50, 74, 85, 102, 107, 114, 128, 132, 134, 135, 137, 139, 155, 161, 162, 163, 165, 166, 167, 169, 171, 172, 175, 179, 180, 183, 184, 185, 190, 191, 193, 196, 197, 200 and 203 were shown to have IC₅₀ values in this assay of less than or equal to 100 nM, and the compounds 4, 5, 7, 14, 19, 22, 23, 24, 28, 30, 32, 43, 52, 54, 56, 62, 63, 65, 67, 68, 69, 70, 80, 81, 82, 89, 97, 101, 103, 113, 118, 119, 125, 126, 127, 129, 130, 136, 141, 142, 143, 146, 148, 149, 150, 153, 156, 157, 159 160, 168, 174, 178, 181, 182, 186, 187, 188, 189, 192, 201, 204, 205, 206 and 207 were shown to have IC₅₀ values in this assay of greater than 100 nM or less than or equal to 1 μM.

### Biological Testing Example 6

### Whole Cell Cytotoxicity

### Assay: Sulforhodamine B

### Reference: Developmental Therapeutics Program NCl/NIH

### http://dtp.nci.nih.gov/branches/btb/ivclsp.html

Human tumor-derived cell lines, HCT116 or MCF7 (ATCC) were plated in a 96 well plate in DMEM containing 10% fetal bovine serum and 2 mM L-glutamine at a density of 500 HCT116 cells or 1,000 MCF7 cells per well and incubated at 37° C., 5% CO₂, for 24 hours prior to the addition of experimental compounds. Compounds were added using the dilution series indicated to duplicate plates and the cells were incubated in media plus compound for 96 hours. An additional plate was fixed in 10% TCA at the time of the addition of compound to provide a measurement of the cell population at time zero, the time of drug addition. Following the 96 hour incubation, cells were fixed in situ by gently aspirating off the culture media and then adding 50 ul of ice cold 10% TCA per well and incubation at 4° C. for 60 minutes. The plates were washed with tap water five times and allowed to air dry for 5 minute. 50 ul of a 0.4% (w/v) Sulforhodamine B solution in 1% (v/v) acetic acid was added per well and the cells were incubated for 30 minutes at room temperature. Following staining, plates were washed four times with 1% acetic acid to remove any unbound dye and then allowed to air dry for 5 minutes. The stain was solubilized with 100 ul of 10 mM Tris pH 10.5 per well and placed on an orbital rotator for 5 minutes. The absorbance was read at 570 nm. Percentage growth was calculated using the absorbance readings from the time zero plate (Tz) and the dilution series plate (C) which included a column of cells grown in media without compound as a control (C) using the formulas:

[(Ti−Tz)/(C−Tz)]×100 for concentrations for which Ti>/=Tz

[(Ti−Tz)/Tz]×100 for concentrations for which Ti<Tz.

Three dose response parameters were calculated for each experimental agent. Growth inhibition of 50% (GI50) was calculated from [(Ti−Tz)/(C−Tz)]×100=50, which was the drug concentration resulting in a 50% reduction in the net protein increase (as measured by SRB staining) in control cells during the drug incubation. The drug concentration resulting in total growth inhibition (TGI) was calculated from Ti=Tz. The LC50 (concentration of drug resulting in a 50% reduction in the measured protein at the end of the drug treatment as compared to that at the beginning) indicating a net loss of cells following treatment ws calculated from [(Ti−Tz)/Tz]×100=−50. Values are calculated for each of these three parameters if the level of activity was reached; however, if the effect was not reached or was exceeded, the value for that parameter is expressed as greater or less than the maximum or minimum concentration tested.

The compounds of the invention were shown to inhibit HCT116 cell growth using the method described above. For example, compounds 1, 2, 4, 6, 8, 9, 11, 12, 13, 15, 16, 32, 33, 36, 38, 39, 41, 42, 49, 50, 52, 54, 56, 68, 74, 85, 86, 95, 97, 103, 113, 135, 137, 139, 141, 142, 146, 155, 157, 160, 162, 163, 165, 166, 168, 169, 170, 172, 173, 178, 179, 190, 191, 196, 197 and 200 were shown to have GI₅₀ values in this assay of less than or equal to 1 μM, and compounds 3, 5, 7, 10, 14, 19, 20, 22, 23, 26, 28, 29, 34, 35, 37, 40, 43, 50, 62, 63, 65, 67, 69, 70, 79, 80, 81, 82, 89, 100, 159 101, 102, 107, 114, 115, 118, 119, 125, 126, 127, 128, 130, 132, 134, 136, 143, 148, 149, 150, 153, 156, 158, 159, 164, 171, 174, 175, 182, 183, 184, 185, 186, 188, 189, 192, 193, 201, 202, 203, 204, 205 were shown to have IC₅₀ values in this assay of greater than 1 μM or less than or equal to 10 μM.

What is claimed is:

1. A compound of the Formula I: 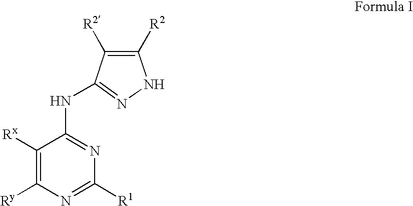
or a pharmaceutically acceptable derivative or prodrug thereof, wherein:
R^{x} is hydrogen, N(R⁴)₂, NO₂ or a C₁₋₁₂ aliphatic group;
R^{y} is hydrogen, N(R⁴)₂, OR, SR, S(O)R, S(O)₂R, N(R⁷)C(═O)R; an optionally substituted 3-10 membered monocyclic or bicyclic heterocyclyl or heteroaryl ring, wherein the 3-10 membered heterocyclyl or heteroaryl ring may have 1-4 ring heteroatoms selected from the group consisting of nitrogen, oxygen and sulfur; or alkyl or dialkyl amino wherein alkyl is optionally substituted with —OR, —SR, amino, alkylamino, dialkylamino or a C₃₋₈ heteroaryl or heterocyclyl ring having 1-4 heteroatoms selected from the group consisting of nitrogen, oxygen and sulfur;
R¹ is an optionally substituted 5-7 membered monocyclic or an 8-10 membered bicyclic aryl or heteroaryl ring, said heteroaryl ring having 1-4 ring heteroatoms selected from the group consisting of nitrogen, oxygen, and sulfur, wherein each substitutable ring carbon of R¹ is optionally independently substituted by oxo, R⁵, and each substitutable ring nitrogen of R¹ is optionally independently substituted by —R⁴;
R² and R^{2′} are independently selected from the group consisting of —R and N(R⁴)₂, OR, SR, S(O)R, S(O)₂R, or R² and R^{2′} taken together with their intervening atoms form a fused, 5-8 membered, unsaturated or partially unsaturated ring having 0-3 ring heteroatoms selected from the group consisting of nitrogen, oxygen, and sulfur, wherein each substitutable ring carbon of said fused ring formed by R² and R^{2′} is independently substituted by halo, oxo, —CN, —NO₂, or R⁷, and each substitutable ring nitrogen of said ring formed by R² and R^{2′} is independently substituted by —R⁴;
each R is independently hydrogen, R⁷ or an optionally substituted group selected from the group consisting of C₁₋₆ aliphatic, C₆₋₁₀ aryl, a heteroaryl ring having 5-10 ring atoms, and a heterocyclyl ring having 5-10 ring atoms;
each R⁴ is independently selected from the group consisting of —R⁷, —COR⁷, —CO₂ (optionally substituted C₁₋₆ aliphatic), —CON(R⁷)₂, and —SO₂R⁷;
each R⁵ is independently selected from the group consisting of —R, halo, —OR, —C(═O)R, —CO₂R, —COCOR, —NO₂, —CN, —S(O)R, —SO₂R, —SR, —N(R⁴)₂, —CON(R⁴)₂, —SO₂N(R⁴)₂, —OC(═O)R, —N(R⁴)COR, —N(R⁴)CO₂R, —N(R)SO₂N(R)₂, —N(R)CON(R)₂, OC(O)N(R)₂, —N(R⁴)N(R⁴)₂, —C═NN(R⁴)₂, —C═N—OR, —N(R⁴)CON(R⁴)₂, —N(R⁴)SO₂N(R⁴)₂, —N(R⁴)SO₂R, and —OC(═O)N(R⁴)₂; and
each R⁷ is independently selected from the group consisting of hydrogen, a C₁₋₆ aliphatic group which may optionally be substituted by OR, SR or N(R)₂; an optionally substituted 3-8 membered heterocyclyl or heteroaryl ring, wherein the 3-8 membered heterocyclyl or heteroaryl ring may have 1-4 ring heteroatoms selected from the group consisting of nitrogen, oxygen and sulfur; or two R⁷ on the same nitrogen are taken together with the nitrogen to form an optionally substituted 3-8 membered heterocyclyl or heteroaryl ring, wherein the 3-8 membered heterocyclyl or heteroaryl ring may have 1-4 ring heteroatoms selected from the group consisting of nitrogen, oxygen and sulfur;
with the proviso that when R^{x} is H and R^{y} is 4-methylpiperazinyl, and R² is methyl and R^{2′} is hydrogen, then R¹ is not unsubstituted indol-2-yl.

2. The compound of claim 1, wherein R^{x} is hydrogen.

3. The compound of claim 1, wherein:
R^{x} is N(R⁴)₂ or NO₂.

4. The compound of claim 1, wherein:
R^{x}═NR(CH₂)ₙN(R)₂, where n is more than 1.

5. The compound of claim 1, wherein:
R^{y} is hydrogen, N(R⁴)₂, OR, SR, an optionally substituted 4-8 membered heterocyclyl or heteroaryl ring, or alkyl or dialkyl amino wherein alkyl is optionally substituted with —OR, —SR, amino, alkylamino, dialkylamino or a C₃₋₈ heteroaryl or heterocyclyl ring having 1-4 heteroatoms selected from the group consisting of nitrogen, oxygen and sulfur.

6. The compound of claim 1, wherein:
R² and R^{2′} are independently hydrogen, alkyl or amino.

7. The compound of claim 1, wherein:
R² and R^{2′} are independently hydrogen or alkyl;
R^{x} is hydrogen or N(R⁴)₂; and
R^{y} is hydrogen, N(R⁴)₂, alkyl or dialkyl amino wherein alkyl is optionally substituted with —OR, —SR, amino, alkylamino, dialkylamino or a C₃₋₈ heteroaryl or heterocyclyl ring having 1-4 heteroatoms selected from the group consisting of nitrogen, oxygen and sulfur; or an optionally substituted 4-8 membered heterocyclyl or heteroaryl ring.

8. The compound of claim 7, wherein R¹ is an optionally substituted 8-10 membered bicyclic heteroaryl ring.

9. The compound of claim 7, wherein R¹ is an optionally substituted monocyclic or bicyclic aryl ring.

10. The compound of claim 7, wherein:
R¹ is selected from the group consisting of 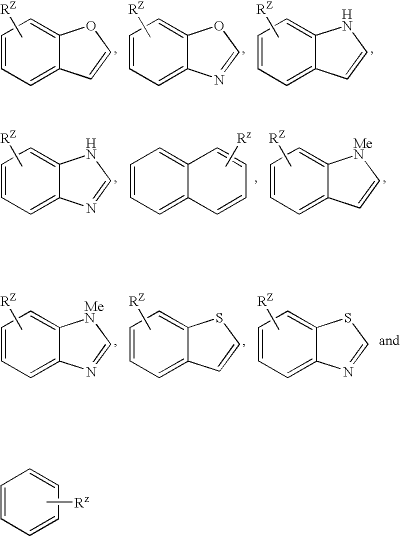
wherein R^{z} is selected from the group consisting of H, alkyl, alkoxy, halogen, CF₃, amino, alkylamino, dialkylamino, cyano and nitro.

11. The compound of claim 7, wherein:
R^{y} is 4-aminotetrahydropyran, 2-methoxyethyl amine, 2-dimethylaminoethyl amine, 2-morpholinoethylamine, 2-(4-methylpiperazin-1-yl)ethylamine, 4-aminotetrahydropyran, 1-pyrrolidinyl, 1-piperidinyl, 1-piperazinyl, 4-methyl-1-piperazinyl, 4-acyl-1-piperazinyl or 4-morpholinyl.

12. The compound of claim 7, wherein R^{y} is optionally substituted alkylamino or dialkylamino.

13. The compound of claim 7, wherein R¹ is optionally substituted a 5-7 membered monocyclic heteroaryl ring.

14. The compound of claim 1, wherein:
R¹ is optionally substituted phenyl, N-methylindolyl, indolyl or benzofuranyl;
R^{x} is hydrogen;
R^{y} is 4-aminotetrahydropyran, N-methyl-N-(2-methoxy)ethyl-amine, N-methyl-N-(2-(dimethylamino)ethyl amine, 1-piperidinyl, 1-piperazinyl or 4-morpholinyl;
R² is alkyl; and
R^{2′} is hydrogen.

15. A compound selected from the group:

| Com- | |
|---|---|
| pound | |
| No. | Structure |
| 1 | 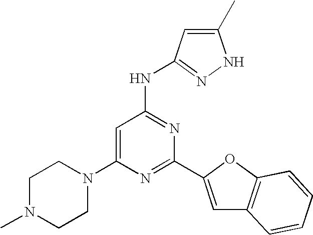 |
| 2 | 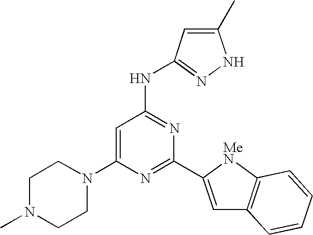 |
| 3 | 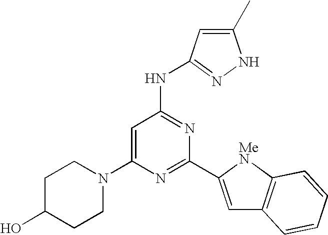 |
| 4 | 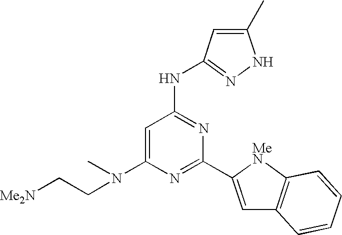 |
| 5 | 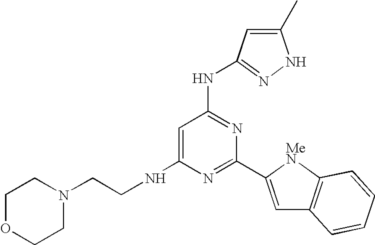 |
| 6 | 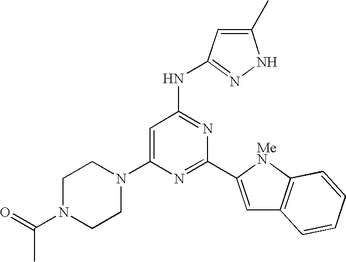 |
| 7 | 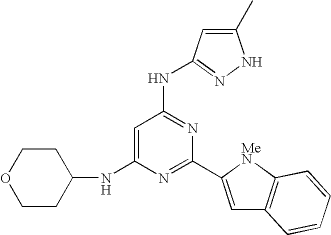 |
| 8 | 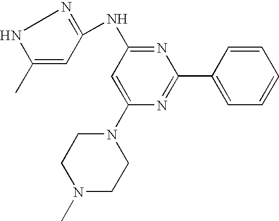 |
| 9 | 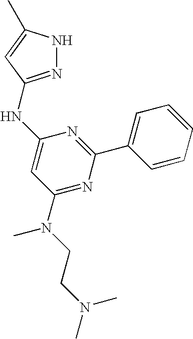 |
| 10 | 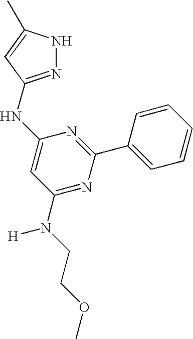 |
| 11 | 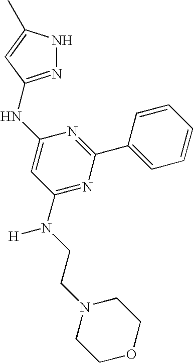 |
| 12 | 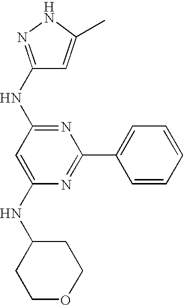 |
| 13 | 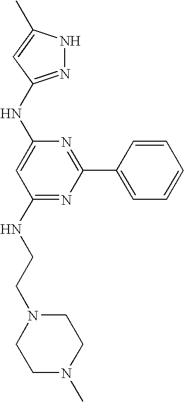 |
| 14 | 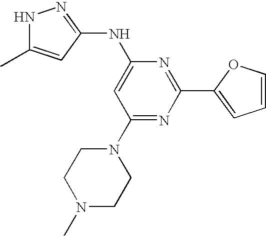 |
| 15 | 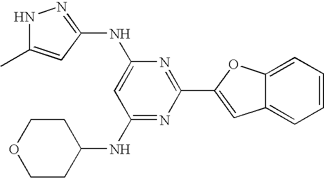 |
| 16 | 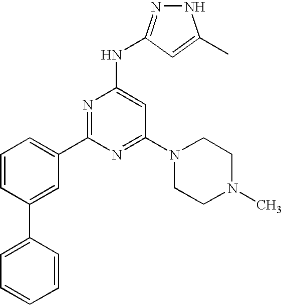 |
| 17 | 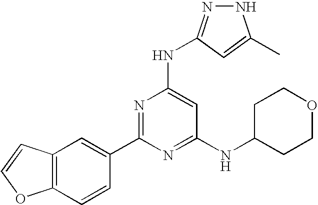 |
| 18 | 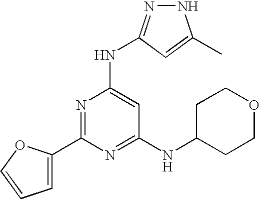 |
| 19 | 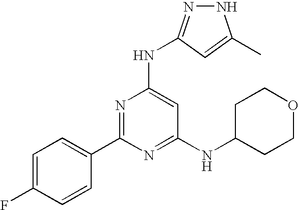 |
| 20 | 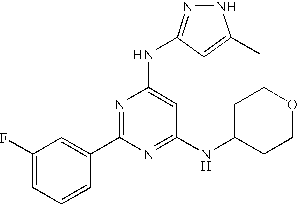 |
| 21 | 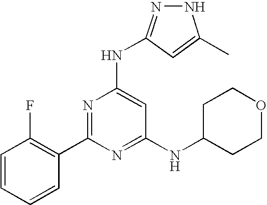 |
| 22 | 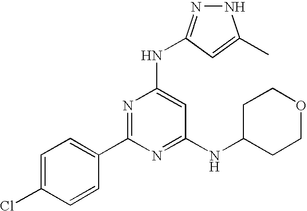 |
| 23 | 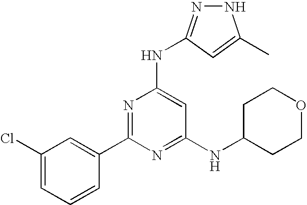 |
| 24 | 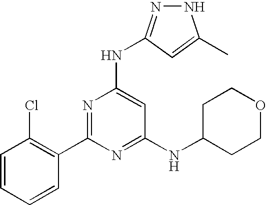 |
| 25 | 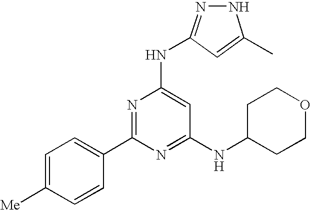 |
| 26 | 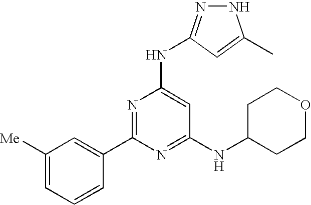 |
| 27 | 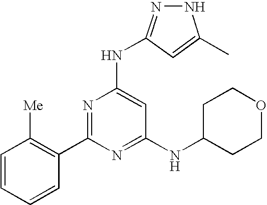 |
| 28 | 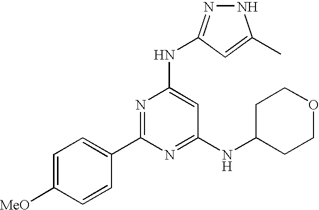 |
| 29 | 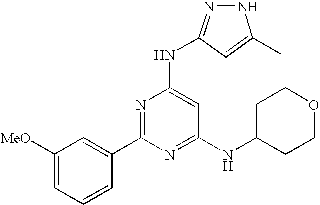 |
| 30 | 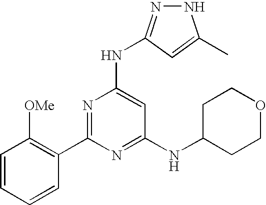 |
| 31 | 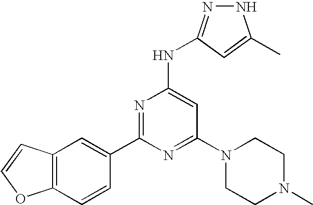 |
| 32 | 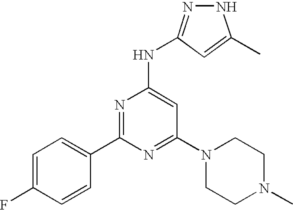 |
| 33 | 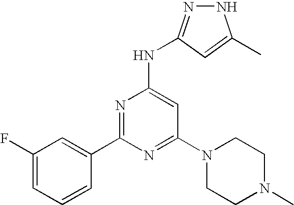 |
| 34 | 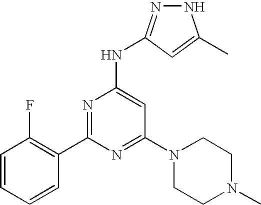 |
| 35 | 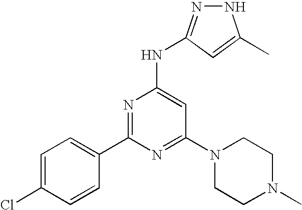 |
| 36 | 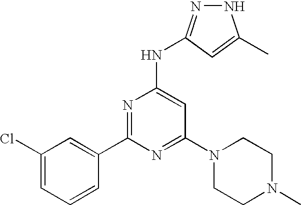 |
| 37 | 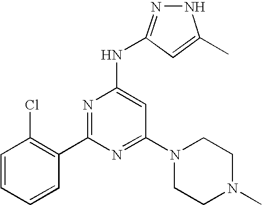 |
| 38 | 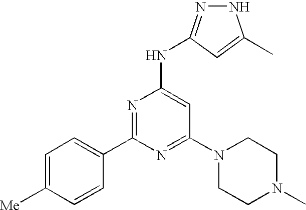 |
| 39 | 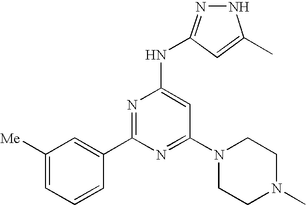 |
| 40 | 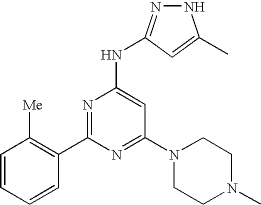 |
| 41 | 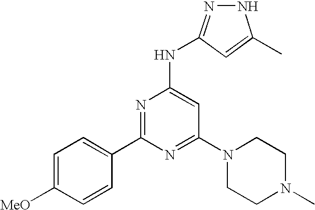 |
| 42 | 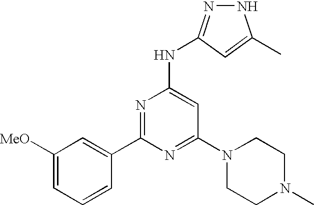 |
| 43 | 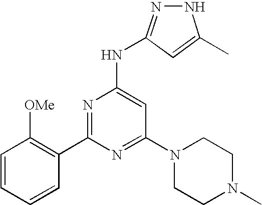 |
| 44 | 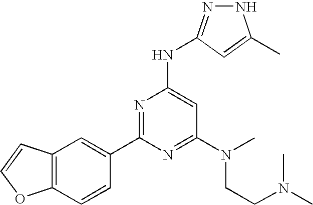 |
| 45 | 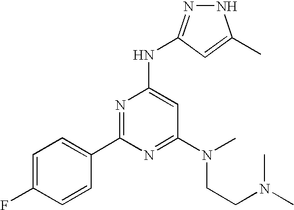 |
| 46 | 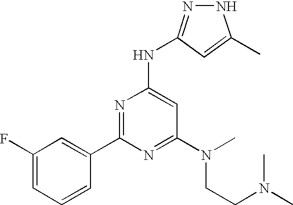 |
| 47 | 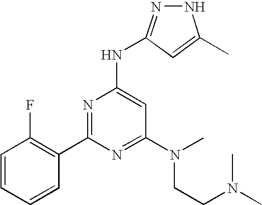 |
| 48 | 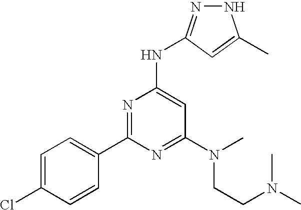 |
| 49 | 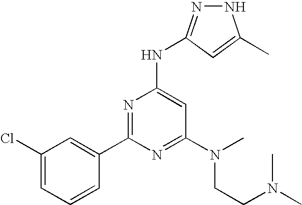 |
| 50 | 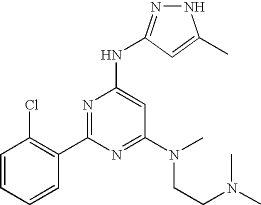 |
| 51 | 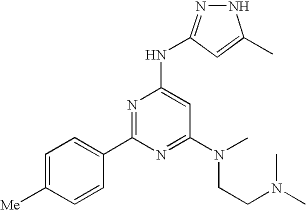 |
| 52 | 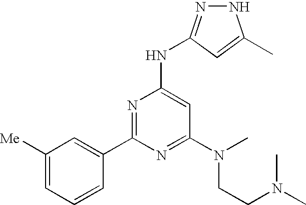 |
| 53 | 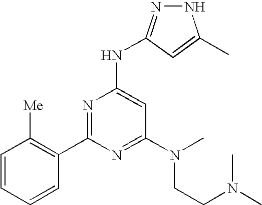 |
| 54 | 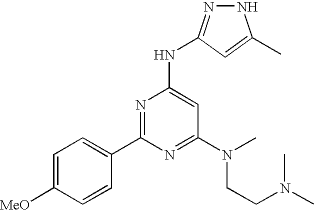 |
| 55 | 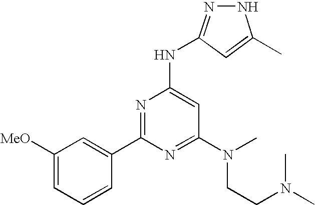 |
| 56 | 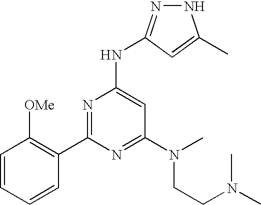 |
| 57 | 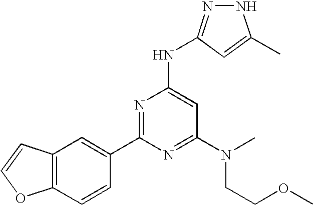 |
| 58 | 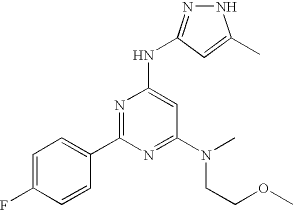 |
| 59 | 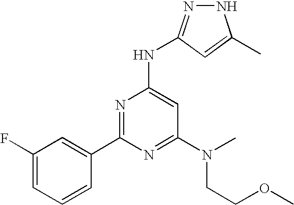 |
| 60 | 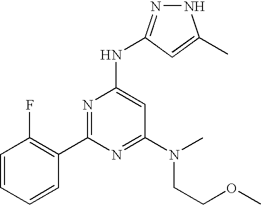 |
| 61 | 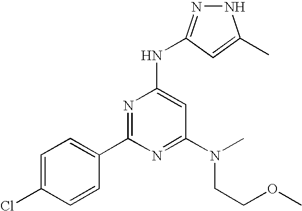 |
| 62 | 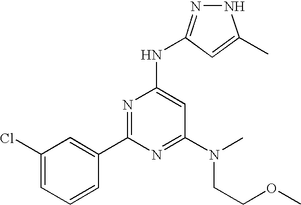 |
| 63 | 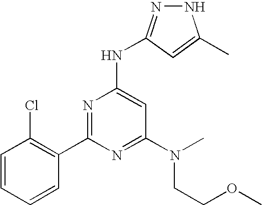 |
| 64 | 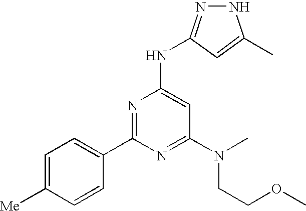 |
| 65 | 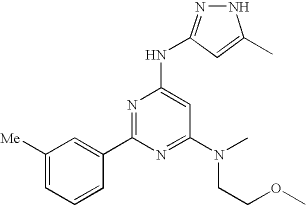 |
| 66 | 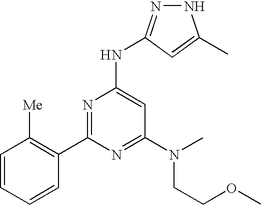 |
| 67 | 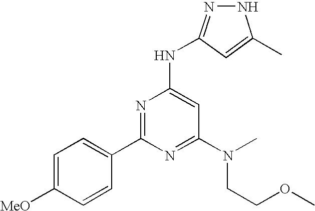 |
| 68 | 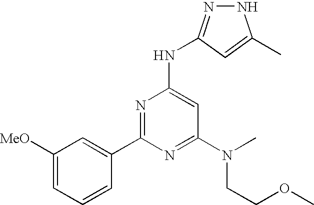 |
| 69 | 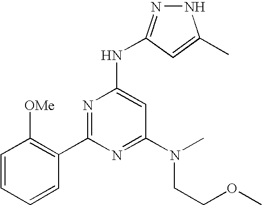 |
| 70 | 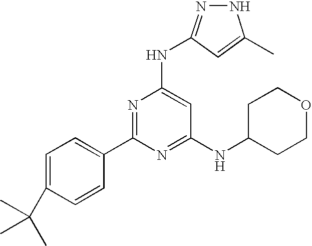 |
| 71 | 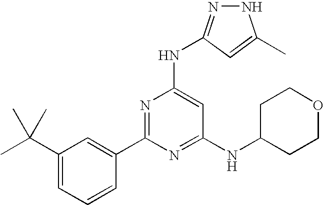 |
| 72 | 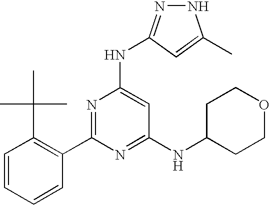 |
| 73 | 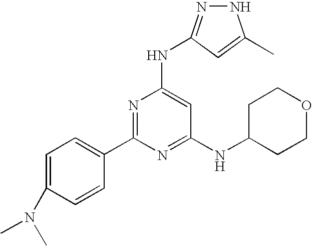 |
| 74 | 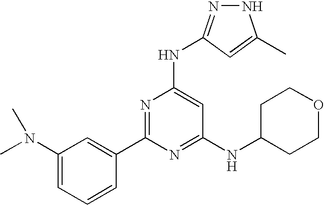 |
| 75 | 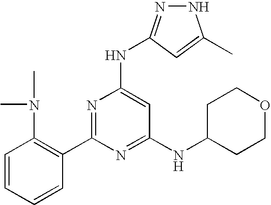 |
| 76 | 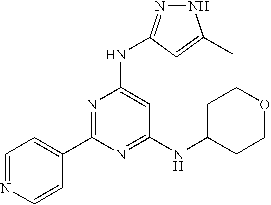 |
| 77 | 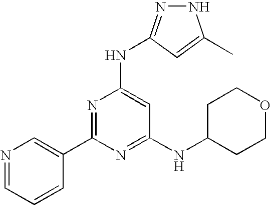 |
| 78 | 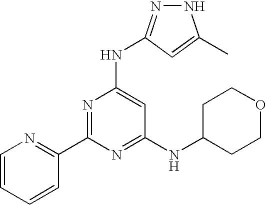 |
| 79 | 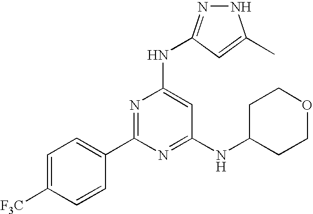 |
| 80 | 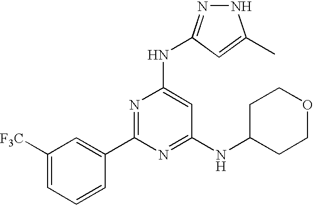 |
| 81 | 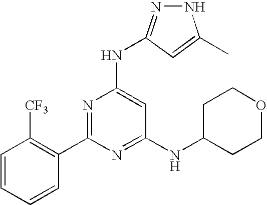 |
| 82 | 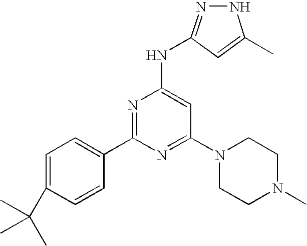 |
| 83 | 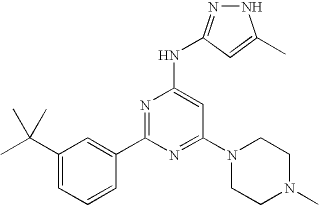 |
| 84 | 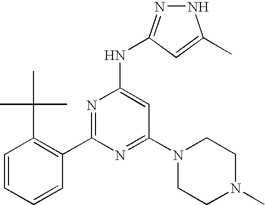 |
| 85 | 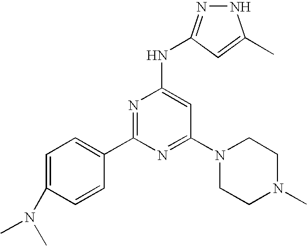 |
| 86 | 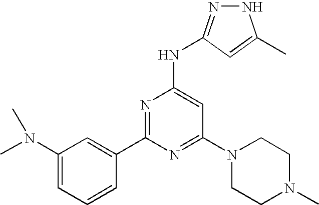 |
| 87 | 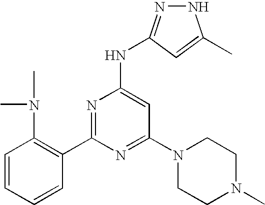 |
| 88 | 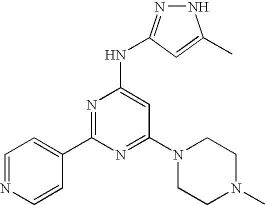 |
| 89 | 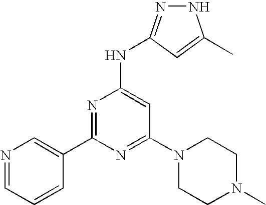 |
| 90 | 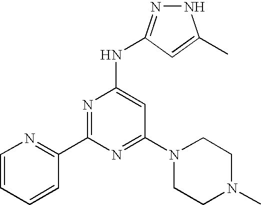 |
| 91 | 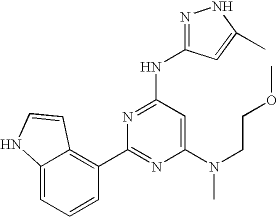 |
| 92 | 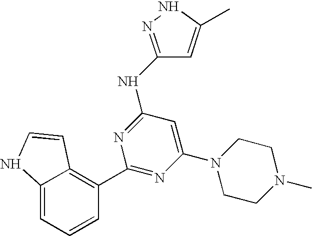 |
| 93 | 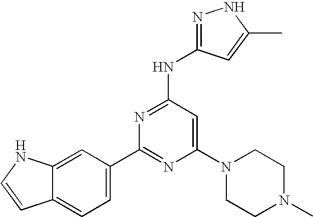 |
| 94 | 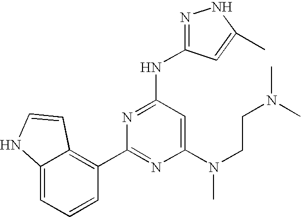 |
| 95 | 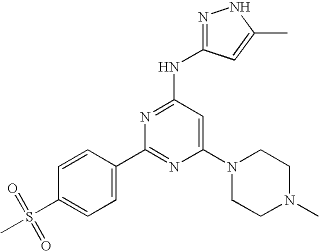 |
| 96 | 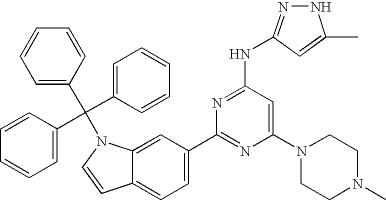 |
| 97 | 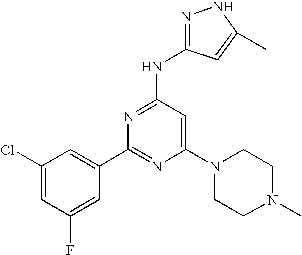 |
| 98 | 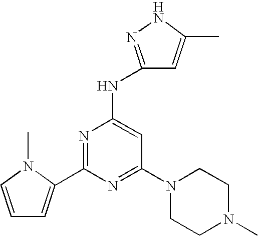 |
| 99 | 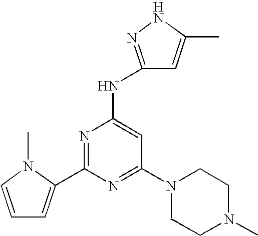 |
| 100 | 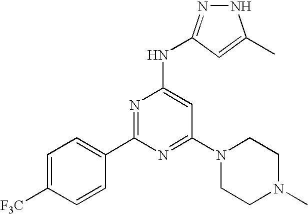 |
| 101 | 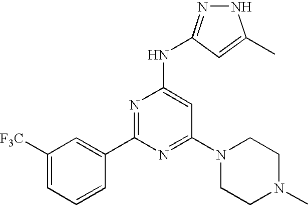 |
| 102 | 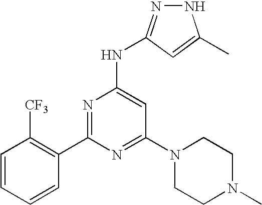 |
| 103 | 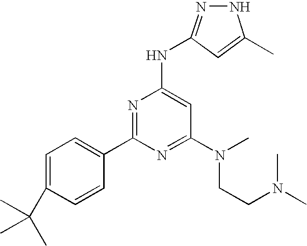 |
| 104 | 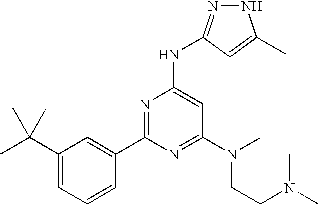 |
| 105 | 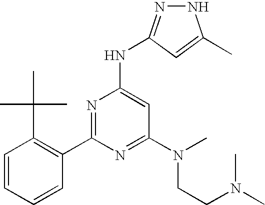 |
| 106 | 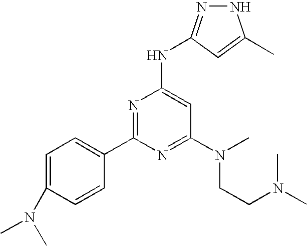 |
| 107 | 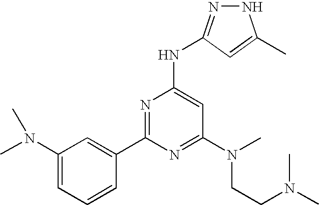 |
| 108 | 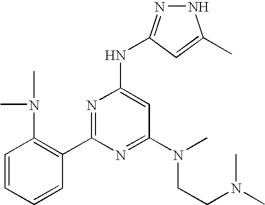 |
| 109 | 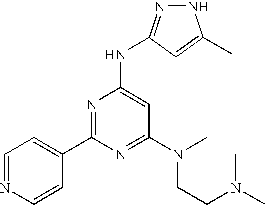 |
| 110 | 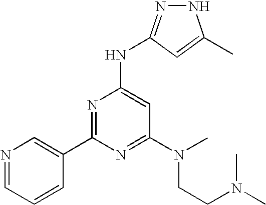 |
| 111 | 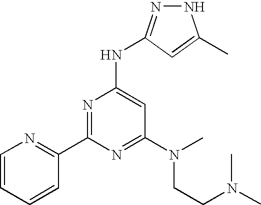 |
| 112 | 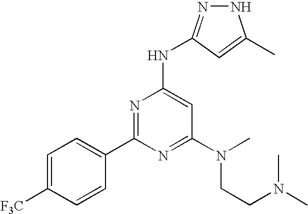 |
| 113 | 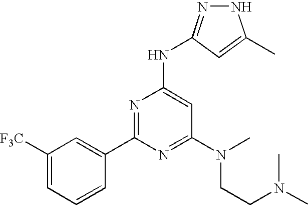 |
| 114 | 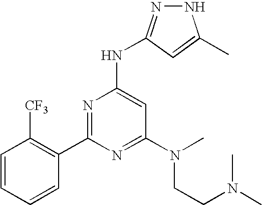 |
| 115 | 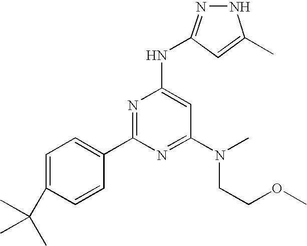 |
| 116 | 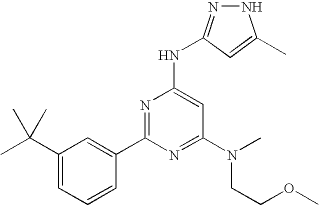 |
| 117 | 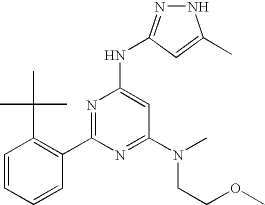 |
| 118 | 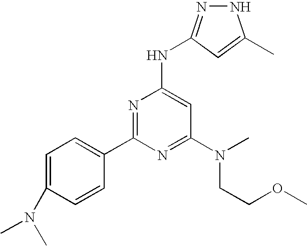 |
| 119 | 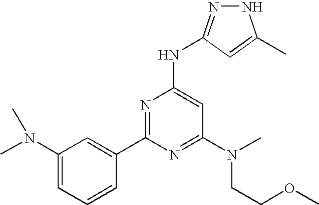 |
| 120 | 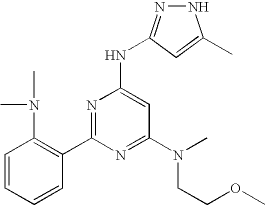 |
| 121 | 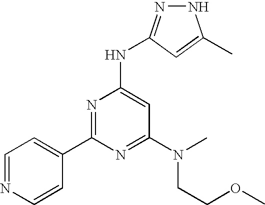 |
| 122 | 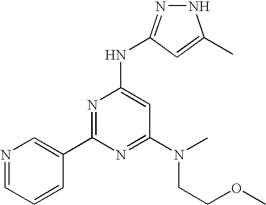 |
| 123 | 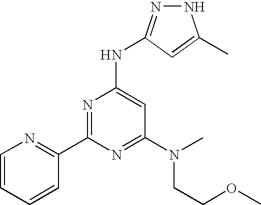 |
| 124 | 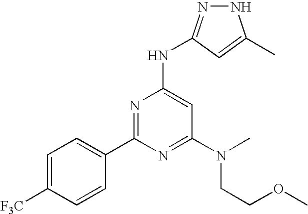 |
| 125 | 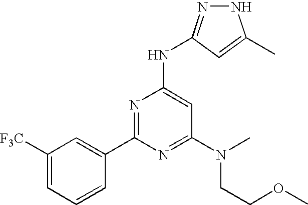 |
| 126 | 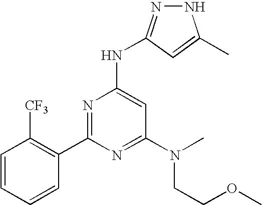 |
| 127 | 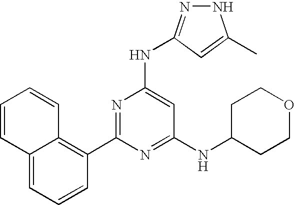 |
| 128 | 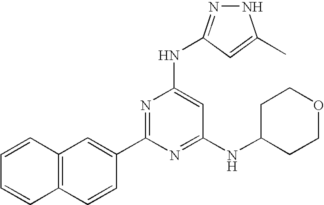 |
| 129 | 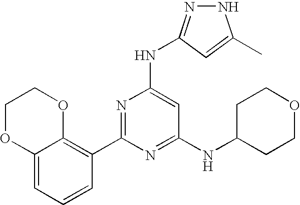 |
| 130 | 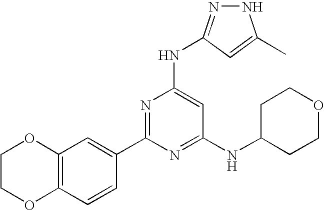 |
| 131 | 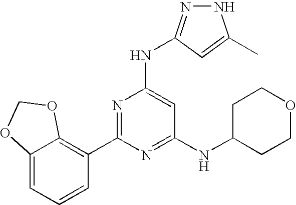 |
| 132 | 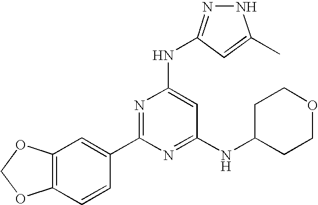 |
| 133 | 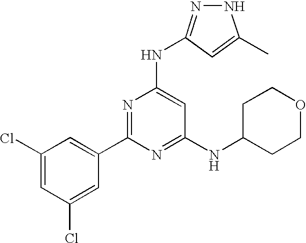 |
| 134 | 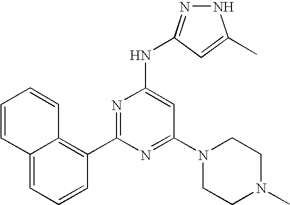 |
| 135 | 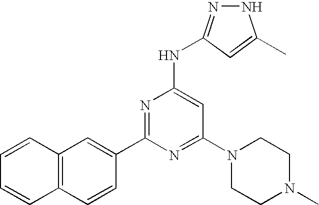 |
| 136 | 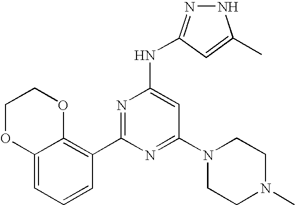 |
| 137 | 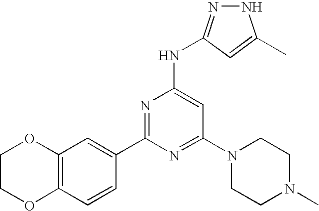 |
| 138 | 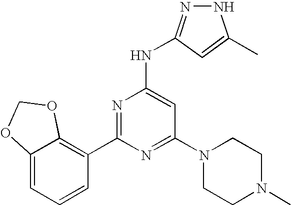 |
| 139 | 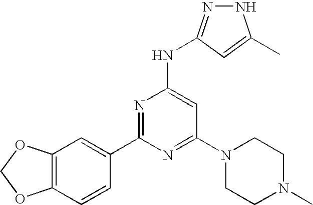 |
| 140 | 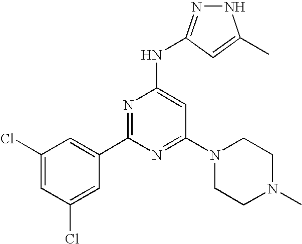 |
| 141 | 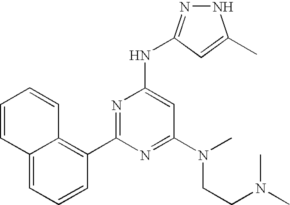 |
| 142 | 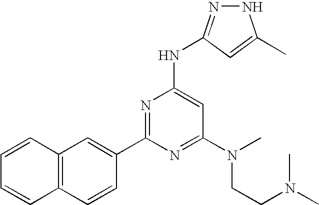 |
| 143 | 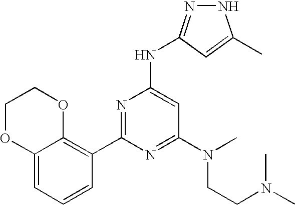 |
| 144 | 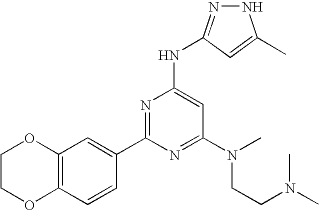 |
| 145 | 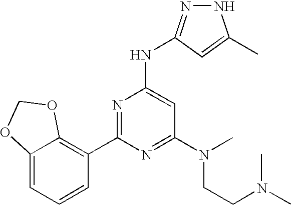 |
| 146 | 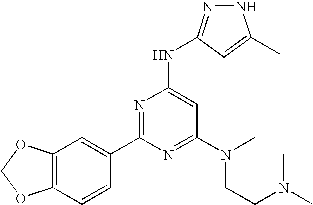 |
| 147 | 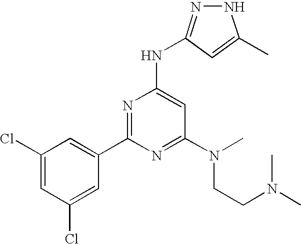 |
| 148 | 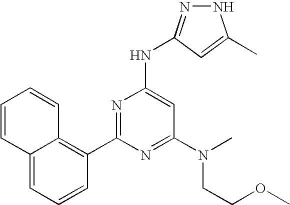 |
| 149 | 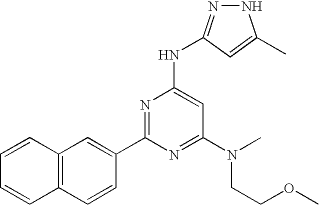 |
| 150 | 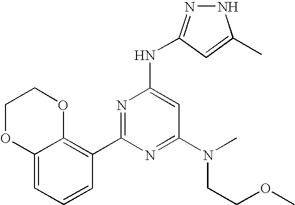 |
| 151 | 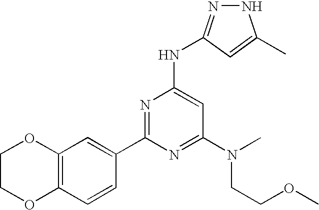 |
| 152 | 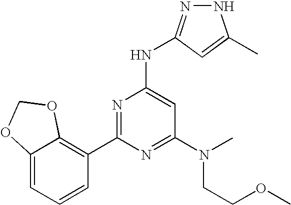 |
| 153 | 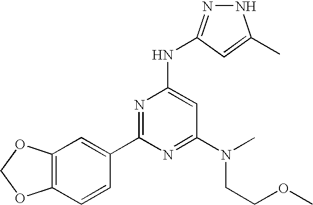 |
| 154 | 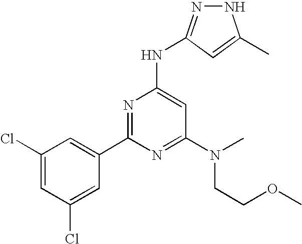 |
| 155 | 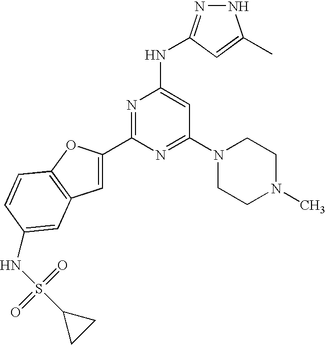 |
| 156 | 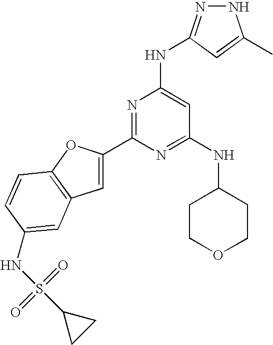 |
| 157 | 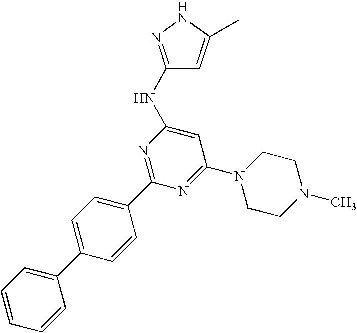 |
| 158 | 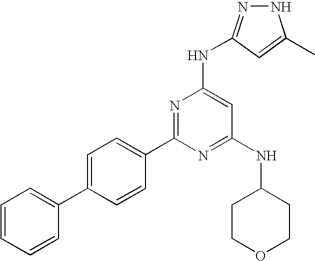 |
| 159 | 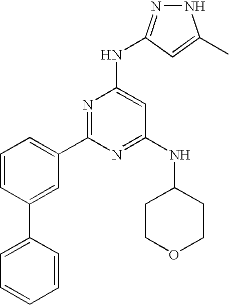 |
| 160 | 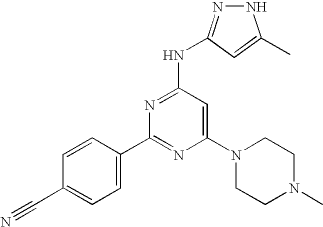 |
| 161 | 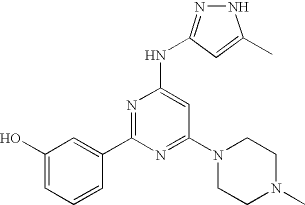 |
| 162 | 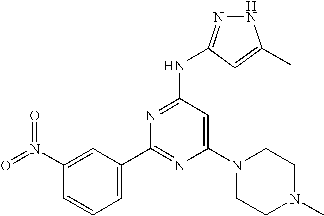 |
| 163 | 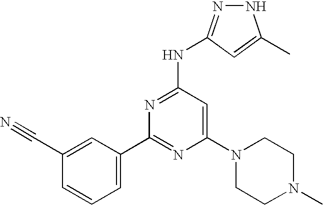 |
| 164 | 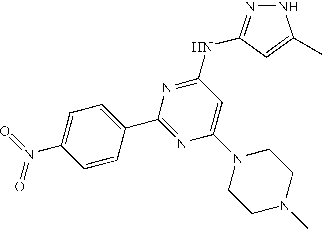 |
| 165 | 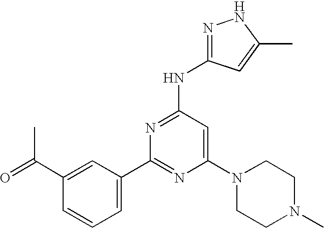 |
| 166 | 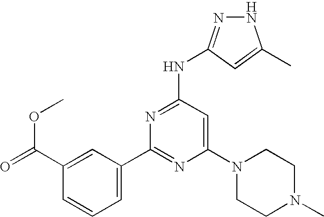 |
| 167 | 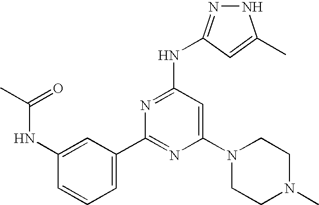 |
| 168 | 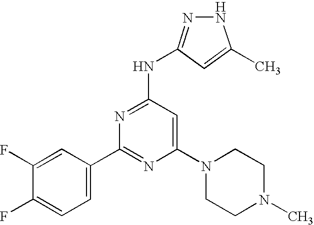 |
| 169 | 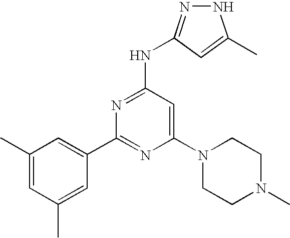 |
| 170 | 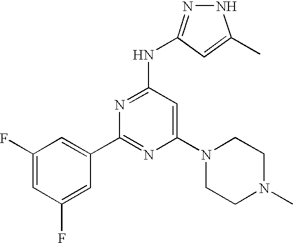 |
| 171 | 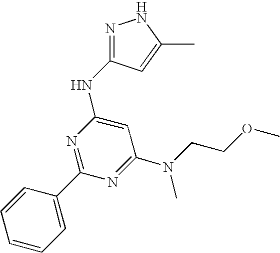 |
| 172 | 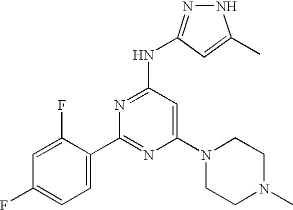 |
| 173 | 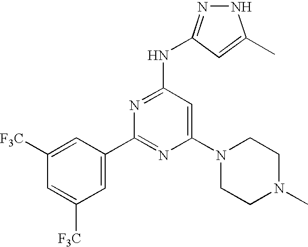 |
| 174 | 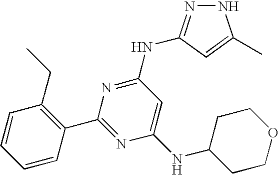 |
| 175 | 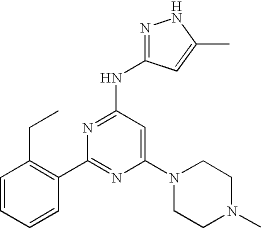 |
| 178 | 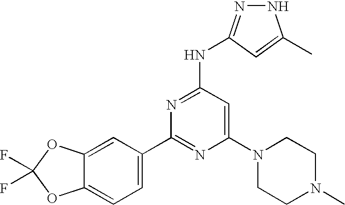 |
| 179 | 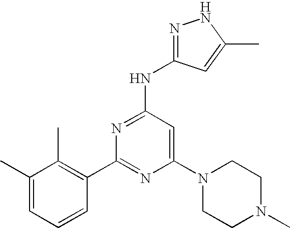 |
| 180 | 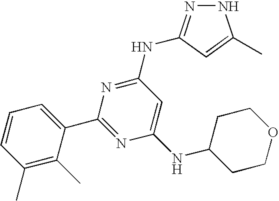 |
| 181 | 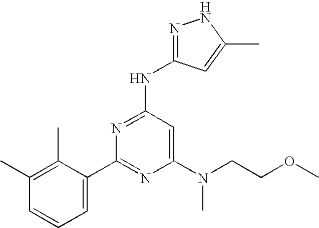 |
| 182 | 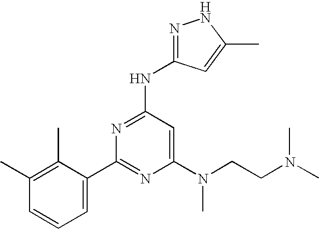 |
| 183 | 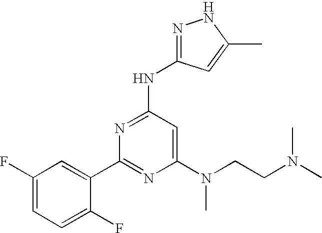 |
| 184 | 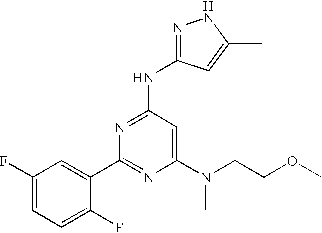 |
| 185 | 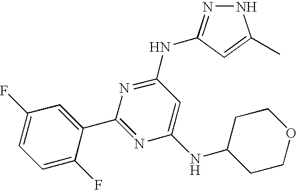 |
| 186 | 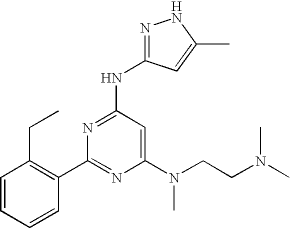 |
| 187 | 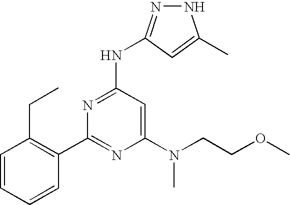 |
| 188 | 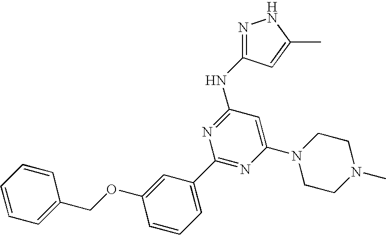 |
| 189 | 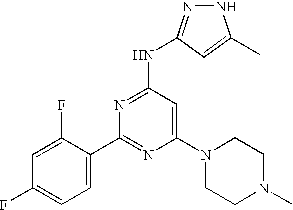 |
| 190 | 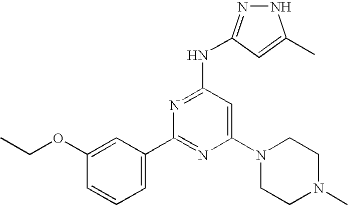 |
| 191 | 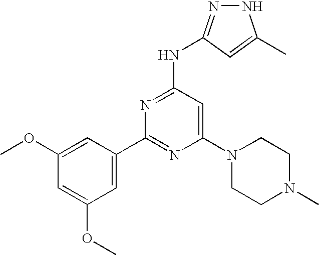 |
| 192 | 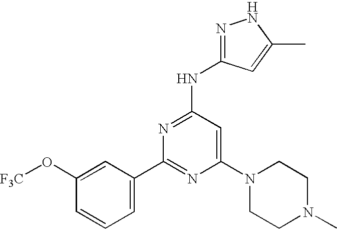 |
| 193 | 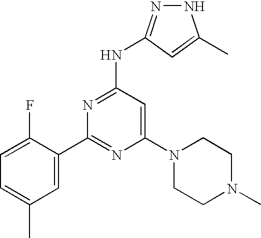 |
| 194 | 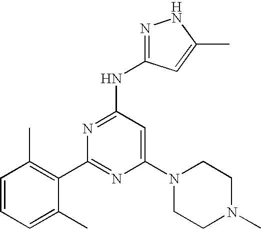 |
| 195 | 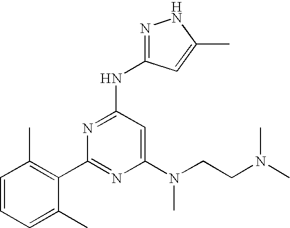 |
| 196 | 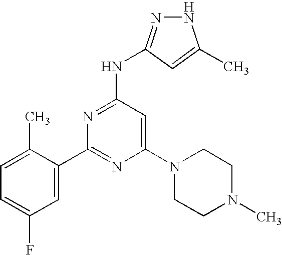 |
| 197 | 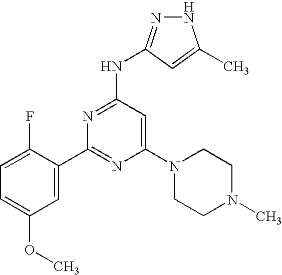 |
| 198 | 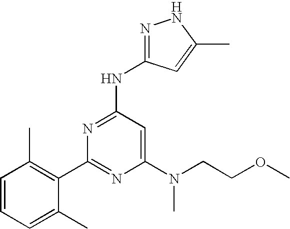 |
| 199 | 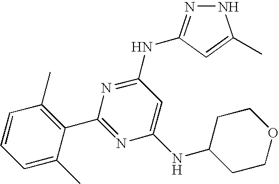 |
| 200 | 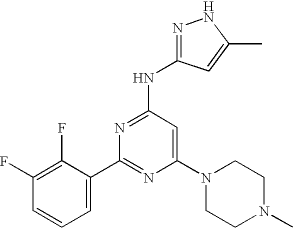 |
| 201 | 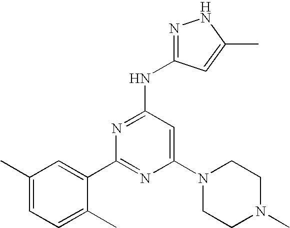 |
| 202 | 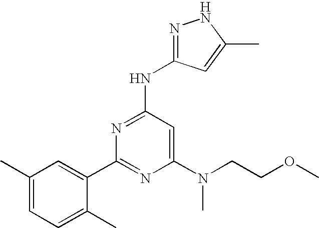 |
| 203 | 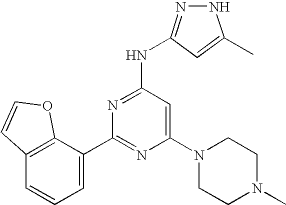 |
| 204 | 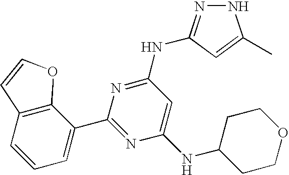 |
| 205 | 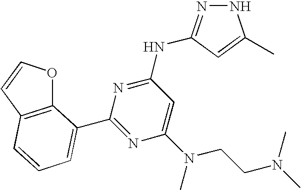 |
| 206 | 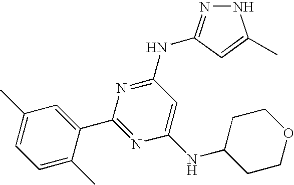 |
| 207 | 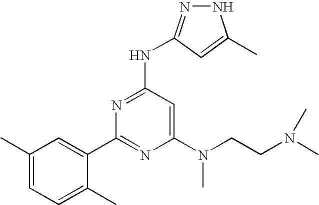 |
| 208 | 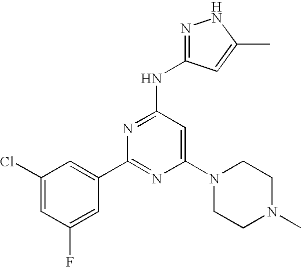 |

and biologically acceptable salts or prodrug thereof.

16. A pharmaceutical composition comprising an Aurora kinase A inhibition effective amount of the compound of claim 1 in combination with a pharmaceutically acceptable carrier, adjuvant or vehicle.

17. The composition of claim 16, wherein the composition comprises particles that are less than about 2 microns average particle size.

18. The composition of claim 16, wherein the composition is incorporated into a biodegradable or non-biodegradable polymer.

19. The composition of claim 16, comprising a compound selected from claim 1 and an additive.

20. The composition of claim 19, wherein the additive is selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

21. The composition of claim 16, wherein the carrier is suitable for oral, parenteral, inhalation, topical, or intradermal administration.

22. A method of treating a patient with a disease comprising administering to the patient with the disease an effective amount of a compound selected from the group of compounds of claim 1, wherein the disease is an autoimmune disease, inflammatory disease, neurological or neurodegenerative disease, cancer, cardiovascular disease, allergy, asthma, or a hormone-related disease.

23. A method of treating a patient with a cancer comprising administering to the patient having the cancer an effective cancer-treating amount of a compound selected from the group of compounds of claim 1.

24. The method of claim 23, wherein the cancer is a solid tumor, blood borne tumor, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity, pharynx, lip, tongue, mouth, pharynx, small intestine, colon-rectum, large intestine, rectum, brain and central nervous system, or leukemia.

25. A method of treating a patient with a disease associated with undesirable neovascularization comprising administering to the patient with the undersirable neovascularization an effective amount of a composition selected from the group of compounds of claim 1.

26. The method of claim 25, wherein the disease associated with undesirable neovasculariation comprises ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, or corneal graph rejection, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, or post-laser complications.

27. A method of treating a patient with an inflammatory disease associated with inflammation comprising administering to the patient with the inflammatory disease an effective amount of a compound selected from the group of compounds of claim 1.

28. The method of claim 27, wherein the inflammatory disease is excessive or abnormal stimulation of endothelial cells, atherosclerosis, vascular malfunctions, abnormal wound healing, inflammatory and immune disorders, Bechet's disease, gout or gouty arthritis, abnormal angiogenesis accompanying rheumatoid arthritis, skin diseases, psoriasis, diabetic retinopathy, retinopathy of prematurity, retrolental fibroplasia, macular degeneration, corneal graft rejection, neovascular glaucoma or Osler Weber syndrome.

29. A method of treating patient with a GSK-3 mediated disease comprising administering to the patient with the GSK-3 mediated disease an effective amount of a compound selected from the group of compounds of claim 1.

30. The method of claim 29, wherein the GSK-3 mediated disease is diabetes, Alzheimer's disease, Huntington's Disease, Parkinson's Disease, AIDS-associated dementia, amyotrophic lateral sclerosis (AML), multiple sclerosis (MS), schizophrenia, cardiomycete hypertrophy, reperfusion/ischemia, or baldness.

31. The method of claim 22, wherein the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.
